(19) United States
(12) Patent Application Publication
(10) Pub. No.: US 2010/0168080 A1
(43) Pub. Date: Jul. 1, 2010

(54) NOVEL COMPOUNDS USEFUL AS CC CHEMOKINE RECEPTOR LIGANDS

(76) Inventors: Uttam KHAMRAI, West Bengal (IN); Sumit Kumar Karak, West Bengal (IN); Matthew Ronsheim, Port Jefferson, NY (US); Ashis Kumar Saha, Stow, MA (US)

Correspondence Address: Forest Laboratories, Inc.;Attn: Charles S. Ryan, 48 Mall Drive, Commack, NY, 11725, US

(21) Appl. No.: 12/639,297

(22) Filed: Dec. 16, 2009

Related U.S. Application Data
(60) Provisional application No. 61/138,179, filed on Dec. 17, 2008.

(51) Int. Cl. C07D 265/30; A61K 31/553; C07D 211/06; A61K 31/5375; C07D 491/00; A61K 31/437; C07D 267/10; C07D 413/06; C07D 498/02; A61K 31/445; A61P 3/06; A61P 29/00

(52) U.S. Cl. 514/211.15; 514/211.01; 514/235.5; 514/237.5; 514/278; 514/327; 540/544; 544/130; 544/165; 546/19; 546/217

(57) ABSTRACT

The present invention relates to novel morpholine, oxazapane and piperidine derivatives that act as ligands for CC chemokine receptors, such as CCR1. The invention also relates to methods of preparing the compounds, compositions containing the compounds, and to methods of treatment using the compounds.

### FIELD OF THE INVENTION

The present invention relates to novel morpholine, oxazapane and piperidine derivatives that act as ligands for CC chemokine receptors, such as CCR1. The invention also relates to methods of preparing the compounds, compositions containing the compounds, and to methods of treatment using the compounds.

### BACKGROUND OF THE INVENTION

The selective accumulation and activation of leukocytes in inflamed tissues contributes to the pathogenesis of inflammatory and autoimmune diseases. Chemokines and their receptors, which belong to a family of seven transmembrane G− protein coupled receptors are involved in the selective accumulation and activation of leukocytes in inflamed tissues, and in the pathogenesis of inflammatory and autoimmune diseases. One such receptor is CCR1 which is a receptor for CC chemokines such as RANTES (regulated on activation normal Tcell expressed), MIP-1α (macrophage inflammatory protein) MPIF-1/CKβ8 and Leukotactin chemokine, among others.

The receptor CCR1 and its chemokine ligands represent significant therapeutic targets (see, e.g., Saeki, et al., Current Pharmaceutical Design, 9, 1201-1208, 2003) since they have been implicated in, for example, rheumatoid arthritis, transplant rejection (see, e.g., DeVries, et al., Semin. Immunol., 11(2), 95-104, 1999), and multiple sclerosis (see, e.g., Fischer, et al., J. Neuroimmunol., 110 (1-2), 195-208, 2000, Izikson, et al., J. Exp. Med., 192(7), 1075-1080, 2000, and Rottman, et al., Eur. J. Immunol., 30(8), 2372-2377, 2000). In vivo studies on mice indicate that CCR1-mediated leukocyte recruitment is important for interstitial inflammation in the kidney and that CCR1 blockade late in renal disease can halt disease progression and improve renal function (see, e.g., NAME, J. Am. Soc. Nephrol., 15, 1504-1513, 2004). Further, an animal model of neutrophil recruitment in response to MIP-1α demonstrates the positive biological and pharmacodynamic activity of CCR1 antagonists (see, e.g., U.S. 2005/0288319).

There is therefore an ongoing need to develop new compounds that can be used in the treatment of diseases mediated by CCR1 signaling.

### SUMMARY OF THE INVENTION

The present invention relates to novel morpholine, oxazapane and piperidine derivatives that act as ligands for CC chemokine receptors, such as CCR1. The invention also relates to methods of preparing the compounds, compositions containing the compounds, and to methods of treatment using the compounds.

### DETAILED DESCRIPTION OF THE INVENTION

In one aspect, the present invention includes compounds having the chemical formula:

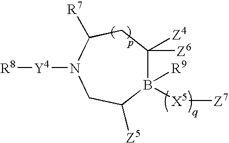

wherein

R⁸ is aryl-X⁴—, heteroaryl-X⁴—, aryl, heterocycle or heteroaryl;

X⁴ is —O—, —S—, —S(O)—, —S(O)₂—, —S(O)₂NR^{o}— or NR^{p}, where R^{o} and R^{p} are each, independently, hydrogen or alkyl;

Y⁴ is —C(O)—, —(CH₂)₂—, —(CH₂)₃—, —CH₂C(O)—, —(CH₂)₂C(O)—, —C(O)CH₂—,

—C(O)(CH₂)₂— or —CH₂CH(OH)CH₂—;

R⁷ is hydrogen or alkyl;

B is —C— or —O—;

R⁹ is hydrogen, hydroxyl or cyano;

X⁵ is —O—, —NR^{e}—, —S—, —S(O)— or —S(O)₂ where R^{e} is hydrogen or alkyl,

Z⁷ is aryl, heteroaryl, arylalkyl, heteroarylalkyl;

p is 0 or 1;

q is 0 or 1;

Z⁴, Z⁵ and Z⁶ are each, independently, hydrogen, alkyl, -arylalkyl, heteroarylalkyl, -(alkylene)-J-aryl or -(alkylene)-J-heteroaryl, where J is —O— or NR^{q}— and R^{q} is hydrogen or alkyl;

provided, however, that when R⁹ is hydroxyl or cyano, then q is 0, B is —C—, and Z⁴, Z⁶ are both alkyl or both hydrogen;

provided, however, that when R⁹ is H, then q is 1, p is 0, and B is —C—;

provided, however, that when B is —O—, and both R⁹ and —X⁵—Z⁷ are absent; then Z⁵ is not hydrogen when p is 0;

provided, however, that when B is —O—, R⁹ and —X⁵—Z⁷ are absent and p is 1; then either Z⁴, Z⁶ or Z⁵ are both not hydrogen;

provided, however, that when B is —C—, Z⁴, Z⁶ are both hydrogen, and p is 0, then R⁹ is cyano, q is 0, Z⁷ is arylalkyl, and Y⁴ is —(CH₂)₃—;

wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{r}C(O)R^{s}, —NR^{t}SO₂R^{u}, where R^{pr} and R^{t} are each, independently, hydrogen or alkyl, R^{s} is amino, aminoalkyl, alkyl or cycloalkyl, and R^{u} is hydrogen or alkyl; and combinations thereof;

and pharmaceutically acceptable salts or solvates or N-oxides thereof, or solvates of pharmaceutically acceptable salts thereof, or solvates of N-oxides thereof, or isomers, or prodrugs thereof;

with the proviso that said compound is not
1-[2-(4-fluorophenoxy)ethyl]-N-(4-fluorophenyl)-4-piperidinamine,
N-(4-chlorophenyl)-1-[2-(4-fluorophenoxy)ethyl]-4-piperidinamine,
N-(4-chlorophenyl)-1-[3-(4-fluorophenoxy)propyl]-4-piperidinamine,
N-(4-chlorophenyl)-1-(3-phenoxypropyl-4-piperidinamine,
N-(4-fluoro-2-methylphenyl)-1-[3-(4-fluorophenoxy)propyl]-4-piperidinamine,
1-[3-(4-fluorophenoxy)propyl]-N-(4-fluorophenyl)-N-methyl-4-piperidinamine,
1-[3-(4-fluorophenoxy)propyl]-N-(4-fluorophenyl)-4-piperidinamine,
N-(5-Chloro-2-{3-[4-(4-chloro-phenoxy)-piperidin-1-yl]-propoxy}-4-methoxy-phenyl)-acetamide,
N-(5-Chloro-2-{3-[4-(4-chloro-phenylsulfanyl)-piperidin-1-yl]-propoxy}-4-methoxy-phenyl)-acetamide,
N-(5-Chloro-2-{3-[4-(4-chloro-benzenesulfonyl)-piperidin-1-yl]-propoxy}-4-methoxy-phenyl)-acetamide,
N-(5-Chloro-2-{3-[4-(4-chloro-phenoxy)-3,3-dimethyl-piperidin-1-yl]-propoxy}-4-methoxy-phenyl)-acetamide,
N-(5-Chloro-2-{3-[4-(4-chloro-phenoxy)-piperidin-1-yl]-2-hydroxy-propoxy}-4-methoxy-phenyl)-acetamide,
N-(5-Chloro-2-{3-[4-(4-cyano-phenoxy)-piperidin-1-yl]-propoxy}-4-methoxy-phenyl)-acetamide,
N-(5-Chloro-2-{3-[4-(4-fluoro-phenoxy)-piperidin-1-yl]-propoxy}-4-methoxy-phenyl)-acetamide,
   or a pharmaceutically acceptable salt thereof.

In another aspect, the present invention includes compounds of formula I:

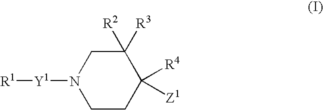

wherein

R¹ is aryl-X¹—, heteroaryl-X¹—, aryl or heteroaryl;

X¹ is —O—, —S—, —S(O)—, —S(O)₂— or —NR^{a}, where R^{a} is hydrogen or alkyl;

Y¹ is —(CH₂)₂— or —(CH₂)₃—;

R² and R³ are both alkyl or both hydrogen;

R⁴ is hydroxyl or cyano; and

Z¹ is aryl, heteroaryl, arylalkyl or heteroarylalkyl;

wherein, when present, any aryl or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{b}C(O)R^{c}, —NR^{b}SO₂R^{d}, where R^{b} is hydrogen or alkyl, R^{c} is amino, aminoalkyl, alkyl or cycloalkyl, and R^{d} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof,

with the proviso that when R² and R³ are both hydrogen, then Y¹ is —(CH₂)₃—, R⁴ is cyano and Z¹ is arylalkyl.

In certain embodiments, R¹ is aryl-X¹— or heteroaryl. For example, R¹ is aryl-O— or heteroaryl. In certain embodiments, R¹ is aryl-X¹, wherein the arylgroup is optionally substituted by halogen (e.g., Cl), carboxyl, alkoxy (e.g., methoxy), amido, alkylamido (e.g., —C(O)NHMe), dialkylamido (e.g., —C(O)NMe₂), NR^{b}C(O)R^{c} (e.g., —NHC(O)NH₂; —NHC(O)alkyl, such as —NHC(O)CH₃, —NHC(O)iPr, —NHC(O)t-Bu; —NHC(O)cycloalkyl, such as —NHC(O)cyclobutyl; —NHC(O)aminoalkyl, such as —NHC(O)CH₂NMe₂), —NR^{b}SO₂R^{d} (e.g., —NHSO₂alkyl, such as —NHSO₂Me), heteroaryl (e.g., isoxazolyl). In certain embodiments, R¹ is aryl-X¹ wherein the aryl group is optionally substituted by halogen (e.g., CO, NR^{b}C(O)R^{c} (e.g., —NHC(O)NH₂; —NHC(O)alkyl, such as —NHC(O)CH₃, —NHC(O)iPr, —NHC(O)t-Bu; —NHC(O)cycloalkyl, such as —NHC(O)cyclobutyl; —NHC(O)aminoalkyl, such as —NHC(O)CH₂NMe₂). In certain embodiments, R¹ is aryl-O—.

In a further embodiment, Y¹ is —(CH₂)₂. In a further embodiment, Y¹ is —(CH₂)₃.

In additional embodiments, R² and R³ are both alkyl. For example, R² and R³ are both methyl.

In additional embodiments R² and R³ are both alkyl (e.g., methyl) and R⁴ is hydroxyl.

In additional embodiments R² and R³ are both hydrogen and R⁴ is cyano.

In further embodiments, Z¹ is aryl or arylalkyl. For example Z¹ is aryl (e.g., optionally substituted phenyl, such as halophenyl (e.g., 4-halophenyl, such as 4-Cl-phenyl, 4-F-phenyl). For further example Z¹ is arylalkyl (e.g., optionally substituted benzyl). For example, Z¹ is halobenzyl (e.g., 4-halobenzyl such as 4-F-benzyl, 4-Cl-benzyl), dihalobenzyl (e.g., 3,4-dihalobenzyl such as 3,4,-difluorobenzyl), or halo(alkoxy)benzyl, such as 3-methoxy-4-chlorobenzyl).

In certain embodiments, the present invention includes compounds of formula I wherein

R¹ is aryl-X¹— or heteroaryl;

X¹ is —O—;

Y¹ is —(CH₂)₂— or —(CH₂)₃—;

R² and R³ are both alkyl or both hydrogen;

R⁴ is hydroxyl or cyano; and

Z¹ is aryl or arylalkyl;

wherein, when present, any aryl or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{b}C(O)R^{c}, —NR^{b}SO₂R^{d}, where R^{b} is hydrogen or alkyl, R^{c} is amino, aminoalkyl, alkyl or cycloalkyl, and R^{d} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof,

with the proviso that when R² and R³ are both hydrogen, then Y¹ is —(CH₂)₃—, R⁴ is cyano and Z¹ is arylalkyl.

In certain embodiments, the present invention includes compounds of formula I wherein

R¹ is aryl-X¹—, or heteroaryl;

X¹ is —O—;

Y¹ is —(CH₂)₂— or —(CH₂)₃—;

R² and R³ are both alkyl or both hydrogen;

R⁴ is hydroxyl or cyano; and

Z¹ is aryl or arylalkyl;

wherein, when present, any aryl or heteroaryl group may optionally be substituted by halogen, amido, alkylamido, dialkylamido, carboxyl, heteroaryl, alkoxy, —NR^{b}C(O)R^{c}, —NR^{b}SO₂R^{d}, where R^{b} is hydrogen or alkyl, R^{c} is amino, aminoalkyl, alkyl or cycloalkyl, and R^{d} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In one aspect, the present invention includes compounds of formula I:

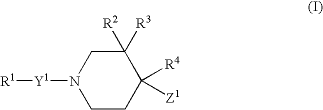

wherein

R¹ is aryl-X¹—, heteroaryl-X—, aryl or heteroaryl;

X¹ is —O—, —S—, —S(O)—, —S(O)₂— or —NR^{a}, where R^{a} is hydrogen or alkyl;

Y¹ is —(CH₂)₂— or —(CH₂)₃—;

R² and R³ are alkyl;

R⁴ is hydroxyl; and

Z¹ is aryl, heteroaryl, arylalkyl or heteroarylalkyl;

wherein, when present, any aryl or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{b}C(O)R^{c}, —NR^{b}SO₂R^{d}, where R^{b} is hydrogen or alkyl, R^{c} is amino, aminoalkyl, alkyl or cycloalkyl, and R^{d} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In additional embodiments, the compound of formula I is represented by subformulas Ia or Ib:

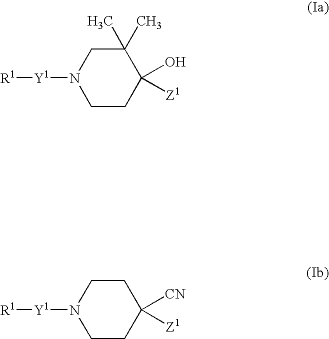

For example, in subformula Ia, R¹ is aryl-X¹—, heteroaryl-X—, aryl or heteroaryl; X¹ is —O—, —S—, —S(O)—, —S(O)₂— or —NR^{a}, where R^{a} is hydrogen or alkyl; Y¹ is —(CH₂)₂— or —(CH₂)₃— and Z¹ is aryl, heteroaryl, arylalkyl or heteroarylalkyl. For example, in subformula Ia, R¹ is aryl-X¹— or heteroaryl; X¹ is —O—, Y¹ is —(CH₂)₂— or —(CH₂)₃— and Z¹ is aryl or arylalkyl.

For example, in subformula Ib, R¹ is aryl-X¹—, heteroaryl-X—, aryl or heteroaryl; X¹ is —O—, —S—, —S(O)—, —S(O)₂— or —NR^{a}, where R^{a} is hydrogen or alkyl; Y¹ is —(CH₂)₂— or —(CH₂)₃— and Z¹ is aryl, heteroaryl, arylalkyl or heteroarylalkyl. For example, in subformula Ib, R¹ is aryl-X¹— or heteroaryl; X¹ is —O—, Y¹ is —(CH₂)₃— and Z¹ is arylalkyl.

In certain embodiments, the compound of formula I is selected from:
(5-Chloro-2-{3-[4-(4-fluoro-benzyl)-4-hydroxy-3,3-dimethyl-piperidin-1-yl]-propoxy}-phenyl)-urea,
(5-Chloro-2-{3-[4-(4-chloro-phenyl)-4-hydroxy-3,3-dimethyl-piperidin-1-yl]-propoxy}-phenyl)-urea,
(5-Chloro-2-{3-[4-(4-fluoro-phenyl)-4-hydroxy-3,3-dimethyl-piperidin-1-yl]-propoxy}-phenyl)-urea,
(5-Chloro-2-{3-[4-(4-chloro-benzyl)-4-hydroxy-3,3-dimethyl-piperidin-1-yl]-propoxy}-phenyl)-urea,
(5-Chloro-2-{3-[4-(4-fluoro-benzyl)-4-cyano-3,3-dimethyl-piperidin-1-yl]-propoxy}-phenyl)-urea,
1-[3-(4-Chloro-2-isoxazol-5-yl-phenoxy)-propyl]-4-(4-fluoro-benzyl)-3,3-dimethyl-piperidin-4-ol,
4-(4-Chloro-benzyl)-1-[3-(4-chloro-2-isoxazol-5-yl-phenoxy)-propyl]-3,3-dimethyl-piperidin-4-ol,
1-[3-(5-Chloro-benzoimidazol-1-yl)-propyl]-4-(4-chloro-benzyl)-3,3-dimethyl-piperidin-4-ol,
1-[2-(5-Chloro-benzoimidazol-1-yl)-ethyl]-4-(4-chloro-benzyl)-3,3-dimethyl-piperidin-4-ol,
N-(5-Chloro-2-{3-[4-(4-chloro-benzyl)-4-hydroxy-3,3-dimethyl-piperidin-1-yl]-propoxy}-phenyl)-acetamide,
N-(5-Chloro-2-{3-[4-(4-chloro-benzyl)-4-hydroxy-3,3-dimethyl-piperidin-1-yl]-propoxy}-phenyl)-2-dimethylamino-acetamide,
N-(5-Chloro-2-{3-[4-(4-chloro-benzyl)-4-hydroxy-3,3-dimethyl-piperidin-1-yl]-propoxy}-phenyl)-isobutyramide,
N-(5-Chloro-2-{3-[4-(4-chloro-benzyl)-4-hydroxy-3,3-dimethyl-piperidin-1-yl]-propoxy}-phenyl)-2,2-dimethyl-propionamide,
Cyclobutanecarboxylic acid (5-chloro-2-{3-[4-(4-chloro-benzyl)-4-hydroxy-3,3-dimethyl-piperidin-1-yl]-propoxy}-phenyl)-amide,
N-(5-Chloro-2-{3-[4-(4-chloro-benzyl)-4-hydroxy-3,3-dimethyl-piperidin-1-yl]-propoxy}-phenyl)-methanesulfonamide,
5-Chloro-2-{3-[4-(4-chloro-benzyl)-4-hydroxy-3,3-dimethyl-piperidin-1-yl]-propoxy}-benzoic acid,
5-Chloro-2-{3-[4-(4-chloro-benzyl)-4-hydroxy-3,3-dimethyl-piperidin-1-yl]-propoxy}-benzamide,
1-[3-(5-Chloro-benzoimidazol-1-yl)-propyl]-4-(4-chloro-phenyl)-3,3-dimethyl-piperidin-4-ol,
1-[3-(5-Chloro-benzoimidazol-1-yl)-propyl]-4-(4-fluoro-benzyl)-piperidine-4-carbonitrile,
N-(5-Chloro-2-{3-[4-(3,4-difluoro-benzyl)-4-hydroxy-3,3-dimethyl-piperidin-1-yl]-propoxy}-phenyl)-acetamide,
N-(5-Chloro-2-{3-[4-(4-chloro-benzyl)-4-hydroxy-3,3-dimethyl-piperidin-1-yl]-propoxy}-4-methoxy-phenyl)-acetamide,
N-(5-Chloro-2-{3-[4-(4-chloro-3-methoxy-benzyl)-4-hydroxy-3,3-dimethyl-piperidin-1-yl]-propoxy}-phenyl)-acetamide,
5-Chloro-2-{3-[4-(4-chloro-benzyl)-4-hydroxy-3,3-dimethyl-piperidin-1-yl]-propoxy}-N,N-dimethyl-benzamide,
5-Chloro-2-{3-[4-(4-chloro-benzyl)-4-hydroxy-3,3-dimethyl-piperidin-1-yl]-propoxy}-N-methyl-benzamide,
1-[2-(6-Chloro-1H-benzoimidazol-2-yl)-ethyl]-4-(4-chloro-benzyl)-3,3-dimethyl-piperidin-4-ol,
N-(5-Chloro-2-{3-[4-(4-chloro-phenyl)-4-hydroxy-3,3-dimethyl-piperidin-1-yl]-propoxy}-phenyl)-acetamide,
N-(5-Chloro-2-{3-[4-(4-chloro-phenyl)-4-hydroxy-3,3-dimethyl-piperidin-1-yl]-propoxy}-4-methoxy-phenyl)-acetamide,
4-(4-Chloro-benzyl)-1-[3-(4-chloro-2-pyrazol-1-yl-phenoxy)-propyl]-3,3-dimethyl-piperidin-4-ol,
(5-Chloro-2-{3-[4-(4-chloro-benzyl)-4-hydroxy-3,3-dimethyl-piperidin-1-yl]-propoxy}-4-methoxy-phenyl)-urea,
(5-Chloro-2-{3-[4-(4-fluoro-benzyl)-4-hydroxy-3,3-dimethyl-piperidin-1-yl]-propoxy}-4-methoxy-phenyl)-urea,
N-(5-Chloro-2-{3-[4-(4-fluoro-benzyl)-4-hydroxy-3,3-dimethyl-piperidin-1-yl]-propoxy}-4-methoxy-phenyl)-acetamide,
N-(5-Chloro-2-{3-[4-(4-chloro-benzyl)-4-hydroxy-3,3-dimethyl-piperidin-1-yl]-propoxy}-4-isopropoxy-phenyl)-acetamide,
N-(5-Chloro-2-{3-[4-(4-fluoro-benzyl)-4-hydroxy-3,3-dimethyl-piperidin-1-yl]-propoxy}-4-isopropoxy-phenyl)-acetamide,
N-(2-{3-[4-(4-Chloro-benzyl)-4-hydroxy-3,3-dimethyl-piperidin-1-yl]-propoxy}-4-methoxy-phenyl)-acetamide,
N-(5-Chloro-2-{3-[4-(4-chloro-phenyl)-4-hydroxy-3,3-dimethyl-piperidin-1-yl]-propoxy}-4-isopropoxy-phenyl)-acetamide,
5-Chloro-2-{3-[4-(4-fluoro-benzyl)-4-hydroxy-3,3-dimethyl-piperidin-1-yl]-2-hydroxy-propoxy}-4-methoxy-benzoic acid,
5-Chloro-2-{3-[4-(4-fluoro-benzyl)-4-hydroxy-3,3-dimethyl-piperidin-1-yl]-2-hydroxy-propoxy}-4-methoxy-N-methyl-benzamide,
5-Chloro-2-{3-[4-(4-fluoro-benzyl)-4-hydroxy-3,3-dimethyl-piperidin-1-yl]-propoxy}-4-methoxy-N-methyl-benzamide,
5-Chloro-2-{3-[4-(4-chloro-phenyl)-4-hydroxy-3,3-dimethyl-piperidin-1-yl]-propoxy}-4-methoxy-benzoic acid methyl ester,
(5-Chloro-2-{3-[4-(4-fluoro-benzyl)-4-hydroxy-3,3-dimethyl-piperidin-1-yl]-propoxy}-4-methoxy-phenyl)-pyrrolidin-1-yl-methanone,
5-Chloro-2-{3-[4-(4-chloro-phenyl)-4-hydroxy-3,3-dimethyl-piperidin-1-yl]-propoxy}-4-methoxy-N-methyl-benzamide,
(5-Chloro-2-{3-[4-(4-chloro-phenyl)-4-hydroxy-3,3-dimethyl-piperidin-1-yl]-propoxy}-4-methoxy-phenyl)-pyrrolidin-1-yl-methanone,
5-Chloro-2-{3-[4-(4-fluoro-benzyl)-4-hydroxy-3,3-dimethyl-piperidin-1-yl]-propoxy}-4-methoxy-benzoic acid,
5-Chloro-2-{3-[4-(4-fluoro-benzyl)-4-hydroxy-3,3-dimethyl-piperidin-1-yl]-propoxy}-4-hydroxy-N-methyl-benzamide,
5-Chloro-2-{3-[4-(4-chloro-phenyl)-4-hydroxy-3,3-dimethyl-piperidin-1-yl]-propoxy}-4-methoxy-N,N-dimethyl-benzamide,
5-Chloro-4-dimethylamino-2-{3-[4-(4-fluoro-benzyl)-4-hydroxy-3,3-dimethyl-piperidin-1-yl]-propoxy}-N-methyl-benzamide,
5-Chloro-2-{3-[4-(4-fluoro-benzyl)-4-hydroxy-3,3-dimethyl-piperidin-1-yl]-propoxy}-N-methyl-4-methylamino-benzamide,
5-Chloro-2-{3-[3-(4-chloro-phenyl)-3-hydroxy-pyrrolidin-1-yl]-propoxy}-4-methoxy-N-methyl-benzamide,
5-Chloro-2-{3-[3-(4-chloro-benzyl)-3-hydroxy-pyrrolidin-1-yl]-propoxy}-4-methoxy-N-methyl-benzamide,
N-[5-Chloro-2-(2-{[1-(4-fluoro-benzyl)-4-hydroxy-piperidin-4-ylmethyl]-amino}-ethoxy)-4-methoxy-phenyl]-acetamide,
5-Chloro-2-(2-{[1-(4-fluoro-benzyl)-4-hydroxy-piperidin-4-ylmethyl]-amino}-ethoxy)-4-methoxy-N-methyl-benzamide,
N-(5-Chloro-2-{3-[3-(4-chloro-phenyl)-3-hydroxy-pyrrolidin-1-yl]-propoxy}-4-methoxy-phenyl)-acetamide,
N-(5-Chloro-2-{3-[3-(4-chloro-benzyl)-3-hydroxy-pyrrolidin-1-yl]-propoxy}-4-methoxy-phenyl)-acetamide,
N-[5-Chloro-2-(2-{[1-(4-chloro-benzyl)-4-hydroxy-piperidin-4-ylmethyl]-amino}-ethoxy)-4-methoxy-phenyl]-acetamide,
5-Chloro-2-{3-[3-(4-fluoro-benzyl)-3-hydroxy-pyrrolidin-1-yl]-propoxy}-4-methoxy-N-methyl-benzamide,
N-(5-Chloro-2-{3-[3-(4-fluoro-benzyl)-3-hydroxy-pyrrolidin-1-yl]-propoxy}-4-methoxy-phenyl)-acetamide,
5-Chloro-2-(2-{[1-(4-chloro-benzyl)-4-hydroxy-piperidin-4-ylmethyl]-amino}-ethoxy)-4-methoxy-N-methyl-benzamide,
5-Chloro-2-{2-[8-(4-fluoro-benzyl)-2-oxo-1-oxa-3,8-diaza-spiro[4.5]dec-3-yl]-ethoxy}-4-methoxy-N-methyl-benzamide,
5-Chloro-2-[4-(4-fluoro-benzyl)-[1,4]oxazepan-2-ylmethoxy]-4-methoxy-N-methyl-benzamide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In another aspect, the present invention includes compounds of formula II:

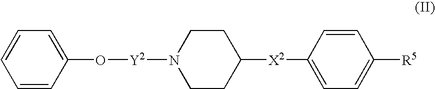

wherein

Y² is —(CH₂)₂—, —CH₂CH(OH)CH₂— or —(CH₂)₃—;

X² is —O—, —NR^{e}, —S—, —S(O)— or —S(O)₂ where R^{e} is hydrogen or alkyl; and

R⁵ is halogen (e.g., Cl);

wherein, any unsubstituted position in a phenyl ring depicted in Formula II may independently optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{f}C(O)R^{g}, —NR^{h}SO₂Rⁱ, where R^{f} and R^{h} are each, independently, hydrogen or alkyl, R^{g} is amino, aminoalkyl, alkyl or cycloalkyl, and Rⁱ is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof;

with the proviso that said compound is not
1-[2-(4-fluorophenoxy)ethyl]-N-(4-fluorophenyl)-4-piperidinamine,
N-(4-chlorophenyl)-1-[2-(4-fluorophenoxy)ethyl]-4-piperidinamine,
N-(4-chlorophenyl)-1-[3-(4-fluorophenoxy)propyl]-4-piperidinamine,
N-(4-chlorophenyl)-1-(3-phenoxypropyl-4-piperidinamine,
N-(4-fluoro-2-methylphenyl)-1-[3-(4-fluorophenoxy)propyl]-4-piperidinamine,
1-[3-(4-fluorophenoxy)propyl]-N-(4-fluorophenyl)-N-methyl-4-piperidinamine,
1-[3-(4-fluorophenoxy)propyl]-N-(4-fluorophenyl)-4-piperidinamine or a pharmaceutically acceptable salt thereof.

In certain embodiments, the present invention relates to compounds of formula II, wherein

Y² is —(CH₂)₂—, —CH₂CH(OH)CH₂— or —(CH₂)₃—;

X² is —O—, —S—, —S(O)— or —S(O)₂; and

R⁵ is halogen (e.g., Cl);

wherein, any unsubstituted position in a phenyl ring depicted in Formula II may independently optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfonyl, arylsulfonyl, heteroarylsulfinyl, heteroarylsulfonyl, alkoxycarbonyl, aryloxycarbonyl, heteroaryloxycarbonyl, —NR^{f}C(O)R^{g}, —NR^{h}SO₂Rⁱ, where R^{f} and R^{h} are each, independently, hydrogen or alkyl, R^{g} is amino, aminoalkyl, alkyl or cycloalkyl, and R′ is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, Y² is —(CH₂)₃— or —CH₂CH(OH)CH₂—. For example, Y² is —(CH₂)₃—

In additional embodiments, X² is —O—, —S—, or —S(O)₂.

In additional embodiments, R⁵ is Cl or F. For example, R⁵ is Cl.

In further embodiments, any unsubstituted position in a phenyl ring depicted in Formula II may independently optionally be substituted by halogen, alkoxy or —NR^{f}C(O)R^{g}, where R^{f} is hydrogen or alkyl and R^{g} is amino, aminoalkyl, alkyl or cycloalkyl. For example, any unsubstituted position in a phenyl ring depicted in Formula II may independently optionally be substituted by halogen, alkoxy or —NR^{f}C(O)R^{g}, where R^{f} is hydrogen and R^{g} is amino or alkyl.

In certain embodiments, the compound of formula II may be represented by formula IIa:

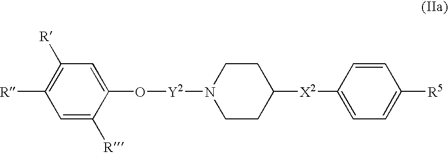

wherein R′, R″ and R′″ are each, independently, hydrogen, halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{f}C(O)R^{g}, —NR^{h}SO₂Rⁱ, where R^{f} and R^{h} are each, independently, hydrogen or alkyl, R^{g} is amino, aminoalkyl, alkyl or cycloalkyl, and Rⁱ is hydrogen or alkyl.

For example, R′, R″ and R′″ are each, independently, halogen, alkoxy or —NR^{f}C(O)R^{g}, where R^{f} is hydrogen or alkyl and R^{g} is amino, aminoalkyl, alkyl or cycloalkyl. For further example, R′, R″ and R′″ are each, independently, halogen, alkoxy or —NR^{f}C(O)R^{g}, where R^{f} is hydrogen and R^{g} is amino or alkyl. In one embodiment, R′ is alkoxy (e.g., methoxy), R″ is halogen (e.g., Cl) and R′″ is NR^{f}C(O)R^{g} (e.g., NHC(O)CH₃.)

In certain embodiments, the present invention includes compounds of formula II wherein

Y² is —CH₂CH(OH)CH₂— or —(CH₂)₃—;

X² is —O—, —S—, or —S(O)₂; and

R⁵ is halogen (e.g., Cl)

wherein, any unsubstituted position in a phenyl ring depicted in Formula II may independently optionally be substituted by halogen, alkoxy, or —NR^{f}C(O)R^{g}, where R^{f} is hydrogen or alkyl, R^{g} is amino or alkyl;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, the compound of formula II is selected from:
N-(5-Chloro-2-{2-[4-(4-fluoro-phenoxy)-piperidin-1-yl]-2-oxo-ethoxy}-4-methoxy-phenyl)-acetamide,
(5-Chloro-2-{2-[4-(4-fluoro-phenoxy)-piperidin-1-yl]-2-oxo-ethoxy}-phenyl)-urea,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In a further aspect, the present invention includes compounds of formula III:

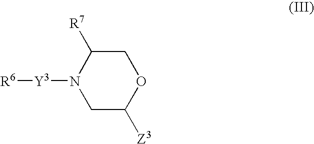

wherein

R⁶ is aryl-X³—, heteroaryl-X³—, aryl, heterocycle or heteroaryl;

X³ is —O—, —S—, —S(O)—, —S(O)₂—, —S(O)₂NR^{h}— or —NRⁱ, where R^{h} and Rⁱ are each, independently, hydrogen or alkyl;

Y³ is —(CH₂)₂—, —(CH₂)₃—, —CH₂C(O)—, —C(O)—, or —(CH₂)₂C(O)—;

R⁷ is hydrogen or alkyl; and

Z³ is arylalkyl, heteroarylalkyl, -(alkylene)-G-aryl or -(alkylene)-G-heteroaryl, where G is —O— or —NR^{j} and R^{j} is alkyl or hydrogen;

wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{k}C(O)R^{l}, —NR^{m}SO₂Rⁿ, where R^{k} and R^{m} are each, independently, hydrogen or alkyl, R¹ is amino, aminoalkyl, alkyl or cycloalkyl, and Rⁿ is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, R⁶ is aryl-X³— or heterocycle. For example, R⁶ is aryl-O— or heterocycle. In certain embodiments, R⁶ is aryl-X³, wherein the aryl group is optionally substituted by halogen (e.g., Br, Cl, F), carboxyl, alkoxy (e.g., methoxy), alkyl, amido, carboxyl, alkylamido (e.g., —C(O)NHMe), dialkylamido (e.g., —C(O)NMe₂), NR^{k}C(O)R^{l} (e.g., —NHC(O)NH₂; —NHC(O)alkyl, such as —NHC(O)CH₃, —NHC(O)iPr, —NHC(O)t-Bu; —NHC(O)cycloalkyl, such as —NHC(O)cyclobutyl; —NHC(O)aminoalkyl, such as —NHC(O)CH₂NMe₂), —NR^{m}SO₂Rⁿ (e.g., —NHSO₂alkyl, such as —NHSO₂Me), heteroaryl (e.g., isoxazolyl), and combinations thereof.

In certain embodiments, R⁶ is aryl-X³, wherein the aryl group is optionally substituted by halogen, hydroxy, aryl or —NR^{k}C(O)R^{l}, where is R^{k} is hydrogen or alkyl and R^{l} is amino, aminoalkyl, alkyl or cycloalkyl, and combinations thereof. For example, R⁶ is aryl-X³ optionally substituted by halogen, hydroxy, aryl or —NR^{k}C(O)R^{l}, where is R^{k} hydrogen R^{l} is amino.

In certain embodiments, X³ is —O—.

In additional embodiments, R⁶ is heterocycle. For example, R⁶ is optionally substituted piperidinyl. For example, R⁶ is piperidinyl optionally substituted by alkyl, hydroxyl, optionally substituted aryl, and combinations thereof. For further example, R⁶ is piperidinyl optionally substituted by alkyl, hydroxyl, halo-substituted aryl (e.g., chlorophenyl, such as 4-chlorophenyl) and combinations thereof.

In a further embodiment, Y³ is —(CH₂)₂—, —(CH₂)₃—, —CH₂C(O)— or —(CH₂)₂C(O)—.

In further embodiments, R⁷ is alkyl (e.g., methyl).

In certain embodiments, Z³ is arylalkyl, or -(alkylene)-G-aryl, where G is —O— or —NR^{j}— and R^{j} is alkyl or hydrogen. For example, Z³ is arylalkyl or -(alkylene)-G-aryl, where G is —O.

In certain embodiments, Z³ is arylalkyl (e.g., benzyl, phenethyl), optionally substituted by halogen (e.g., F, Cl). For example Z³ is 4-fluorobenzyl, 4-chlorobenzyl, 3,4-difluorobenzyl, 4-fluorophenethyl.

In yet further embodiments, Z³ is -(alkylene)-G-aryl where G is —O— or —NR^{j}— and R^{j} is alkyl or hydrogen. For example, Z³ is -(alkylene)-G-aryl where G is —O (e.g., —CH²—O-aryl, such as —CH²—O-p-fluorophenyl.)

In certain embodiments, the compound of Formula III is represented by subformula IIIa:

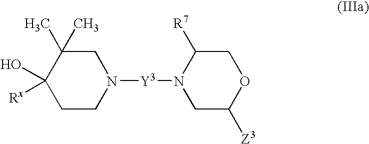

wherein R^{x} is optionally substituted aryl. For example, R^{x} is optionally substituted phenyl, such as halo-substituted phenyl (e.g., chlorophenyl, such as 4-chlorophenyl).

In on example of subformula Ma, Y³ is —CH₂—C(O)— or —(CH₂)₂—, R^{x} is halo-substituted phenyl (e.g., p-chlorophenyl), R⁷ is alkyl and Z³ is arylalkyl (e.g., benzyl, such as 4-fluorobenzyl).

In certain embodiments, the present invention includes compounds of formula III

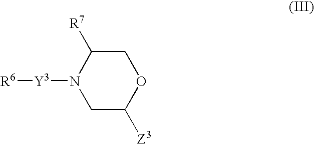

wherein

R⁶ is aryl-X³— or heterocycle;

X³ is —O—;

Y³ is —(CH₂)₂—, —(CH₂)₃—, —CH₂C(O)— or —(CH₂)₂C(O)—;

R⁷ is alkyl; and

Z³ is arylalkyl, or -(alkylene)-G-aryl;

wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{k}C(O)R^{l}, —NR^{m}SO₂Rⁿ, where R^{k} and R^{m} are each, independently, hydrogen or alkyl, R^{l} is amino, aminoalkyl, alkyl or cycloalkyl, and Rⁿ is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, the compound of formula III is selected from:
(5-Chloro-2-{2-[(2R,5S)-2-(4-fluoro-benzyl)-5-methyl-morpholin-4-yl]-2-oxo-ethoxy}-phenyl)-urea,
(5-Chloro-2-{2-[(2R,5R)-2-(4-fluoro-phenoxymethyl)-5-methyl-morpholin-4-yl]-2-oxo-ethoxy}-phenyl)-urea,
(5-Chloro-2-{2-[(2S,5R)-2-(4-fluoro-phenoxymethyl)-5-methyl-morpholin-4-yl]-2-oxo-ethoxy}-phenyl)-urea,
[5-Chloro-2-(2-{(2S,5R)-2-[2-(4-fluoro-phenyl)-ethyl]-5-methyl-morpholin-4-Y^{1}}-2-oxo-ethoxy)-phenyl]-urea,
5-Chloro-2-(2-{(2R,5R)-2-[2-(4-fluoro-phenyl)-ethyl]-5-methyl-morpholin-4-yl}-2-oxo-ethoxy)-phenyl]-urea,
(5-Chloro-2-{3-[(2R,5S)-2-(4-fluoro-benzyl)-5-methyl-morpholin-4-yl]-3-oxo-propoxy}-phenyl)-urea,
(5-Chloro-2-{3-[(2S,5S)-2-(4-fluoro-benzyl)-5-methyl-morpholin-4-yl]-3-oxo-propoxy}-phenyl)-urea,
(5-Chloro-2-{2-[(2R,5S)-2-(4-fluoro-benzyl)-5-methyl-morpholin-4-yl]-ethoxy}-phenyl)-urea,
1-[(2R,5S)-2-(4-Fluoro-benzyl)-5-methyl-morpholin-4-yl]-2-(4-fluoro-phenoxy)-ethanone,
2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
2-[(R)-4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
2-[(S)-4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
(S)-4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
(R)-4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
5-Chloro-2-{3-[(2R,5S)-2-(4-fluoro-benzyl)-5-methyl-morpholin-4-yl]-propoxy}-phenyl)-urea,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In additional embodiments, the compound of formula III is selected from:
(5-Chloro-2-{2-[2-(4-fluoro-benzyl)-5-methyl-morpholin-4-yl]-2-oxo-ethoxy}-phenyl)-urea,
(5-Chloro-2-{2-[2-(4-fluoro-phenoxymethyl)-5-methyl-morpholin-4-yl]-2-oxo-ethoxy}-phenyl)-urea,
[5-Chloro-2-(2-{2-[2-(4-fluoro-phenyl)-ethyl]-5-methyl-morpholin-4-yl}-2-oxo-ethoxy)-phenyl]-urea,
(5-Chloro-2-{3-[2-(4-fluoro-benzyl)-5-methyl-morpholin-4-yl]-3-oxo-propoxy}-phenyl)-urea,
(5-Chloro-2-{2-[2-(4-fluoro-benzyl)-5-methyl-morpholin-4-yl]-ethoxy}-phenyl)-urea,
1-[2-(4-Fluoro-benzyl)-5-methyl-morpholin-4-yl]-2-(4-fluoro-phenoxy)-ethanone,
2-[4-(4-Chloro-phenyl)-4-hydroxy-3,3-dimethyl-piperidin-1-yl]-1-[2-(4-fluoro-benzyl)-5-methyl-morpholin-4-yl]-ethanone,
[2-(4-fluoro-benzyl)-5-methyl-morpholin-4-yl]-propan-1-one-3-[4-(4-Chloro-phenyl)-4-hydroxy-3,3-dimethyl-piperidine],
5-Chloro-2-{3-[2-(4-fluoro-benzyl)-5-methyl-morpholin-4-yl]-propoxy}-phenyl)-urea,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In another aspect, the present invention includes compounds of formula IV:

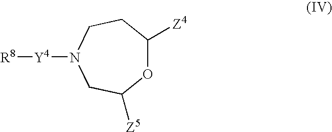

wherein

R⁸ is aryl-X⁴—, heteroaryl-X⁴—, aryl, heterocycle or heteroaryl;

X⁴ is —O—, —S—, —S(O)—, —S(O)₂—, —S(O)₂NR^{o}— or —NR^{p}, where R^{o} and R^{p} are each, independently, hydrogen or alkyl;

Y⁴ is —C(O)—, —(CH₂)₂—, —(CH₂)₃—, —CH₂C(O)—, —(CH₂)₂C(O)—, —C(O)CH₂—, C(O)—(CH₂)₂— or —CH₂CH(OH)CH₂—; and

Z⁴ are Z⁵ are each, independently, hydrogen, arylalkyl, heteroarylalkyl, -(alkylene)-J-aryl or -(alkylene)-J-heteroaryl, where J is —O— or —NR^{q}— and R^{q} is hydrogen or alkyl; with the proviso that at least one of Z⁴ or Z⁵ is other than hydrogen;

wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{r}C(O)R^{s}, —NR^{t}SO₂R^{u}, where R^{pr} and R^{t} are each, independently, hydrogen or alkyl, R^{s} is amino, aminoalkyl, alkyl or cycloalkyl, and R^{u} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, R⁸ is aryl-X⁴— or heterocycle. For example, R⁸ is aryl-O— or heterocycle. In certain embodiments, R⁸ is aryl-X⁴, wherein the aryl group is optionally substituted by halogen (e.g., Br, Cl, F), alkoxy (e.g., methoxy), alkyl, amido, carboxyl, alkylamido (e.g., —C(O)NHMe), dialkylamido (e.g., —C(O)NMe₂), NWC(O)R^{s} (e.g., —NHC(O)NH₂; —NHC(O)alkyl, such as —NHC(O)CH₃, —NHC(O)iPr, —NHC(O)t-Bu; —NHC(O)cycloalkyl, such as —NHC(O)cyclobutyl; —NHC(O)aminoalkyl, such as —NHC(O)CH₂NMe₂), —NR^{t}SO₂R^{u} (e.g., —NHSO₂alkyl, such as —NHSO₂Me), heteroaryl (e.g., isoxazolyl), and combinations thereof.

For example, R⁸ is aryl-X⁴, wherein the aryl group is optionally substituted by halogen (e.g., Br, Cl, F), alkoxy (e.g., methoxy), alkyl, amido, carboxyl or NR^{r}C(O)R^{s} (e.g., —NHC(O)NH₂) or heteroaryl (e.g., isoxazolyl).

In additional embodiments, X⁴ is —O— or —S(O)₂NR^{o}— where R^{o} is hydrogen or alkyl. For example, X⁴ is —O—, —SO₂NH— or —SO₂N(CH₃)—.

In additional embodiments, R⁸ is heterocycle. For example, R⁸ is optionally substituted piperidinyl. For example, R⁸ is piperidinyl optionally substituted by alkyl, hydroxyl, optionally substituted aryl, and combinations thereof. For further example, R⁸ is piperidinyl optionally substituted by alkyl, hydroxyl, halo-substituted aryl (e.g., chlorophenyl, such as 4-chlorophenyl) and combinations thereof.

In further embodiments, Y⁴ is —(CH₂)₂—, —(CH₂)₃—, —CH₂C(O)—, —(CH₂)₂C(O)—, —C(O)CH₂—, —C(O)—(CH₂)₂— or —CH₂CH(OH)CH₂—.

In yet further embodiments, Z⁴ are Z⁵ are each, independently, hydrogen or arylalkyl, with the proviso that at least one of Z⁴ or Z⁵ is other than hydrogen. In certain embodiments, one of Z⁴ and Z⁵ is hydrogen, and the other of Z⁴ and Z⁵ is arylalkyl. For example Z⁴ is hydrogen and Z⁵ is arylalkyl (e.g., benzyl, such as 4-fluorobenzyl). For further example, Z⁵ is hydrogen and Z⁴ is arylalkyl (e.g., benzyl, such as 4-fluorobenzyl).

In certain embodiments, the compound of Formula IV is represented by subformula IVa:

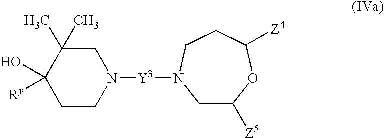

wherein R^{y} is optionally substituted aryl. For example, R^{y} is optionally substituted phenyl, such as halo-substituted phenyl (e.g., chlorophenyl, such as 4-chlorophenyl).

For example, in subformula IVa, Y³ is —(CH₂)₂C(O)— or —(CH₂)₂—, R^{y} is halo-substituted phenyl (e.g., 4-chlorophenyl), R⁷ is alkyl one of Z⁴ and Z5 is hydrogen, and the other of Z⁴ and Z⁵ is arylalkyl (e.g., benzyl, such as 4-fluorobenzyl).

In certain embodiments, the present invention includes compounds of formula IV:

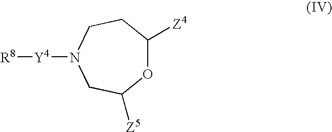

wherein

R⁸ is aryl-X⁴— or heterocycle;

X⁴ is —O—, or —S(O)₂NR^{m}— where R^{m} is hydrogen or alkyl;

Y⁴ is —(CH₂)₂—, —(CH₂)₃—, —CH₂C(O)—, —(CH₂)₂C(O)—, —C(O)CH₂—, —C(O)—(CH₂)₂— or —CH₂CH(OH)CH₂—; and

Z⁴ are Z⁵ are each, independently, hydrogen or arylalkyl, with the proviso that at least one of Z⁴ or Z⁵ is other than hydrogen;

wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{r}C(O)R^{s}, —NR^{t}SO₂R^{u}, where R^{r}, and R^{t} are each, independently, hydrogen or alkyl, R^{s} is amino, aminoalkyl, alkyl or cycloalkyl, and R^{u} is hydrogen or alkyl; and combinations thereof;

and pharmaceutically acceptable salts or solvates (e.g., hydrates) or N-oxides thereof, or solvates of pharmaceutically acceptable salts thereof, or pharmaceutically acceptable salts or solvates of N-oxides thereof; or prodrugs thereof.

In certain embodiments, the compound of formula IV is selected from:
(5-Chloro-2-{3-[2-(4-fluoro-benzyl)-[1,4]oxazepan-4-yl]-3-oxo-propoxy}-phenyl)-urea,
(5-Chloro-2-{2-[2-(4-fluoro-benzyl)-[1,4]oxazepan-4-yl]-2-oxo-ethoxy}-phenyl)-urea,
(5-Chloro-2-{3-[2-(4-fluoro-benzyl)-[1,4]oxazepan-4-yl]-propoxy}-phenyl)-urea,
3-[4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
3-[(S)-4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
3-[(R)-4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
4-(4-Chloro-phenyl)-1-{2-[(R)-2-(4-fluoro-benzyl)-[1,4]oxazepan-4-yl]-ethyl}-3,3-dimethyl-piperidin-4-ol,
(S)-4-(4-Chloro-phenyl)-1-{2-[(R)-2-(4-fluoro-benzyl)-[1,4]oxazepan-4-yl]-ethyl}-3,3-dimethyl-piperidin-4-ol,
(R)-4-(4-Chloro-phenyl)-1-{2-[(R)-2-(4-fluoro-benzyl)-[1,4]oxazepan-4-yl]-ethyl}-3,3-dimethyl-piperidin-4-ol,
(5-Chloro-2-{2-[7-(4-fluoro-benzyl)-[1,4]oxazepan-4-yl]-2-oxo-ethoxy}-phenyl)-urea,
(5-Chloro-2-{3-[7-(4-fluoro-benzyl)-[1,4]oxazepan-4-yl]-3-oxo-propoxy}-phenyl)-urea,
(5-Chloro-2-{3-[7-(4-fluoro-benzyl)-[1,4]oxazepan-4-yl]-propoxy}-phenyl)-urea,
(5-Chloro-2-{2-[7-(4-fluoro-benzyl)-[1,4]oxazepan-4-yl]-ethoxy}-phenyl)-urea,
4-[3-(4-Chloro-phenoxy)-propyl]-2-(4-fluoro-benzyl)-[1,4]oxazepane,
(5-Chloro-2-{2-[2-(4-fluoro-benzyl)-[1,4]oxazepan-4-yl]-ethoxy}-phenyl)-urea,
3-[2-(4-Fluoro-benzyl)-[1,4]oxazepan-4-yl]-N-(3-fluoro-phenyl)-propionamide,
N-(3-Chloro-phenyl)-3-[2-(4-fluoro-benzyl)-[1,4]oxazepan-4-yl]-propionamide,
N-(5-Chloro-2-methoxy-phenyl)-3-[2-(4-fluoro-benzyl)-[1,4]oxazepan-4-yl]-propionamide,
(5-Chloro-2-{3-[2-(3,4-difluoro-benzyl)-[1,4]oxazepan-4-yl]-3-oxo-propoxy}-phenyl)-urea,
(5-Chloro-2-{3-[2-(3,4-difluoro-benzyl)-[1,4]oxazepan-4-yl]-propoxy}-phenyl)-urea,
(5-Chloro-2-{3-[2-(3-fluoro-benzyl)-[1,4]oxazepan-4-yl]-3-oxo-propoxy}-phenyl)-urea,
(5-Chloro-2-{3-[2-(3-fluoro-benzyl)-[1,4]oxazepan-4-yl]-propoxy}-phenyl)-urea,
2,6-Dichloro-N-{3-[2-(4-fluoro-benzyl)-[1,4]oxazepan-4-yl]-propyl}-benzenesulfonamide,
4-[3-(2-Bromo-4-chloro-phenoxy)-propyl]-2-(4-fluoro-benzyl)-[1,4]oxazepane,
5-chloro-2-{(R)-3-[(R)-2-(4-fluoro-benzyl)-[1,4]oxazepan-4-yl]-2-hydroxy-propoxy}-benzamide,
5-chloro-2-{(S)-3-[(R)-2-(4-fluoro-benzyl)-[1,4]oxazepan-4-yl]-2-hydroxy-propoxy}-benzamide,
5-chloro-2-{(R)-3-[(S)-2-(4-fluoro-benzyl)-[1,4]oxazepan-4-yl]-2-hydroxy-propoxy}-benzamide,
5-chloro-2-{(S)-3-[(S)-2-(4-fluoro-benzyl)-[1,4]oxazepan-4-yl]-2-hydroxy-propoxy}-benzamide,
N-(3-Chloro-2-methyl-phenyl)-2-[2-(4-fluoro-benzyl)-[1,4]oxazepan-4-yl]-acetamide,
4-[3-(2,6-Dichloro-phenoxy)-propyl]-2-(4-fluoro-benzyl)-[1,4]oxazepane,
2,6-Dichloro-N-{2-[2-(4-fluoro-benzyl)-[1,4]oxazepan-4-yl]-ethyl}-benzenesulfonamide,
N-{3-[2-(4-Fluoro-benzyl)-[1,4]oxazepan-4-yl]-propyl}-4-methoxy-benzenesulfonamide,
4-Chloro-N-{3-[2-(4-fluoro-benzyl)-[1,4]oxazepan-4-yl]-propyl}-benzenesulfonamide,
5-Chloro-2-{3-[2-(4-fluoro-benzyl)-[1,4]oxazepan-4-yl]-propoxy}-benzoic acid,
(5-Chloro-2-{3-[2-(4-chloro-benzyl)-[1,4]oxazepan-4-yl]-propoxy}-phenyl)-urea, and
2-(4-Chloro-2-isoxazol-5-yl-phenoxy)-1-[7-(4-fluoro-benzyl)-[1,4]oxazepan-4-yl]-ethanone,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

In additional embodiments, the compound of formula IV is selected from:
3-[4-(4-Chloro-phenyl)-4-hydroxy-3,3-dimethyl-piperidin-1-yl]-1-[2-(4-fluoro-benzyl)-[1,4]oxazepan-4-yl]-propan-1-one,
4-(4-Chloro-phenyl)-1-{2-[2-(4-fluoro-benzyl)-[1,4]oxazepan-4-yl]-ethyl}-3,3-dimethyl-piperidin-4-ol, and
4-chloro-2-{3-[2-(4-fluoro-benzyl)-[1,4]oxazepan-4-yl]-2-hydroxy-propoxy}-benzamide,

wherein free base forms listed above can also be in the form of a pharmaceutically acceptable salt,

wherein a compound listed above (in either a free base form or in the form of a pharmaceutically acceptable salt) can also be in the form of a solvate (such as a hydrate),

wherein a compound listed above (in either a free base form or in the form of a pharmaceutically acceptable salt) can also be in the form of an N-oxide,

wherein a compound listed above (in a free base form or solvate or N-oxide thereof, or in the form of a pharmaceutically acceptable salt or solvate thereof,) can also be in the form of a polymorph, and

wherein if the compound exhibits chirality it can be in the form of a mixture of enantiomers such as a racemate or a mixture of diastereomers, or can be in the form of a single enantiomer or a single diastereomer.

As used herein the term “halogen” means F, Cl, Br, and I.

The term “alkyl” means a substituted or unsubstituted saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms. Suitable alkyl groups include, but are not limited to, methyl, ethyl, propyl, isopropyl, butyl, sec-butyl, tert-butyl, pentyl, hexyl, heptyl, octyl, nonyl, decyl, undecyl, and dodecyl. Other examples of suitable alkyl groups include, but are not limited to, 1-, 2- or 3-methylbutyl, 1,1-, 1,2- or 2,2-dimethylpropyl, 1-ethylpropyl, 1-, 2-, 3- or 4-methylpentyl, 1,1-, 1,2-, 1,3-, 2,2-, 2,3- or 3,3-dimethylbutyl, 1- or 2-ethylbutyl, ethylmethylpropyl, trimethylpropyl, methylhexyl, dimethylpentyl, ethylpentyl, ethylmethylbutyl, dimethylbutyl, and the like.

Substituted alkyl groups are alkyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “halogenated alkyl” means a saturated hydrocarbon radical which may be straight-chain or branched-chain and may comprise about 1 to about 20 carbon atoms, for instance 1 to 12 carbon atoms, such as 1 to 8 carbon atoms, e.g., 1 to 4 carbon atoms, that is substituted by one or more halogens, such as, but not limited to, —CF₃, CF₂CF₃, CHF₂, CH₂F, and the like. The use of the term “halogenated alkyl” should not be construed to mean that a “substituted alkyl” group may not be substituted by one or more halogens.

The term “alkenyl” means a substituted or unsubstituted hydrocarbon radical which may be straight-chain or branched-chain, which contains one or more carbon-carbon double bonds, and which may comprise about 1 to about 20 carbon atoms, such as 1 to 12 carbon atoms, for instance 1 to 6 carbon atoms. Suitable alkenyl groups include ethenyl, propenyl, butenyl, etc.

Substituted alkenyl groups are alkenyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

The term “alkynyl” means a substituted or unsubstituted aliphatic hydrocarbon radical which may be straight-chain or branched-chain and which contains one or more carbon-carbon triple bonds. Preferably the alkynyl group contains 2 to 15 carbon atoms, such as 2 to 12 carbon atoms, e.g., 2 to 8 carbon atoms. Suitable alkynyl groups include ethynyl, propynyl, butynyl, etc.

Substituted alkynyl groups are alkynyl groups as described above which are substituted in one or more positions by, e.g., halogen, hydroxyl, amino, carboxy, alkylamino, dialkylamino, aryl, heteroaryl, alkoxy, nitro and cyano, and combinations thereof.

The term “amino” means —NH₂.

The term “alkylamino” means —NH(alkyl), wherein alkyl is as described above.

The term “dialkylamino” means —N(alkyl)₂, wherein alkyl is as described above.

The term “aryl” means a substituted or unsubstituted aromatic monocyclic or bicyclic ring system comprising about 5 to about 14 carbon atoms, e.g., about 6 to about 10 carbon atoms. Suitable aryl groups include, but are not limited to, phenyl, naphthyl, anthracenyl.

Substituted aryl groups include the above-described aryl groups which are substituted one or more times by, for example, but not limited to, halogen, hydroxyl, amino, amido, alkylamido, —C(O)-heterocyclyl, ureido, carboxy, carboxylic acid ester, alkylamino, dialkylamino, aryl, heteroaryl, alkoxy, substituted alkoxy, nitro and cyano, and combinations thereof.

The term “arylamino” means —NH(aryl), wherein aryl is as described above.

The term “diarylamino” means —N(aryl)₂, wherein aryl is as described above.

The term “amido” means —CONH₂.

The term “ureido” means —NHCONH₂.

The term “—C(O)-heterocyclyl” means a substituted or unsubstituted non-aromatic monocyclic or bicyclic ring system comprising 3 to 10 atoms wherein at least one of the ring atoms is a N, O or S atom, and wherein the ring heteroatom is bonded directly to the C(O) moiety.

The term “alkylamido” means a —CONH(alkyl) group, wherein alkyl is as described above.

The term “dialkylamido” means a —CON(alkyl)₂ group, wherein alkyl is as described above.

The term “aminoalkyl” means a -(alkylene)-amino, -(alkylene)-alkylamino or -(alkylene)-dialkylamino group, wherein the various groups are as described above.

The term “arylalkyl” refers to an -(alkylene)-aryl group in which the aryl and alkylene portions are in accordance with the previous descriptions. Suitable examples include, but are not limited to, benzyl, 1-phenethyl, 2-phenethyl, phenpropyl, phenbutyl, phenpentyl, and napthylmethyl.

The term “carboxyl” means —C(O)OH.

The term “cycloalkyl” means a monocyclic, bicyclic or tricyclic nonaromatic saturated hydrocarbon radical having 3 to 10 carbon atoms, such as 3 to 8 carbon atoms, for example, 3 to 6 carbon atoms. Suitable cycloalkyl groups include, but are not limited to, cyclopropyl, cyclobutyl, cyclopentyl, cyclohexyl, cycloheptyl, cyclooctyl, norbornyl, 1-decalin, adamant-1-yl, and adamant-2-yl. Other suitable cycloalkyl groups include, but are not limited to, spiropentyl, bicyclo[2.1.0]pentyl, bicyclo[3.1.0]hexyl, spiro[2.4]heptyl, spiro[2.5]octyl, bicyclo[5.1.0]octyl, spiro[2.6]nonyl, bicyclo[2.2.0]hexyl, spiro[3.3]heptyl, bicyclo[4.2.0]octyl, and spiro[3.5]nonyl. Preferred cycloalkyl groups include cyclopropyl, cyclobutyl, cyclopentyl and cyclohexyl. The cycloalkyl group can be substituted, for example, by one or more halogens and/or alkyl groups.

The term “cycloalkylalkyl” means a -(alkylene)-cycloalkyl in which the cycloalkyl group is as previously described; e.g., cyclopropylmethyl, cyclobutylmethyl, cyclopentylethyl, or cyclohexylmethyl, and the like.

The term “heteroaryl” means a substituted or unsubstituted aromatic monocyclic or multicyclic ring system comprising 5 to 14 ring atoms, preferably about 5 to about 10 ring atoms and most preferably 5 or 6 ring atoms, wherein at least one of the ring atoms is an N, O or S atom. Suitable heteroaryl groups include, but are not limited to furyl, thienyl, pyrrolyl, pyrazolyl, imidazolyl, pyridyl, pyrimidinyl, benzimidazolyl, indazolyl, indolyl, quinolinyl, isoquinolinyl, naphthyridinyl and the like.

Substituted heteroaryl groups include the above-described heteroaryl groups which are substituted one or more times by, for example, but not limited to, halogen, hydroxyl, amino, carboxy, alkylamino, dialkylamino, aryl, heteroaryl, alkoxy, nitro and combinations thereof.

The term “heteroarylalkyl” refers to a -(alkylene)-heteroaryl group wherein the heteroaryl and alkylene portions are in accordance with the previous discussions. Suitable examples include, but are not limited to, pyridylmethyl, thiazolylmethyl, thienylmethyl, pyrimidinylmethyl, pyrazinylmethyl, and isoquinolinylmethyl, and the like.

The term “heterocycle” means a substituted or unsubstituted non-aromatic mono- or multicyclic ring system comprising 3 to 10 atoms, preferably 5 or 6, wherein at least one of the ring atoms is an N, O or S atom. Suitable heterocyle groups include, but are not limited to tetrahydropyranyl, tetrahydrothienyl, tetrahydropyranyl, dihydropyranyl, pyrrolidinyl, piperidinyl, piperazinyl, thiomorpholinyl, morpholinyl, isoxazolinyl, and the like

Substituted heterocycle groups include the above-described heterocycle groups which are substituted one or more times by, for example, halogen, amino, alkyl, hydroxy, carboxy, and combinations thereof. Heterocycle groups may also be substituted by, e.g., aryl or heteroaryl.

The term “heterocyclealkyl” refers to a -(alkylene)-heterocycle group wherein the heterocycle and alkylene portions are in accordance with the previous discussions.

The term “aroyl” means an aryl-C(O)—, in which the aryl group is as previously described. Suitable aroyl groups include, but are not limited to, benzoyl and 1-naphthoyl.

The term “acyl” means an HC(O)—, alkyl-C(O)—, cycloalkyl-C(O)—, aryl-C(O)—, or heteroalkyl-C(O)—, in which the various groups are as previously described, e.g., acetyl, propionyl, benzoyl, pyridinylcarbonyl, and the like.

The term “alkoxy” means alkyl-O— groups in which the alkyl portion is in accordance with the previous discussion. Suitable alkoxy groups include, but are not limited to, methoxy, ethoxy, n-propoxy, iso-propoxy, n-butoxy, t-butoxy, pentoxy, hexoxy, heptoxy, octoxy, and the like. For example, the alkoxy can be methoxy or ethoxy.

The term “substituted alkoxy” means alkyl-O-groups in which the alkyl group is substituted in accordance with the previous discussion.

The term “aryloxy” means an aryl-O— group, in which the aryl group is as previously described.

The term “heteroaryloxy” means an heteroaryl-O— group, in which the heteroaryl group is as previously described.

The term “cycloalkylalkyloxy” means a —O-(alkylene)-cycloalkyl group, in which the cycloalkyl and alkylene groups are as previously described.

The term “alkylthio” means an alkyl-S— group, in which the alkyl group is as previously described.

The term “arylthio” means an aryl-S— group, in which the aryl group is as previously described.

The term “alkylsulfinyl” means a —SOR radical where R is alkyl as defined above, e.g., methylsulfinyl, ethylsulfinyl, and the like.

The term “alkylsulfonyl” means a —SO₂R radical where R is alkyl as defined above, e.g., methylsulfonyl, ethylsulfonyl, and the like.

The term “arylsulfinyl” means a —SOR radical where R is aryl as defined above, e.g., phenylsulfinyl, and the like.

The term “arylsulfonyl” means a —SO₂R radical where R is aryl as defined above, e.g., phenylsulfonyl, and the like.

The term “heteroarylsulfinyl” means a —SOR radical where R is heteroaryl as defined above.

The term “heteroarylsulfonyl” means a —SO₂R radical where R is heteroaryl as defined above.

The term “alkoxycarbonyl” means an alkyl-O—C(O)— group, in which the alkyl group is as previously described.

The term “aryloxycarbonyl” means an aryl-O—C(O)— group, in which the aryl group is as previously described.

The term “heteroaryloxycarbonyl” means an heteroaryl-O—C(O)— group, in which the heteroaryl group is as previously described.

The term “cycloalkyloxy” means a —O-cycloalkyl group in which the cycloalkyl group is as previously described, e.g., cyclopropyloxy, cyclobutyloxy, cyclopentyloxy, cyclohexyloxy, and the like

The term “arylalkyloxy” means —O-(alkylene)-aryl group, in which the aryl and alkylene groups are as previously described.

The term “heteroarylalkyloxy” means —O-(alkylene)-heteroaryl group, in which the heteroaryl and alkylene groups are as previously described.

One of ordinary skill in the art will recognize that compounds of the present invention can exist in different tautomeric and geometrical isomeric forms. All of these compounds, including cis isomers, trans isomers, diastereomic mixtures, racemates, nonracemic mixtures of enantiomers, substantially pure, and pure enantiomers, are within the scope of the present invention. Substantially pure enantiomers contain no more than 5% w/w of the corresponding opposite enantiomer, such as no more than 2%, for example, no more than 1%.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, dibenzoyltartaric, ditoluoyltartaric and camphorsulfonic acid. Mixtures of diastereoisomers can be separated into their individual diastereomers on the basis of their physical and/or chemical differences by methods known to those skilled in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. Optically active compounds of the present invention can likewise be obtained by utilizing optically active starting materials in chiral synthesis processes under reaction conditions which do not cause racemization.

In addition, one of ordinary skill in the art will recognize that the compounds can be used in different enriched isotopic forms, e.g., enriched in the content of ²H, ³H, ¹¹C, ¹³C and/or ¹⁴C. In one particular embodiment, the compounds are deuterated. Such deuterated forms can be made the procedure described in U.S. Pat. Nos. 5,846,514 and 6,334,997. As described in U.S. Pat. Nos. 5,846,514 and 6,334,997, deuteration can improve the efficacy and increase the duration of action of drugs.

Deuterium substituted compounds can be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] (2000), 110 pp. CAN 133:68895 AN 2000:473538 CAPLUS; Kabalka, George W.; Varma, Rajender S. The synthesis of radiolabeled compounds via organometallic intermediates. Tetrahedron (1989), 45(21), 6601-21, CODEN: TETRAB ISSN:0040-4020. CAN 112:20527 AN 1990:20527 CAPLUS; and Evans, E. Anthony. Synthesis of radiolabeled compounds, J. Radioanal. Chem. (1981), 64 (1-2), 9-32. CODEN: JRACBN ISSN:0022-4081, CAN 95:76229 AN 1981:476229 CAPLUS.

Where applicable, the present invention also relates to useful forms of the compounds as disclosed herein, such as base free forms, and pharmaceutically acceptable salts or prodrugs of all the compounds of the present invention for which salts or prodrugs can be prepared. Pharmaceutically acceptable salts include those obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

The following are further examples of acid salts that can be obtained by reaction with inorganic or organic acids: acetates, aDIPEAtes, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride, a hydrobromide, a hydroformate, or a maleate. For further example, the pharmaceutically acceptable salt is a hydrochloride.

Preferably, the salts formed are pharmaceutically acceptable for administration to mammals. However, pharmaceutically unacceptable salts of the compounds are suitable as intermediates, for example, for isolating the compound as a salt and then converting the salt back to the free base compound by treatment with an alkaline reagent. The free base can then, if desired, be converted to a pharmaceutically acceptable acid addition salt.

One of ordinary skill in the art will also recognize that some of the compounds of the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

One of ordinary skill in the art will further recognize that compounds of the present invention can exist in different solvate forms. Solvates of the compounds of the invention may also form when solvent molecules are incorporated into the crystalline lattice structure of the compound molecule during the crystallization process.

The present invention also includes prodrugs of compounds of formulas I-IV. The term prodrug is intended to represent covalently bonded carriers, which are capable of releasing the active ingredient of formulas I-IV when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of formulas I-IV include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of formulas I-IV), amides (e.g., trifluoroacetylamino, acetylamino, and the like), and the like. Prodrugs of compounds of formulas I-IV are also within the scope of this invention.

The present invention also provides processes for preparing the compounds of formulas I-IV. Suitable general reaction schemes are shown below.

Compounds of formula I may be prepared as shown in general scheme 1.

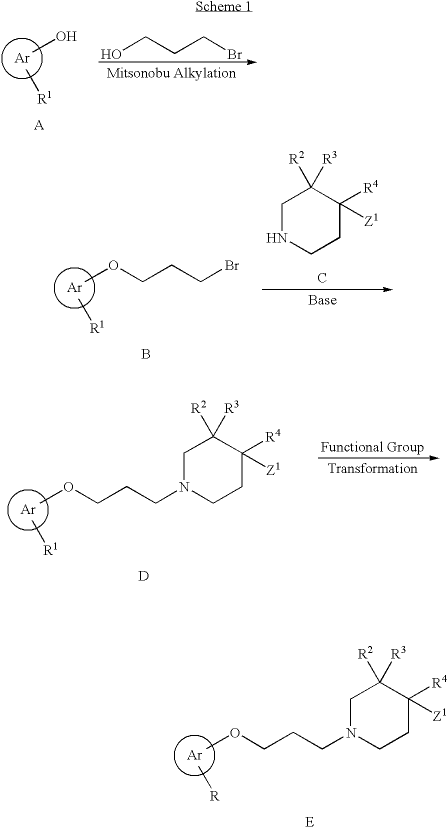

Compound B in scheme 1 may be prepared by Mitsunobu alkylation of Compound A using a suitable hydroxyhaloalkyl halide in the presence of a di-isopropyl azodicarboxylate and triphenyl phosphine. N-Alkylation of Compound C with Compound B in the presence of a suitable base (e.g. trietheyl amine) affords Compound D, which may be converted to Compound E via appropriate standard functional group transformation reactions (e.g. conversion of a nitro group to an acetamide group by reducing the nitro to an aminemoiety using zinc/ammonium chloride followed by acetylation using acetyl chloride and triethyl amine etc.)

Compounds of formula II may be prepared as shown in general schemes 2 and 3.

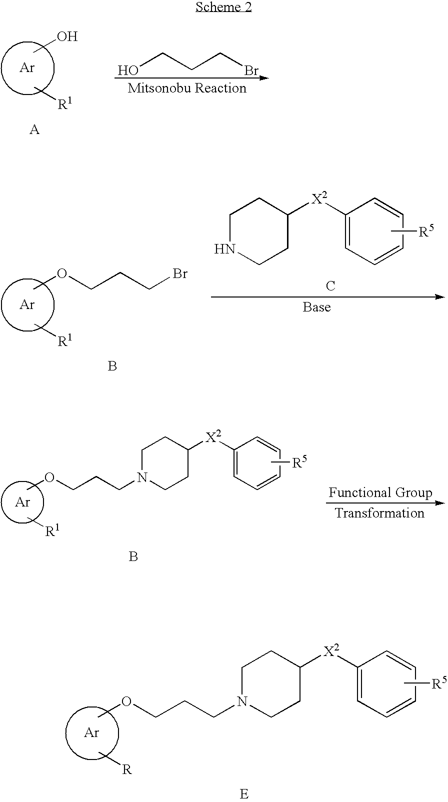

Compound B in scheme 2 may be prepared by Mitsunobu alkylation of Compound A using a suitable hydroxyhaloalkyl halide in the presence of a di-isopropyl azodicarboxylate and triphenyl phosphine. N-Alkylation of Compound C with Compound B in the presence of a suitable base (e.g. trietheyl amine) affords Compound D, which may be converted to Compound E via appropriate standard functional group transformation reactions (e.g. conversion of a nitro group to an acetamide group by reducing the nitro to an aminemoiety using zinc/ammonium chloride followed by acetylation using acetyl chloride and triethyl amine, etc.)

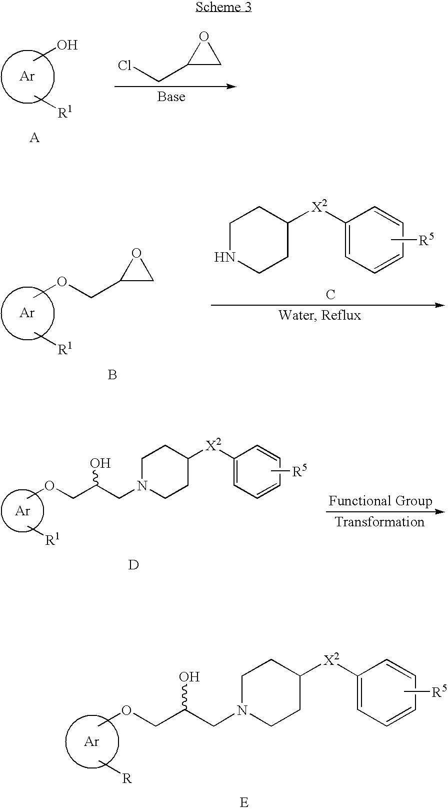

Compound B in scheme 3 may be prepared by treating compound A with epi-chlorohydrin in the presence of a suitable base (e.g. cesium carbonate). Regio-selective epoxide opening by refluxing Compound B with Compound C in an aprotic solvent (e.g., water) affords Compound D, which may be converted to Compound E via appropriate standard functional group transformation reactions (e.g. conversion of a nitro group to an acetamide group by reducing the nitro to an aminemoiety using zinc/ammonium chloride followed by acetylation using acetyl chloride and triethyl amine, etc.)

Compounds of formula III may be prepared as shown in general schemes 4-7.

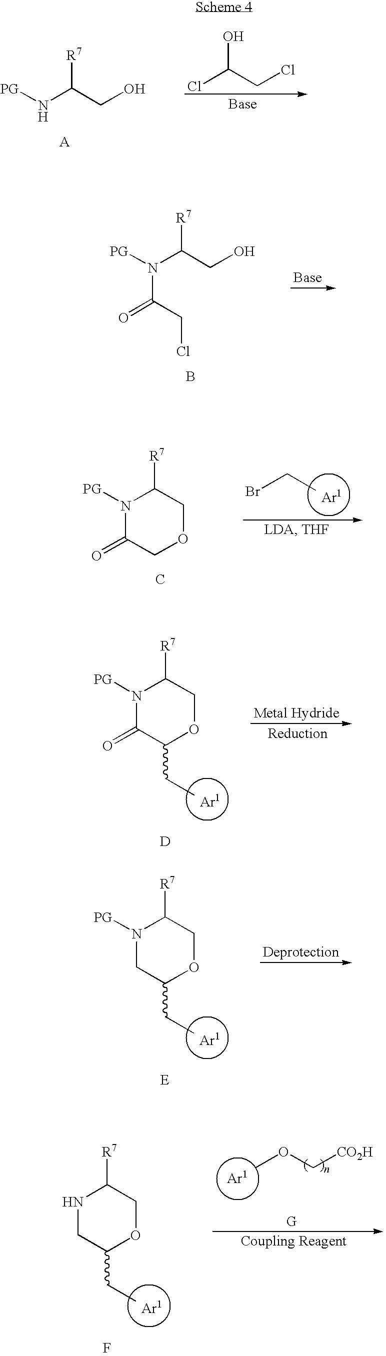

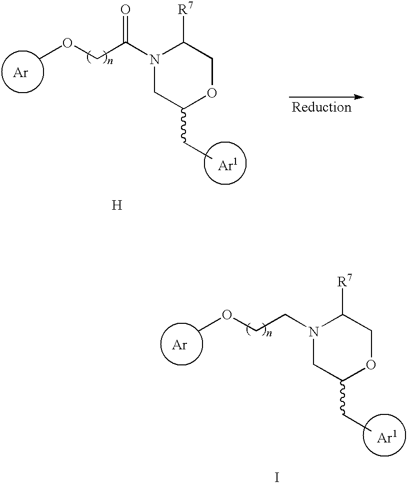

Compound B in scheme 4 may be prepared by treating Compound A with a haloalkyl acid chloride in the presence of a suitable base (e.g. triethyl amine). Intramolecular cyclization of Compound B in the presence of an anhydrous base (e.g. sodium hydride) affords Compound C, which is converted to Compound D via base (e.g., lithium di-isopropylamide) mediated alkylation with an appropriate arylalkyl halide. Reduction of Compound D with a metal hydride (e.g. lithium alluminium hydride) followed by removal of the protecting group (PG) affords Compound F. Coupling Compound F with Compound G in the presence of suitable coupling reagent (e.g. EDC/HOBt) affords Compound H. Reduction of Compound H (e.g. using borohydrides such as borane-dimethyl sulfide complex) complex affords Compound I.

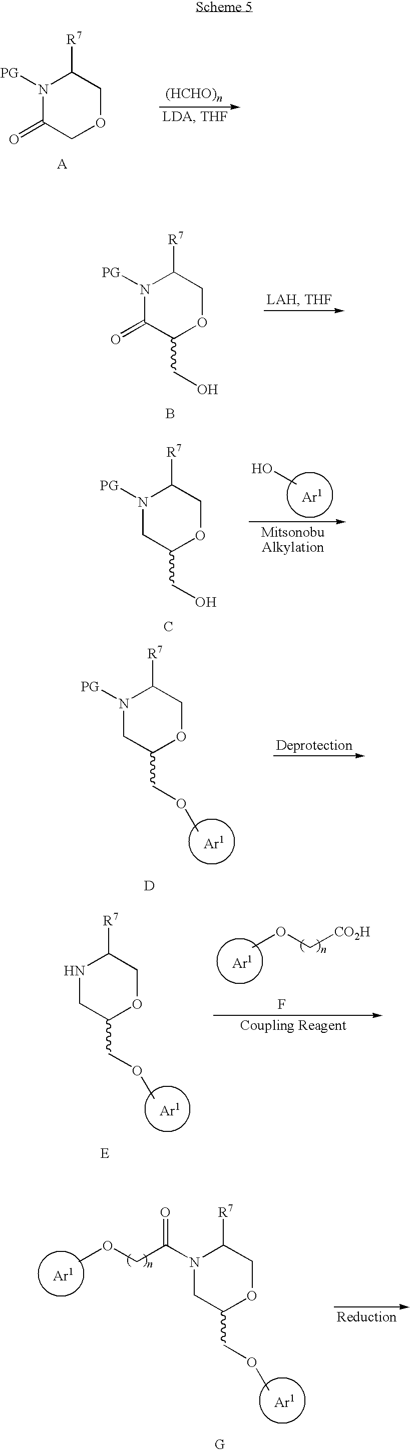

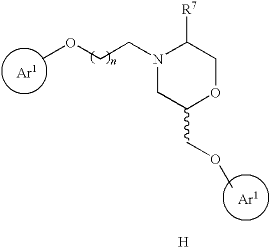

In scheme 5, hydroxymethylation of Compound A using para-formaldehyde and a suitable base (e.g. lithium di-isopropylamide), followed by metal hydride (e.g. lithium aluminum hydride) reduction of intermediate Compound B affords Compound C. Compound C may be converted to aryl ether Compound D via standard alkylation procedures (e.g. Mitsunobu alkylation using triphenyl phosphine and di-isopropyl azodicarboxylate). Deprotection of Compound D followed by amide coupling with Compound F using suitable coupling reagents (e.g. EDC/HOBt) affords Compound G. Reduction of Compound G (e.g. using borohydrides such as borane-dimethyl sulfide complex) affords Compound H.

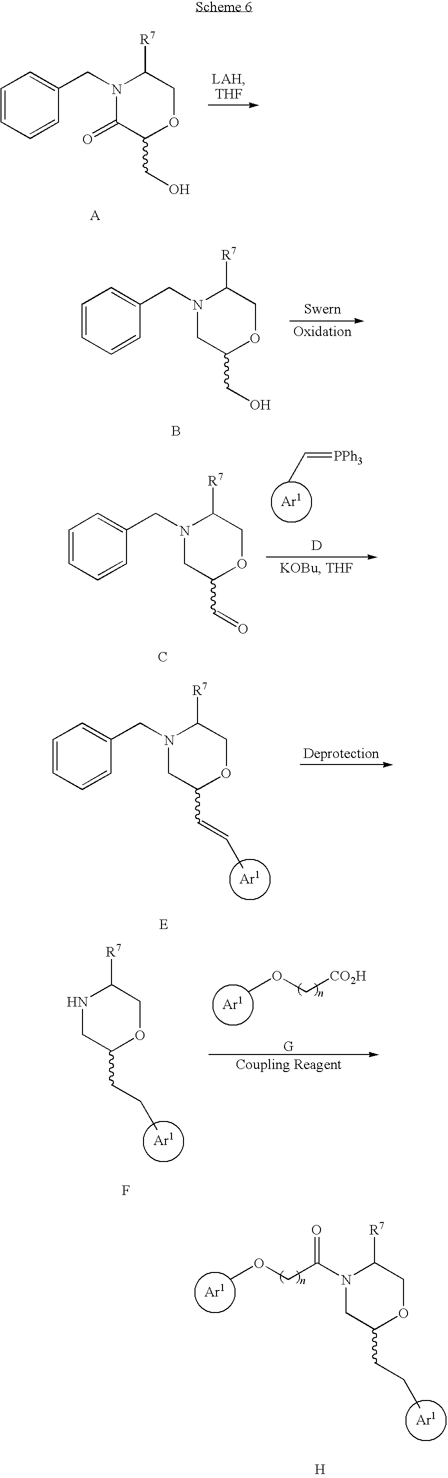

In scheme 6, reduction of Compound A (using, e.g., a metal hydride such as lithium aluminum hydride) affords Compound B, which may subsequently be oxidized (using, e.g., oxalyl chloride/DMSO/triethyl amine (Swern oxidation) conditions) to afford Compound C. A Wittig reaction between Compound C and an appropriate phosphorane (Compound D) affords Compound E. Reductive deprotection of Compound E (e.g., debenzylation in the presence of hydrogen/Pd—C) followed by amide coupling of the resulting Compound F with Compound G using suitable coupling reagents (e.g. EDC/HOBt) affords Compound H.

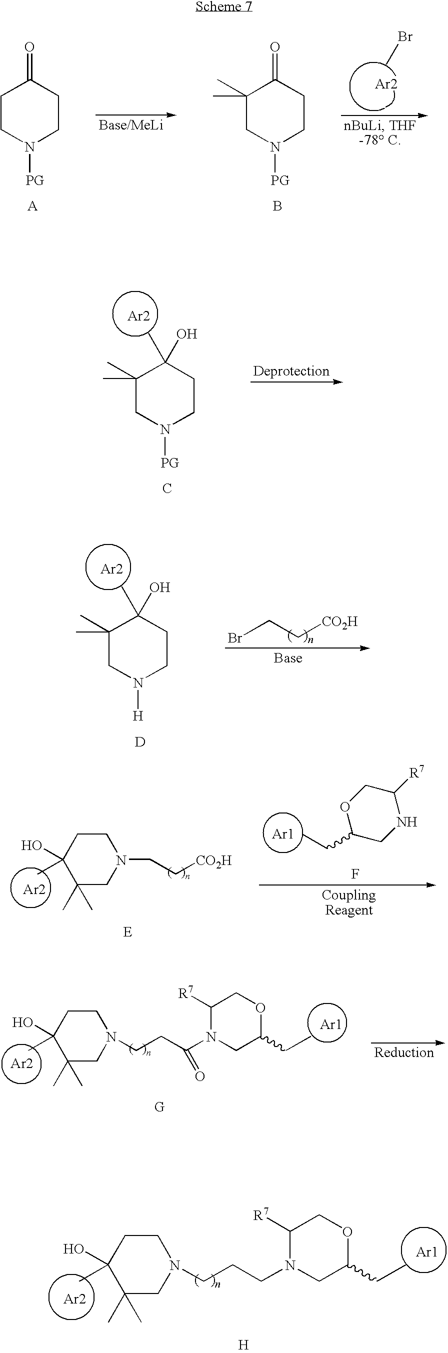

Compound B in scheme 7 may be prepared by base mediated methylation of Compound A (e.g. using sodium tert-butoxide/MeI). Treatment of Compound B with an appropriate aryl lithium reagent (e.g. phenyl lithim, which may be generated from bromobenzene/BuLi) affords Compound C. Removal of the protecting group (e.g. removal of a Boc group by trifluoroacetic acid), followed by N-alkylation of the intermediate Compound D with a haloalkyl acid in the presence of a suitable base (e.g. triethylamine) affords Compound E. Coupling Compound E with an appropriate morpholino Compound F in presence of a suitable coupling agent (e.g. EDC/HOBt) affords Compound G. Selective reduction of Compound G (e.g. borohydride reduction using borane-dimethyl sulfide complex) affords Compound H.

Compounds of formula IV may be prepared as shown in general schemes 8-13.

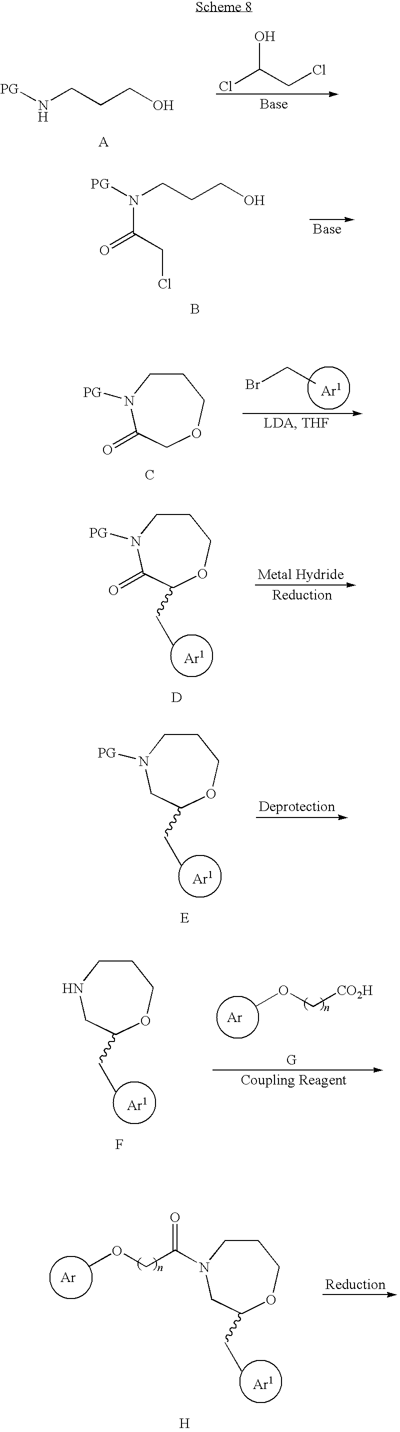

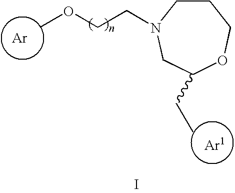

Compound B in scheme 8 may be prepared by treating Compound A with a suitable haloalkyl acid chloride (e.g. chloroacetyl chloride) in the presence of a base (e.g. triethyl amine). Intramolecular cyclization of Compound B in the presence of an anhydrous base (e.g., sodium hydride) affords Compound C, which may be alkylated with an appropriate alkylaryl halide (e.g. benzyl bromide) in the presence of a suitable base (e.g. lithium di-isopropylamide) to afford Compound D. Reduction of Compound D (e.g., using a metal hydride such as lithium aluminum hydride) followed by removal of the protecting group (PG) (e.g. reductive deprotection of a benzyl group with hydrogen/Pd—C) affords Compound F. Coupling Compound F with Compound G using suitable coupling agents (e.g., EDC/HOBt) affords Compound H. Reduction of Compound H (e.g., using brorohydride reagents, such as borane-dimethyl sulfide complex) affords Compound I.

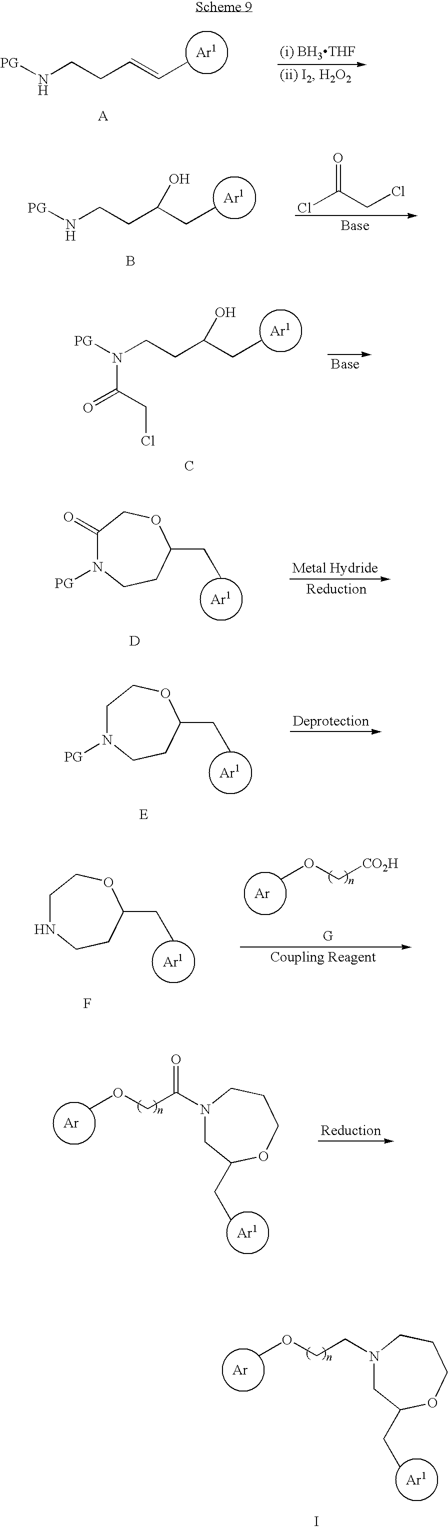

Compound B in scheme 9 may be prepared from Compound A following a modified procedure similar to that described in J. Am. Chem. Soc., 125, 10502-10503, 2003. Treatment of Compound B with a haloalkyl acid chloride affords Compound C, which may be treated with anhydrous base (e.g. sodium hydride) to afford Compound D. Reduction of Compound D (e.g., using a metal hydride such as lithium aluminum hydride) affords Compound E, which may be converted to Compound F by removal of the protecting group (e.g. by reductive deprotection of a benzyl group with hydrogen/Pd—C). Coupling Compound F with Compound G in the presence of a suitable coupling agent (e.g. EDC/HOBt) affords Compound H. Reduction of Compound H (e.g., using a brorohydride reagent such as borane-dimethyl sulfide complex) affords Compound I.

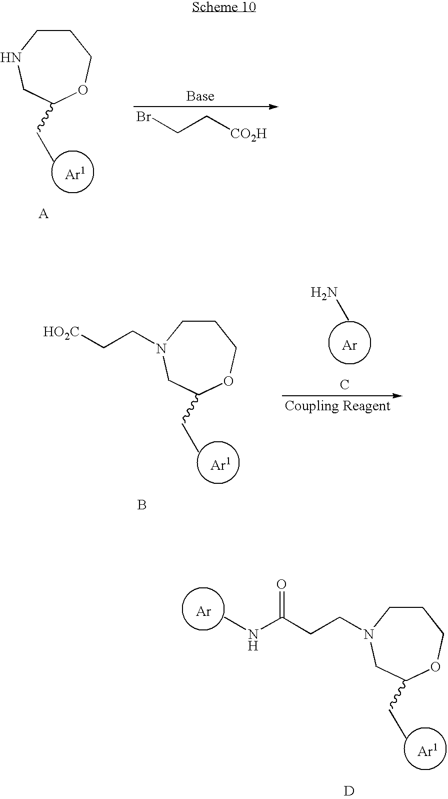

Compound B in scheme 10 may be prepared from Compound A (which itself may be synthesized in a manner similar to Compound F in scheme 8) by reaction with a haloalkyl acid (e.g. bromopropionic acid) in the presence of a base such as triethyl amine. Amide coupling between Compound B and an appropriate amine (Compound C) in the presence of a suitable coupling agent (e.g. EDC/HOBt) affords Compound D.

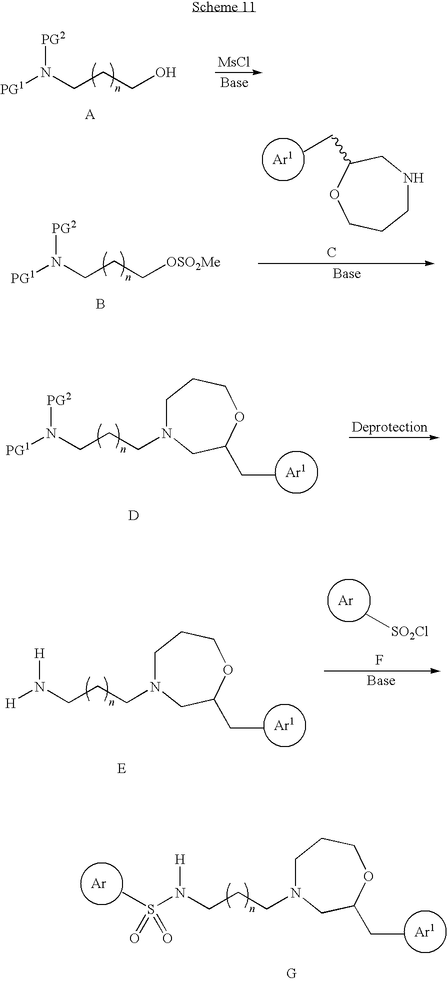

The hydroxyl group of Compound A in scheme 11 may be converted to a suitable leaving group (e.g. mesylate) by reaction with, e.g., mesyl chloride in presence of triethyl amine, to afford Compound B. Reaction of Compound B with an appropriate Compound C affords Compound D. Removal of the protecting groups (e.g. by treatment with trifluoro acetic acid to remove Boc protecting groups) followed by reductive deprotection of a benzyl group with hydrogen/Pd—C affords Compound E. Recation with an appropriate aryl sulfonyl chloride (Compound F) affords Compound G.

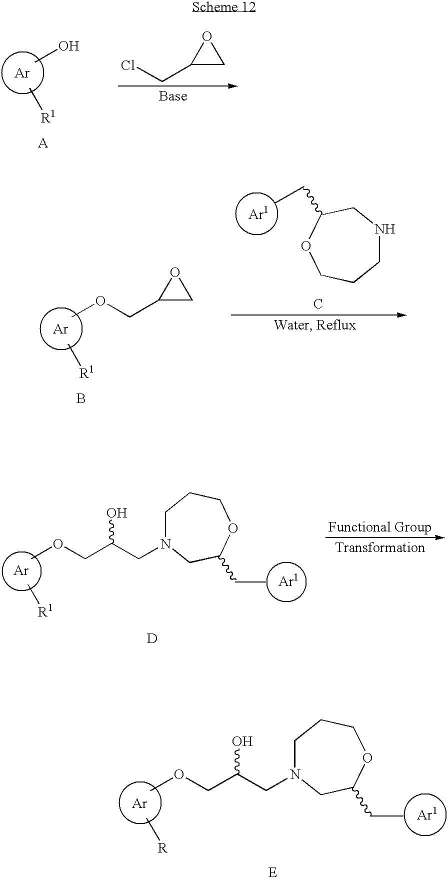

Compound B in scheme 12 may be prepared by reacting Compound A with epi-chlorohydrin in the presence of a suitable base (e.g., cesium carbonate). Regioselective ring opening (by refluxing Compound B with Compound C) in a protic solvent such as water affords Compound D. Compound D may be converted to the desired Compound E by standard functional group transformation reactions (e.g. conversion of a nitro group to an acetamide group by reducing the nitro to an aminemoiety using zinc/ammonium chloride followed by acetylation using acetyl chloride and triethyl amine, etc.)

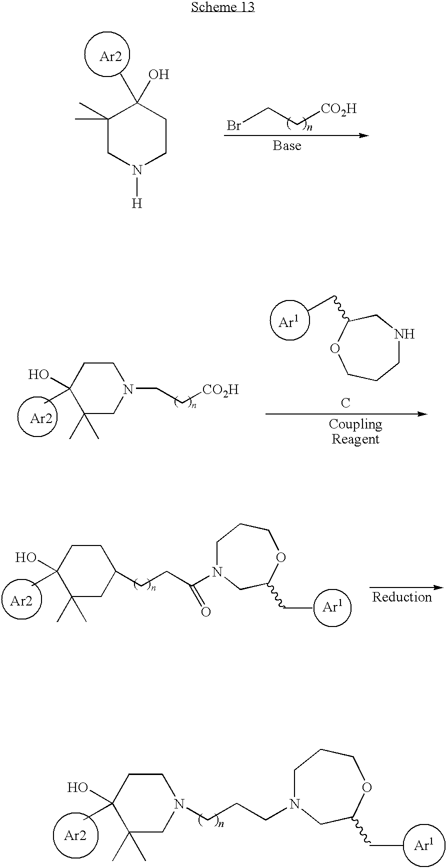

Compound B in scheme 13 may be prepared by treating Compound A with an appropriate haloalkyl carboxylic acid in presence of suitable base such as triethyl amine. Amide coupling between Compound B and an appropriate Compound C in the presence of a suitable coupling agent (e.g. EDC/HOBt) affords Compound D. Reduction of Compound D. e.g., using brorohydride reagents (e.g. borane-dimethyl sulfide complex) affords Compound E.

The compounds of the invention can be administered alone or as an active ingredient of a formulation. Thus, the present invention also includes pharmaceutical compositions of compounds of formulas I, II, III or IV, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the compounds of the present invention may be accomplished according to patient needs, for example, orally, nasally, parenterally (subcutaneously, intraveneously, intramuscularly, intrasternally and by infusion) by inhalation, rectally, vaginally, topically and by ocular administration.

Various solid oral dosage forms can be used for administering compounds of the invention including such solid forms as tablets, gelcaps, capsules, caplets, granules, lozenges and bulk powders. The compounds of the present invention can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present invention.

Various liquid oral dosage forms can also be used for administering compounds of the inventions, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The compounds of the present invention may be injected, for example, intravenously, in the form of an isotonic sterile solution. Other preparations are also possible.

Suppositories for rectal administration of the compounds of the present invention can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration the pharmaceutical composition can be in the form of creams, ointments, liniments, lotions, emulsions, suspensions, gels, solutions, pastes, powders, sprays, and drops suitable for administration to the skin, eye, ear or nose. Topical administration may also involve transdermal administration via means such as transdermal patches.

Aerosol formulations suitable for administering via inhalation also can be made. For example, the compounds of formulas I, II, III or IV can be administered by inhalation in the form of a powder (e.g., micronized) or in the form of atomized solutions or suspensions. The aerosol formulation can be placed into a pressurized acceptable propellant.

### Methods of Treatment

The compounds of formulas I, II, III or IV may be useful as ligands for CC chemokine receptors, for example, CCR1. Therefore, compounds of formulas I, II, III or IV may be useful in the treatment of conditions mediated by CC chemokine receptors, for example, CCR1. In certain embodiments, the compounds of the present invention may be useful in the treatment of conditions that respond to a CCR1 receptor agonist, inverse agonist or antagonist, for example, conditions that respond to a CCR1 antagonist.

Therefore, the present invention also provides methods of treating CC chemokine receptor (e.g., CCR1) mediated conditions or diseases by administering to a patient having such a disease or condition, a therapeutically effective amount of a compound of formula I, II, III or IV, or a combination or mixture thereof.

CCR1 provides a target for interfering with or promoting specific aspects of immune cell functions, or more generally, with functions associated with CCR1 expression on a wide range of cell types in a mammal, such as a human. Compounds that inhibit CCR1, are particularly useful for modulating monocyte, macrophage, lymphocyte, granulocyte, NK cell, mast cells, dendritic cell, neutrophils, and certain immune derived cell (for example, osteoclasts) function for therapeutic purposes. Accordingly, the present invention is directed to compounds which are useful in the prevention, treatment and/or management of a wide variety of inflammatory and immunoregulatory disorders and diseases (see Saeki, et al., Current Pharmaceutical Design, 9, 1201-1208, 2003).

There are also provided, in accordance with embodiments of the invention, methods of treating or preventing inflammatory or autoimmune diseases comprising administering a compound of formula I, II, III or IV, or a combination or mixture thereof. In some embodiments the inflammatory disease or autoimmune disease is rheumatoid arthritis or multiple sclerosis.

Compounds of the genera are CCR1 antagonists. As such, they have utility in treating and preventing autoimmune disease and inflammatory diseases. In particular, CCR1 antagonists are therapeutic targets for the treatment and prevention of a variety of diseases, including, but not limited to, autoimmune diseases (such as rheumatoid arthritis, Takayasu arthritis, psoriatic arthritis, ankylosing spondylitis, type 1 diabetes (recent onset), lupus, inflammatory bowel disease, Crohn's disease, optic neuritis, psoriasis, multiple sclerosis, polymyalgia rheumatica, uveitis, thyroiditis and vasculitis); fibrosis (e.g. pulmonary fibrosis (i.e. idiopathic pulmonary fibrosis, interstitial pulmonary fibrosis), fibrosis associated with end-stage renal disease, fibrosis caused by radiation, tubulo interstitial fibrosis, subepithelial fibrosis, scleroderma (progressive systemic sclerosis), hepatic fibrosis (including that caused by alcoholic or viral hepatitis), primary and secondary biliary cirrhosis); diabetic nephropathy; allergic conditions (such as asthma, contact dermatitis and atopic dermatitis); acute and chronic lung inflammation (such as chronic bronchitis, chronic obstructive pulmonary disease, adult Respiratory Distress Syndrome, Respiratory Distress Syndrome of infancy, immune complex alveolitis); atherosclerosis; vascular inflammation resulting from tissue transplant or during restenosis (including, but not limited to restenosis following angioplasty and/or stent insertion); other acute and chronic inflammatory conditions (such as synovial inflammation caused by arthroscopy, hyperuremia, or trauma, osteoarthritis, ischemia reperfusion injury, glomerulonephritis, nasal polyosis, enteritis, Behcet's disease, preeclampsia, oral lichen planus, Guillian-Barre syndrome); acute and/or chronic transplant rejection (including xenotransplantation); HIV Infectivity (co-receptor usage); granulomatous diseases (including sarcoidosis, leprosy and tuberculosis); conditions associated with leptin production (such as obesity, cachexia, anorexia, type II diabetes, hyperlipidemia and hypergonadism); Alzheimer's disease and other neurodegenerative diseases; osteolytic lesion and sequelae associated with certain cancers such as multiple myeloma; diagnosis and treatment of endometriosis; analgesia.

Compounds of formulas I, II, III or IV, or combinations or mixtures thereof may also inhibit the production of metallo proteinases and cytokines at inflammatory sites (including but not limited to MMP9, TNF, IL-1, and IL-6) either directly or indirectly (as a consequence of decreasing cell infiltration) thus providing benefit for diseases or conditions linked to these cytokines (such as joint tissue damage, hyperplasia, pannus formation and bone resorption, hepatic failure, Kawasaki syndrome, myocardial infarction, acute liver failure, septic shock, congestive heart failure, pulmonary emphysema or dyspnea associated therewith). Compounds of formulas I, II, III or IV, or combinations or mixtures thereof, may also be used to prevent or lessen tissue damage caused by inflammation induced by infectious agents (such as viral induced encephalomyelitis or demyelination, viral inflammation of the lung or liver (e.g. caused by influenza or hepatitis or respiratory syncytial virus), gastrointestinal inflammation (for example, resulting from H. pylori infection), inflammation resulting from: bacterial meningitis, HIV-1, HIV-2, HIV-3, cytomegalovirus (CMV), adenoviruses, Herpes viruses (Herpes zoster and Herpes simplex) fungal meningitis, lyme disease, malaria), arterial remodeling characterized by neointima formation and medial thickening for mediating inflammatory cell recruitment and endothelial dysfunction.

There are also provided, in accordance with additional embodiments of the invention, methods of treating or preventing or managing a disease disease or condition selected from, for example, hepatocellular carcinoma, respiratory synctial virus (RSV), kidney disease, allergic asthma, Alport disease (which includes glumerulosclerosis and progressive renal fibrosis), prion diseases, sepsis, T-cell mediated liver diseases, severe respiratory viruses, chronic renal injury, and transplant and cardio allograft vascalopathy (chronic rejection) comprising administering a compound of formulas I, II, III or IV, or a combination or mixture thereof. In some embodiments the Alport disease is renal fibrosis.

There are also provided, in accordance with embodiments of the invention, methods of treating or preventing endometriosis comprising administering a compound of formulas I, II, III or IV, or a combination or mixture thereof.

In certain embodiments, the compounds of the invention are useful in the treatment, management or prevention of elevated levels of lipids, cardiovascular diseases, diabetes, obesity, and metabolic syndrome.

In the treatment or prevention of conditions which require chemokine receptor modulation, an appropriate dosage level will generally be about 0.001 to 100 mg per kg patient body weight per day, which can be administered in single or multiple doses. For example, the dosage level will be about 0.01 to about 25 mg/kg per day; such as about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions may be provided in the form of tablets containing about 1 to about 1000 milligrams of the active ingredient, such as about 1, about 5, about 10, about 15, about 20, about 25, about 50, about 75, about 100, about 150, about 200, about 250, about 300, about 400, about 500, about 600, about 750, about 800, about 900 or about 1000 milligrams of the active ingredient. The compounds of the present invention may be administered on a regimen of 1 to 4 times per day, for example, once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex and diet of the subject, as well as the mode and time of administration, rate of excretion, drug combination, and the severity of the particular condition for the subject undergoing therapy.

The compounds and compositions of the present invention can be combined with other compounds and compositions having related utilities to prevent and treat the conditions described herein, such as, for example, inflammatory or autoimmune disorders, conditions and diseases, including inflammatory bowel disease, rheumatoid arthritis, osteoarthritis, psoriatic arthritis, polyarticular arthritis, multiple sclerosis, allergic diseases, psoriasis, atopic dermatitis and asthma.

For example, in the treatment or prevention of inflammation or autimmunity or for example arthritis associated bone loss, the present compounds and compositions may be used in conjunction with, for example, an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds and compositions may be administered with an analgesic listed above, a potentiator such as caffeine, an H2 antagonist (e.g., ranitidine), simethicone, aluminum or magnesium hydroxide; a decongestant such as phenylephrine, phenylpropanolamine, pseudoephedrine, oxymetazoline, ephinephrine, naphazoline, xylometazoline, propylhexedrine, or levo desoxy ephedrine; an antitussive such as codeine, hydrocodone, caramiphen, carbetapentane, or dextromethorphan; a diuretic; and a sedating or non sedating antihistamine.

Such other drug(s) may be administered, by a route and in an amount commonly used therefor, contemporaneously or sequentially with a compound of formulas I, II, III or IV. When a compound of formulas I, II, III or IV is used contemporaneously with one or more other drugs, a pharmaceutical unit dosage form containing such other drugs in addition to a compound of formulas I, II, III or IV may be employed. Accordingly, the pharmaceutical compositions of the present invention include those that also contain one or more other active ingredients, in addition to a compound of formulas I, II, III or IV.

The term “treating” means to relieve, alleviate, delay, reduce, reverse, improve, manage or prevent at least one symptom of a condition in a subject. The term “treating” may also mean to arrest, delay the onset (i.e., the period prior to clinical manifestation of a disease) and/or reduce the risk of developing or worsening a condition.

An “effective amount” means the amount of a compound of formulas I, II, III or IV, or a combination or mixture thereof, that, when administered to a patient (e.g., a mammal) for treating a disease, is sufficient to effect such treatment for the disease to achieve the objectives of the invention. The “effective amount” will vary depending on the compound, the disease and its severity and the age, weight, etc., of the patient to be treated.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, such as up to 10%, for example, up to 5%.

### EXAMPLES

The present invention will now be further described by way of the following non-limiting examples. In applying the disclosure of these examples, it should be kept clearly in mind that other and different embodiments of the methods and synthetic schemes disclosed according to the present invention will no doubt suggest themselves to those of skill in the relevant art.

In the foregoing and in the following examples, all temperatures are set forth uncorrected in degrees Celsius; and, unless otherwise indicated, all parts and percentages are by weight.

The following abbreviations may be used herein: Ac (CH₃CO), Bn (benzyl), DCM (dichloromethane), DMF (dimethylformamide), DIPEA (N,N-diisopropyl ethyl amine), EDCI (1-ethyl-3-[3-dimethylaminopropyl]carbodiimide hydrochloride), Et (ethyl), HOBT (1-hydroxybenzotriazole), Me (methyl), TFA (trifluoroacetic acid), THF (tetrahydrofuran), EtOH (ethanol), EtOAc (ethyl acetate), MeOH (methanol), K₂CO₃ (potassium carbonate), Pd/C (palladium on carbon), Boc (tert-butoxycarbonyl), Na₂SO₄ (sodium sulphate), CHCl₃ (chloroform), NaOH (sodium hydroxide), DMAP (dimethyl amino pyridine), NMR (nuclear magnetic resonance), DMSO-d₆ (deuterated dimethyl sulfoxide), CDCl₃ (deuterated chloroform), LC-MS (liquid chromatography-mass spectrometry), LDA (lithium diisopropylamine), HPLC (high pressure liquid chromatography or high performance liquid chromatography), Me₂NH (dimethyalamine), RT (room temperature), NaH (sodium hydride), tBuOK (potassiume tert-butoxide), NH₄Cl (ammonium chloride), LiOH (lithium hydroxide), H₂O₂ (hydrogen peroxide), NaHCO₃ (sodium hydrogen carbonate), DMS (dimethyl sulfoxide), GCMS (gas chromatography-mass spectrometry), Si-gel (silica gel), DIAD (diisopropyl azido dicarboxylate), LiAlH₄ (lithium aluminum hydride), Cs₂CO₃ (caesium carbonate), Na₂S₂O₄ (sodium hydrosulfite).

### Example 1

### (5-Chloro-2-{2-[(2R,5S)-2-(4-fluoro-benzyl)-5-methyl-morpholin-4-yl]-2-oxo-ethoxy}-phenyl)-urea

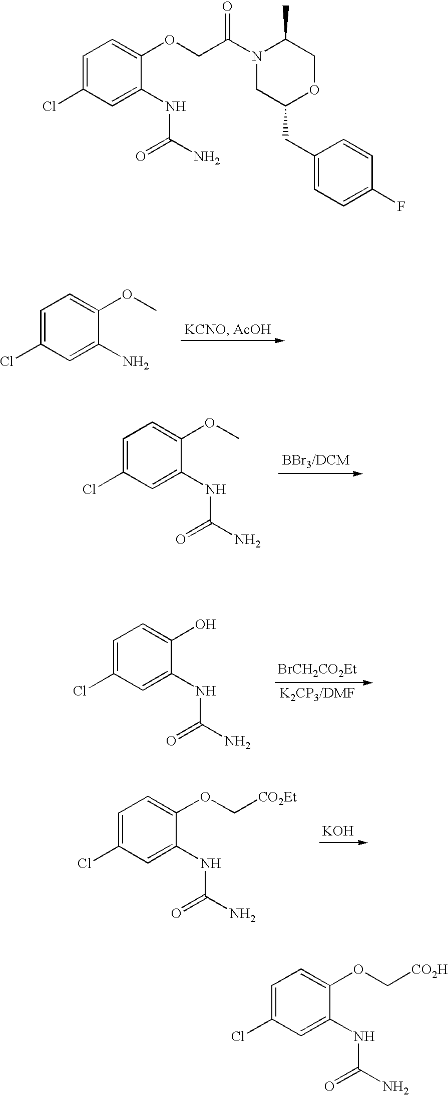

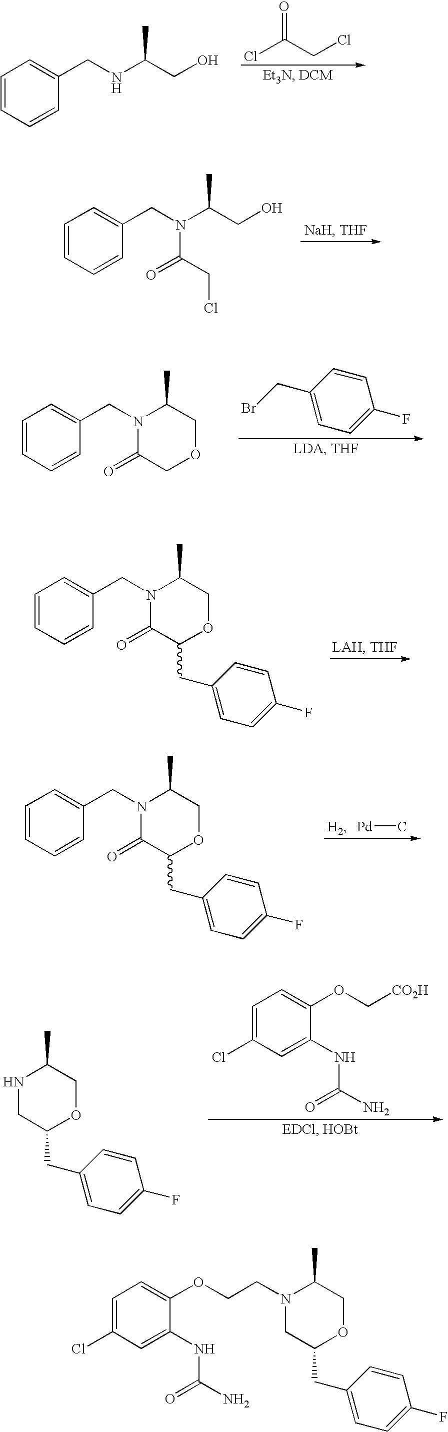

### Step 1: (5-Chloro-2-methoxy-phenyl)-urea

To a solution of 5-chloro-2-methoxyaniline (20 g, 0.13 mol) in DMF was added acetic acid (17.4 ml, 0.3 mol) and potassium isocyanate (22.6 g, 0.28 mol). The solution was stirred overnight with water (3 eqv.) at room temperature. The solution was diluted with ice-water. The precipitated solid was filtered and dried. The crude solid was washed with hexane to afford 8.2 g (32%) of (5-Chloro-2-methoxy-phenyl)-urea. LC/MS [M+H]⁺: 201.2. ¹H-NMR (400 MHz, DMSO-d₆) δ (ppm): 8.20 (d, 1H), 8.09 (s, 1H), 6.96 (d, J=2.5 Hz, 1H), 6.89 (dd, J=8.7, 2.5 Hz, 1H), 6.32 (s, 2H), 3.83 (s, 3H)

### Step-2: (5-Chloro-2-hydroxy-phenyl)-urea

To a solution of (5-Chloro-2-methoxy-phenyl)-urea (9 g, 44.9 mmol) in DCM was added BBr₃ (8.5 ml, 89.7 mmol) at 0° C. The solution was stirred for 4 h at room temperature. The solution was diluted with water. The precipitated solid was filtered and dried. The crude solid was washed with hexane to afford 3.7 g (96%) of (5-Chloro-2-hydroxy-phenyl)-urea. LC/MS [M+H]⁺: 187.1. ¹H-NMR (400 MHz, DMSO-d₆) δ (ppm): 10.09 (s, 1H), 8.06 (d, J=2.0 Hz, 1H), 8.04 (s, 1H), 6.53 (m, 2H), 6.29 (s, 2H)

### Step 3: Methyl-(4-Chloro-2-ureido-phenoxy)-acetate

To a solution of 4-chloro-2-isoxazol-5-yl-phenol (3 g, 16 mmol) in DMF was added potassium carbonate (6.63 g, 48 mmol) and methyl bromoacetate (1.6 ml, 17.7 mmol) at 0° C. The solution was stirred overnight at room temperature. The DMF solution was diluted with ice-water and extracted with ethyl acetate. The organic layer was concentrated to afford 4 g (72%) of methyl-(4-Chloro-2-ureido-phenoxy)-acetate. LC/MS [M+H]⁺: 259.1. ¹H-NMR (400 MHz, DMSO-d₆) δ (ppm): 8.2 (s, 1H), 8.17 (s, 1H), 6.90-6.80 (m, 2H), 6.36 (s, 2H), 4.89 (s, 2H), 3.70 (s, 3H)

### Step-4: (4-Chloro-2-ureido-phenoxy)-acetic acid

To a solution of methyl-(4-Chloro-2-ureido-phenoxy)-acetate (4.15 g, 15 9 mmol) in THF-water (4:1) was added LiOH (660 mg, 15.9 mmol) at 0° C. and stirred for 4 h at room temperature. The reaction mixture was concentrated and dissolved in a minimum amount of water. The pH of the solution was adjusted to 2 with 1(N)HCl, extracted with ethyl acetate and the organic phase was concentrated to afford 3.4 g (78%) of (4-chloro-2-ureido-phenoxy)-acetic acid. LC/MS [M+H]⁺: 245. ¹H-NMR (400 MHz, DMSO-d₆) δ (ppm): 13.1 (brs, 1H), 8.2 (s, 1H), 8.12 (s, 1H), 6.86 (s, 2H), 6.35 (s, 2H), 4.6 (s, 2H)

### Step-5: N-Benzyl-2-chloro-N-((S)-2-hydroxy-1-methyl-ethyl)-acetamide

To a solution of (S)-2-Benzylamino-propan-1-ol (4.1 g, 0.025 mol) in DCM was added triethylamine (3.8 ml, 0.028 mol) and chloroacetyl chloride (1.97 ml, 0.025 mol) at 0° C. The solution was stirred for 2 h at room temperature. The reaction mixture was concentrated and extracted with ethyl acetate. The organic layer was dried over sodium sulfate and concentrated to afford 560 mg (95%) of N-Benzyl-2-chloro-N-((S)-2-hydroxy-1-methyl-ethyl)-acetamide. LC/MS [M+H]⁺: 242.2

### Step 6: (S)-4-Benzyl-5-methyl-morpholin-3-one

To solution of N-Benzyl-2-chloro-N4S)-2-hydroxy-1-methyl-ethyl)-acetamide (4 g, 0.016 mol) in THF was added sodium hydride (0.52 g, 0.02 mol) portion wise at 0° C. The suspended solution was stirred overnight. Excess sodium hydride was quenched with saturated NH₄Cl solution and extracted with ethyl acetate. The organic layer was washed with water and brine solution. The combined organic phase was dried over sodium sulfate, concentrated under reduced pressure and purified through column chromatography (Si-gel, 10% ethyl acetate-hexane) to afford 2.3 g (68%) of (S)-4-Benzyl-5-methyl-morpholin-3-one. LC/MS [M+H]⁺: 206.3

### Step-7: (S)-4-Benzyl-2-(4-fluoro-benzyl)-5-methyl-morpholin-3-one

To a solution of (S)-4-Benzyl-5-methyl-morpholin-3-one (3.68 g, 0.018 mol) in THF was added LDA solution (0.043 mol) at −78° C. The solution was stirred for 1 h at −78° C. followed by addition of 4-fluorobenzyl bromide (3.4 g, 0.018 mol). The temperature of the solution was raised from −78° C. to room temperature over a period of 4 hours. The reaction mixture was quenched with saturated NH₄Cl solution and extracted with ethyl acetate. The organic layer was washed with water and brine solution. The organic layer was concentrated in vacuum and purified through column chromatography (Si-gel, 50% DCM-hexane) to afford 5.6 g (100%) of (S)-4-Benzyl-2-(4-fluoro-benzyl)-5-methyl-morpholin-3-one. LC/MS [M+H]⁺: 314.2+22 (Na)

### Step-8: (S)-4-Benzyl-2-(4-fluoro-benzyl)-5-methyl-morpholine

To a solution of (S)-4-Benzyl-2-(4-fluoro-benzyl)-5-methyl-morpholin-3-one (4.5 g, 0.014 mol) in THF was added lithium aluminum hydride (1.88 g, 0.49 mol) portion wise at 0° C. The suspended solution was refluxed for 2 hour and excess LiAlH₄ was quenched with aq. NaOH solution at 0° C. The precipitated solid was filtered off through celite bed. The organic layer was concentrated and purified through Si-gel (230-400) column (3% ethyl acetate-hexane) to afford 1.09 g (25.3%) of (2R,5S)-4-Benzyl-2-(4-fluoro-benzyl)-5-methyl-morpholine and 1.28 g (30%) of (2S,5S)-4-Benzyl-2-(4-fluoro-benzyl)-5-methyl-morpholine. LC/MS [M+H]⁺: 300.4

### Step-9: (2R,5S)-2-(4-Fluoro-benzyl)-5-methyl-morpholine

(2R,5S)-4-Benzyl-2-(4-fluoro-benzyl)-5-methyl-morpholine (100 mg, 0.33 mmol) was hydrogenated with 10% Pd—C (15 mg) in ethanol overnight. The catalyst was removed by filtration through a celite bed. The reaction mixture was concentrated to afford 70 mg (100%) of (2R,5S)-2-(4-Fluoro-benzyl)-5-methyl-morpholine. LC/MS [M+H]⁺: 210.2

### Step-10: (5-Chloro-2-{2-[(2R,5S)-2-(4-fluoro-benzyl)-5-methyl-morpholin-4-yl]-2-oxo-ethoxy}-phenyl)-urea

To a solution of (4-chloro-2-ureido-phenoxy)-acetic acid (210 mg, 0.86 mmol) in DCM was added of (2R,5S)-2-(4-Fluoro-benzyl)-5-methyl-morpholine (180 mg, 0.86 mmol), EDCI (214 mg, 1.1 mmol), HOBt (58 mg, 0.43 mmol) and DIPEA (0.4 ml, 2.6 mmol) at 5-10° C. The reaction mixture was stirred at room temperature for 16 hours. The reaction mixture was diluted with water and extracted with ethyl acetate. The organic layer was concentrated in vacuum and purified through column chromatography (Si-gel, 1-2% MeOH-DCM) to afford 70 mg (19%) of (5-Chloro-2-{2-[(2R,5S)-2-(4-fluoro-benzyl)-5-methyl-morpholin-4-yl]-2-oxo-ethoxy}-phenyl)-urea. LC/MS [M+H]⁺: 458.2 (436+Na). ¹H-NMR (400 MHz, DMSO-d₆) δ (ppm): 8.17 (s, 1H, 8.00 (s, 1H), 7.29-6.83 (m, 6H), 6.08 (s, 2H), 4.85 (br s, 2H), 3.64 (m, 1H), 3.55 (br s, 2H), 2.78 (m, 2H), 1.26 (br s, 3H). HPLC purity: 99.4%

### Example 2

### (5-Chloro-2-{2-[(R)-2-(4-fluoro-phenoxymethyl)-5-methyl-morpholin-4-yl]-2-oxo-ethoxy}-phenyl)-urea

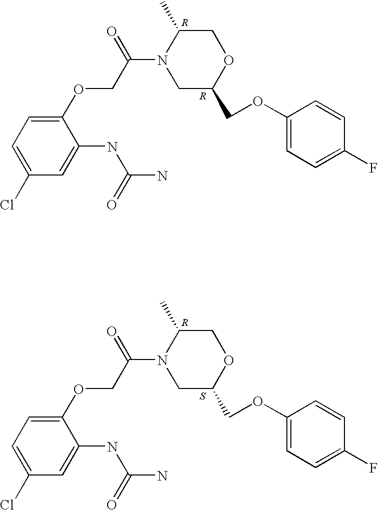

Diastereomeric mixture of (5-Chloro-2-{2-[(2R,5R)-2-(4-fluoro-phenoxymethyl)-5-methyl-morpholin-4-yl]-2-oxo-ethoxy}-phenyl)-urea and (5-Chloro-2-{2-[(2S,5R)-2-(4-fluoro-phenoxymethyl)-5-methyl-morpholin-4-yl]-2-oxo-ethoxy}-phenyl)-urea

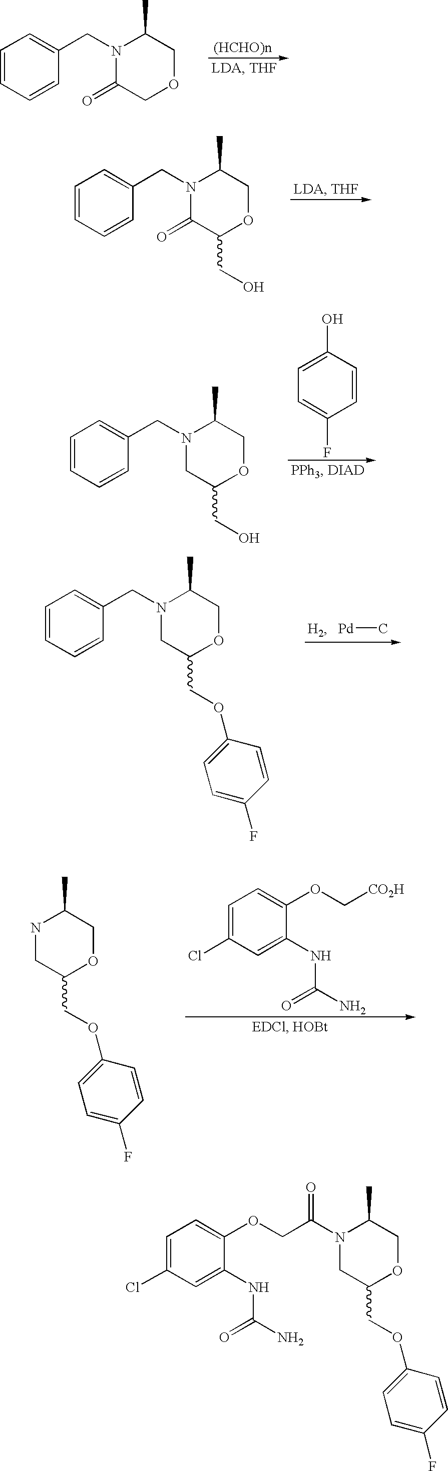

(4-chloro-2-ureido-phenoxy)-acetic acid and (S)-4-Benzyl-5-methyl-morpholin-3-one were prepared as described in Example 1.

### Step-1: (S)-4-Benzyl-2-hydroxymethyl-5-methyl-morpholin-3-one

To a solution of (S)-4-Benzyl-5-methyl-morpholin-3-one (1 g, 4.87 mmol) in THF was added LDA solution (6.8 mmol) at −78° C. The solution was stirred for 1 hour at −78° C. followed by addition of paraformaldehyde (1.5 g). The temperature of the solution was raised from −78° C. to room temperature over a period of 4 hours. The reaction mixture was quenched with saturated NH₄Cl solution and extracted with ethyl acetate. The organic layer was washed with water and brine solution. The organic phase was concentrated in vacuum and purified through column chromatography (Si-gel, 1% MeOH-DCM) to afford 400 mg (35%) (S)-4-Benzyl-2-hydroxymethyl-5-methyl-morpholin-3-one. GCMS [m/z]: 235

### Step-2: ((S)-4-Benzyl-5-methyl-morpholin-2-yl)-methanol

To a solution of (S)-4-Benzyl-2-hydroxymethyl-5-methyl-morpholin-3-one (400 mg, 1.7 mmol) in THF was added LiAlH₄ (129 mg, 3.4 mmol) portion wise at 0° C. The suspended solution was stirred at room temperature overnight. Excess LiALH₄ was quenched with aq. NaOH solution at 0° C. The precipitated solid was filtered off through celite bed. The organic layer was concentrated and purified through column chromatography (Si-gel, 1% MeOH-DCM) to afford 180 mg (48%) of ((S)-4-Benzyl-5-methyl-morpholin-2-yl)-methanol. GCMS [m/z]: 221

### Step-3: (S)-4-Benzyl-2-(4-fluoro-phenoxymethyl)-5-methyl-morpholine

To a solution of triphenylphosphine (243.1 mg, 0.93 mmol) in THF was added DIAD (0.18 ml, 0.93 mmol) at 0° C. and the mixture was stirred at room temperature 1 hour. To the reaction mixture was added a solution of ((S)-4-Benzyl-5-methyl-morpholin-2-yl)-methanol (180 mg, 0.82 mmol) and 4-fluorophenol (103.9 mg, 0.82 mmol) at 0° C. The reaction mixture was stirred at room temperature for 12 hours. The mixture was concentrated, diluted with water and extracted with ethyl acetate. The organic layer was dried over sodium sulfate and concentrated. The crude reaction mass was purified through column chromatography (Si-gel, 1% MeOH-DCM) to afford 240 mg (94%) of (S)-4-Benzyl-2-(4-fluoro-phenoxymethyl)-5-methyl-morpholine. LC/MS [M+H]⁺: 316.4

### Step-4: (S)-2-(4-Fluoro-phenoxymethyl)-5-methyl-morpholine

(S)-4-Benzyl-2-(4-fluoro-phenoxymethyl)-5-methyl-morpholine (240 mg, 0.76 mmol) was stirred with 10% Pd—C (15 mg) under an atmosphere of hydrogen in ethanol overnight. The catalyst was removed by filtration through celite bed. Concentration of the ethanol solution and crystallization afforded 50 mg (29%) of (S)-2-(4-Fluoro-phenoxymethyl)-5-methyl-morpholine. LC/MS [M+H]⁺: 226.1

### Step-5: (5-Chloro-2-{2-[(S)-2-(4-fluoro-phenoxymethyl)-5-methyl-morpholin-4-yl]-2-oxo-ethoxy}-phenyl)-urea

To a solution of (4-chloro-2-ureido-phenoxy)-acetic acid (54.3 mg, 0.22 mmol) in dichloromethane was added of (S)-2-(4-Fluoro-phenoxymethyl)-5-methyl-morpholine (50 mg, 0.22 mmol), EDCI (55.3 mg, 0.29 mmol), HOBt (15 mg, 0.11 mmol) and DIPEA (0.11 ml, 0.66 mmol) at 5-10° C. The reaction mixture was stirred at room temperature for 16 hours. The reaction mixture was diluted with water and extracted with ethyl acetate. The organic layer was concentrated in vacuum and purified through column chromatography (Si-gel, 1-2% MeOH-DCM) to afford 30 mg (15%) of (5-Chloro-2-{2-[(S)-2-(4-fluoro-phenoxymethyl)-5-methyl-morpholin-4-yl]-2-oxo-ethoxy}-phenyl)-urea. LC/MS [M+H]⁺: 452.4. ¹H-NMR (400 MHz, CDCl₃) δ (ppm): 8.97 (s, 1H), 8.27 (s, 1H), 7.01-6.81 (m, 8H), 4.82-3.34 9m, 10H), 1.31 (d, 3H). HPLC purity: 88.4%

### Example 3

### [5-Chloro-2-(2-{(S)-2-[2-(4-fluoro-phenyl)-ethyl]-5-methyl-morpholin-4-yl}-2-oxo-ethoxy)-phenyl]-urea

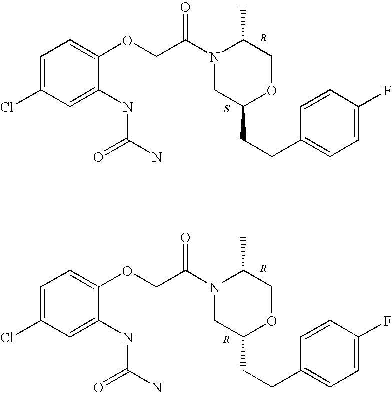

Diastereomeric mixture of [5-Chloro-2-(2-{(2S,5R)-2-[2-(4-fluoro-phenyl)-ethyl]-5-methyl-morpholin-4-yl}-2-oxo-ethoxy)-phenyl]-urea and [5-Chloro-2-(2-{(2R,5R)-2-[2-(4-fluoro-phenyl)-ethyl]-5-methyl-morpholin-4-yl}-2-oxo-ethoxy)-phenyl]-urea

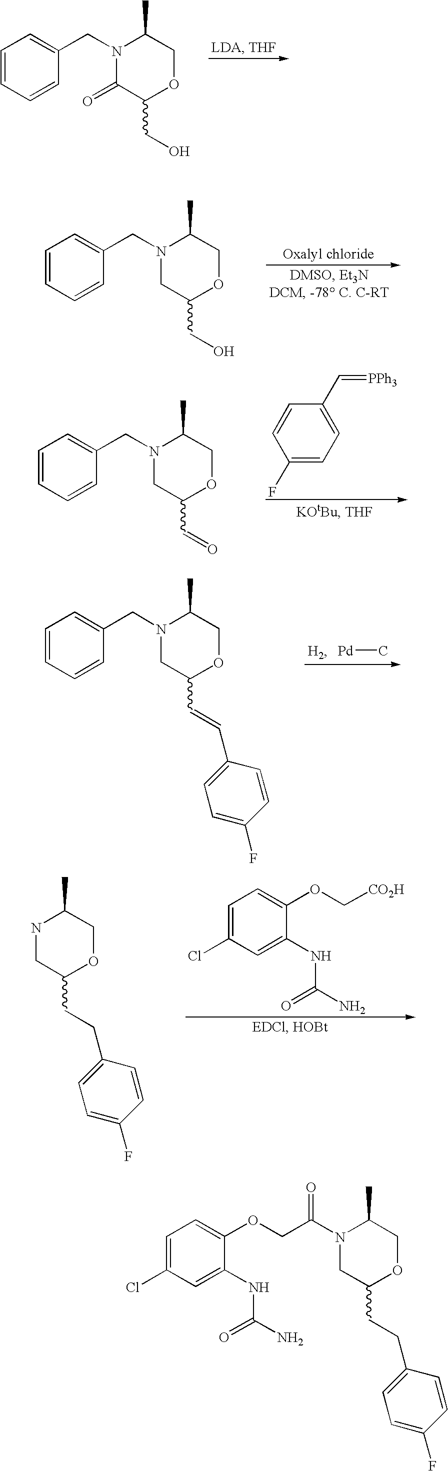

(S)-4-Benzyl-2-hydroxymethyl-5-methyl-morpholin-3-one was prepared by a process as described in example 2.

### Step-1: ((S)-4-Benzyl-5-methyl-morpholin-2-yl)-methanol

To a solution of (S)-4-Benzyl-2-hydroxymethyl-5-methyl-morpholin-3-one (400 mg, 1.7 mmol) in THF was added lithium aluminum hydride (129 mg, 3.4 mmol) portion wise at 0° C. The suspended solution was stirred at room temperature overnight. Excess LiALH₄ was quenched with aq. NaOH solution at 0° C. The reaction mixture was filtered through a celite bed. The organic layer was concentrated and purified through column chromatography (Si-gel, 1% MeOH-DCM) to afford 180 mg (48%) of ((S)-4-Benzyl-5-methyl-morpholin-2-yl)-methanol. GCMS [m/z]: 221

### Step-2: (S)-4-Benzyl-5-methyl-morpholine-2-carbaldehyde

A solution of DMSO (0.24 ml, 3.39 mmol) in anhydrous DCM (5 ml) was added drop wise to a stirred solution of oxalyl chloride (0.13 ml, 1.49 mmol) under an atmosphere of nitrogen at −72° C. and the reaction mixture was stirred for 30 min. A solution of ((S)-4-Benzyl-5-methyl-morpholin-2-yl)-methanol. (300 mg, 1.36 mmol) in DCM (5 ml) was drop wise added to the reaction mixture over a period of 10 min and stirring was continued for 1 hour. To this was added triethyl amine (0.95 ml, 6.78 mmol) and stirring was continued for 5 min at −78° C. followed by stirring at RT for 30 min. The reaction mixture was diluted with water and extracted with dichloromethane. The combined organics were washed with brine, water, 5% sodium bicarbonate, dried and concentrated. The crude mass was purified on silica gel (60-120) using 5% MeOH-DCM as an eluent to afford 250 g (84%) of (S)-4-Benzyl-5-methyl-morpholine-2-carbaldehyde. GCMS [m/z]: 219

### Step-3: (S)-4-Benzyl-2-[(E)-2-(4-fluoro-phenyl)-vinyl]-5-methyl-morpholine

To a solution of 4-fluorobenzyl-triphenylphosphonium bromide (495.1 mg, 1.33 mmol) in THF was added tBuOK (149.3 mg, 1.33 mmol) at 0° C. and the mixture was stirred for 1 hour at room temperature. A solution of (S)-4-Benzyl-5-methyl-morpholine-2-carbaldehyde (265 mg, 1.2 mmol) was added to at 0° C. and the mixture stirred at room temperature for 5 hours. The reaction mixture was then concentrated, diluted with water and extracted with dichloromethane. The organic layer was concentrated and purified over Si-gel (3% ethyl acetate-hexane) to afford 125 mg (33%) of (S)-4-Benzyl-2-[(E)-2-(4-fluoro-phenyl)-vinyl]-5-methyl-morpholine. LC/MS [M+H]⁺: 312.1

### Step-4: (S)-2-[2-(4-Fluoro-phenyl)-ethyl]-5-methyl-morpholine

A solution of (S)-4-Benzyl-2-[(E)-2-(4-fluoro-phenyl)-vinyl]-5-methyl-morpholine (120 mg, 0.4 mmol) in ethanol was stirred with 10% Pd—C (18 mg) under an atmosphere of hydrogen overnight. The catalyst was removed by filtration through a celite bed. Concentration of the ethanol solution afforded 50 mg (56%) of (S)-2-[2-(4-Fluoro-phenyl)-ethyl]-5-methyl-morpholine. LC/MS [M+H]⁺: 224.2

### Step-5: [5-Chloro-2-(2-{(S)-2-[2-(4-fluoro-phenyl)-ethyl]-5-methyl-morpholin-4-yl}-2-oxo-ethoxy)-phenyl]-urea

To a solution of (4-chloro-2-ureido-phenoxy)-acetic acid (49.3 mg, 0.2 mmol) in dichloromethane was added of (S)-2-[2-(4-Fluoro-phenyl)-ethyl]-5-methyl-morpholine (45 mg, 0.2 mmol), EDCI (50 mg, 0.26 mmol), HOBt (14 mg, 0.1 mmol) and DIPEA (0.1 ml, 0.6 mmol) at 5-10° C. The reaction mixture was stirred at room temperature for 16 hours. The reaction mixture was diluted with water and extracted with ethyl acetate. The organic layer was concentrated in vacuum and purified through column chromatography (Si-gel, 1% MeOH-DCM) to afford 20 mg (22%) of [5-Chloro-2-(2-{(S)-2-[2-(4-fluoro-phenyl)-ethyl]-5-methyl-morpholin-4-yl}-2-oxo-ethoxy)-phenyl]-urea. LC/MS [M+H]⁺: 450.3. ¹H-NMR (400 MHz, CDCl₃) δ (ppm): 8.96 9s, 1H), 8.26 (s, 1H), 7.28-6.75 (m, 8H), 4.80-2.61 (m, 12H), 1.32 (d, 3H). HPLC purity: 85%

### Example 4

### (5-Chloro-2-{3-[(2R,5S)-2-(4-fluoro-benzyl)-5-methyl-morpholin-4-yl]-3-oxo-propoxy}-phenyl)-urea

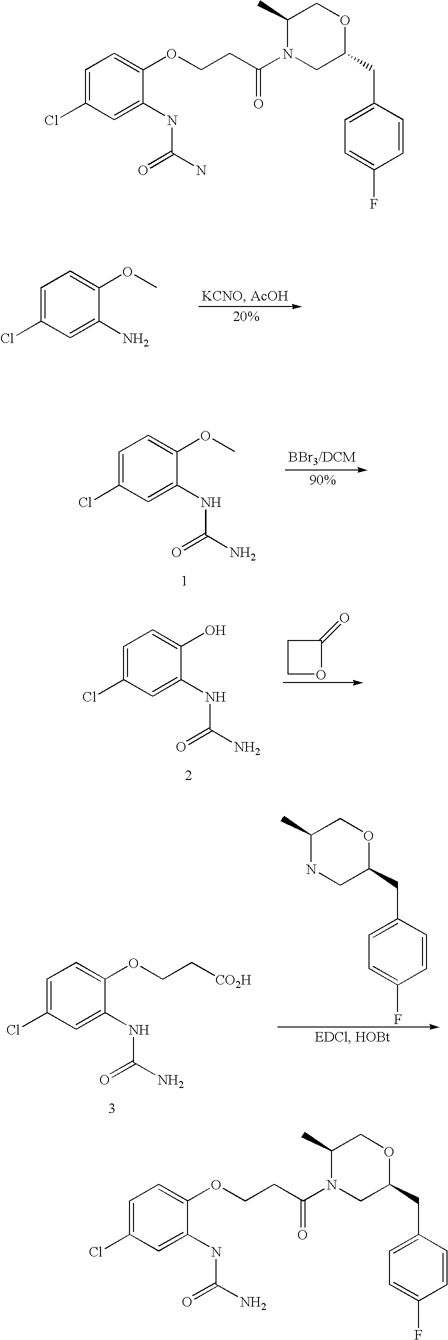

(2R,5S)-2-(4-Fluoro-benzyl)-5-methyl-morpholine was prepared by a process as described in example 1.

### Step 1: (5-Chloro-2-methoxy-phenyl)-urea

To a solution of 5-chloro-2-methoxyaniline (20 g, 0.13 mol) in DMF and was added acetic acid (17.4 ml, 0.3 mol) and potassium isocyanate (22.6 g, 0.28 mol)). The solution was stirred overnight with water (3 eqv.) at room temperature. The solution was diluted with iced water. The precipitated solid was filtered. The crude mass was washed with hexane to afford 8.2 g (32%) of (5-Chloro-2-methoxy-phenyl)-urea. LC/MS [M+H]⁺: 201.2. ¹H-NMR (400 MHz, DMSO-d₆) δ (ppm): 8.20 (d, 1H), 8.09 (s, 1H), 6.96 (d, J=2.5 Hz, 1H), 6.89 (dd, J=8.7, 2.5 Hz, 1H), 6.32 (s, 2H), 3.83 (s, 3H)

### Step 2: (5-Chloro-2-hydroxy-phenyl)-urea

To a solution of (5-Chloro-2-methoxy-phenyl)-urea (9 g, 44.9 mmol) in DCM at 0° C. was added BBr₃ (8.5 ml, 89.7 mmol). The solution was stirred for 4 hours, allowing the temperature to rise from 0° C. to room temperature. The solution was then diluted with water. The precipitated solid was filtered. The crude mass was dried and washed with hexane to afford 3.7 g (96%) of (5-Chloro-2-hydroxy-phenyl)-urea. LC/MS [M+H]⁺: 187.1. ¹H-NMR (400 MHz, DMSO-d₆) δ (ppm): 10.09 (s, 1H), 8.06 (d, J=2.0 Hz, 1H), 8.04 (s, 1H), 6.53 (m, 2H), 6.29 (s, 2H).

### Step-3: (5-Chloro-2-oxo-phenyl)-urea-propionic acid

To a solution of (5-Chloro-2-hydroxy-phenyl)-urea (3 g, 16.1 mmol) in THF at 0° C. was added tBuOK (1.9 g, 16.8 mmol) portion wise. The solution was stirred for half an hour at room temperature. The solution was cooled to 0° C. and propiolactone (1.1 ml, 16.9 mmol) was added drop wise. The solution was then warmed at 60° C. for 2 days. After 2 days, the reaction mixture was concentrated and diluted with water. The excess starting material was removed by ethyl acetate extraction. The aqueous solution was adjusted to pH 2 with 1(N)HCl. The precipitated solid was filtered and dried to afford 2.1 g (51%) of (5-Chloro-2-oxo-phenyl)-urea-propionic acid. LC/MS [M-FH]⁺: 259.0. ¹H-NMR (400 MHz, DMSO-d₆) δ (ppm): 12.42 (s, 1H), 8.19 (d, J=2.5 Hz, 1H), 7.89 (s, 1H), 7.09-6.86 (m, 3H), 6.37 (s, 1H), 4.20 (t, J=6.0 Hz, 2H), 2.77 (t, J=6.1 Hz, 2H)

### Step-4: (5-Chloro-2-{3-[(2R,5S)-2-(4-fluoro-benzyl)-5-methyl-morpholin-4-yl]-3-oxo-propoxy}-phenyl)-urea

To a solution of (5-Chloro-2-oxo-phenyl)-urea-propionic acid (100 mg, 0.39 mmol) in DMF was added (2R,5S)-2-(4-Fluoro-benzyl)-5-methyl-morpholine (80.9 mg, 0.39 mmol), EDCI (97.2 mg, 0.51 mmol), HOBt (26.3 mg, 0.19 mmol) and DIPEA (0.19 ml, 1.17 mmol) at 5-10° C. The reaction mixture was stirred at room temperature for 16 hours. The mixture was then diluted with water and extracted with ethyl acetate. The organic layer was concentrated in vacuum and purified through column chromatography (Si-gel, 1-2% MeOH-DCM) to afford 50 mg (29%) of (5-Chloro-2-{3-[(2R,5S)-2-(4-fluoro-benzyl)-5-methyl-morpholin-4-yl]-3-oxo-propoxy}-phenyl)-urea. LC/MS [M+H]⁺: 450.2. ¹H-NMR (400 MHz, CDCl₃) δ (ppm): 9.28 (m, 1H), 8.38 (s, 1H), 7.20-6.8 (m, 8H), 5.06-2.63 (m, 12H), 1.23 (m, 3H). HPLC purity: 95.04%

### Example 5

### (5-Chloro-2-{3-[(2S,5S)-2-(4-fluoro-benzyl)-5-methyl-morpholin-4-yl]-3-oxo-propoxy}-phenyl)-urea

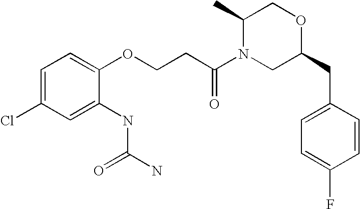

(5-Chloro-2-{3-[(2S,5S)-2-(4-fluoro-benzyl)-5-methyl-morpholin-4-yl]-3-oxo-propoxy}-phenyl)-urea was prepared by a procedure similar to that described in Example 4. LC/MS [M+H]⁺: 450.3. ¹H-NMR (400 MHz, DMSO-d₆) δ (ppm): 9.25 br s, 1H), 8.4 (s, 1H), 7.18-6.82 (m, 8H), 5.03-2.71 (m, 12H), 1.38 (br s, 3H). HPLC purity: 93.6%

### Example 6

### (5-Chloro-2-{3-[2-(4-fluoro-benzyl)-[1,4]oxazepan-4-yl]-3-oxo-propoxy}-phenyl)-urea

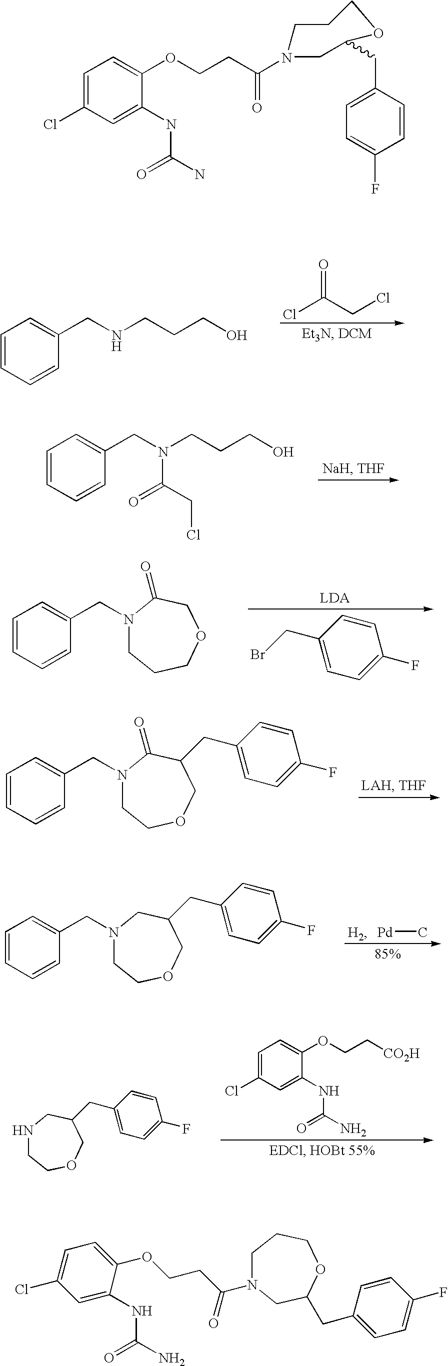

(5-Chloro-2-oxo-phenyl)-urea-propionic acid was prepared as described in Example 4.

### Step-1: N-Benzyl-2-chloro-N-(3-hydroxy-propyl)-acetamide

To a solution of 3-Benzylamino-propan-1-ol (28 g, 0.17 mol) in DCM was added triethylamine (25.9 ml, 0.19 mol) and chloroacetyl chloride (13.4 ml, 0.17 mol) at 0° C. The solution was stirred for 2 h at room temperature. The reaction mixture was concentrated and extracted with ethyl acetate. The organic layer was dried over sodium sulfate and concentrated to afford 41 g (93%) of N-Benzyl-2-chloro-N-(3-hydroxy-propyl)-acetamide. LC/MS [M+H]⁺: 242.3

### Step-2: 4-Benzyl-[1,4]oxazepan-3-one

To a solution of N-Benzyl-2-chloro-N-(3-hydroxy-propyl)-acetamide (30 g, 0.12 mol) in THF was added sodium hydride (6.25 g, 0.13 mol) portion wise at 0° C. The suspended solution was stirred overnight at room temperature. Excess sodium hydride was quenched with saturated NH₄Cl solution and the product extracted with ethyl acetate. The organic layer was washed with water and brine solution. The combined organic layers were concentrated under reduced pressure and purified through column chromatography (Si-gel, 1-2% MeOH-DCM) to afford 16 g (68%) of 4-Benzyl-[1,4]oxazepan-3-one. LC/MS [M+H]⁺: 206.2. ¹H-NMR (400 MHz, DMSO-d₆) δ (ppm): 7.35-7.24 (m, 5H), 4.51 (s, 2H), 4.19 (s, 2H), 3.73 (t, J=5.6 Hz, 2H), 3.43 (t, J=4.9 Hz, 2H), 1.69 (m, 2H)

### Step-3: 4-Benzyl-2-(4-fluoro-benzyl)-[1,4]oxazepan-3-one

To a solution of 4-benzyl-[1,4]oxazepan-3-one (1 g, 4.9 mmol) in THF was added LDA solution (5.9 mmol) at −78° C. The solution was stirred for 1 hour at −78° C., then a solution of 4-fluorobenzyl bromide (920 mg, 4.9 mmol) in THF was added to the reaction mixture. The temperature of the solution was raised from −78° C. to room temperature over a period of 4 hours. The reaction mixture was quenched with saturated NH₄Cl solution and extracted with ethyl acetate. The organic layer was washed with water and brine solution. The organic layer was concentrated in vacuum and purified through column chromatography (Si-gel, 5-10% ethyl acetate-hexane) to afford 1.1 g (49%) of 4-benzyl-2-(4-fluoro-benzyl)-[1,4]oxazepan-3-one. LC/MS [M+H]⁺: 314.2. ¹H-NMR (400 MHz, DMSO-d₆) δ (ppm): 7.35-7.06 (9 Ar H), 4.59-4.41 (m, 3H), 3.92 (m, 1H), 3.69 (m, 1H), 3.41 (m, 1H), 3.3 (m, 1H), 3.04 (dd, J=14.5, 3.7 Hz, 1H), 2.80 (m, 1H), 1.53 (s, 2H).

### Step-4: 4-Benzyl-2-(4-fluoro-benzyl)-[1,4]oxazepane

To a solution of 4-benzyl-2-(4-fluoro-benzyl)-[1,4]oxazepan-3-one (3.08 g, 9.6 mmol) in THF was added LiAlH₄ (726 mg, 19.1 mmol) portion wise at 0° C. The suspended solution was heated to reflux for 2 hours, then excess LiALH₄ was quenched with aq. NaOH solution at 0° C. The precipitated solid was filtered through a celite bed. The organic layer was concentrated to afford 2.5 g (89%) of 4-Benzyl-2-(4-fluoro-benzyl)-[1,4]oxazepane. LC/MS [M+H]⁺: 300.2. ¹H-NMR (400 MHz, DMSO-d₆) δ (ppm): 7.29-7.01 (9 Ar H), 3.73 (m, 2H), 3.56 (m, 3H), 2.78-2.31 9m, 5H), 1.79-1.70 (2H).

### Step-5: 2-(4-Fluoro-benzyl)-[1,4]oxazepane

A solution of 4-Benzyl-2-(4-fluoro-benzyl)-[1,4]oxazepane (2.5 g) in ethanol was stirred with 10% Pd—C (200 mg) for 5-10 h under an atmosphere of hydrogen. The catalyst was removed by filtration through a celite bed and the ethanol solution was concentrated to afford 1.8 g (97%) of 2-(4-Fluoro-benzyl)-[1,4]oxazepane. LC/MS [M+H]⁺: 210.3. ¹H-NMR (400 MHz, DMSO-d₆) δ (ppm): 7.22-7.05 (4 Ar H), 3.75 (m, 1H), 3.54 (m, 2H), 3.88 (m, 2H), 2.63 (m, 3H), 2.41 9m, 1H), 1.68 (m, 2H).

### Step-6 (5-Chloro-2-{3-[2-(4 fluoro-benzyl)-[1,4]oxazepan-4-yl]-3-oxo-propoxy}-phenyl)-urea

To a solution of 2-(4-Fluoro-benzyl)-[1,4]oxazepane (200 mg, 0.77 mmol) in DMF was added 2-(4-Fluoro-benzyl)-[1,4]oxazepane (161 mg, 0.77 mmol), EDCI (192 mg, 1 mmol), HOBt (53 mg, 0.39 mmol) and DIPEA (0.5 ml, 2.7 mmol) at 5-10° C. The reaction mixture was stirred at room temperature for 16 hours. The reaction mixture was diluted with water and extracted with ethyl acetate. The ethyl acetate layer was concentrated in vacuum and subjected to column chromatography (Si-gel, 1-2% MeOH-DCM) to afford 200 mg (58%) of (5-Chloro-2-{3-[2-(4-fluoro-benzyl)-[1,4]oxazepan-4-yl]-3-oxo-propoxy}-phenyl)-urea. LC/MS [M+H]⁺: 450.1. ¹H-NMR (400 MHz, CDCl₃) δ (ppm): 8.2 (s, 1H), 8.12-6.86 (7 Ar H), 6.29 (s, 2H), 4.23 (m, 2H), 4.02-2.67 (m, 11H), 1.75 (br s, 2H). HPLC: 99.4%

### Example 7

### (5-Chloro-2-{2-[2-(4-fluoro-benzyl)-[1,4]oxazepan-4-yl]-2-oxo-ethoxy}-phenyl)-urea

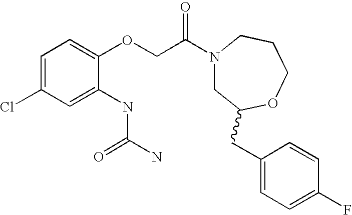

2-(4-Fluoro-benzyl)-[1,4]oxazepane was prepared as described in Example 6.

(5-Chloro-2-{2-[2-(4-fluoro-benzyl)-[1,4]oxazepan-4-yl]-2-oxo-ethoxy}-phenyl)-urea was prepared by a procedure similar to that described in Example 1. LC/MS [M+H]⁺: 436.3. ¹H-NMR (400 MHz, CDCl₃) δ (ppm): 8.90 (br s, 2H), 8.27 (s, 1H), 7.19-6.55 (m, 8H), 4.78-2.01 (13H). HPLC: 97.7%

### Example 8

### (5-Chloro-2-{2-[(2R,5S)-2-(4-fluoro-benzyl)-5-methyl-morpholin-4-yl]-ethoxy}-phenyl)-urea

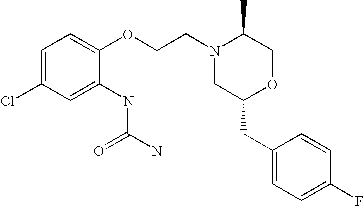

To a solution of (5-Chloro-2-{2-[(2R,5S)-2-(4-fluoro-benzyl)-5-methyl-morpholin-4-yl]-2-oxo-ethoxy}-phenyl)-urea (20 mg, 0.05 mmol) in THF was added BH₃-DMS (0.02 ml, 0.14 mmol). The solution was stirred overnight. The reaction mixture was then heated to reflux in MeOH for 1 hour. The reaction mixture was concentrated and purified through column chromatography (Si-gel, 1-2% MeOH-DCM) to afford 10 mg (52%) of (5-Chloro-2-{2-[(2R,5S)-2-(4-fluoro-benzyl)-5-methyl-morpholin-4-yl]-ethoxy}-phenyl)-urea. LC/MS [M+H]⁺: 422.3. ¹H-NMR (400 MHz, CDCl₃) δ (ppm): 8.21 (s, 1H), 8.11 (s, 1H), 7.17-6.7 (m, 8H), 5.01 (s, 2H), 4.02 (m, 2H), 3.77-3.47 (m, 4H), 2.95-2.43 (m, 4H), 1.01 (d, 3H). HPLC: 91.1%

### Example 9

### 1-[(2R,5S)-2-(4-Fluoro-benzyl)-5-methyl-morpholin-4-yl]-2-(4-fluoro-phenoxy)-ethanone

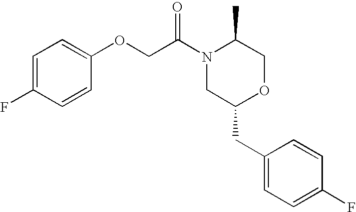

1-[(2R,5S)-2-(4-Fluoro-benzyl)-5-methyl-morpholin-4-yl]-2-(4-fluoro-phenoxy)-ethanone was prepared by a procedure similar to that described in Example 1. LC/MS [M+H]⁺: 362.3. ¹H-NMR (400 MHz, DMSO-d₆) δ (ppm): 7.18-66.71 (m, 8H), 4.64-2.69 (m, 10H), 1.22 (d, 3H). HPLC: 97.4%

### Example-10

### 2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone

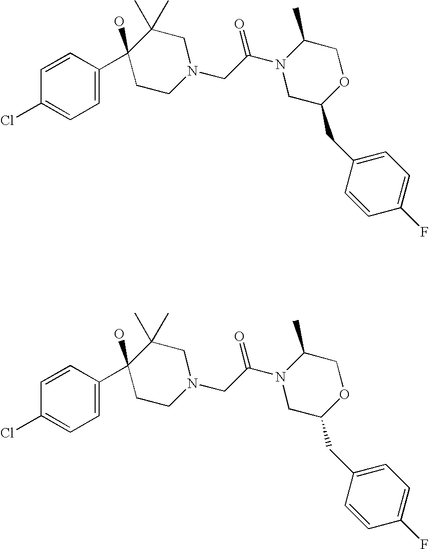

Diastereomeric mixture of 2-[(S)-4-(4-chloro-phenyl)-4-hydroxy-3,3-dimethyl-piperidin-1-yl]-1-[(2R,5S)-2-(4-fluoro-benzyl)-5-methyl-morpholin-4-yl]-ethanone and 2-[(R)-4-(4-chloro-phenyl)-4-hydroxy-3,3-dimethyl-piperidin-1-yl]-1-[(2R,5S)-2-(4-fluoro-benzyl)-5-methyl-morpholin-4-yl]-ethanone.

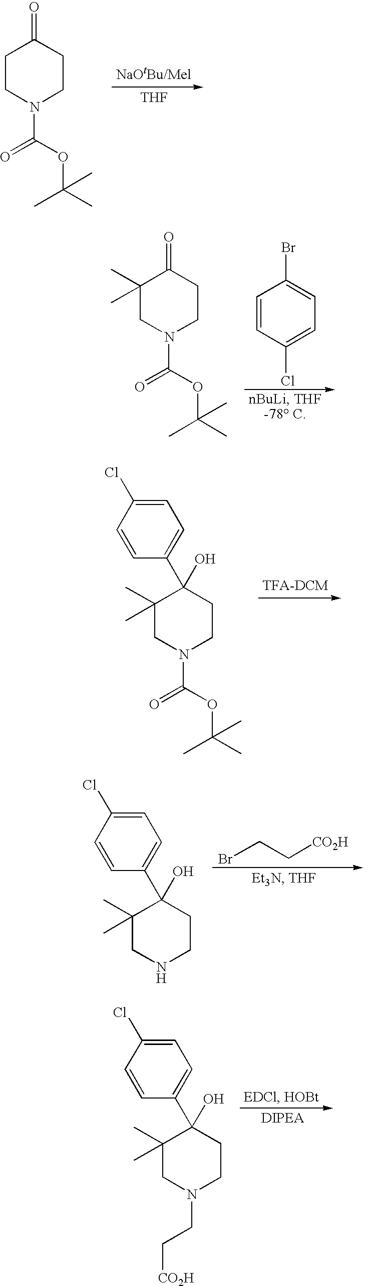

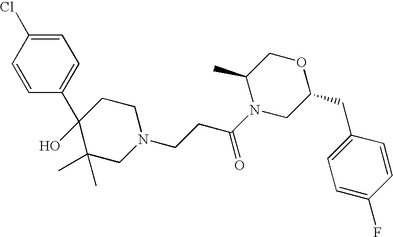

(2R,5S)-2-(4-Fluoro-benzyl)-5-methyl-morpholine was prepared by a process as described in example 1.

### Step-1: 3,3 dimethyl-4-oxo-piperidine-1-carboxylic acid tert-butyl ester

To a solution of 4-oxo-piperidine-1-carboxylic acid tert-butyl ester (18 g, 90.4 mmol) in THF was added sodium tert-butoxide (20.9 g, 217 mmol) portion wise at 0° C. The reaction mixture was stirred for 1 hour at room temperature. To the reaction mixture was added methyl iodide (11.8 ml, 189 mmol) and the mixture was heated to reflux for 2 hours. The mixture was then concentrated, diluted with NH₄Cl/water and extracted with ethyl acetate. The organic layer was concentrated and purified over Si-gel (2% ethyl acetate-hexane) to afford 7 g (34%) of 3,3 dimethyl-4-oxo-piperidine-1-carboxylic acid tert-butyl ester. ¹H-NMR (400 MHz, CDCl₃) δ (ppm): 3.73 (t, 2H), 3.43 (br s, 2H), 2.49 (t, 2H), 1.49 (s, 9H), 1.13 (s, 6H).

### Step-2: 4-(4-Chloro-phenyl)-4-hydroxy-3,3-dimethyl-piperidine-1-carboxylic acid-tert-butyl ester

To a solution of 4-bromo-chlorobenzene (21 g, 0.11 mol) in THF was added butyl lithium (2.13M in hexane, 51.6 ml, 0.11 mol) drop wise at −78° C. The reaction mixture was stirred at −78° C. for 1 hour. A solution of 3,3 dimethyl-4-oxo-piperidine-1-carboxylic acid tert-butyl ester (10 g, 0.04 mol) in THF was then added at −78° C. and the mixture was stirred for 2 hours. The mixture was quenched with saturated aqueous solution NH₄Cl and allowed to warm to room temperature. The mixture was then diluted with water and extracted with ethyl acetate. The organic layer was dried over sodium sulfate and concentrated. The crude mass was triturated with ether and hexane to afford 10 g (67%) of 4-(4-Chloro-phenyl)-4-hydroxy-3,3-dimethyl-piperidine-1-carboxylic acid-tert-butyl ester. ¹H-NMR (400 MHz, CDCl₃) δ (ppm): 7.39 (d, 2H), 7.31 (d, 2H), 4.30-4.00 9m, 1H), 3.70-3.10 (m, 3H), 2.67 (m, 1H), 1.49 (s, 9H), 1.44-1.34 (m, 2H), 0.82 (s, 6H).

### Step-3: 4-(4-Chloro-phenyl)-3,3-dimethyl-piperidin-4-ol

To a solution of 4-(4-Chloro-phenyl)-4-hydroxy-3,3-dimethyl-piperidine-1-carboxylic acid-tert-butyl ester (17.5 g, 0.052 mol) in DCM was added trifluoroacetic acid (31.6 ml, 0.41 mol) at 0° C. and the mixture was stirred for 4 hours at room temperature. The mixture was then concentrated. The TFA salt was diluted with a minimum volume of water, neutralized with aq. NaOH solution at 5-10° C. and extracted with ethyl acetate. The organic layer was dried over sodium sulfate and concentrated to afford 11 g (93%) of 4-(4-Chloro-phenyl)-3,3-dimethyl-piperidin-4-ol. LC/MS [M+H]⁺: 240

### Step-4: 3-[4-(4-Chloro-phenyl)-4-hydroxy-3,3-dimethyl-piperidin-1-yl]-propionic acid

To 4-(4-Chloro-phenyl)-3,3-dimethyl-piperidin-4-ol 1 (1 g, 4.2 mmol) in THF was added 1-bromopropionic acid (638 mg, 4.2 mmol) and triethylamine (2 ml, 14.6 mmol). The solution was stirred overnight at room temperature. The reaction mixture was concentrated to dryness and diluted with water. The mixture was then filtered and dried to afford 760 mg (36%) of 3-[4-(4-Chloro-phenyl)-4-hydroxy-3,3-dimethyl-piperidin-1-yl]-propionic acid. LC/MS [M+H]⁺: 311.0. ¹H-NMR (400 MHz, DMSO-d₆) δ (ppm): 7.48-7.34 (4 Ar H), 4.86 (s, 1H), 3.04 br. d 1H), 2.87 (br d, 1H), 2.69-2.56 (m, 4H), 2.36 (m, 3H), 0.79 (s, 3H), 0.63 (s, 3H)

### Step-5: [(2R,5S)-2-(4-fluoro-benzyl)-5-methyl-morpholin-4-yl]-propan-1-one-3-[4-(4-Chloro-phenyl)-4-hydroxy-3,3-dimethyl-piperidine]

To a solution of [3-[4-(4-Chloro-phenyl)-4-hydroxy-3,3-dimethyl-piperidin-1-yl]-propionic acid (100 mg, 0.34 mmol) in DMF was added (2R,5S)-2-(4-fluoro-benzyl)-5-methyl-morpholine (70 mg, 0.34 mmol), EDCI (84 mg, 0.44 mmol), HOBt (23 mg, 0.17 mmol) and DIPEA (0.2 ml, 1.2 mmol) at 5-10° C. The reaction mixture was stirred at room temperature for 16 hours, then concentrated and extracted with ethyl acetate. The organic layer was concentrated under reduced pressure and purified through Si-gel column chromatography (80% ethyl acetate-hexane) to afford 85 mg (52%) of 2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone LC/MS [M+H]⁺: 489.3. ¹H-NMR (400 MHz, DMSO-d₆) δ (ppm): 7.39-6.96 (m, 8H), 4.29-2.03 (m, 12H), 1.37 (m, 2H), 1.24 (s, 6H), 0.91 (m, 2H), 0.75 (d, 3H). HPLC: 99.5%

### Example 11

### [(2R,5S)-2-(4-fluoro-benzyl)-5-methyl-morpholin-4-yl]-propan-1-one-3-[4-(4-Chloro-phenyl)-4-hydroxy-3,3-dimethyl-piperidine]

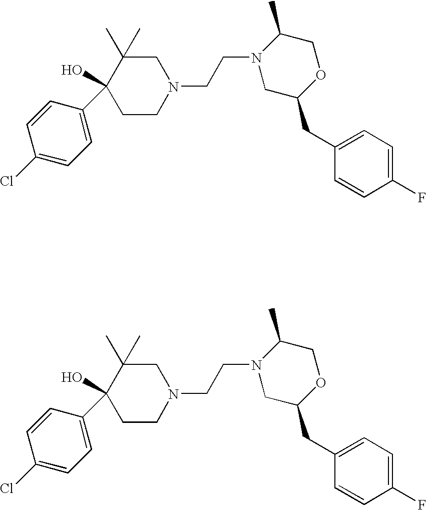

Diastereomeric mixture of (R)-4-(4-chloro-phenyl)-1-{2-[(2R,5S)-2-(4-fluoro-benzyl)-5-methyl-morpholin-4-yl]-ethyl}-3,3-dimethyl-piperidin-4-ol and (S)-4-(4-chloro-phenyl)-1-{2-[(2R,5S)-2-(4-fluoro-benzyl)-5-methyl-morpholin-4-yl]-ethyl}-3,3-dimethyl-piperidin-4-ol.

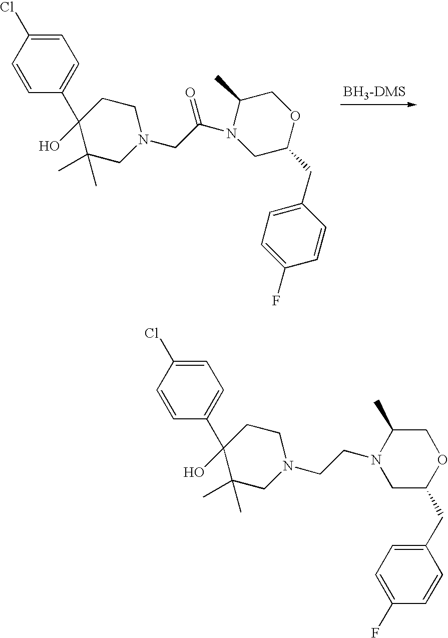

2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone was prepared as described above in Example 10.

To a solution of 2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone (90 mg, 0.18 mmol) in THF was added BH₃-DMS (0.05 ml, 0.55 mmol). The reaction mixture was stirred overnight then heated to reflux in MeOH for 1 hour. The mixture was concentrated and purified through column chromatography (Si-gel, 2% MeOH-DCM) to afford 34 mg (39%) of 4-(4-Chloro-phenyl)-1-{2-[(2R,5S)-2-(4-fluoro-benzyl)-5-methyl-morpholin-4-yl]-ethyl}-3,3-dimethyl-piperidin-4-ol. LC/MS [M+H]⁺: 475.4. ¹H-NMR (400 MHz, CDCl₃) δ (ppm): 7.39 (d, J=8.4 Hz, 2H), 7.28 (m, 2H), 7.17 (m, 2H), 6.96 (t, J=8.8 Hz, 2H), 3.75 (m, 2H), 3.66 (m, 2H), 2.49 (s, 2H), 2.86 (m, 3H), 2.71 (m, 2H), 2.50 (m, 3H), 2.40 (m, 3H), 1.46 (m, 2H), 1.06 (d, J=6.5 Hz, 3H), 0.88 (br s, 3H), 0.76 (br s, 3H). HPLC purity: 98.3%

### Example 12

### (5-Chloro-2-{3-[2-(4-fluoro-benzyl)-[1,4]oxazepan-4-yl]-propoxy}-phenyl)-urea

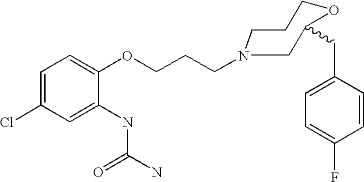

(5-Chloro-2-{3-[2-(4-fluoro-benzyl)-[1,4]oxazepan-4-yl]-3-oxo-propoxy}-phenyl)-urea was prepared as described above in Example 6.

To a solution of (5-Chloro-2-{3-[2-(4-fluoro-benzyl)-[1,4]oxazepan-4-yl]-3-oxo-propoxy}-phenyl)-urea (200 mg, 0.44 mmol) in THF was added BH₃-DMS (0.24 ml, 2.2 mmol). The solution was stirred for overnight then heated to reflux in MeOH for 1 hour. The mixture was concentrated and purified through column chromatography (Si-gel, 1-2% MeOH-DCM) to afford 40 mg (21%) of (5-Chloro-2-{3-[2-(4-fluoro-benzyl)-[1,4]oxazepan-4-yl]-propoxy}-phenyl)-urea. LC/MS [M+H]⁺: 436.1. ¹H-NMR (400 MHz, DMSO-d₆) δ (ppm): 8.19 (s, 1H), 7.87 (m, 1H), 7.22 (br s, 2H), 7.07 (br s, 2H), 6.95 (m, 1H), 6.87 (m, 1H), 6.42 (br s, 2H), 4.06 (s, 2H), 3.71 (br s, 2H), 3.55 (br s, 2H), 2.76 (m, 2H), 2.62 (m, 3H), 2.32 (m, 1H), 2.17 (m, 1H), 1.88 (m, 41-1). HPLC purity: 95.4%

### Example 13

### (5-Chloro-2-{3-[(2R,5S)-2-(4-fluoro-benzyl)-5-methyl-morpholin-4-yl]-propoxy}-phenyl)-urea

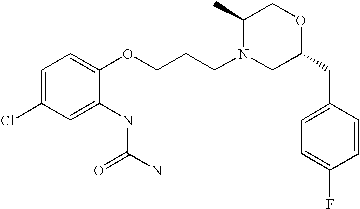

(5-Chloro-2-{3-[(2R,5S)-2-(4-fluoro-benzyl)-5-methyl-morpholin-4-yl]-propoxy}-phenyl)-urea was prepared by a procedure similar to that described in Example 12 using (5-Chloro-2-{3-[(2R,5S)-2-(4-fluoro-benzyl)-5-methyl-morpholin-4-yl]-3-oxo-propoxy}-phenyl)-urea (Example-4). LC/MS [M+H]⁺: 436.2. ¹H-NMR (400 MHz, CDCl₃) δ (ppm): 8.16 (s, 1H), 7.17-6.69 (m, 7H), 4.83 (br s, 2H), 4.03-3.67 (m, 5H), 2.95-2.39 (m, 71-1), 1.95 (m, 2H), 1.08 (d, 2H). HPLC purity: 94.2%

### Example 14

### 3-[4-(4-Chloro-phenyl)-4-hydroxy-3,3-dimethyl-piperidin-1-yl]-1-[2-(4-fluoro-benzyl)-[1,4]oxazepan-4-yl]-propan-1-one

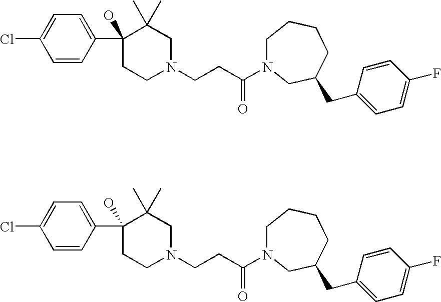

Racemic diastereomeric mixture of 3-[(S)-4-(4-chloro-phenyl)-4-hydroxy-3,3-dimethyl-piperidin-1-yl]-1-[(S)-2-(4-fluoro-benzyl)-[1,4]oxazepan-4-yl]-propan-1-one and 3-[(R)-4-(4-chloro-phenyl)-4-hydroxy-3,3-dimethyl-piperidin-1-yl]-1-[(S)-2-(4-fluoro-benzyl)-[1,4]oxazepan-4-yl]-propan-1-one.

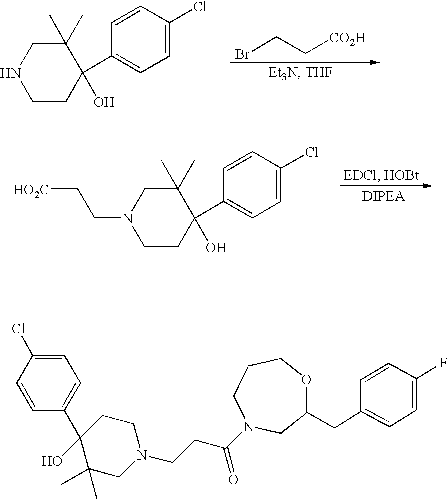

4-(4-Chloro-phenyl)-3,3-dimethyl-piperidin-4-ol was prepared as described in Example 10. LC/MS [M+H]⁺: 240.0. ¹H-NMR (400 MHz, CD₃OD) δ (ppm): 749-7.31 (4 Ar H), 3.35-2.64 (m, H), 1.41 (m, 2H), 0.91 (s, 3H), 0.78 (s, 3H).

### Step-1: 4-(4-Chloro-phenyl)-3,3-dimethyl-piperidin-4-ol-propionic acid

To 4-(4-Chloro-phenyl)-3,3-dimethyl-piperidin-4-ol 1 (1 g, 4.2 mmol) in THF was added 3-bromopropionic acid (638 mg, 4.2 mmol) and triethylamine (2 ml, 14.6 mmol). The solution was stirred overnight at room temperature. The reaction mixture was concentrated to dryness and diluted with water. The resulting precipitate was filtered and dried to afford 760 mg (36%) of 4-(4-Chloro-phenyl)-3,3-dimethyl-piperidin-4-ol-propionic acid. LC/MS [M+H]⁺: 311.0. ¹H-NMR (400 MHz, DMSO-d₆) δ (ppm): 7.48-7.34 (4 Ar H), 4.86 (s, 1H), 3.04 br. d 1H), 2.87 (br d, 1H), 2.69-2.56 (m, 4H), 2.36 (m, 3H), 0.79 (s, 3H), 0.63 (s, 3H)

### Step-2

### 3-[4-(4-Chloro-phenyl)-4-hydroxy-3,3-dimethyl-piperidin-1-yl]-1-[2-(4-fluoro-benzyl)-[1,4]oxazepan-4-yl]-propan-1-one

To a solution of 4-(4-Chloro-phenyl)-3,3-dimethyl-piperidin-4-ol-propionic acid (100 mg, 0.32 mmol) in DMF was added 2-(4-Fluoro-benzyl)-[1,4]oxazepane (74 mg, 0.35 mmol), EDCI (80 mg, 042 mmol), HOBt (22 mg, 0.16 mmol) and DIPEA (0.2 ml, 0.96 mmol) at 5-10° C. The reaction mixture was stirred at room temperature for 14-16 hours. The mixture was then diluted with water and extracted with ethyl acetate. The organic layer was concentrated under reduced pressure and purified through column chromatography (2-3% MeOH-DCM) to afford 45 mg (28%) of 3-[4-(4-Chloro-phenyl)-4-hydroxy-3,3-dimethyl-piperidin-1-yl]-1-[2-(4-fluoro-benzyl)-[1,4]oxazepan-4-yl]-propan-1-one. LC/MS [M+H]⁺: 503.2. ¹H-NMR (400 MHz, DMSO-d₆) δ (ppm): 7.43 (t, J=8.6 Hz, 2H), 7.34 (m, 2H), 7.27 (m, 2H), 7.10 (m, 2H), 4.64 (s, 1H), 4.01-2.66 (m, 12H), 2.40-1.22 (m, 9H), 0.76 (s, 3H), 0.62 (s, 3H). HPLC purity: 83.1%

### Example 15

### 4-(4-Chloro-phenyl)-1-{2-[2-(4-fluoro-benzyl)-[1,4]oxazepan-4-yl]-ethyl}-3,3-dimethyl-piperidin-4-ol

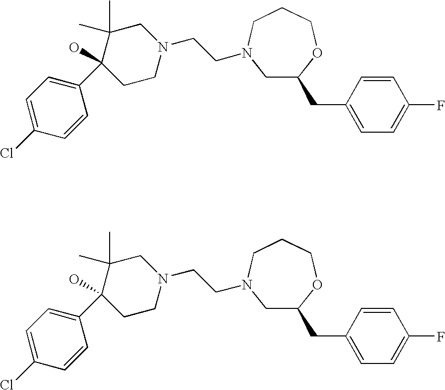

Racemic diastereomeric mixture of (S)-4-(4-chloro-phenyl)-1-{2-[(S)-2-(4-fluoro-benzyl)-[1,4]oxazepan-4-yl]-ethyl}-3,3-dimethyl-piperidin-4-ol and (R)-4-(4-chloro-phenyl)-1-{2-[(S)-2-(4-fluoro-benzyl)-[1,4]oxazepan-4-yl]-ethyl}-3,3-dimethyl-piperidin-4-ol.
2-(4-Fluoro-benzyl)-[1,4]oxazepane was prepared as described in Example 6.

4-(4-Chloro-phenyl)-1-{2-[2-(4-fluoro-benzyl)-[1,4]oxazepan-4-yl]-ethyl}-3,3-dimethyl-piperidin-4-ol was prepared by a procedure similar to that described in Example 11. LC/MS [M+H]⁺: 475.5. ¹H-NMR (400 MHz, DMSO-d₆) δ (ppm): 7.46-7.11 (m, 8H), 5.16 (br s, 1H), 3.75-1.86 (m, 21H), 0.79 (s, 3H), 0.68 (s, 31-1). HPLC purity: 96.7%

### Example 16

### (5-Chloro-2-{2-[7-(4-fluoro-benzyl)-[1,4]oxazepan-4-yl]-2-oxo-ethoxy}-phenyl)-urea

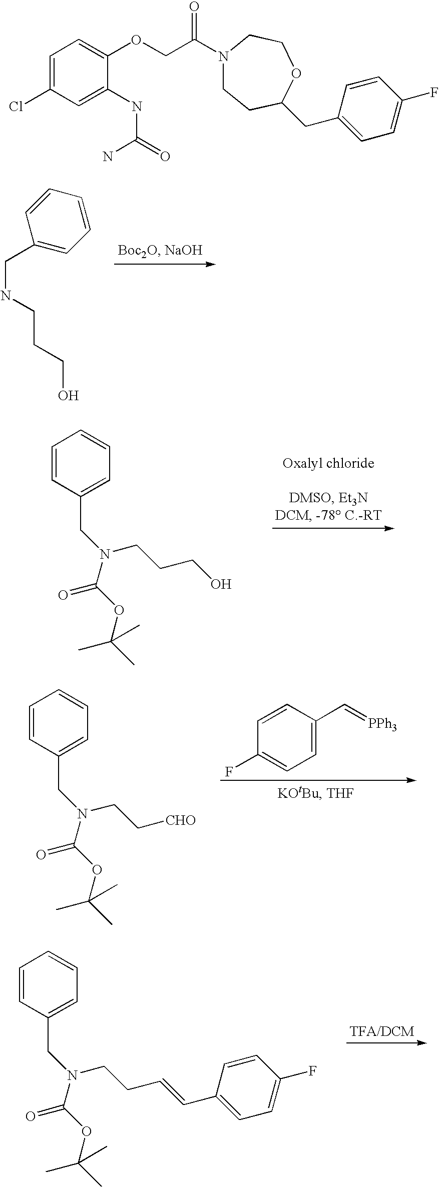

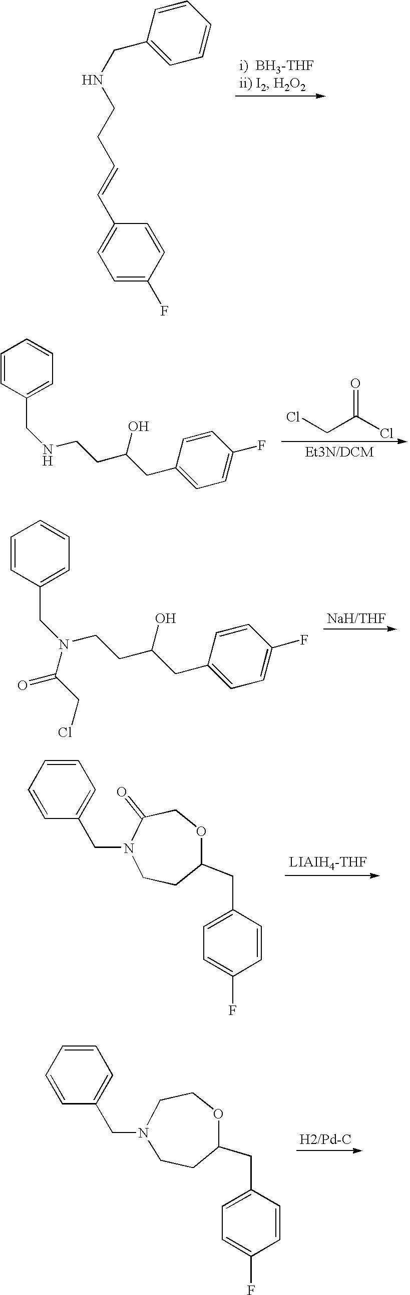

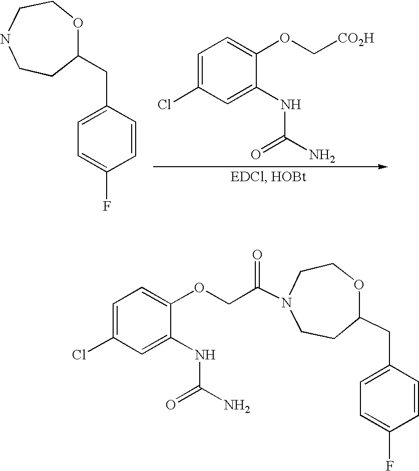

(4-(4-chloro-2-ureido-phenoxy)-acetic acid was prepared as described in Example 1.

### Step-1: (3-Hydroxy-propyl)-[(E)-((Z)-2-propenyl)-penta-2,4-dienyl]-carbamic acid tent-butyl ester

To a solution of 3-benzylamino-propan-1-ol (10 g, 0.06 mol) in DCM was added 1(N) NaOH and Boc-anhydride (13.5 ml, 0.06 mol) at 0° C. The solution was stirred overnight at room temperature. The organic layer was washed with water and concentrated under reduced pressure. The crude product was purified through column chromatography (Si-gel, 15% EtOAc-hexane) to afford 15.1 g (99%) of (3-Hydroxy-propyl)-[(E)-((Z)-2-propenyl)-penta-2,4-dienyl]-carbamic acid tert-butyl ester. LC/MS [M+H]⁺: 266.4. ¹H-NMR (400 MHz, CDCl₃) δ (ppm): 7.33-7.21 (m, 5-ArH), 4.37 (s, 2H), 3.71 (s, 1H), 3.55 (br s, 3H), 3.37 (br. s, 3H), 1.45 (s, 9H).

### Step-2 Benzyl-(3-oxo-propyl)-carbamic acid tert-butyl ester

To a solution of DMSO (13.5 ml, 0.19 mol) in anhydrous DCM was added drop wise (at −78° C.) a solution of oxalyl chloride (7.2 ml, 0.08 mol) in anhydrous DCM under an atmosphere of nitrogen. The resulting mixture was stirred for 15 minutes. A solution of (3-hydroxypropyl)-[(E)-((Z)-2-propenyl)-penta-2,4-dienyl]-carbamic acid tert-butyl ester (20.2 g, 0.07 mol) in DCM (85 ml) was then added dropwise over a period of 10 minutes and stirring was continued for 2 h. Triethyl amine (53 ml, 0.38 mol) was then added and stirring was continued for 5 minutes at −78° C. followed by stirring at RT for 30 minutes. The reaction mixture was then diluted with water and extracted with dichloromethane. The combined organic layers were washed with brine, water, 5% sodium bicarbonate and dried over anhydrous sodium sulfate. The organics were concentrated under reduced pressure to afford 18.1 g (92%) of benzyl-(3-oxo-propyl)-carbamic acid tert-butyl ester. ¹H-NMR (400 MHz, DMSO-d₆) δ (ppm): 9.63 (s, 1H), 7.37-7.22 (m, 5H), 4.37 (s, 2H), 3.41 (br. s, 2H), 2.60 (t, J=1.0 Hz, 2H), 1.38 (s, 9H).

### Step-3: Benzyl-[(E)-4-(4-fluoro-phenyl)-but-3-enyl]-carbamic acid tert-butyl ester

To a solution of (4-fluorobenzyl)triphenylphosphonium bromide (28 g, 0.075 mol) in THF was added t-BuOK (8.5 g, 0.075 mol) portion wise at 0° C. and the mixture was stirred for 1 hour at room temperature. A solution of benzyl-(3-oxo-propyl)-carbamic acid tert-butyl ester (18 g, 0.68 mol) was added drop wise at 0° C. The reaction mixture was stirred for 5 hours at room temperature then extracted with ethyl acetate. The organic layer was concentrated and purified through column chromatography (Si-gel, 3% ethyl acetate-hexane) to afford 10 g (62%) of benzyl-[(E)-4-(4-fluoro-phenyl)-but-3-enyl]hcarbamic acid tert-butyl ester. LC/MS [M+H]⁺: 356.2. ¹H-NMR (400 MHz, CDCl₃) δ (ppm): 7.33-6.93 (m, 9 ArH), 6.45 (d, J=11.6 Hz, 1H), 6.02 (br s, 1H), 4.43 (br s, 2H) 3.29 (br, 2H), 2.42 (br, 2H), 1.44 (s, 9H).

### Step-4: Benzyl-[(E)-4-(4-fluoro-phenyl)-but-3-enyl]-amine

To a solution of benzyl-[(E)-4-(4-fluoro-phenyl)-but-3-enyl]-carbamic acid tert-butyl ester (10 g, 0.028 mol) in DCM was added trifluoroacetic acid (21.5 ml, 0.28 mol)) and the resulting solution was stirred for 2 hours. The solution was then concentrated and washed with dry ether. The TFA salt was diluted with a minimum volume of water and neutralized with aq. NaOH solution at 5-10° C. Extraction of the aqueous solution with ethyl acetate followed by concentration of the organic layer afforded 6.8 g (95%) of benzyl-[(E)-4-(4-fluoro-phenyl)-but-3-enyl]-amine. LC/MS [M+H]⁺: 256. ¹H-NMR (400 MHz, DMSO-d₆) δ (ppm): 8.95 (s, 1H), 7.52-7.14 (m, 8 ArH), 6.53 (t, J=16.2 Hz, 1H), 6.21 (m, 1H), 4.18 (d, J=13.4 Hz, 2H), 3.05 (m, 2H), 2.65 (m, 1H), 2.54 (m, 1H).

### Step-5: 4-Benzylamino-1-(4-fluoro-phenyl)-butan-2-ol

The compound was prepared following a procedure similar to that described in J. Am. Chem. Soc., 125, 10502-10503, 2003.

To a solution of benzyl-[(E)-4-(4-fluoro-phenyl)-but-3-enyl]-amine (14 g, 0.027 mol) in 100 ml THF was added BH₃-THF (1.5 M, 18.2 ml) drop wise and the solution was stirred for 45 minutes at 0° C. After warming to room temperature, the reaction mixture was concentrated under reduced pressure. A solution of iodine (2 g, 0.013 mol) in dichloromethane was added drop wise to a solution of the crude amine-borane complex (14 g, 0.26 mol) at room temperature. After 30 minutes the solvent was evaporated to afford the crude hydroborated product. Oxidation was achieved by adding MeOH (200 ml) followed by 20% NaOH (70 ml) and 30% H₂O₂ (63 ml). The resulting white suspension was stirred overnight at room temperature. The reaction mixture was concentrated and extracted with dichloromethane. The combined organic layers were dried over sodium sulfate and the solvent was removed. The crude reaction mass was purified through Si-gel (3% meOH-DCM) to afford 4 g (54.2%) of 4-Benzylamino-1-(4-fluoro-phenyl)-butan-2-ol. LC/MS [M+H]⁺: 274.2

### Step-6: N-Benzyl-2-chloro-N-[4-(4-fluoro-phenyl)-3-hydroxy-butyl]-acetamide

To a solution of 4-Benzylamino-1-(4-fluoro-phenyl)-butan-2-ol (2 g, 7.3 mmol) in dichloromethane was added triethylamine (1.1 ml, 8.03 mmol) and chloroacetyl chloride (0.58 ml, 7.3 mmol) at 0° C. The resulting mixture was stirred for 2 hours at room temperature. The mixture was then concentrated, diluted with water and extracted with ethyl acetate. The organic layer was concentrated to afford 2.78 g N-Benzyl-2-chloro-N-[4-(4-fluoro-phenyl)-3-hydroxy-butyl]-acetamide. LC/MS [M+H]⁺: 350. ¹H-NMR (400 MHz, DMSO-d₆) δ (ppm): 7.38-7.04 (m, 9H), 4.74 (d, J=6.1 Hz, 1H), 4.62-4.35 (m, 5H), 3.57 (br s. 1H), 3.27 (m, 1H), 2.59 (d, J=6.1 Hz, 2H), 1.72 (m, 1H).

### Step-7: 4-Benzyl-7-(4-fluoro-benzyl)-[1,4]oxazepan-3-one

To a solution of N-Benzyl-2-chloro-N-[4-(4-fluoro-phenyl)-3-hydroxy-butyl]-acetamide (2.9 g, 8.3 mmol) in THF was added sodium hydride (0.26 g, 10.7 mmol) portion wise at 0° C. and the resulting mixture was stirred at room temperature overnight. Excess NaH was quenched with saturated aqueous solution of ammonium chloride at 0° C. and the product was extracted with ethyl acetate. The organic layer was concentrated to afford 2.65 g (80%) of 4-Benzyl-7-(4-fluoro-benzyl)-[1,4]oxazepan-3-one. LC/MS [M+H]⁺: 314. ¹H-NMR (400 MHz, CDCl₃) δ (ppm): 7.34-6.99 (m, 9H), 4.90-4.26 (m, 5H), 3.39 (d, J=13.5 Hz, 1H), 2.06 (m, 1H), 1.74 (m, 1H).

### Step-8: 4-Benzyl-7-(4-fluoro-benzyl)[1,4]oxazepane

To a solution of 4-Benzyl-7-(4-fluoro-benzyl)-[1,4]oxazepan-3-one (2.65 g, 8.55 mmol) in THF was added LiAlH₄ (0.65 g, 17.1 mmol) at 0° C. and the mixture was heated to reflux for 2 hours. Excess LiALH₄ was quenched with saturated aqueous sodium sulfate solution. The resulting precipitate was filtered through a celite bed. The filtrate was concentrated, diluted with water and extracted with ethyl acetate. The organic layer was concentrated and purified over Si-gel column (10% ethyl acetate-hexane) to afford 1.3 g (50%) of 4-Benzyl-7-(4-fluoro-benzyl)-[1,4]oxazepane. LC/MS [M+H]⁺: 300.2. ¹H-NMR (400 MHz, DMSO-d₆) δ (ppm): 7.32-7.05 (m, 9H), 3.88-3.70 (m, 2H), 3.57 (s, 2H), 3.44 (m, 1H), 2.72-2.52 (m, 5H), 1.81 (m, 1H), 1.68 (m, 1H)

### Step-9: 7-(4-Fluoro-benzyl)-[1,4]oxazepane

A solution of 4-Benzyl-7-(4-fluoro-benzyl)-[1,4]oxazepane (1.3 g, 4.3 mmol) in ethanol was stirred with 10% Pd—C (140 mg) under an atmosphere of hydrogen for 12 hours. The catalyst was removed by filtration through a celite bed. The ethanol solution was concentrated to afford 850 mg (93%) of 7-(4-Fluoro-benzyl)-[1,4]oxazepane. LC/MS [M+H]⁺: 210.4. ¹H-NMR (400 MHz, DMSO-d₆) δ (ppm): 7.16-6.92 (m, 4 ArH), 3.84 (m, 2H), 3.48 (m, 1H), 2.92 (m, 3H), 2.87 (m, 1H), 2.65 (m, 1H), 1.89 (m, 1 h, 1.62 (m, 1H).

### Step-10: (5-Chloro-2-{2-[7-(4-fluoro-benzyl)-[1,4]oxazepan-4-yl]-2-oxo-ethoxy}-phenyl)-urea

To a solution of (4-Chloro-2-ureido-phenoxy)-acetic acid (200 mg, 0.82 mmol) in DMF was added 7-(4-fluoro-benzyl)-[1,4]oxazepane (171 mg, 0.82 mmol), EDCI (203 mg, 1.1 mmol), HOBt (55 mg, 0.4 mmol) and DIPEA (0.4 ml, 2.5 mmol) at 5-10° C. The reaction mixture was stirred at room temperature for 14-16 hours. The mixture was then diluted with water and extracted with ethyl acetate. The organic layer was concentrated under reduced pressure and purified through column chromatography (Si-gel, 1-2% MeOH-DCM) to afford 60 mg (17%) of (5-Chloro-2-{2-[7-(4-fluoro-benzyl)-[1,4]oxazepan-4-yl]-2-oxo-ethoxy}-phenyl)-urea. LC/MS [M+H]⁺: 436.1. ¹H-NMR (400 MHz, CDCl₃) δ (ppm): 8.96 (s, 1H), 8.27 (s, 1H), 7.12 (t, J=5.7 Hz, 2H), 6.96 (t, J=8.6 Hz, 2H), 6.81 (m, 2H), 4.78 (s, 2H), 4.66 (m, 2H), 3.99 (m, 2H), 3.59 (d, J=6.2 Hz, 1H), 3.52 (s, 1H), 3.44 (m, 3H), 2.81 (m, 1H), 2.69 m, 1H), 1.94 (m, 1H), 1.71 (m, 1H). HPLC purity: 98.1%

### Example 17

### (5-Chloro-2-{3-[7-(4-fluoro-benzyl)-[7,4]oxazepan-4-yl]-3-oxo-propoxy}-phenyl)-urea

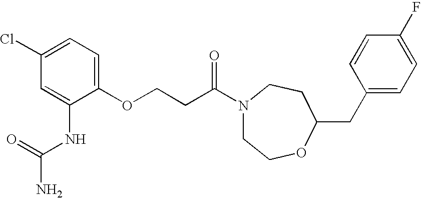

7-(4-Fluoro-benzyl)-[1,4]oxazepane was prepared as described in Example 16.

(5-Chloro-2-{3-[7-(4-fluoro-benzyl)-[1,4]oxazepan-4-yl]-3-oxo-propoxy}-phenyl)-urea was prepared by a procedure similar to that described in Example 6. LC/MS [M+H]⁺: 450.1. ¹H-NMR (400 MHz, CDCl₃) δ (ppm): 9.22 9s, 1H), 8.38 (m, 1H), 7.14-6.81 (m, 6H), 5.08 (br s, 2H), 4.07-1.97 (m, 15H). HPLC purity: 96.3%

### Example 18

### (5-Chloro-2-{3-[7-(4-fluoro-benzyl)-[1,4]oxazepan-4-yl]-propoxy}-phenyl)-urea

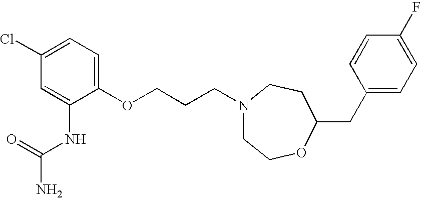

7-(4-Fluoro-benzyl)-[1,4]oxazepane was prepared as described in Example 16.

(5-Chloro-2-{3-[7-(4-fluoro-benzyl)-[1,4]oxazepan-4-yl]-propoxy}-phenyl)-urea was prepared by a procedure similar to that described in Example 13 using (5-Chloro-2-{3-[7-(4-fluoro-benzyl)-[1,4]oxazepan-4-yl]-3-oxo-propoxy}-phenyl)-urea. LC/MS [M+H]⁺: 436.0. ¹H-NMR (400 MHz, DMSO-d₆) δ (ppm): 8.18 (s, 1H), 7.30-6.70 (m, 7H), 4.99 (br s, 2H), 4.02-3.84 (m, 4H), 3.57 (m, 1H), 2.83-2.56 (m, 9H), 1.99-1.96 (m, 3H). HPLC purity: 98.3%

### Example 19

### (5-Chloro-2-{2-[7-(4-fluoro-benzyl)-[1,4]oxazepan-4-yl]-ethoxy}-phenyl)-urea

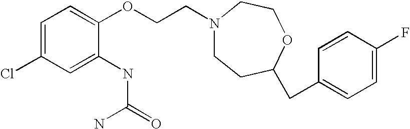

(5-Chloro-2-{2-[7-(4-fluoro-benzyl)-[1,4]oxazepan-4-yl]-2-oxo-ethoxy}-phenyl)-urea was prepared as described in Example 16.

To a solution of (5-Chloro-2-{2-[7-(4-fluoro-benzyl)-[1,4]oxazepan-4-yl]-2-oxo-ethoxy}-phenyl)-urea (50 mg, 0.12 mmol) in THF was added BH₃-DMS (0.05 ml, 5 eqv.). The solution was stirred overnight then heated to reflux in MeOH for 1 hour. The reaction mixture was concentrated and purified through column chromatography (Si-gel, 1-2% MeOH-DCM) to afford 15 mg (31%) of (5-Chloro-2-{2-[7-(4-fluoro-benzyl)-[1,4]oxazepan-4-yl]-ethoxy}-phenyl)-urea. LC/MS [M+H]⁺: 421.8. ¹H-NMR (400 MHz, CDCl₃) δ (ppm): 8.49 (s, 1H), 8.20 (s, 1H), 7.12 (t, J=5.76 Hz, 2H), 6.96 (t, J=8.6 Hz, 2H), 6.86 (dd, J=8.5, 2.1 Hz, 1H), 6.79 (d, J=8.6 Hz, 1H), 5.25 (s, 2H), 4.07 (t, J=4.7 Hz, 2H), 3.98 (br s, 1H), 3.82 (m, 1H), 3.65 (m, 1H), 2.97 (m, 2H), 2.82 (m, 3H), 2.67 (m, 3H), 1.90 (br s, 2H). HPLC purity: 99%

### Example 20

### 4-[3-(4-Chloro-phenoxy)-propyl]-2-(4-fluoro-benzyl)-[1,4]oxazepane

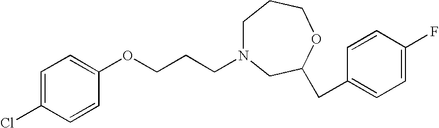

4-[3-(4-Chloro-phenoxy)-propyl]-2-(4-fluoro-benzyl)-[1,4]oxazepane was prepared by a procedure similar to that described in Example 12. LC/MS [M+H]⁺: 378.1. ¹H-NMR (400 MHz, DMSO-d₆) δ (ppm): 7.31-6.91 (m, 8H), 3.98 (m, 2H), 3.7 (m, 2H), 3.50 (m, 1H), 2.79-2.31 (m, 8H), 1.78 (m, 4H). HPLC purity: 96.1%

### Example 21

### (5-Chloro-2-{2-[2-(4-fluoro-benzyl)-[1,4]oxazepan-4-yl]-ethoxy}-phenyl)-urea

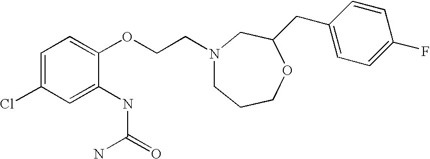

(5-Chloro-2-{2-[2-(4-fluoro-benzyl)-[1,4]oxazepan-4-yl]-ethoxy}-phenyl)-urea was prepared by a procedure similar to that described in Example 8 from (5-chloro-2-{2-[2-(4-fluoro-benzyl)-[1,4]oxazepan-4-yl]-2-oxo-ethoxy}-phenyl)-urea (Example 6). LC/MS [M+H]⁺: 421.9. ¹H-NMR (400 MHz, CDCl₃) δ (ppm): 8.57 (br s, 2H), 7.17-6.65 (m, 6H), 4.07 9br s, 2H), 3.91-3.48 (m, 5H), 2.75 (m, 6H), 1.99 (m, 2H), 0.92 (m, 2H). HPLC purity: 97.2

### Example 22

### 3-[2-(4-Fluoro-benzyl)-[1,4]oxazepan-4-yl]-N-(3-fluoro-phenyl)-propionamide

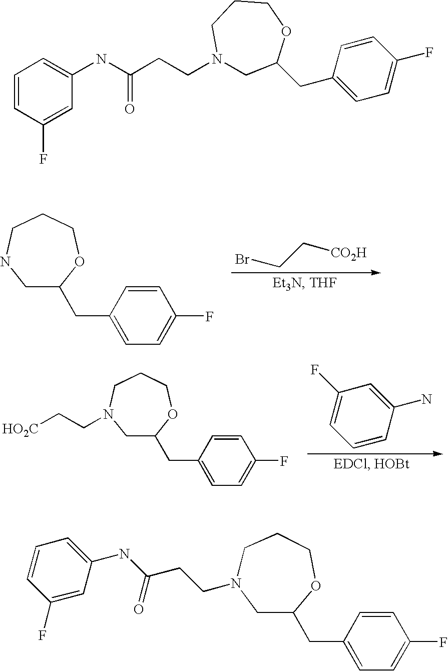

2-(4-fluoro-benzyl)-[1,4]oxazepane was prepared as described in Example 6.

### Step-1: 3-[2-(4-Fluoro-benzyl)-[1,4]oxazepan-4-yl]-propionic acid

To a solution of 2-(4-fluoro-benzyl)-[1,4]oxazepane (342 mg, 1.6 mmol) in THF was added triethylamine (0.45 ml, 3.2 mmol) and 3-bromopropionic acid (250 mg, 1.6 mmol). The reaction mixture was stirred overnight at room temperature. The resulting precipitate was removed by filtration and the filtrate was concentrated to afford 150 mg (33%) of 3-[2-(4-Fluoro-benzyl)-[1,4]oxazepan-4-yl]-propionic acid. LC/MS [M+H]⁺: 288.3

### Step-2: 3-[2-(4-Fluoro-benzyl)-[1,4]oxazepan-4-yl]-N-(3-fluoro-phenyl)-propionamide

To a solution of 3-[2-(4-Fluoro-benzyl)-[1,4]oxazepan-4-yl]-propionic acid (500 mg, 1.77 mmol) in DCM was added 3-fluoroaniline (197 mg, 1.77 mmol), EDCI (443 mg, 2.3 mmol), HOBt (120 mg, 0.88 mmol) and DIPEA (0.9 ml, 5.3 mmol) at 5-10° C. The reaction mixture was stirred at room temperature for 16 hours. The mixture was then concentrated and extracted with ethyl acetate. The organic layer was concentrated in vacuo and purified through column chromatography (Si-gel, 1-2% MeOH-DCM) to afford 100 mg (15%) of 3-[2-(4-Fluoro-benzyl)-[1,4]oxazepan-4-yl]-N-(3-fluoro-phenyl)-propionamide. LC/MS [M+H]⁺: 375.2. ¹H-NMR (400 MHz, DMSO-d₆) δ (ppm): 10.24 (s, 1H), 7.58 (d, J=11.7 Hz, 1H), 7.31 (m, 1H), 7.22 (m, 3H), 7.05 (t, J=8.8 Hz, 2H), 6.85 (m, 1H), 3.71 (m, 2H), 3.50 (m, 1H), 2.80 (m, 4H), 2.62 (m, 2H), 2.58 (m, 1H), 2.43 (m, 3H), 1.74 (m, 2H). HPLC purity: 95.2%

### Example 23

### N-(3-Chloro-phenyl)-3-[2-(4-fluoro-benzyl)-[1,4]oxazepan-4-yl]-propionamide

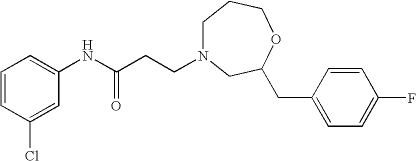

N-(3-Chloro-phenyl)-3-[2-(4-fluoro-benzyl)-[1,4]oxazepan-4-yl]-propionamide was prepared by a procedure similar to that described in Example 22. LC/MS [M+H]⁺: 391.1. ¹H-NMR (400 MHz, DMSO-d₆) δ (ppm): 10.22 (s, 1H), 7.81 (s, 1H), 7.40 (d, J=7.9 Hz, 1H), 7.31 (t, J=8.1 Hz, 1H), 7.21 (t, J=8.2 Hz, 1H), 7.06 (m, 3H), 3.70 (m, 2H), 3.59 (m, 1H), 3.49 (m, 1H), 2.81 (m, 3H), 2.63 (m, 2H), 2.44 (m, 3H), 1.73 (m, 3H). HPLC purity: 95.1%

### Example 24

### N-(5-Chloro-2-methoxy-phenyl)-3-[2-(4 fluoro-benzyl)-[1,4]oxazepan-4-yl]-propionamide

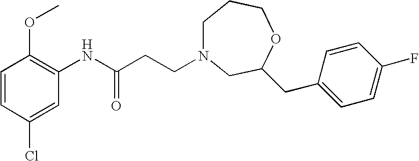

N-(5-Chloro-2-methoxy-phenyl)-3-[2-(4-fluoro-benzyl)-[1,4]oxazepan-4-yl]-propionamide was prepared by a procedure similar to that described in Example 22. LC/MS [M+H]⁺: 421.2. ¹H-NMR (400 MHz, DMSO-d₆) δ (ppm): 10.35 (s, 1H), 8.24 (s, 1H), 7.22 (t, J=7.9 Hz, 2H), 7.05 (m, 4H), 3.81 (s, 3H), 3.77 (m, 2H), 3.55 (m, 1H), 2.91 (m, 2H), 2.76 (m, 2H), 2.65 (m, 2H), 2.60 (m, 1H), 2.42 (m, 1H), 2.0-1.77 (m, 3H), 1.34 (m, 1H). HPLC purity: 92.6%

### Example 25

### (5-Chloro-2-{3-[2-(3,4-difluoro-benzyl)-[1,4]oxazepan-4-yl]-3-oxo-propoxy}-phenyl)-urea

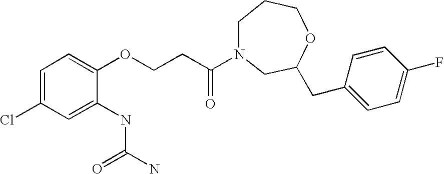

(5-Chloro-2-{3-[2-(3,4-difluoro-benzyl)-[1,4]oxazepan-4-yl]-3-oxo-propoxy}-phenyl)-urea was prepared by a procedure similar to that described in Example 6. LC/MS [M+H]⁺: 468.2. ¹H-NMR (400 MHz, DMSO-d₆) δ (ppm): 8.19-8.09 (m, 2H), 7.31 (m, 2H), 7.07-6.87 (m, 3H), 6.29 (s, 2H), 4.25-2.70 (m, 13H), 1.74 (m, 2H). HPLC purity: 95.1%

### Example 26

### (5-Chloro-2-{3-[2-(3,4-difluoro-benzyl)-[1,4]oxazepan-4-yl]-propoxy}-phenyl)-urea

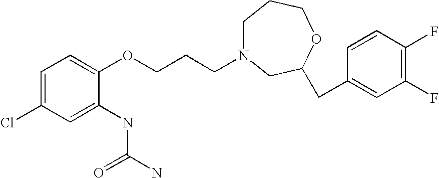

(5-Chloro-2-{3-[2-(3,4-difluoro-benzyl)-[1,4]oxazepan-4-yl]-propoxy}-phenyl)-urea was prepared from (5-Chloro-2-{3-[2-(3,4-difluoro-benzyl)-[1,4]oxazepan-4-yl]-3-oxo-propoxy}-phenyl)-urea by a procedure similar to that described in Example 12. LC/MS [M+H]⁺: 453.9. ¹H-NMR (400 MHz, DMSO-d₆) δ (ppm): 8.18 (s, 1H), 7.87 (br s, 1H), 7.27 (br s, 2H), 7.03 (br s, 1H), 6.96 (d, J=8.5 Hz, 1H), 6.88 (m, 1H), 6.39 (br s, 2H), 4.05 (s, 2H), 3.71 (br s, 2H), 3.50 (br s, 1H), 3.31 (br s, 1H), 2.77 (m, 2H), 2.62 (m, 3H), 2.32 (m, 1H), 2.17 (m, 1H), 1.88 (m, 4H). HPLC purity: 93.5%

### Example 27

### (5-Chloro-2-{3-[2-(3-fluoro-benzyl)-[1,4]oxazepan-4-yl]-3-oxo-propoxy}-phenyl)-urea

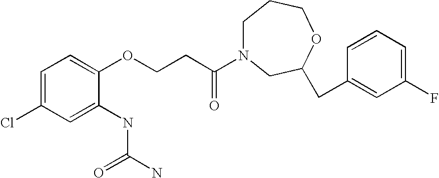

(5-Chloro-2-{3-[2-(3-fluoro-benzyl)-[1,4]oxazepan-4-yl]-3-oxo-propoxy}-phenyl)-urea was prepared using a procedure similar to that described in Example 6. LC/MS [M+H]⁺: 450.0. ¹H-NMR (400 MHz, DMSO-d₆) δ (ppm): 8.20-8.10 (m, 2H), 7.34-6.87 (m, 6H), 6.29 9s, 2H), 4.25-2.49 (m, 13H), 1.75 (m, 2H). HPLC purity: 97.8%

### Example 28

### (5-Chloro-2-{3-[2-(3-fluoro-benzyl)-[1,4]oxazepan-4-yl]-propoxy}-phenyl)-urea

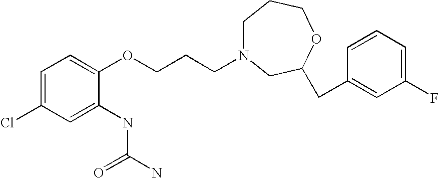

(5-Chloro-2-{3-[2-(3-fluoro-benzyl)-[1,4]oxazepan-4-yl]-propoxy}-phenyl)-urea was prepared from (5-Chloro-2-{3-[2-(3-fluoro-benzyl)-[1,4]oxazepan-4-yl]-3-oxo-propoxy}-phenyl)-urea by a procedure similar to that described in Example 12. LC/MS [M+H]⁺: 436.1. ¹H-NMR (400 MHz, CDCl₃) δ (ppm): 8.2 (s, 1H), 7.20-6.69 (m, 7H), 4.98 (br s, 2H), 4.02-3.63 (m, 5H), 2.91-2.52 (m, 8H), 1.97-1.96 (m, 4H). HPLC purity: 97.2%

### Example 29

### 2,6-Dichloro-N-{3-[2-(4-fluoro-benzyl)-[1,4]oxazepan-4-yl]-propyl}-benzenesulfonamide

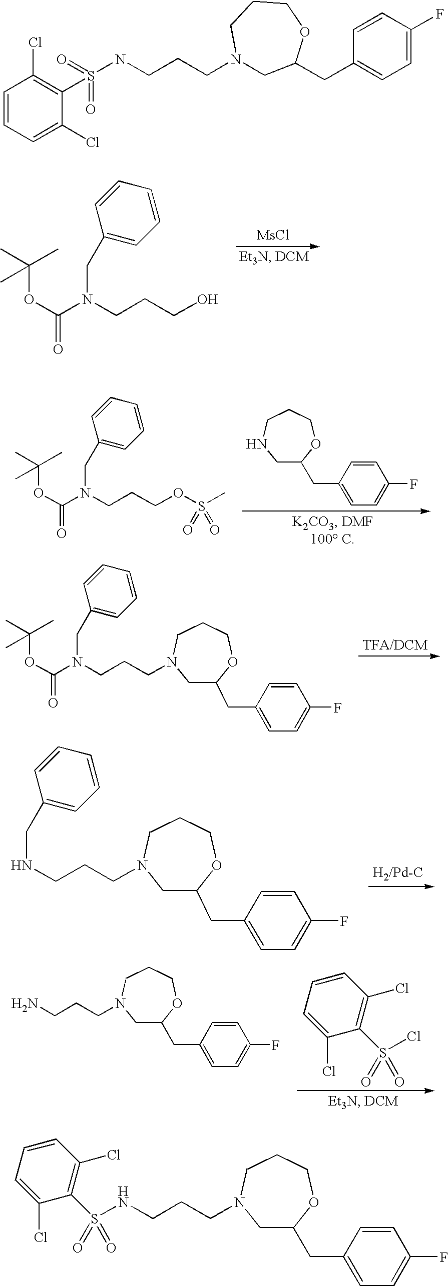

2-(4-Fluoro-benzyl)-[1,4]oxazepane was prepared as described in Example 6.

### Step-1: Methanesulfonic acid 3-(benzyl-tert-butoxycarbonyl-amino)-propyl ester

To a solution of benzyl-(3-hydroxy-propyl)-carbamic acid tert-butyl ester (10 g, 37.7 mmol) in DCM was added triethylamine (7.9 ml, 56.5 mmol) and mesityl chloride (3.52 ml, 45.2 mmol) at 0° C. The reaction mixture was stirred overnight. The mixture was then concentrated, diluted with water and extracted with ethyl acetate. The organic layer was concentrated in vacuo and purified through column chromatography (Si-gel, 15% ethyl acetate-hexane) to afford 5 g (44%) methanesulfonic acid 3-(benzyl-tert-butoxycarbonyl-amino)-propyl ester. LC/MS [M+H]: 344. ¹H-NMR (400 MHz, DMSO-d₆) δ (ppm): 7.36-7.20 (m, 5H), 4.38 (s, 2H), 4.16 (t, J=6.1 Hz, 2H), 3.22 (brs, 2H), 3.14 (s, 3H), 1.85 (t, J=6.1 Hz, 2H), 1.39 (s, 9H).

### Step-2: Benzyl-{3-[2-(4-fluoro-benzyl)-[1,4]oxazepan-4-yl]-propyl}-carbamic acid tert-butyl ester

To a solution of methanesulfonic acid 3-(benzyl-tert-butoxycarbonyl-amino)-propyl ester (2.46 g, 7.2 mmol) in DMF was added potassium carbonate (1.48 g, 10.7 mmol)) and 2-(4-Fluoro-benzyl)-[1,4]oxazepane (1.5 g, 7.2 mmol) at 0° C. The solution was heated to 100° C. overnight. The DMF solution was diluted with iced water and extracted with ethyl acetate. The organic layer was concentrated under reduced pressure and purified through column chromatography (Si-gel, 1-2% MeOH-DCM) to afford 1.1 g (34%) of benzyl-{3-[2-(4-fluoro-benzyl)-[1,4]oxazepan-4-yl]-propyl}-carbamic acid tert-butyl ester. LC/MS [M+H]⁺: 457.5. ¹H-NMR (400 MHz, CDCl₃) δ (ppm): 7.32-6.92 (m, 9H), 4.38 (brs, 2H), 3.84-2.31 (m, 15H), 1.80 (brs, 2H), 1.43 (s, 9H)

### Step-3: Benzyl-{3-[2-(4-fluoro-benzyl)-[1,4]oxazepan-4-yl]-propyl}-amine

To a solution of benzyl-{3-[2-(4-fluoro-benzyl)-[1,4]oxazepan-4-yl]-propyl}-carbamic acid tert-butyl ester (1.1 g, 2.4 mmol) in DCM was added trifluoroacetic acid (1.9 ml, 24.1 mmol) and stirred the solution for about 5 h. The reaction mixture was concentrated and washed with dry ether. The TFA salt was diluted with a minimum volume of water, neutralized with aq. NaOH solution at 5-10° C. and extracted with ethyl acetate. The organic layer was concentrated to afford 760 mg (89%) of benzyl-{3-[2-(4-fluoro-benzyl)-[1,4]oxazepan-4-yl]-propyl}-amine. LC/MS [M+H]⁺: 357.2

### Step-4: 3-[2-(4-fluoro-benzyl)[1,4]oxazepan-4-yl]-propylamine

Benzyl-{3-[2-(4-fluoro-benzyl)-[1,4]oxazepan-4-yl]-propyl}-amine (750 mg, 2.1 mmol) was stirred with 10% Pd—C under an atmosphere of hydrogen in ethanol for 5-10 h. The catalyst was removed by filtration through a celite bed and the ethanol solution was evaporated to dryness to afford 450 mg (81%) of 3-[2-(4-fluoro-benzyl)-[1,4]oxazepan-4-yl]-propylamine. LC/MS [M+H]⁺: 267.4. ¹H-NMR (400 MHz, DMSO-d₆) δ (ppm): 7.23-7.05 (m, 4H), 3.7 (brs, 2H), 3.5 (brs, 1H), 2.77-2.29 (m, 9H), 1.73 (m, 2H), 1.44 (m, 2H), 1.23 (s, 1H).

### Step-5:2,6-Dichloro-N-{3-[2-(4-fluoro-benzyl)-[1,4]oxazepan-4-yl]-propyl}-benzenesulfonamide

To a solution of 3-[2-(4-fluoro-benzyl)-[1,4]oxazepan-4-yl]-propylamine (250 mg, 0.94 mmol) in THF was added triethylamine (0.26 ml, 1.9 mmol) and 2,6-dichlorobenzenesulfonyl chloride (230 mg, 0.94 mmol) at 0° C. The reaction mixture was stirred overnight then concentrated, diluted with water and extracted with ethyl acetate. The organic layer was concentrated in vacuo and purified through column chromatography (Si-gel, 1% MeOH-DCM) to afford 126 mg (29%) of 2,6-Dichloro-N-{3-[2-(4-fluoro-benzyl)-[1,4]oxazepan-4-yl]-propyl}-benzenesulfonamide. LC/MS [M+H]⁺: 475.1. ¹H-NMR (400 MHz, DMSO-d₆) δ (ppm): 7.43 (d, J=8.0 Hz, 2H), 7.31 (m, 1H), 7.16 (t, J=12.0 Hz, 2H), 6.97 (t, J=8.4 Hz, 2H), 3.83 (m, 2H), 3.63 (m, 1H), 3.14 (m, 1H), 3.06 (m, 1H), 2.85 (m, 2H), 2.75 (m, 1H), 2.63-2.50 (m, 4H), 2.31 (m, 1H), 1.89-1.86 (m, 2H), 1.62 (m, 1H), 1.60 (m, 1H). HPLC purity: 92%

### Example 30

### (5-Chloro-2-{3-[4-(4-fluoro-benzyl)-4-hydroxy-3,3-dimethyl-piperidin-1-yl]-propoxy}-phenyl)-urea

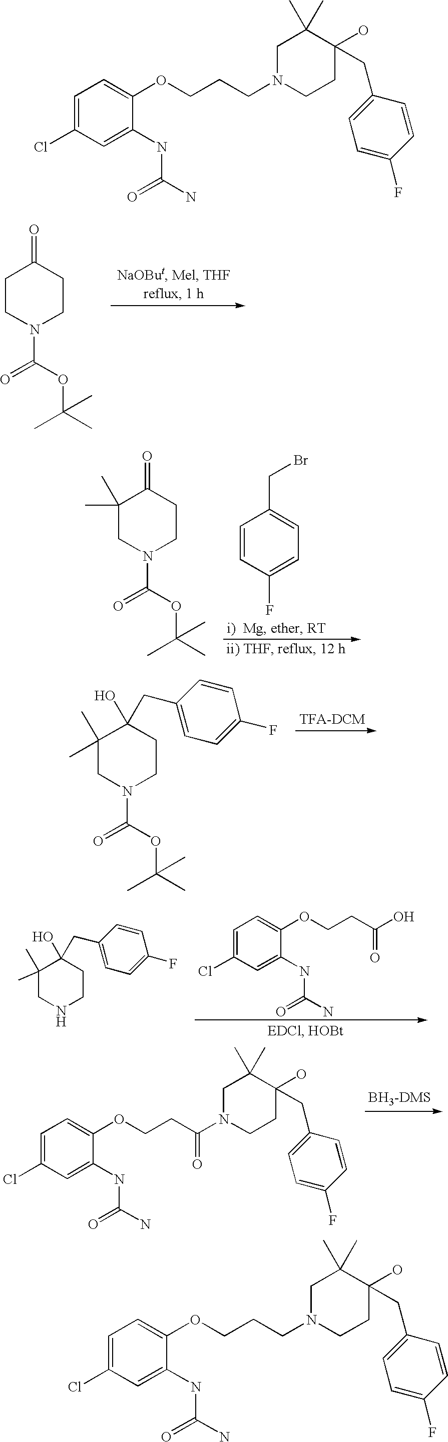

(5-Chloro-2-oxo-phenyl)-urea-propionic acid was prepared as described in Example 4.

### Step-1: 3,3-Dimethyl-4-oxo-piperidine-1-carboxylic acid tert-butyl ester

To a solution of 4-Oxo-piperidine-1-carboxylic acid tert-butyl ester (18 g, 0.09 mol) was added methyl iodide (11.8 ml, 0.19 mol) at 0° C. Sodium tert butoxide (20.9 g, 0.22 mol) was then added at 0° C. and the resulting mixture was heated to reflux for 1 hour. The mixture was concentrated under reduced pressure, diluted with water and extracted with ethyl acetate. The organic layer was washed with brine, dried over sodium sulfate, concentrated and purified through column chromatography (Si-gel, 2% ethyl acetate-hexane) to afford 7 g (37%) of 3,3-Dimethyl-4-oxo-piperidine-1-carboxylic acid tert-butyl ester. ¹H-NMR (400 MHz, CDCl₃) δ (ppm): 3.7 (m, 2H), 3.41 (m, 2H), 2.47 (t, 2H), 1.48 (s, 9H), 1.09 (s, 6H).

### Step-2: 4-(4-Fluoro-benzyl)-4-hydroxy-3,3-dimethyl-piperidine-1-carboxylic acid tert-butyl ester

To a solution of 4-chlorobenzyl bromide (3.52 g, 17.2 mmol) in ether was added Mg turning (500 mg, 20.57 mmol) and the resulting mixture was stirred at room temperature for 1 hour. To the resulting Grignard solution was added a solution of 3,3-Dimethyl-4-oxo-piperidine-1-carboxylic acid tert-butyl ester (3.0 g, 13.2 mmol) in THF drop wise at room temperature. The reaction mixture was heated to reflux overnight, cooled then diluted with aqueous saturated NH4Cl solution and extracted with ethyl acetate. The organic layer was washed with water, dried over sodium sulfate, concentrated and purified over Si-gel (5% ether-hexane) to afford 1.8 g (39%) of 4-(4-Chloro-benzyl)-4-hydroxy-3,3-dimethyl-piperidine-1-carboxylic acid tert-butyl ester. LC/MS [M+H]⁺: 354.3

### Step-3: 4-(4-Fluoro-benzyl)-3,3-dimethyl-piperidin-4-ol

To a solution of 4-(4-Chloro-benzyl)-4-hydroxy-3,3-dimethyl-piperidine-1-carboxylic acid tert-butyl ester (1.8 g, 5.08 mmol)) in DCM was added trifluoroacetic acid (3.8 ml, 50 mmol). The resulting mixture was stirred for about 5 hours then concentrated and washed with dry ether. The TFA salt was diluted with a minimum volume of water, neutralized with aq. NaOH solution at 5-10° C. and extracted with ethyl acetate. The organic layer was dried over sodium sulfate and concentrated to afford 1 g (78%) of 4-(4-Chloro-benzyl)-3,3-dimethyl-piperidin-4-ol. LC/MS [M+H]⁺: 254.2

### Step-4: (5-Chloro-2-{3-[4-(4-fluoro-benzyl)-4-hydroxy-3,3-dimethyl-piperidin-1-yl]-3-oxo-propoxy}-phenyl)-urea

To a solution of 3-(4-chloro-2-ureido-phenoxy)-propionic acid (150 mg, 0.58 mmol) in DMF was added 4-(4-Fluoro-benzyl)-3,3-dimethyl-piperidin-4-ol (138 mg, 0.58 mmol), EDCI (144 mg, 0.75 mmol), HOBt (39 mg, 0.29 mmol) and DIPEA (0.23 ml, 1.7 mmol) at 5-10° C. The reaction mixture was stirred at room temperature for 14-16 hours then concentrated and extracted with ethyl acetate. The organic layer was concentrated under reduced pressure and purified by column chromatography (Si-gel, 2% MeOH-DCM) to afford 70 mg (25%) of (5-Chloro-2-{3-[4-(4-fluoro-benzyl)-4-hydroxy-3,3-dimethyl-piperidin-1-yl]-3-oxo-propoxy}-phenyl)-urea. LC/MS [M+H]⁺: 478.4. ¹H-NMR (400 MHz, DMSO-d₆) δ (ppm): 8.22-8.18 (m, 2H), 7.26-6.86 (6 Ar H), 6.3 (s, 2H), 4.18 (m, 3H), 4.00-2.61 (m, 10H), 1.01 (d, J=15.2 Hz, 1H), 0.99 (s, 3H, 0.95 (s, 3H)

### Step-5: (5-Chloro-2-{3-[4-(4-fluoro-benzyl)-4-hydroxy-3,3-dimethyl-piperidin-1-yl]-propoxy}-phenyl)-urea

To a solution of (5-Chloro-2-{3-[4-(4-fluoro-benzyl)-4-hydroxy-3,3-dimethyl-piperidin-1-yl]-3-oxo-propoxy}-phenyl)-urea (70 mg, 0.15 mmol) in THF was added BH₃-DMS (0.15 ml). The resulting solution was stirred overnight then heated to reflux in MeOH for 1 hour. The mixture was then concentrated and purified through column chromatography (Si-gel, 1-2.5% MeOH-DCM) to afford 30 mg (44%) of (5-Chloro-2-{3-[4-(4-fluoro-benzyl)-4-hydroxy-3,3-dimethyl-piperidin-1-yl]-propoxy}-phenyl)-urea. LC/MS [M+H]⁺: 464.2. ¹H-NMR (400 MHz, DMSO-d₆) δ (ppm): 8.18 (s, 1H), 7.87 (s, 1H), 7.24 (s, 2H), 7.05 (m, 2H), 6.95 (m, 1H), 6.88 (m, 1H), 6.40 (s, 2H), 4.06 (br s, 2H), 3.8 (s, 1H), 3.66 (m, 3H), 2.34 (m, 2H), 2.22 (m, 2H), 2.08 (m, 1H), 1.89 (m, 2H), 1.23 (br s, 2H), 1.00 (s, 3H), 090 (s, 3H). HPLC purity: 94.13%

### Example 31

### 4-[3-(2-Bromo-4-chloro-phenoxy)-propyl]-2-(4-fluoro-benzyl)-[1,4]oxazepane

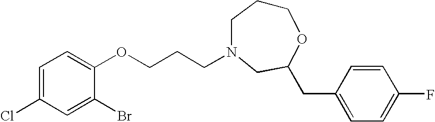

4-[3-(2-Bromo-4-chloro-phenoxy)-propyl]-2-(4-fluoro-benzyl)-[1,4]oxazepane was prepared by a procedure similar to that described in Example 12. LC/MS [M+H]⁺: 457.8. ¹H-NMR (400 MHz, DMSO-d₆) δ (ppm): 7.67-7.03 (m, 7H), 4.05 (m, 2H), 3.70 (m, 2H), 3.50 (m, 2H), 2.75-2.59 (m, 6H), 2.38 (m, 1H), 1.81 (m, 4H). HPLC purity: 95.3%

### Example 32

### (5-Chloro-2-{3-[4-(4-chloro-phenyl)-4-hydroxy-3,3-dimethyl-piperidin-1-yl]-propoxy}-phenyl)-urea

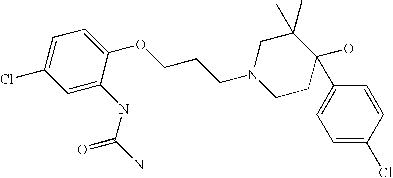

4-(4-Chloro-phenyl)-3,3-dimethyl-piperidin-4-ol was prepared as described in Example 10.
(5-Chloro-2-oxo-phenyl)-urea-propionic acid was prepared as described in Example 4.

(5-Chloro-2-{3-[4-(4-chloro-phenyl)-4-hydroxy-3,3-dimethyl-piperidin-1-yl]-propoxy}-phenyl)-urea was prepared by a procedure similar to that described in Example 31. LC/MS [M+H]⁺: 466.0. ¹H-NMR (400 MHz, DMSO-d₆) δ (ppm): 8.19 (d, 1H), 7.89 (s, 1H), 7.48-6.88 (m, 6H), 6.41 (br s, 2H), 4.66 (s, 1H), 4.09 (m, 2H), 2.67-1.93 (m, 9H), 1.43 (m, 1H), 0.78 (s, 3H), 0.64 (s, 3H). HPLC purity: 98.8%

### Example 33

### (5-Chloro-2-{3-[4-(4-fluoro-phenyl)-4-hydroxy-3,3-dimethyl-piperidin-1-yl]-propoxy}-phenyl)-urea

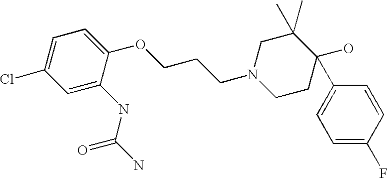

4-(4-Fluoro-phenyl)-3,3-dimethyl-piperidin-4-ol was prepared as described in Example 10.
(5-Chloro-2-oxo-phenyl)-urea-propionic acid was prepared as described in Example 4.

(5-Chloro-2-{3-[4-(4-fluoro-phenyl)-4-hydroxy-3,3-dimethyl-piperidin-1-yl]-propoxy}-phenyl)-urea was prepared by a procedure similar to that described in Example 31. LC/MS [M+H]⁺: 450.0. ¹H-NMR (400 MHz, DMSO-d₆) δ (ppm): 8.20 (br s, 1H), 7.9 (d, 1H), 7.48-6.4 (m, 8H), 4.60 (s, 1H), 4.10 (br s, 2H), 2.83-1.08 (m, 10H), 0.94-0.76 (m, 6H). HPLC purity: 92%

### Example 34

### 4-chloro-2-{3-[2-(4-fluoro-benzyl)-[1,4]oxazepan-4-yl]-2-hydroxy-propoxy}-benzamide

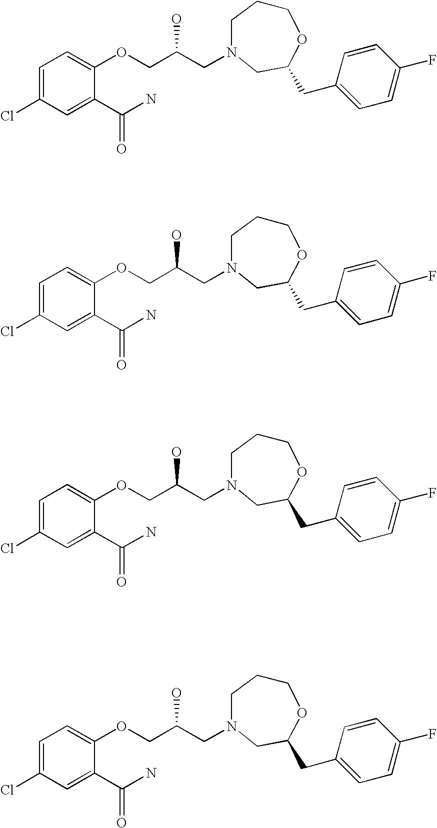

Isomeric mixture of 5-chloro-2-{(R)-3-[(R)-2-(4-fluoro-benzyl)-[1,4]oxazepan-4-yl]-2-hydroxy-propoxy}-benzamide, 5-chloro-2-{(S)-3-[(R)-2-(4-fluoro-benzyl)-[1,4]oxazepan-4-yl]-2-hydroxy-propoxy}-benzamide, 5-chloro-2-{(R)-3-[(S)-2-(4-fluoro-benzyl)-[1,4]oxazepan-4-yl]-2-hydroxy-propoxy}-benzamide and 5-chloro-2-{(S)-3-[(S)-2-(4-fluoro-benzyl)-[1,4]oxazepan-4-yl]-2-hydroxy-propoxy}-benzamide.

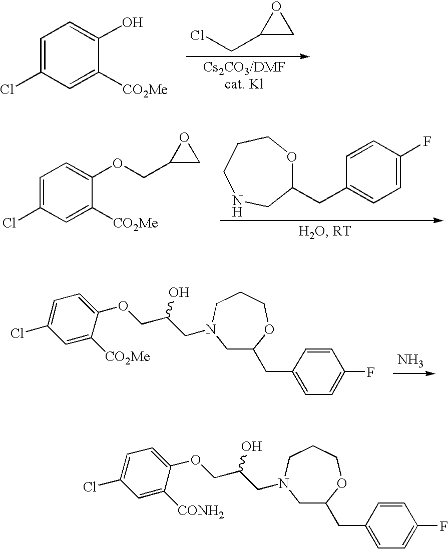

2-(4-Fluoro-benzyl)-[1,4]oxazepane was prepared as described in Example 6.

### Step 1: Methyl-4-chloro-(phenoxymethyloxirane)-2-carboxllate

To a solution of 5-Chloro-2-hydroxy-benzoic acid methyl ester (700 mg, 3.76 mmol) in DMF was added Cs₂CO₃ (1.83 g, 5.65 mmol), racemic epichlorohydrine (0.4 ml, 5.65 mmol) and a catalytic amount of potassium iodide. The reaction mixture was stirred overnight at room temperature. The reaction mixture was diluted with iced water and extracted with ethyl acetate. The organic solution was concentrated in vacuo and purified through Si-gel column chromatography (30% ethyl acetate-hexane) to afford 200 mg (22%) of methyl-4-chloro-(phenoxymethyloxirane)-2-carboxllate. LC/MS [M+H]⁺: 243.2. ¹H-NMR (400 MHz, DMSO-d₆) δ (ppm): 7.65 (s, 1H), 7.57 (d, J=8.8 Hz, 1H), 7.21 (d, J=8.9 Hz, 1H), 4.41 (d, J=11.4 Hz, 1H), 3.98 (m, 1H), 3.8 (s, 3H), 2.82 (t, J=4.6 Hz, 1H), 2.77 (brs, 1H).

### Step 2: Methyl-{1-(4-Chloro-phenoxy)-3-[2-(4-fluoro-benzyl)-[1,4]oxazepan-4-yl]-propan-2-ol}benzoate

To a solution of methyl-4-chloro-(phenoxymethyloxirane)-2-carboxllate (100 mg, 0.41 mmol) in water was added 2-(4-Fluoro-benzyl)-[1,4]oxazepane (104 mg, 0.49 mmol) and the resulting mixture was stirred overnight at room temperature then extracted with ethyl acetate. The organic layer was dried over sodium sulfate and evaporated to dryness under reduced pressure to afford 140 mg (75%) of methyl-{1-(4-Chloro-phenoxy)-3-[2-(4-fluoro-benzyl)-[1,4]oxazepan-4-yl]-propan-2-ol}benzoate. LC/MS [M+H]⁺: 451.9. ¹H-NMR (400 MHz, DMSO-d₆) δ (ppm): 7.63-7.04 (7Ar H), 4.73 (s, 1H), 3.99 (m, 2H), 2.82 (m, 1H), 3.76 (s, 3H), 3.69 (m, 3H), 3.48 (m, 2H), 2.79 (m, 2H), 2.7-2.56 (m, 5H), 1.76 (brs, H), 1.68 (brs, 1H).

### Step 3

A solution of methyl-{1-(4-Chloro-phenoxy)-3-[2-(4-fluoro-benzyl)-[1,4]oxazepan-4-yl]-propan-2-ol}benzoate (140 mg, 0.31 mmol) in methanol (15 ml) was saturated with ammonia by purging with ammonia gas at atmospheric pressure at −5° C. The resulting mixture was heated at 60° C. overnight in a sealed tube. The solution was then concentrated under reduced pressure and purified through column chromatography (Si-gel, 3% MeOH-DCM) to afford 100 mg (74%) of 4-chloro-2-{3-[2-(4-fluoro-benzyl)-[1,4]oxazepan-4-yl]-2-hydroxy-propoxy}-benzamide. LC/MS [M+H]⁺: 437.3. ¹H-NMR (400 MHz, DMSO-d₆) δ (ppm): 7.86 (s, 1H), 7.80 (d, J=2.4 Hz, 1H), 7.75 (s, 1H), 7.50 (dd, J=8.8, 2.4 Hz, 1H), 7.20 (m, 3H), 7.04 (t, J=8.8 Hz, 2H), 5.08 (d, J=4.0 Hz, 1H), 4.16 (m, 1H), 4.05 (m, 1H), 3.9 (br s, 1H), 3.71 (m, 2H), 3.5 (m, 1H), 2.82 (m, 2H), 2.58 (m, 4H), 1.9-1.6 (m, 2H), 1.23 (br s, 2H). HPLC purity: 99.1%.

### Example 35

### N-(3-Chloro-2-methyl-phenyl)-2-[2-(4-fluoro-benzyl)-[1,4]oxazepan-4-yl]-acetamide

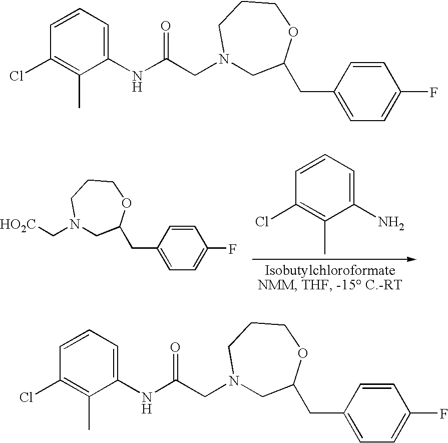

2-(4-Fluoro-benzyl)-[1,4]oxazepane-acetic acid was prepared as described in Example 22.

### Step-1: N-(3-Chloro-2-methyl-phenyl)-2-[2-(4-fluoro-benzyl)-[1,4]oxazepan-4-yl]-acetamide

To a solution of 2-(4-Fluoro-benzyl)-[1,4]oxazepane-actetic acid (100 mg, 0.37 mmol) in THF was added isobutyl chloroformate (0.05 ml, 3.74 mmol) and N-methyl morpholine (0.1 ml, 0.75 mmol) at −15° C. The reaction mixture was stirred at room temperature for 30 minutes. A solution of 3-Chloro-2-methyl-phenylamine (0.05 ml, 0.37 mmol) in THF was then added and the solution was stirred overnight at room temperature. The mixture was then concentrated, diluted with water and extracted with ethyl acetate. The organic layer was washed with 0.1 (N)HCl, saturated aq. NaHCO₃ and water, dried over sodium sulfate and concentrated. The crude product was purified through Si-gel column chromatography (40-50% ethyl acetate hexane) to afford 31 mg (21%) of N-(3-Chloro-2-methyl-phenyl)-2-[2-(4-fluoro-benzyl)-[1,4]oxazepan-4-yl]-acetamide. LC/MS [M+H]⁺: 391.4. ¹H-NMR (400 MHz, DMSO-d₆) δ (ppm): 9.50 9s, 1H), 7.52-7.04 (m, 7H), 3.86-3.59 (m, 3H), 2.90-2.54 (m, 8H), 2.19 (s, 3H), 1.79-1.77 (m, 2H). HPLC purity: 94.4%

### Example 36

### 4-[3-(2,6-Dichloro-phenoxy)-propyl]-2-(4-fluoro-benzyl)-[1,4]oxazepane

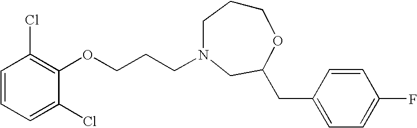

4-[3-(2,6-Dichloro-phenoxy)-propyl]-2-(4-fluoro-benzyl)-[1,4]oxazepane was prepared by procedures similar to those described in Examples 6 and 12. LC/MS [M+H]⁺: 412.1. ¹H-NMR (400 MHz, DMSO-d₆) δ (ppm): 7.49-7.05 (m, 7H), 4.02-3.52 (m, 7H), 2.79-2.62 (m, 6H), 2.34 (m, 1H). 1.86-1.84 (m, 3H). HPLC purity: 90.7%

### Example 37

### 2,6-Dichloro-N-{2-[2-(4-fluoro-benzyl)-[1,4]oxazepan-4-yl]-ethyl}-benzenesulfonamide

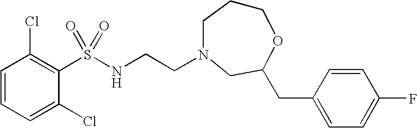

2,6-Dichloro-N-{2-[2-(4-fluoro-benzyl)-[1,4]oxazepan-4-yl]-ethyl}-benzenesulfonamide was prepared by a procedure similar to that described in Example 29. LC/MS [M+H]⁺: 461.0. ¹H-NMR (400 MHz, DMSO-d₆) δ (ppm): 7.84 (br s, 1H), 7.63-7.05 (m, 7H), 3.64-3.36 (m, 3H), 2.98 (m, 2H), 2.59-2.19 (m, 8H), 1.61 (m, 2H). HPLC purity: 90.7%

### Example 38

### N-{3-[2-(4-Fluoro-benzyl)-[1,4]oxazepan-4-yl]-propyl}-4-methoxy-benzenesulfonamide

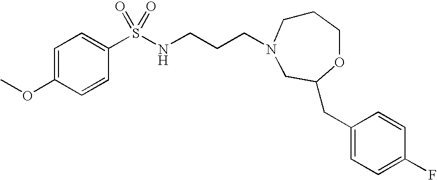

N-{3-[2-(4-Fluoro-benzyl)-[1,4]oxazepan-4-yl]-propyl}-4-methoxy-benzenesulfonamide was prepared by a procedure similar to that described in Example 29. LC/MS [M+H]⁺: 437.0. ¹H-NMR (400 MHz, DMSO-d₆) δ (ppm): 7.71-7.07 (m, 8H), 3.82 (s, 3H), 3.65 (m, 2H), 3.45 (m, 1H), 2.72-2.34 (m, 10H), 1.66 (m, 2H). HPLC purity: 97.3%

### Example 39

### 4-Chloro-N-{3-[2-(4-fluoro-benzyl)-[1,4]oxazepan-4-yl]-propyl}-benzenesulfonamide

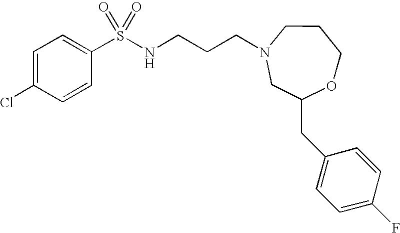

4-Chloro-N-{3-[2-(4-fluoro-benzyl)-[1,4]oxazepan-4-yl]-propyl}-benzenesulfonamide was prepared by a procedure similar to that described in Example 29. LC/MS [MA-1]⁺: 441.3. ¹H-NMR (400 MHz, DMSO-d₆) δ (ppm): 7.79-7.05 (m, 8H), 3.65 (m, 2H), 3.45 (m, 1H), 2.78-2.33 (m, 10H), 1.66 (m, 2H). HPLC purity: 95.5%

### Example 40

### 5-Chloro-2-{3-[2-(4-fluoro-benzyl)-[1,4]oxazepan-4-yl]-propoxy}-benzoic acid

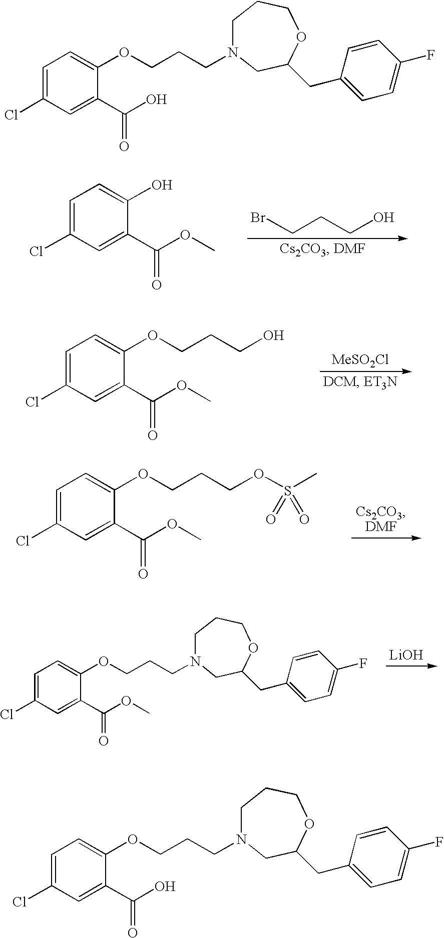

2-(4-Fluoro-benzyl)-[1,4]oxazepane was prepared as described in Example 6.

### Step 1: 5-Chloro-2-(3-hydroxy-propoxy)-benzoic acid methyl ester

To a solution of 5-Chloro-2-hydroxy-benzoic acid methyl ester (100 mg, 0.54 mmol) in DMF was added Cs₂CO₃ (615.7 mg, 1.89 mmol) and 3-bromopropanol (0.05 ml, 0.56 mmol) at room temperature, and the resulting mixture was stirred at 60° C. overnight. The mixture was then diluted with water and extracted with ethyl acetate. The organic layer was washed with water, dried over sodium sulfate and concentrated to afford 125 mg (95%) of 5-chloro-2-(3-hydroxy-propoxy)-benzoic acid methyl ester.

### Step 2: 5-Chloro-2-(3-methanesulfonyloxy-propoxy)-benzoic acid methyl ester

To a solution of 5-Chloro-2-(3-hydroxy-propoxy)-benzoic acid methyl ester (200 mg, 0.82 mmol) in dichloromethane was added triethylamine (0.17 ml, 1.23 mmol) and methane sulfonyl chloride (0.09 ml, 0.98 mmol) at 0° C. The reaction mixture was stirred overnight at room temperature then concentrated, diluted with water and extracted with ethyl acetate. The organic layer was washed with brine solution, dried over sodium sulfate, concentrated and purified through Si-gel column chromatography (5% ethyl acetate-hexane) to afford 90 mg (34%) of 5-Chloro-2-(3-methanesulfonyloxy-propoxy)-benzoic acid methyl ester. ¹H-NMR (400 MHz, DMSO-d₆) δ (ppm): 7.67-7.21 (m, 3H), 4.37 (m, 2H), 4.14 (m, 2H), 3.81 (s, 3H), 3.17 (s, 3H), 2.12 (m, 2H).

### Step 3: 5-Chloro-2-{3-[2-(4-fluoro-benzyl)-[1,4]oxazepan-4-yl]-propoxy}-benzoic acid methyl ester

To a solution of 5-Chloro-2-(3-methanesulfonyloxy-propoxy)-benzoic acid methyl ester (90 mg, 0.28 mmol) in DMF was added Cs₂CO₃ (182.5 mg, 0.56 mmol) and 2-(4-Fluoro-benzyl)-[1,4]oxazepane (64.2 mg, 0.31 mmol). The reaction mixture was stirred at 60° C. overnight then diluted with water and extracted with ethyl acetate. The organic layer was washed with water, dried over sodium sulfate, concentrated and purified through Si-gel column (8%-ethyl acetate-hexane) to afford 45 mg (37%) of 5-Chloro-2-{3-[2-(4-fluoro-benzyl)-[1,4]oxazepan-4-yl]-propoxy}-benzoic acid methyl ester. LC/MS [M+H]⁺: 436.2

### Step 4: 5-Chloro-2-{3-[2-(4-fluoro-benzyl)-[1,4]oxazepan-4-yl]-propoxy}-benzoic acid

To a solution of 5-Chloro-2-{3-[2-(4-fluoro-benzyl)-[1,4]oxazepan-4-yl]-propoxy}-benzoic acid methyl ester (40 mg, 0.09 mmol) in THF: H₂O (2:1) was added LiOH (10 mg, 0.18 mmol) and the solution was stirred for 5 hours. The reaction mixture was then concentrated and the crude product was dissolved in a minimum amount water, neutralized with 6(N)HCl solution and extracted with ethyl acetate. The organic layer was washed with water, dried over sodium sulfate, concentrated and washed with hexane to afford 15 mg (40%) of 5-Chloro-2-{3-[2-(4-fluoro-benzyl)-[1,4]oxazepan-4-yl]-propoxy}-benzoic acid. LC/MS [M+H]⁺: 422.1. ¹H-NMR (400 MHz, CDCl₃) δ (ppm): 7.81 (s, 1H), 7.34-6.82 (m, 6H), 4.28 (br s, 1H), 4.15 (br s, 2H), 3.78-2.06 (m, 14H). HPLC purity: 95.2%

### Example 41

### (5-Chloro-2-{3-[2-(4-chloro-benzyl)-[1,4]oxazepan-4-yl]-propoxy}-phenyl)-urea

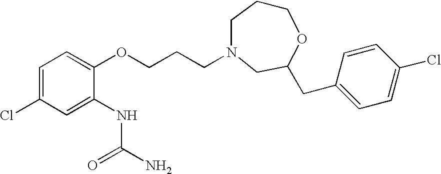

(5-Chloro-2-{3-[2-(4-chloro-benzyl)-[1,4]oxazepan-4-yl]-propoxy}-phenyl)-urea was prepared by procedures similar to those described in Examples 6 and Example 12. LC/MS [M+H]⁺: 452.1. ¹H-NMR (400 MHz, DMSO-d₆) δ (ppm): 8.18 (d, 1H), 7.87 (s, 1H), 7.31-6.86 (m, 6H), 6.39 (br s, 2H), 4.55 (m, 1H), 4.06 (m, 2H), 3.70 (m, 2H), 3.48 (m, 2H), 3.41 (m, 1H), 2.81-1.71 (m, 9H). HPLC purity: 92.6%

### Example 42

### 2-(4-Chloro-2-isoxazol-5-yl-phenoxy)-1-[4-(4-fluoro-benzyl)-[1,4]diazepan-1-yl]ethanone

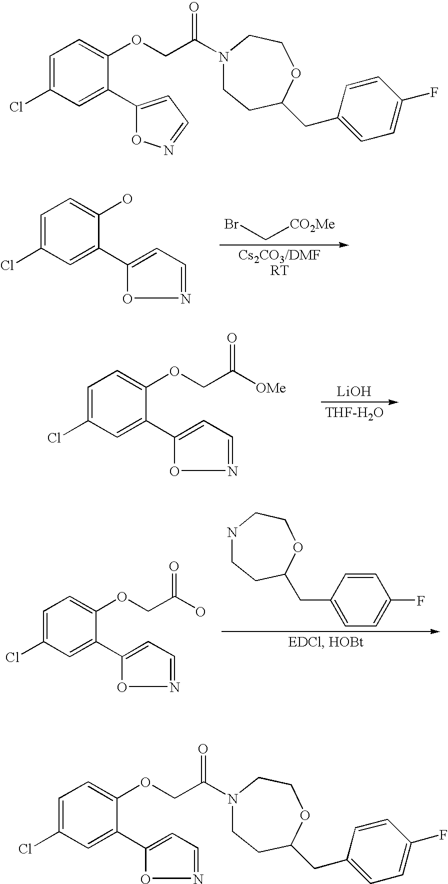

### Step 1: Methyl-(4-Chloro-2-isoxazol-5-yl-phenoxy)-acetate

To a solution of 4-chloro-2-isoxazol-5-yl-phenol (500 mg, 2.6 mmol) in DMF was added potassium carbonate (706 mg, 5.1 mmol) and methyl bromoacetate (0.4 ml, 3.8 mmol) at 0° C. The resulting solution was stirred overnight at room temperature then diluted with iced water and extracted with ethyl acetate. The organic layer was dried over sodium sulfate and concentrated to afford 700 mg (99%) of methyl-(4-Chloro-2-isoxazol-5-yl-phenoxy)-acetate. LC/MS [M+H]: 268.2. ¹H-NMR (400 MHz, DMSO-d₆) δ (ppm): 8.72 (d, J=1.8 Hz, 1H), 7.87 (d, J=2.8 Hz, 1H), 7.53 (dd, J=9.2, 2.6 Hz, 1H), 7.23-7.2 (d, 2H), 5.03 (s, 2H), 3.70 (s, 3H).

### Step-2: (4-Chloro-2-isoxazol-5-yl-phenoxy)-acetic acid

To a solution of methyl-(4-Chloro-2-isoxazol-5-yl-phenoxy)-acetate (700 mg, 2.6 mmol) in THF-water (4:1) was added LiOH (132 mg, 3.14 mmol) at 0° C. and the mixture was stirred for 4 hours, then concentrated. The residue was diluted with water, the pH of the solution was adjusted to 2 with 1(N)HCl and the product was extracted with ethyl acetate. The organic layer was dried over sodium sulfate and concentrated to afford 600 mg (90%) of (4-Chloro-2-isoxazol-5-yl-phenoxy)-acetic acid. LC/MS [M+H]: 254.2. ¹H-NMR (400 MHz, DMSO-d₆) δ (ppm): 13.29 (br s, 1H), 8.73 (d, J=1.8 Hz, 1H), 7.87 (d, J=2.8 Hz, 1H), 7.53 (dd, J=8.8, 2.1 Hz, 1H), 7.28 (d, J=1.68 Hz, 1H), 7.20 (d, J=1.68 Hz, 1H), 4.91 (s, 2H).

### Step-3: 2-(4-Chloro-2-isoxazol-5-yl-phenoxy)-1-[7-(4-fluoro-benzyl)-[1,4]oxazepan-4-yl]-ethanone

To a solution of (4-Chloro-2-isoxazol-5-yl-phenoxy)-acetic acid (100 mg, 0.39 mmol) in DMF was added 7-(4-Fluoro-benzyl)-[1,4]oxazepane (83 mg, 0.39 mmol), EDCI (100 mg, 0.51 mmol), HOBt (30 mg, 0.19 mmol) and DIPEA (0.25 ml, 1.18 mmol) at 5-10° C. The reaction mixture was stirred at room temperature for 16 hours then diluted with water and extracted with ethyl acetate. The organic layer was concentrated under reduced pressure and purified through column chromatography (Si-gel, 2% MeOH-DCM) to afford 85 mg (49%) of 2-(4-Chloro-2-isoxazol-5-yl-phenoxy)-1-[4-(4-fluoro-benzyl)-[1,4]diazepan-1-yl]ethanone. LC/MS [M+H]⁺: 445.3. ¹H-NMR (400 MHz, DMSO-d₆) δ (ppm): 8.70 (s, 1H), 7.86 (s, 1H0, 7.55 (m, 2H), 7.28 (m, 3H), 7.08 (m, 2H), 5.06 (m, 2H), 3.88-3.37 (m, 6H), 2.71 (m, 2H), 1.90-1.51 (m, 3H). HPLC purity: 96.2%

### Example 43

### (5-Chloro-2-{3-[4-(4-chloro-benzyl)-4-hydroxy-3,3-dimethyl-piperidin-1-yl]-propoxy}-phenyl)-urea

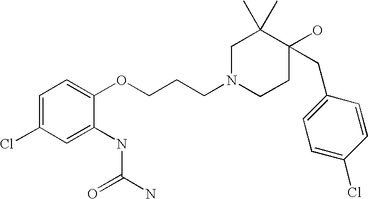

(5-Chloro-2-{3-[4-(4-chloro-benzyl)-4-hydroxy-3,3-dimethyl-piperidin-1-yl]-propoxy}-phenyl)-ure was prepared by a procedure similar to that described in Example 30. LC/MS [M+H]⁺: 480.3. ¹H-NMR (400 MHz, DMSO-d₆) δ (ppm): 8.17 (br s, 1H), 7.74 (br s, 1H), 7.26-6.64 (m, 6H), 5.42 (br s, 2H), 3.95 (m, 2H), 3.2-1.23 (m, 15H), 1.16 (s, 3H), 1.00 (s, 3H). ¹³C-NMR (400 MHz, DMSO-d₆) δ (ppm): 156.24, 145.64, 135.2, 132.54, 132.23, 129.93, 128.31, 126.00, 121.37, 118.82, 111.51, 72.65, 66.59, 61.97, 54.52, 49.95, 40.59, 38.25, 32.29, 26.77, 24.02, and 21.61. HPLC purity: 95.54%

### Example 44

### (5-Chloro-2-{3-[4-(4-fluoro-benzyl)-4-cyano-3,3-dimethyl-piperidin-1-yl]-propoxy}-phenyl)-urea

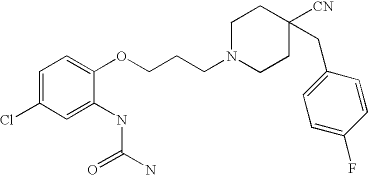

### Preparation of 4-Cyano-4-(4-fluoro-benzyl)-piperidine

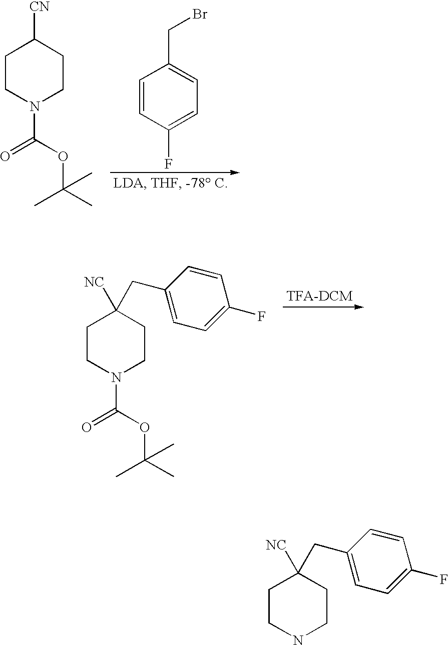

### Step-1: 4-Cyano-4-(4-fluoro-benzyl)-piperidine-1-carboxylic acid tert butyl ester

To a solution of diisopropylamine (3.2 ml, 22.8 mmol) in THF was added a solution of n-butyl lithium in hexane (1.6M, 15.8 ml, 26.6 mmol) at 0° C. The reaction mixture was stirred at room temperature for 1 hour then cooled to −78° C. A solution of 4-cyano-1-boc piperidine (4.0 g, 19 mmol) in THF was then added drop wise at −78° C. and the mixture was allowed to −40° C. over a period of 1 hour. The mixture was recooled to −78° C. and a solution of 4-fluorobenzyl bromide in THF was added drop wise. The reaction mixture was stirred at −78° C. for 4 hours then quenched at 0° C. with a saturated aqueous solution of ammonium chloride. The product was extracted with ethyl acetate. The organic layer was washed with brine solution, dried over sodium sulfate and concentrated. The crude product was crystallized from hexane to afford 3 g (50%) of 4-Cyano-4-(4-fluoro-benzyl)-piperidine-1-carboxylic acid tert butyl ester. LC/MS [M+H]⁺: 319.4

### Step-2: 4-Cyano-4-(4-fluoro-benzyl)-piperidine

To a solution of 4-Cyano-4-(4-fluoro-benzyl)-piperidine-1-carboxylic acid tert butyl ester (3 g, 9.4 mmol)) in DCM was added trifluoroacetic acid (5.7 ml, 75.2 mmol) and the solution was stirred for about 5 hours. The mixture was then concentrated and the residue was washed with dry ether. The TFA salt was diluted with a minimum volume of water and neutralized with aq. NaOH solution at 5-10° C. The neutralized solution was extracted with ethyl acetate. The organic layer was dried over sodium sulfate and concentrated to afford 2.1 mg (98%) of 4-Cyano-4-(4-fluoro-benzyl)-piperidine. LC/MS [M+H]⁺: 219.4

(5-Chloro-2-{3-[4-(4-fluoro-benzyl)-4-cyano-3,3-dimethyl-piperidin-1-yl]-propoxy}-phenyl)-urea was prepared by a procedure similar to that described in Example 30. LC/MS [M+H]⁺: 445.2¹H-NMR (400 MHz, DMSO-d₆) δ (ppm): 8.18 (s, 1H), 7.86 9s, 1H), 7.32 (m, 2H), 7.17 (t, 2H), 6.95 (d, 1H), 6.88 (m, 1H), 6.40 (br s, 2H), 4.04 (m, 2H), 2.88 (m, 4H), 2.04 (m, 2H), 1.91 (m, 2H), 1.74 (m, 2H), 1.64 (m, 2H), 1.23 (m, 2H). HPLC purity: 92.5%

### Example 45

### 1-[3-(4-Chloro-2-isoxazol-5-yl-phenoxy)-propyl]-4-(4-fluoro-benzyl)-3,3-dimethyl-piperidin-4-ol

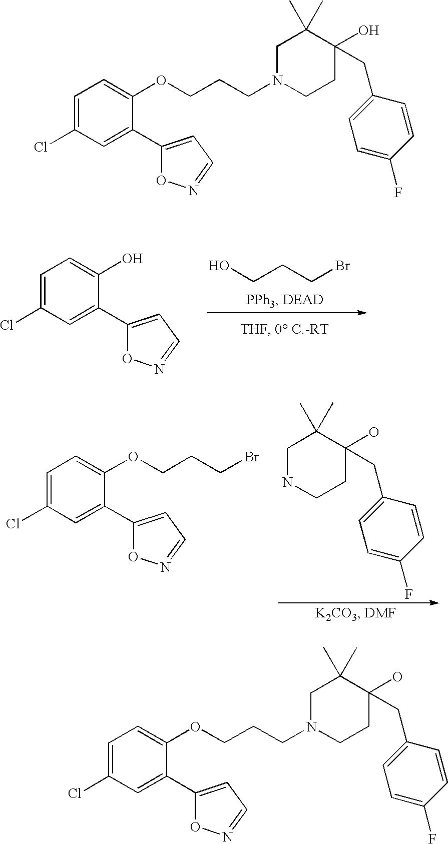

4-(4-fluoro-benzyl)-3,3-dimethyl-piperidin-4-ol was prepared as described in Example 30.

### Step-1: 5-[2-(3-Bromo-propoxy)-5-chloro-phenyl]-isoxazole

To a solution of triphenyl phosphine (536 mg, 2.04 mmol) in THF was added DIAD (0.3 ml, 1.53 mmol) at 0° C. and the mixture was stirred for 1 hour. A solution of 3-bromopropanol (0.1 ml, 1.02 mmol) and 4-Chloro-2-isoxazol-5-yl-phenol (200 mg, 1.02 mmol) in THF was then added at 0° C. and the resulting mixture was stirred at room temperature overnight. The mixture was concentrated and purified through Si-gel column (4% ethyl acetate-hexane) to afford 330 mg (100%) of 5-[2-(3-Bromo-propoxy)-5-chloro-phenyl]-isoxazole. LC/MS [M+H]⁺: 317.8

### Step-2: 4-(4-Chloro-benzyl)-1-[3-(4-chloro-2-isoxazol-5-yl-phenoxy)-propyl]-3,3-dimethyl-piperidin-4-ol

To a solution of 5-[2-(3-Bromo-propoxy)-5-chloro-phenyl]-isoxazole (100 mg, 0.32 mmol) in DMF was added K₂CO₃ (65.5 mg, 0.47 mmol) and 4-(4-fluoro-benzyl)-3,3-dimethyl-piperidin-4-ol (75 mg, 0.32 mmol) at room temperature. The mixture was stirred at room temperature overnight then diluted with water and extracted with ethyl acetate. The organic layer was washed with water and brine solution, dried over sodium sulfate and concentrated. The crude product was purified through Si-gel column (1.5% MeOH-DCM) to afford 26 mg (18%) of 4-(4-Chloro-benzyl)-1-[3-(4-chloro-2-isoxazol-5-yl-phenoxy)-propyl]-3,3-dimethyl-piperidin-4-ol. LC/MS [M+H]⁺: 473.4. ¹H-NMR (400 MHz, DMSO-d₆) δ (ppm): 8.69 (s, 1H), 7.83 (s, 1H), 7.52 (m, 1H), 7.24 (m, 3H), 7.04 (t, 2H), 6.90 (s, 1H), 4.20 (m, 2H), 3.81 (br s, 1H), 3.32 (m, 2H), 2.70 (m, 1H), 2.62 (m, 1H), 2.36 (m, 3H), 2.24 (m, 1H), 2.06 (m, 1H), 1.94 (m, 2H), 1.08 (m, 1H), 1.02 (s, 3H), 0.89 (s, 3H). HPLC purity: 98.1%

### Example 46

### 4-(4-Chloro-benzyl)-1-[3-(4-chloro-2-isoxazol-5-yl-phenoxy)-propyl]-3,3-dimethyl-piperidin-4-ol

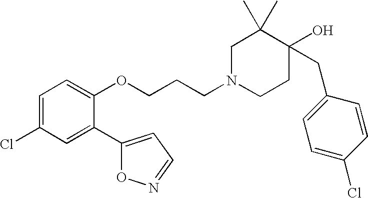

4-(4-Chloro-benzyl)-1-[3-(4-chloro-2-isoxazol-5-yl-phenoxy)-propyl]-3,3-dimethyl-piperidin-4-ol was prepared by a procedure similar to that described in Example 45. LC/MS [M+H]⁺: 489.3. ¹H-NMR (400 MHz, DMSO-d₆) δ (ppm): 8.69 (s, 1H), 7.83 (s, 1H), 7.52 (d, 2H), 7.26 (m, 4H), 6.89 (s, 1H), 4.19 (m, 2H), 3.85 (br s, 1H), 3.00 (br m, 6H), 2.66 (m, 1H), 2.32-1.92 (m, 6H), 1.02 (s, 3H), 0.89 (s, 3H). HPLC purity: 94.3%

### Example 47

### 1-[3-(5-Chloro-benzoimidazol-1-yl)-propyl]-4-(4-chloro-benzyl)-3,3-dimethyl-piperidin-4-ol

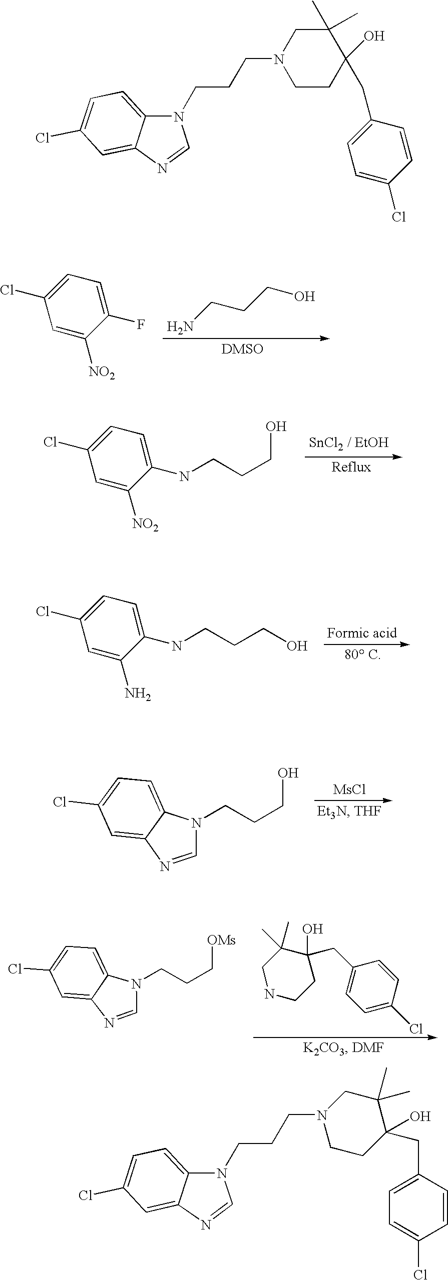

4-(4-Chloro-benzyl)-3,3-dimethyl-piperidin-4-ol was prepared by a procedure similar to that described in Example 30.

### Step 1: 3-(4-Chloro-2-nitro-phenylamino)-propan-1-ol

To a solution of 2-Fluoro-5-chloronitrobenzene (1.0 g, 5.7 mmol) in DMSO was added 3-aminopropanol (0.52 ml, 6.83 mmol) at 0° C. The resulting mixture was stirred at room temperature for 1 hour then diluted with iced water and extracted with ethyl acetate. The organic layer was washed with brine solution, dried over Na₂SO₄ and concentrated under reduced pressure to afford 1.2 g (91%) of 3-(4-Chloro-2-nitro-phenylamino)-propan-1-ol. LC/MS [M+H]⁺: 231.3. ¹H-NMR (400 MHz, DMSO-d₆) δ (ppm): 8.37 (br s, 1H), 8.05 (d, J=2.4 Hz, 1H), 7.57 (dd, J=9.2, 2.4 Hz, 1H), 7.11 (d, J=9.2 Hz, 1H), 4.72 (t, J=4.8 Hz, 1H), 3.52 (q, J=5.56 Hz, 2H), 3.42 (q, J=6.4 Hz, 2H), 1.76 (m, 2H).

### Step 2: 3-(2-Amino-4-chloro-phenylamino)-propan-1-ol

To a solution of 3-(4-Chloro-2-nitro-phenylamino)-propan-1-ol (800 mg, 3.47 mmol) in ethanol was added SnCl₂.2H₂O (3.91 g, 17.34 mmol). The resulting mixture was heated to reflux for 2 hours then cooled, concentrated under reduced pressure and diluted with water. The reaction mass was basified with dilute NaOH solution and extracted with ethyl acetate. The organic layer was washed with water and brine solution then dried over Na₂SO₄ and concentrated under reduced pressure to afford 700 mg (100%) of 3-(2-Amino-4-chloro-phenylamino)-propan-1-ol. LC/MS [M+H]⁺: 201.1. ¹H-NMR (400 MHz, CDCl₃) δ (ppm): 6.75 (dd, J=8.4, 1.6 Hz, 1H), 6.68 (d, J=1.2 Hz, 1H), 6.56 (d, J=8.4 Hz, 1H), 3.82 (t, J=5.8 Hz, 2H), 3.22 (t, J=6.48 Hz, 2H), 1.90 (m, 2H).

### Step 3: 3-(5-Chloro-benzoimidazol-1-yl)-propan-1-ol

A solution of 3-(2-Amino-4-chloro-phenylamino)-propan-1-ol (200 mg, 1 mmol) in formic acid was heated at 100° C. overnight. The reaction mixture was then poured into an ammonical solution at 0° C. and extracted with ethyl acetate. The organic layer was washed with water and brine solution, dried over Na₂SO₄ and concentrated under reduced pressure. The resulting white solid was triturated with hexane to afford 170 mg (81%) of 3-(5-Chloro-benzoimidazol-1-yl)-propan-1-ol. LC/MS [M+H]⁺: 211.0. ¹H-NMR (400 MHz, DMSO-d₆) δ (ppm): 8.28 (s, 1H), 7.70 (s, 1H), 7.64 (d, J=8.6 Hz, 1H), 7.28 (d, J=8.18 Hz, 1H), 4.66 (m, 1H), 4.31 (t, J=6.92 Hz, 2H, 3.36 (m, 2H), 1.92 (m, 2H).

### Step 4: Methanesulfonic acid 3-(5-chloro-benzoimidazol-1-yl)-propyl ester

To a solution of 3-(5-Chloro-benzoimidazol-1-yl)-propan-1-ol (220 mg, 1.04 mmol) in DCM was added triethylamine (0.73 ml, 5.2 mmol) and methane sulfonyl chloride (0.12 ml, 1.56 mmol) at 0° C. The reaction mixture was stirred for 30 min at 0° C. then concentrated, diluted with water, extracted with ethyl acetate and washed with water and brine solution. The organic layer was dried over Na₂SO₄ and concentrated under reduced pressure to afford 270 mg (90%) of methanesulfonic acid 3-(5-chloro-benzoimidazol-1-yl)-propyl ester. LC/MS [M+H]⁺: 289.2. ¹H-NMR (400 MHz, DMSO-d₆) δ (ppm): 8.32 (s, 1H), 7.72 (d, J=1.6 Hz, 1H), 7.68 (d, J=8.2, 1H), 7.31 (dd, J=8.2, 1H), 4.37 (t, J=7.0 Hz, 2H), 4.20 (t, 6.0 Hz, 2H), 3.17 (s, 3H), 2.22 (m, 2H).

### Step-5: 1-[3-(5-chloro-benzoimidazol-1-yl)-propyl]-4-(4-chloro-benzyl)-3,3-dimethyl-piperidin-4-ol

To a solution of methanesulfonic acid 3-(5-chloro-benzoimidazol-1-yl)-propyl ester (150 mg, 0.52 mmol) in DMF was added anhydrous K₂CO₃ (143.5 mg, 1.04 mmol) and 4-(4-Chloro-benzyl)-3,3-dimethyl-piperidin-4-ol (131.8 mg, 0.52 mmol). The reaction mixture was stirred at room temperature overnight. The mixture was then diluted with iced water and the resulting precipitate was filtered and dried. The crude product was triturated with hexane to afford 120 mg (52%) of 1-[3-(5-Chloro-benzoimidazol-1-yl)-propyl]-4-(4-chloro-benzyl)-3,3-dimethyl-piperidin-4-ol. LC/MS [M+H]⁺: 446.2. ¹H-NMR (400 MHz, DMSO-d₆) δ (ppm): 8.26 (s, 1H), 7.70 (s, 1H), 7.65 (d, J=8.5 Hz, 1H), 7.28 (m, 5H), 4.27 (t, J=6.52 Hz, 2H), 3.86 (s, 1H), 2.73 (m, 1H), 2.63 (m, 1H), 2.32 (m, 1H), 2.06 (m, 5H), 1.90 (m, 2H), 1.08 (s, 3H), 0.91 (s, 31-1). HPLC purity: 95.01%

### Example 48

### 1-[2-(5-Chloro-benzoimidazol-1-yl)-ethyl]-4-(4-chloro-benzyl)-3,3-dimethyl-piperidin-4-ol

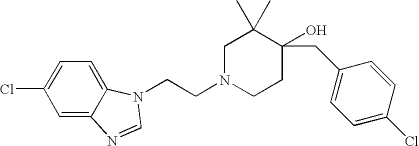

1-[2-(5-Chloro-benzoimidazol-1-yl)-ethyl]-4-(4-chloro-benzyl)-3,3-dimethyl-piperidin-4-ol was prepared by a procedure similar to that described in Example 47. LC/MS [M+H]⁺: 432.3. ¹H-NMR (400 MHz, DMSO-d₆) δ (ppm): 8.98 (br s, 1H), 8.38 (s, 1H), 7.26 (br d, 2H), 7.39-7.27 (m, 4H), 4.68 (m, 2H), 3.24 (m, 1H), 3.12 (m, 3H), 2.98 (m, 1H), 2.81 (m, 1H), 2.64 (m, 1H), 1.89 (m, 1H), 1.2 (m, 2H), 1.18 (s, 3H), 0.98 (m, 3H). HPLC purity: 99.8%

### Example 49

### N-(5-Chloro-2-{3-[4-(4-chloro-benzyl)-4-hydroxy-3,3-dimethyl-piperidin-1-yl]-propoxy}-phenyl)-acetamide

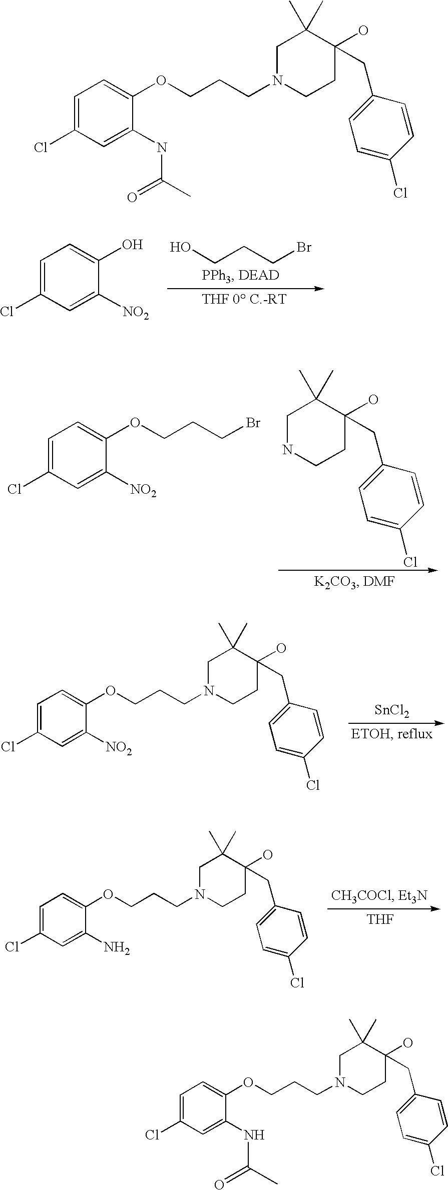

4-(4-Chloro-benzyl)-3,3-dimethyl-piperidin-4-ol was prepared by a procedure similar to that described in Example 30.

### Step-1:1-(3-Bromo-propoxy)-4-chloro-2-nitro-benzene

To a solution of triphenyl phosphine (3.02 g, 11.5 mmol) in THF was added DIAD (1.75 g, 8.6 mmol) at 0° C. and the mixture was stirred for 1 hour. A solution of 3-bromopropanol (0.5 ml, 5.7 mmol) and 4-chloro-2-nitrophenol (1 g, 05.76 mmol) in THF was then added at 0° C. and the mixture was stirred at room temperature overnight. The mixture was concentrated and purified through Si-gel column (2% ethyl acetate-hexane) to afford 1 g (60%) of 1-(3-Bromo-propoxy)-4-chloro-2-nitro-benzene. ¹H-NMR (400 MHz, DMSO-d₆) δ (ppm): 8.03 (d, 1H), 7.72 (m, 1H), 7.42 (d, 1H), 4.27 (t, 2H), 3.63 (t, 2H), 2.25 (m, 2H).

### Step-2: 1-[3-(4-Chloro-2-nitro-phenoxy)-propyl]-4-(4-chloro-benzyl)-3,3-dimethyl-piperidin-4-ol

To a solution of 1-(3-Bromo-propoxy)-4-chloro-2-nitro-benzene (465 mg, 1.57 mmol) in DMF was added K₂CO₃ (436 mg, 3.15 mmol) and 4-(4-Chloro-benzyl)-3,3-dimethyl-piperidin-4-ol (400 mg, 1.57 mmol) at room temperature. The mixture was stirred at room temperature overnight then diluted with water and extracted with ethyl acetate. The organic layer was washed with water and brine solution, dried over sodium sulfate and concentrated. The crude product was purified through Si-gel column (3% MeOH-DCM) to afford 680 mg (93%) of 1-[3-(4-Chloro-2-nitro-phenoxy)-propyl]-4-(4-chloro-benzyl)-3,3-dimethyl-piperidin-4-ol. LC/MS [M+H]⁺: 466.9

### Step-3: 1-[3-(2-Amino-4-chloro-phenoxy)-propyl]-4-(4-chloro-benzyl)-3,3-dimethyl-piperidin-4-ol

To a solution of 1-[3-(4-Chloro-2-nitro-phenoxy)-propyl]-4-(4-chloro-benzyl)-3,3-dimethyl-piperidin-4-ol (680 mg, 1.45 mmol) in ethanol was added SnCl₂.2H₂O (1.65 g, 7.27 mmol) and the resulting mixture was heated to reflux for 4 hours. The mixture was then concentrated, diluted with water and basified the solution with aq. NaOH. The basified solution was extracted with ethyl acetate. The organics were washed with water and brine solution, dried over sodium sulfate and concentrated to afford 600 mg (95%) of 1-[3-(2-Amino-4-chloro-phenoxy)-propyl]-4-(4-chloro-benzyl)-3,3-dimethyl-piperidin-4-ol. LC/MS [M+H]⁺: 437.3

### Step-4: N-(5-Chloro-2-{3-[4-(4-chloro-benzyl)-4-hydroxy-3,3-dimethyl-piperidin-1-yl]-propoxy}-phenyl)-acetamide

To a solution of 1-[3-(2-Amino-4-chloro-phenoxy)-propyl]-4-(4-chloro-benzyl)-3,3-dimethyl-piperidin-4-ol (100 mg, 0.23 mmol) in THF was added triethylamine (2 ml) at 10° C. Acetyl chloride (0.01 ml, 0.25 mmol) was then added and the solution was stirred at room temperature overnight. The mixture was concentrated, diluted with water and extracted with ethyl acetate. The organic layer was dried over sodium sulfate and purified through Si-gel column (1% MeOH-DCM) to afford 35 mg (32%) of N-(5-Chloro-2-{3-[4-(4-chloro-benzyl)-4-hydroxy-3,3-dimethyl-piperidin-1-yl]-propoxy}-phenyl)-acetamide. LC/MS [M+H]⁺: 479.1. ¹H-NMR (400 MHz, DMSO-d₆) δ (ppm): 9.03 (s, 1H), 8.03 (s, 1H), 7.27 (m, 4H), 7.05 (m, 2H), 4.06 (m, 2H), 3.84 (s, 1H), 2.70-1.88 (m, 13H), 1.48 (m, 2H), 1.02 (s, 3H), 0.90 (s, 3H). HPLC purity: 94.1%

### Example 50

### N-(5-Chloro-2-{3-[4-(4-chloro-benzyl)-4-hydroxy-3,3-dimethyl-piperidin-1-yl]propoxy}-phenyl)-2-dimethylamino-acetamide

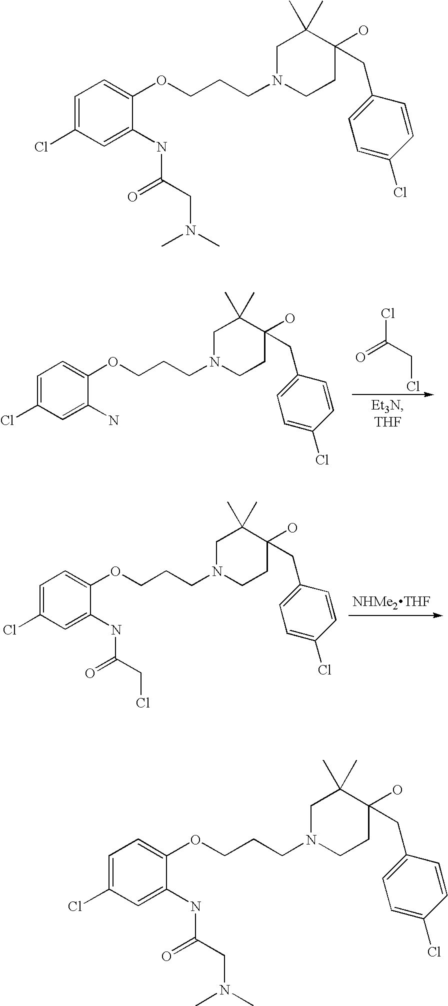

1-[3-(2-Amino-4-chloro-phenoxy)-propyl]-4-(4-chloro-benzyl)-3,3-dimethyl-piperidin-4-ol was prepared as described in Example 49.

### Step-1: 2-Chloro-N-(5-chloro-2-{3-[4-(4-chloro-benzyl)-4-hydroxy-3,3-dimethyl-piperidin-1-yl]-propoxy}-phenyl)-acetamide

To a solution of 1-[3-(2-Amino-4-chloro-phenoxy)-propyl]-4-(4-chloro-benzyl)-3,3-dimethyl-piperidin-4-ol (100 mg, 0.23 mmol) in DCM was added triethylamine (0.04 ml, 0.25 mmol) at 0° C. Chloroacetyl chloride (0.02 ml, 0.23 mmol) was then added and the solution was stirred for 1 hour. The reaction mixture was then concentrated, diluted with water and extracted with ethyl acetate. The organic layer was dried over sodium sulfate and purified through Si-gel column (1% MeOH-DCM) to afford 100 mg (85%) of 2-Chloro-N-(5-chloro-2-{3-[4-(4-chloro-benzyl)-4-hydroxy-3,3-dimethyl-piperidin-1-yl]-propoxy}-phenyl)-acetamide. LC/MS [M+H]⁺: 513.2

### Step-2: N-(5-Chloro-2-{3-[4-(4-chloro-benzyl)-4-hydroxy-3,3-dimethyl-piperidin-1-yl]-propoxy}-phenyl)-2-dimethylamino-acetamide

To a solution of 2-Chloro-N-(5-chloro-2-{3-[4-(4-chloro-benzyl)-4-hydroxy-3,3-dimethyl-piperidin-1-yl]-propoxy}-phenyl)-acetamide (100 mg, 0.195 mmol) in THF was added a solution of dimethyamine in THF and the resulting solution was stirred overnight at 60° C. The reaction mixture was then concentrated and purified through Si-gel (1.5% MeOH-DCM) to afford 68 mg (67%) of N-(5-Chloro-2-{3-[4-(4-chloro-benzyl)-4-hydroxy-3,3-dimethyl-piperidin-1-yl]-propoxy}-phenyl)-2-dimethylamino-acetamide. LC/MS [M+H]⁺: 522.2. ¹H-NMR (400 MHz, DMSO-d₆) δ (ppm): 9.71 (s, 1H), 8.42 (s, 1H), 7.25 (m, 2H), 7.10 (d, 2H), 6.97 (m, 1H), 6.76 (d, 1H), 4.06 (m, 2H), 3.07 (s, 2H), 2.86 (m, 1H), 2.62-1.60 (m, 16H), 1.40 (m, 1H), 1.12 (s, 3H), 0.99 (s, 3H). HPLC purity: 94.2%

### Example 51

### N-(5-Chloro-2-{3-[4-(4-chloro-benzyl)-4-hydroxy-3,3-dimethyl-piperidin-1-yl]-propoxy}-phenyl)-isobutyramide

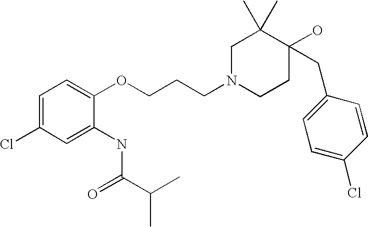

N-(5-Chloro-2-{3-[4-(4-chloro-benzyl)-4-hydroxy-3,3-dimethyl-piperidin-1-yl]-propoxy}-phenyl)-isobutyramide was prepared by a procedure similar to that described in Example 49. LC/MS [M+H]⁺: 507.2. ¹H-NMR (400 MHz, DMSO-d₆) δ (ppm): 8.93 (1,1H), 7.98 (s, 1H), 7.29-7.02 (m, 6H), 4.06 (m, 2H), 3.83 (br s, 1H), 2.76-2.67 (m, 4H), 2.36-1.87 (m, 8H), 1.52 (m, 1H), 1.09 (d, 6H), 1.01 (s, 3H), 0.89 (s, 3H). HPLC purity: 95.9%

### Example 52

### N-(5-Chloro-2-{3-[4-(4-chloro-benzyl)-4-hydroxy-3,3-dimethyl-piperidin-1-yl]-propoxy}-phenyl)-2,2-dimethyl-propionamide

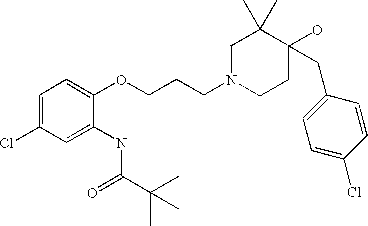

N-(5-Chloro-2-{3-[4-(4-chloro-benzyl)-4-hydroxy-3,3-dimethyl-piperidin-1-yl]-propoxy}-phenyl)-2,2-dimethyl-propionamide was prepared by a procedure similar to that described in Example 49. LC/MS [M+H]⁺: 521.6. ¹H-NMR (400 MHz, DMSO-d₆) δ (ppm): 8.43 (s, 1H), 7.95 (s, 1H), 7.29-7.03 (m, 6H), 4.06 (t, 2H), 3.85 (br s, 1H), 3.00 (m, 2H), 2.74-2.06 (m, 6H), 1.87 (m, 2H), 1.50 (m, 1H), 1.33-1.23 (s, 9H), 1.02 (s, 3H), 0.89 (s, 3H). HPLC purity: 93.8%

### Example 53

### Cyclobutanecarboxylic acid (5-chloro-2-{3-[4-(4-chloro-benzyl)-4-hydroxy-3,3-dimethyl-piperidin-1-yl]-propoxy}-phenyl)-amide

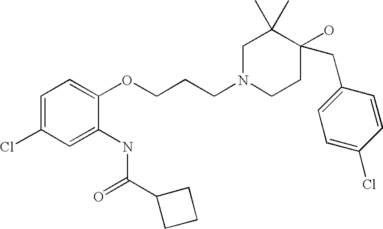

Cyclobutanecarboxylic acid (5-chloro-2-{3-[4-(4-chloro-benzyl)-4-hydroxy-3,3-dimethyl-piperidin-1-yl]-propoxy}-phenyl)-amide was prepared by a procedure similar to that described in Example 49. LC/MS [M+H]⁺: 519.3. ¹H-NMR (400 MHz, DMSO-d₆) δ (ppm): 8.81 (s, 1H), 7.27-7.04 (m, 6H), 4.05 (t, 2H), 3.86 (br s, 1H), 2.7-1.80 (m, 18H), 1.52 (m, 1H), 1.01 (s, 3H), 0.89 (s, 3H). HPLC purity: 92.1%

### Example 54

### N-(5-Chloro-2-{3-[4-(4-chloro-benzyl)-4-hydroxy-3,3-dimethyl-piperidin-1-yl]-propoxy}-phenyl)-methanesulfonamide

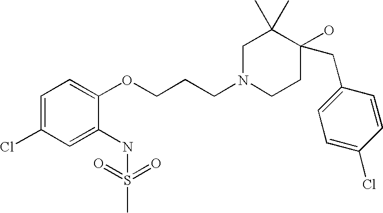

N-(5-Chloro-2-{3-[4-(4-chloro-benzyl)-4-hydroxy-3,3-dimethyl-piperidin-1-yl]-propoxy}-phenyl)-methanesulfonamide was prepared by a procedure similar to that described in Example 49. LC/MS [M+H]⁺: 515.3. ¹H-NMR (400 MHz, DMSO-d₆) δ (ppm): 9.09 (s, 1H), 9.00 (br s, 1H), 7.36-7.07 (m, 6H), 4.07 (m, 2H), 3.-2.64 (m, 14H), 2.18 (m, 2H), 1.22 (s, 3H), 0.98 (s, 3H). HPLC purity: 85.6%

### Example 55

### 5-Chloro-2-{3-[4-(4-chloro-benzyl)-4-hydroxy-3,3-dimethyl-piperidin-1-yl]-propoxy}-benzoic acid

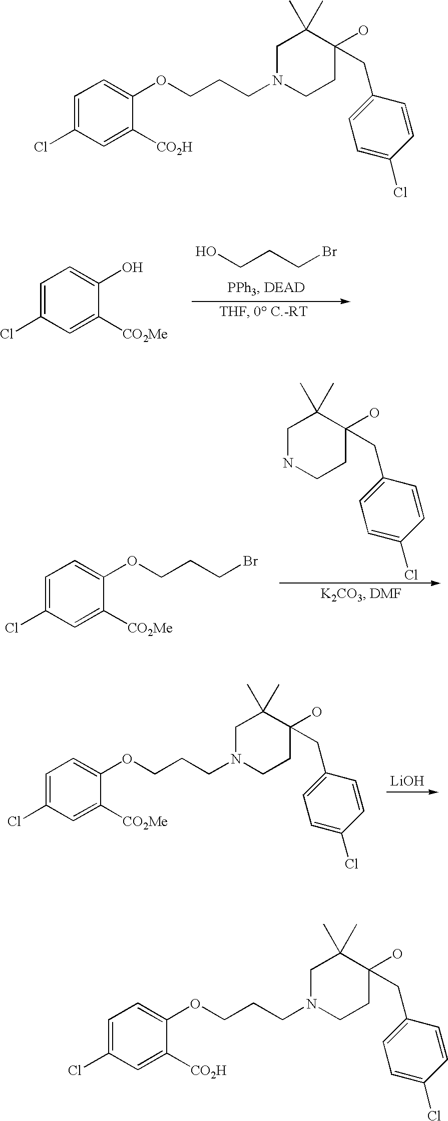

### Step-1: 2-(3-Bromo-propoxy)-5-chloro-benzoic acid methyl ester

To a solution of triphenyl phosphine (1.4 g, 5.35 mmol) in THF was added DIAD (0.8 ml, 4.02 mmol) at 0° C. and stirred for 1 hour. A solution of 3-bromopropanol (0.25 ml, 2.68 mmol) and methyl-3-chloro-salicylate (500 mg, 2.68 mmol) in THF was added at 0° C. and the mixture was stirred at room temperature overnight. The mixture was then concentrated and purified through Si-gel column (2% ethyl acetate-hexane) to afford 500 g (61%) of 2-(3-Bromo-propoxy)-5-chloro-benzoic acid methyl ester. ¹H-NMR (400 MHz, DMSO-d₆) δ (ppm): 7.67-7.21 (m, 3H), 4.15 (t, 2H), 3.80 (s, 3H), 3.71 (t, 2H), 2.23 (m, 2H).

### Step-2: 5-Chloro-2-{3-[4-(4-chloro-benzyl)-4-hydroxy-3,3-dimethyl-piperidin-1-yl]-propoxy}-benzoic acid methyl ester

To a solution of 2-(3-Bromo-propoxy)-5-chloro-benzoic acid methyl ester (370 mg, 1.2 mmol) in DMF was added K₂CO₃ (332 mg, 2.4 mmol) and 4-(4-Chloro-benzyl)-3,3-dimethyl-piperidin-4-ol (332 mg, 1.2 mmol) at room temperature. The resulting mixture was stirred at room temperature overnight then diluted with water and extracted with ethyl acetate. The organic layer was washed with water and brine solution, dried over sodium sulfate and concentrated. The crude product was purified through Si-gel column (5% MeOH-DCM) to afford 500 mg (86.5%) of 5-Chloro-2-{3-[4-(4-chloro-benzyl)-4-hydroxy-3,3-dimethyl-piperidin-1-yl]-propoxy}-benzoic acid methyl ester. LC/MS [M+H]⁺: 479.8

### Step-3: 5-Chloro-2-{3-[4-(4-chloro-benzyl)-4-hydroxy-3,3-dimethyl-piperidin-1-yl]-propoxy}-benzoic acid

To a solution of 5-Chloro-2-{3-[4-(4-chloro-benzyl)-4-hydroxy-3,3-dimethyl-piperidin-1-yl]-propoxy}-benzoic acid methyl ester (100 mg, 0.21 mmol) in THF: H₂O (2:1) was added LiOH (30 mg, 0.62 mmol) and the resulting solution was stirred for 5-6 hours. The mixture was then concentrated, diluted with water, neutralized with 6(N)HCl solution and extracted with ethyl acetate. The organic layer was washed with water, dried over sodium sulfate and concentrated. The residue was washed with hexane to afford 80 mg (81%) of 5-Chloro-2-{3-[4-(4-chloro-benzyl)-4-hydroxy-3,3-dimethyl-piperidin-1-yl]-propoxy}-benzoic acid. LC/MS [M+H]⁺: 466.4. ¹H-NMR (400 MHz, DMSO-d₆) δ (ppm): 7.63 (s, 1H), 7.40 (d, 1H), 7.28 (m, 4H), 7.10 (d, 1H), 4.22 (br s, 1H), 4.03 (m, 3H), 2.83-1.89 (m, 11H), 1.02 (s, 3H), 0.92 (s, 3H). HPLC purity: 99.3%

### Example 56

### 5-Chloro-2-{3-[4-(4-chloro-benzyl)-4-hydroxy-3,3-dimethyl-piperidin-1-yl]-propoxy}-benzamide

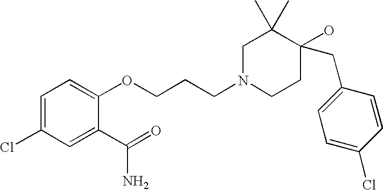

5-Chloro-2-{3-[4-(4-chloro-benzyl)-4-hydroxy-3,3-dimethyl-piperidin-1-yl]-propoxy}-benzoic acid methyl ester was prepared as described in Example 55.

A solution of 5-chloro-2-{3-[4-(4-chloro-benzyl)-4-hydroxy-3,3-dimethyl-piperidin-1-yl]-propoxy}-benzoic acid methyl ester (100 mg, 0.21 mmol) in methanol was saturated with ammonia by purging with ammonia gas at atmospheric pressure at −5° C. The mixture was heated at 60° C. overnight in a sealed tube. The mixture was then concentrated and washed with hexane to afford 90 mg (92%) of 5-chloro-2-{3-[4-(4-chloro-benzyl)-4-hydroxy-3,3-dimethyl-piperidin-1-yl]-propoxy}-benzamide. LC/MS [M+H]⁺: 465.0. ¹H-NMR (400 MHz, DMSO-d₆) δ (ppm): 7.72 (br s, 2H), 7.59 (br s, 1H), 7.49 (dd, 1H), 7.25 (m, 4H), 7.17 (d, 1H), 4.14 (t, 4.15), 3.85 (br s, 1H), 2.70-1.89 (m, 12H), 1.02 (s, 3H), 0.89 (s, 3H). HPLC purity: 96.9%

### Example 57

### 1-[3-(5-Chloro-benzoimidazol-1-yl)-propyl]-4-(4-chloro-phenyl)-3,3-dimethyl-piperidin-4-ol

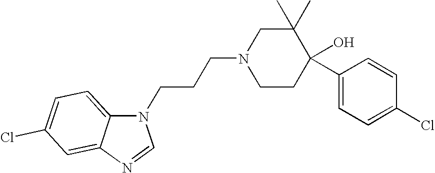

1-[3-(5-Chloro-benzoimidazol-1-yl)-propyl]-4-(4-chloro-phenyl)-3,3-dimethyl-piperidin-4-ol was prepared by a procedure similar to that described in Example 47. LC/MS [M+H]⁺: 432.4. ¹H-NMR (400 MHz, DMSO-d₆) δ (ppm): 8.28 (s, 1H), 7.67 (m, 2H), 7.45 (d, 2H), 7.31 (m, 3H), 4.29 (t, 2H), 2.56 (m, 2H), 2.27 (m, 2H), 2.13 (m, 3H), 1.93 (m, 2H), 1.40 (m, 1H), 0.81 (s, 3H), 0.61 (s, 3H). HPLC purity: 90%

### Example 58

### 1-[3-(5-Chloro-benzoimidazol-1-yl)-propyl]-4-(4-fluoro-benzyl)-piperidine-4-carbonitrile

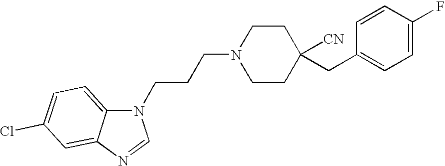

1-[3-(5-Chloro-benzoimidazol-1-yl)-propyl]-4-(4-fluoro-benzyl)-piperidine-4-carbonitrile was prepared by a procedure similar to that described in Example 47. LC/MS [M+H]⁺: 411.4 ¹H-NMR (400 MHz, DMSO-d₆) δ (ppm): 8.28 9s, 1H), 7.71-7.16 (m, 7H), 4.26 (t, 2H), 2.86 (s, 2H), 2.74 (d, 2H), 2.21 (t, 2H), 1.95 (m, 4H), 1.69-1.54 (m, 4H). HPLC purity: 85%

### Example 59

### N-(5-Chloro-2-{3-[4-(3,4-difluoro-benzyl)-4-hydroxy-3,3-dimethyl-piperidin-1-yl]-propoxy}-phenyl)-acetamide

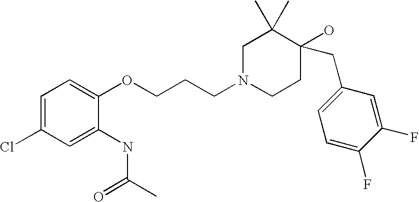

N-(5-Chloro-2-{3-[4-(3,4-difluoro-benzyl)-4-hydroxy-3,3-dimethyl-piperidin-1-yl]-propoxy}-phenyl)-acetamide was prepared by a procedure similar to that described in Example 49. LC/MS [M+H]⁺: 481.4. ¹H-NMR (400 MHz, DMSO-d₆) δ (ppm): 9.09 (s, 1H), 8.03 (s, 1H), 7.39-6.97 (s, 5H), 4.07 (t, 2H), 3.87 (br s, 1H), 2.70-1.86 (m, 14H), 1.58 (m, 1H), 1.05 (s, 3H), 0.91 (s, 3H). HPLC purity: 90.2%

### Example 60

### N-(5-Chloro-2-{3-[4-(4-chloro-benzyl)-4-hydroxy-3,3-dimethyl-piperidin-1-yl]-propoxy}-4-methoxy-phenyl)-acetamide

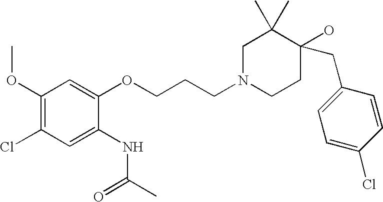

### Preparation of 4-Chloro-5-methoxy-2-nitrophenol

### Step 1: 2-chloro-5-fluoro-4-nitroanisole

2-Chloro-5-fluoroanisole (5 g, 0.031 mol) was dissolved in conc. H₂SO₄ (50 ml). Potassium nitrate (3.15 g, 0.031 mol) was then added portion wise at 0° C. and the mixture was stirred for 1 hour. The reaction mixture was then poured into ice and the resuling precipitate was extracted with ethyl acetate. The organic layer was washed with water, saturated bicarbonate solution and brine then dried over Na₂SO₄ and concentrated under reduce pressure to obtain 5.5 g (86%) of 2-chloro-5-fluoro-4-nitroanisole. GCMS [m/z]: 205. ¹H-NMR (400 MHz, DMSO-d₆) δ (ppm): 8.30 (d, J=7.9 Hz, 1H), 7.48 (d, J=13.4 Hz, 1H), 4.01 (s, 3H).

### Step 2: 4-Chloro-5-methoxy-2-nitrophenol

A solution of 2-chloro-5-fluoro-4-nitroanisole (2 g, 9.73 mmol) in 6 (N) NaOH solution was heated to reflux overnight. The pH of the solution was then adjusted to 1 with 6(N)HCl. The product was extracted with ethyl acetate, which was washed with water, saturated bicarbonate solution and brine. The organic layer was dried over Na₂SO₄ and concentrated under reduced pressure to afford 1.5 g (76%) of 4-Chloro-5-methoxy-2-nitrophenol. GCMS [m/z]: 203.5. ¹H-NMR (400 MHz, DMSO-d₆) δ (ppm): 10.9 (s, 1H), 8.14 (s, 1H), 6.59 (s, 1H), 3.97 (s, 3H).

N-(5-Chloro-2-{3-[4-(4-chloro-benzyl)-4-hydroxy-3,3-dimethyl-piperidin-1-yl]-propoxy}-4-methoxy-phenyl)-acetamide was prepared from 4-chloro-5-methoxy-2-nitrophenol by a procedure similar to that described in Example 49. LC/MS [M+H]⁺: 509.4. ¹H-NMR (400 MHz, DMSO-d₆) δ (ppm): 8.99 (s, 1H), 7.83 (s, 1H), 7.26 (m, 4H), 6.79 (s, 1H), 4.09 (t, 2H), 3.84 (s, 3H), 2.74-1.88 (m, 14H), 1.51 (1H), 1.23 (s, 1H), 1.03 (s, 3H), 0.90 (s, 3H). HPLC purity: 91.3%

### Example 61

### N-(5-Chloro-2-{3-[4-(4-chloro-3-methoxy-benzyl)-4-hydroxy-3,3-dimethyl-piperidin-1-yl]-propoxy}-phenyl)-acetamide

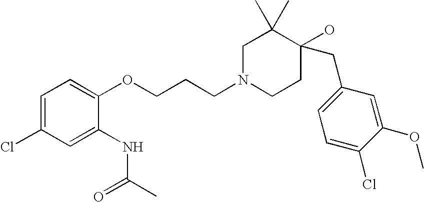

N-(5-Chloro-2-{3-[4-(4-chloro-3-methoxy-benzyl)-4-hydroxy-3,3-dimethyl-piperidin-1-yl]-propoxy}-phenyl)-acetamide was prepared by a procedure similar to that described in Example 49. LC/MS [M+H]⁺: 509.4 ¹H-NMR (400 MHz, DMSO-d₆) δ (ppm): 9.10 (s, 1H), 8.60 (br s, 1H), 8.00 (s, 1H), 7.32-6.75 (m, 4H), 4.75 (t, 2H), 4.02 (s, 1H), 3.84 (s, 3H), 3.03-1.85 (m, 11H), 1.32 9m, 2H), 1.17 (s, 3H0, 0.83 (s, 3H). HPLC purity: 98.1%

### Example 62

### 5-Chloro-2-{3-[4-(4-chloro-benzyl)-4-hydroxy-3,3-dimethyl-piperidin-1-yl]-propoxy}-N,N-dimethyl-benzamide

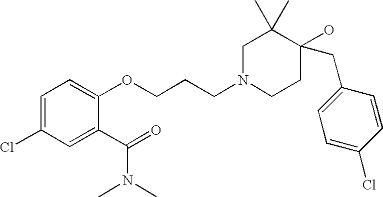

5-Chloro-2-{3-[4-(4-chloro-benzyl)-4-hydroxy-3,3-dimethyl-piperidin-1-yl]-propoxy}-N,N-dimethyl-benzamide was prepared by a procedure similar to that described in Example 56. LC/MS [M+H]⁺: 493.4. ¹H-NMR (400 MHz, DMSO-d₆) δ (ppm): 7.41-7.08 (m, 7H), 4.04 (t, 2H), 3.85 (s, 1H), 2.95 (s, 3H), 2,75 (s, 3H), 2.70-1.78 (m, 12H), 1.03 (s, 3H), 0.89 (s, 3H). HPLC purity: 90.3%

### Example 63

### 5-Chloro-2-{3-[4-(4-chloro-benzyl)-4-hydroxy-3,3-dimethyl-piperidin-1-yl]-propoxy}-N-methyl-benzamide

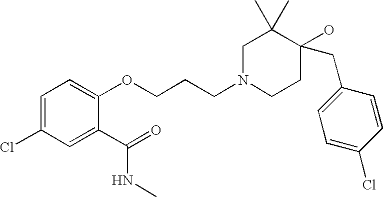

5-Chloro-2-{3-[4-(4-chloro-benzyl)-4-hydroxy-3,3-dimethyl-piperidin-1-yl]-propoxy}-N-methyl-benzamide was prepared by a procedure similar to that described in Example 56. LC/MS [M+H]⁺: 479.4. ¹H-NMR (400 MHz, DMSO-d₆) δ (ppm): 8.06 (m, 1H), 7.64 (d, 1H), 7.48 (m, 1H), 7.27 (m, 4H), 7.15 (d, 1H), 4.12 (t, 2H), 3.84 (s, 1H), 2.78-1.86 (m, 14H), 1.50 (m, 1H), 1.02 (s, 3H), 0.89 (s, 3H). HPLC purity: 96.9%

### Example 64

### N-(5-Chloro-2-{3-[4-(4-chloro-phenoxy)-piperidin-1-yl]-propoxy}-4-methoxy-phenyl)-acetamide

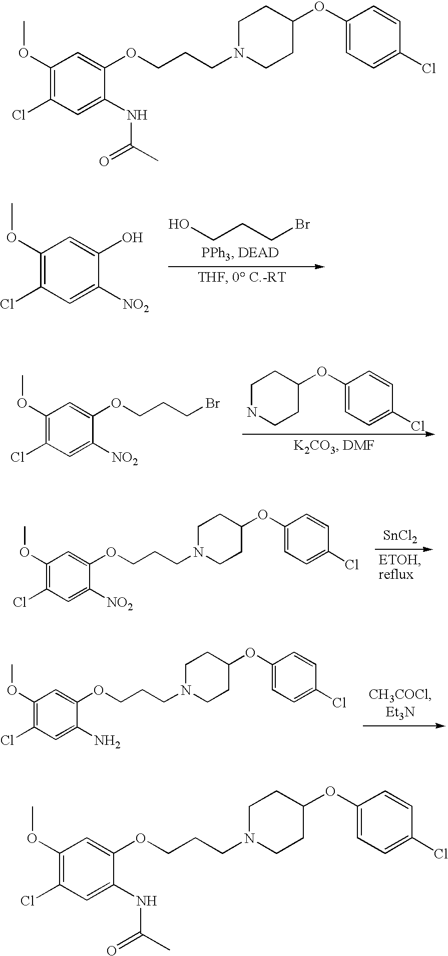

### Preparation of 4-(4-chloro-phenoxy)-piperidine

To a solution of triphenyl phosphine (1.3 g, 4.96 mmol) in THF was added DIAD (753 mg, 3.72 mmol) at 0° C. and the mixture was stirred for 1 hour. A solution of 4-hydroxy-piperidine-1-carboxylic acid tert-butyl ester (500 mg, 2.48 mmol) and 4-chlorophenol (320 mg, 2.48 mmol) in THF was then added at 0° C. and the mixture was stirred at room temperature overnight. The mixture was then concentrated and purified through Si-gel column (2% ethyl acetate-hexane) to afford 450 mg (58%) of 4-(4-Chloro-phenoxy)-piperidine-1-carboxylic acid tert-butyl ester.

To a solution of 4-(4-Chloro-phenoxy)-piperidine-1-carboxylic acid tert-butyl ester (450 mg, 1.45 mmol)) in DCM was added trifluoroacetic acid (1.1 ml, 14.5 mmol) and the solution was stirred for about 5 hours. The reaction mass was then concentrated and washed with dry ether. The TFA salt was diluted with a minimum volume of water and neutralized with aq. NaOH solution at 5-10° C. Extraction of the neutralized solution followed by concentration afforded 300 mg (99%) of 4-(4-Chloro-phenoxy)-piperidine. LC/MS [M+H]⁺: 212.3

### Step-1:1-(3-Bromo-propoxy)-4-chloro-2-nitro-benzene

To a solution of triphenyl phosphine (3.02 g, 11.5 mmol) in THF was added DIAD (1.75 g, 8.6 mmol) at 0° C. and the mixture was stirred for 1 hour. A solution of 3-bromopropanol (0.5 ml, 5.7 mmol) and 4-chloro-2-nitrophenol (1 g, 05.76 mmol) in THF was then added at 0° C. and the mixture was stirred at room temperature overnight. The mixture was then concentrated and purified through Si-gel column (2% ethyl acetate-hexane) to afford 1 g (60%) of 1-(3-Bromo-propoxy)-4-chloro-2-nitro-benzene. ¹H-NMR (400 MHz, DMSO-d₆) δ (ppm): 8.03 (d, 1H), 7.72 (m, 1H), 7.42 (d, 1H), 4.27 (t, 2H), 3.63 (t, 2H), 2.25 (m, 2H).

### Step-2: 5-Chloro-2-{3-[4-(4-chloro-phenoxy)-piperidin-1-yl]-propoxy}-4-methoxy-phenylamine

To a solution of 1-(3-Bromo-propoxy)-4-chloro-2-nitro-benzene (200 mg, 0.62 mmol) in DMF was added K₂CO₃ (171 mg, 1.2 mmol) and 4-(4-chloro-phenoxy)-piperidine (131 mg, 0.62 mmol) at room temperature. The resulting mixture was stirred at room temperature overnight then diluted with water and extracted with ethyl acetate. The organic layer was washed with water and brine solution then dried over sodium sulfate and concentrated. The crude product was purified through Si-gel column (3% MeOH-DCM) to afford 210 mg (74%) of 1-[3-(4-Chloro-5-methoxy-2-nitro-phenoxy)-propyl]-4-(4-chloro-phenoxy)-piperidine. LC/MS [M+H]⁺: 455.2

### Step-3: 1-[3-(2-Amino-4-chloro-phenoxy)-propyl]-4-(4-chloro-benzyl)-3,3-dimethyl-piperidin-4-ol

To a solution of 1-[3-(4-Chloro-5-methoxy-2-nitro-phenoxy)-propyl]-4-(4-chloro-phenoxy)-piperidine (210 mg, 0.46 mmol) in ethanol was added SnCl₂.2H₂O (521 mg, 2.3 mmol) and the resulting solution was heated to reflux for 4 hours. The mixture was then concentrated, diluted with water and basified with aq. NaOH. The product was extracted with ethyl acetate, which was washed with water and brine solution. The organic layer was dried over sodium sulfate and concentrated to afford 160 mg (82%) of 1-[3-(2-Amino-4-chloro-phenoxy)-propyl]-4-(4-chloro-benzyl)-3,3-dimethyl-piperidin-4-ol. LC/MS [M+H]⁺: 425.6

### Step-4: N-(5-Chloro-2-{3-[4-(4-chloro-phenoxy)-piperidin-1-yl]-propoxy}-4-methoxy-phenyl)-acetamide

To a solution of 1-[3-(2-Amino-4-chloro-phenoxy)-propyl]-4-(4-chloro-benzyl)-3,3-dimethyl-piperidin-4-ol (160 mg, 0.38 mmol) in THF was added triethylamine (0.13 ml, 0.94 mmol) at 10° C. Acetyl chloride (0.03 ml, 0.41 mmol) was then added and the solution was stirred at room temperature overnight. The mixture was then concentrated, diluted with water and extracted with ethyl acetate. The organic layer was dried over sodium sulfate and purified through Si-gel column (1% MeOH-DCM) to afford 120 mg (66%) of N-(5-Chloro-2-{3-[4-(4-chloro-phenoxy)-piperidin-1-yl]-propoxy}-4-methoxy-phenyl)-acetamide. LC/MS [M+H]⁺: 467.3. ¹H-NMR (400 MHz, DMSO-d₆) δ (ppm): 8.97 (s, 1H), 7.82 (s, 1H), 7.29 (d, 2H), 6.96 (d, 2H), 6.81 (s, 1H), 4.39 (m, 1H), 4.10 (m, 2H), 3.84 (s, 3H), 2.66 (m, 2H), 2.22-1.33 (m, 13H). HPLC purity: 93.3%

### Example 65

### 1-[2-(6-Chloro-1H-benzoimidazol-2-yl)-ethyl]-4-(4-chloro-benzyl)-3,3-dimethyl-piperidin-4-ol

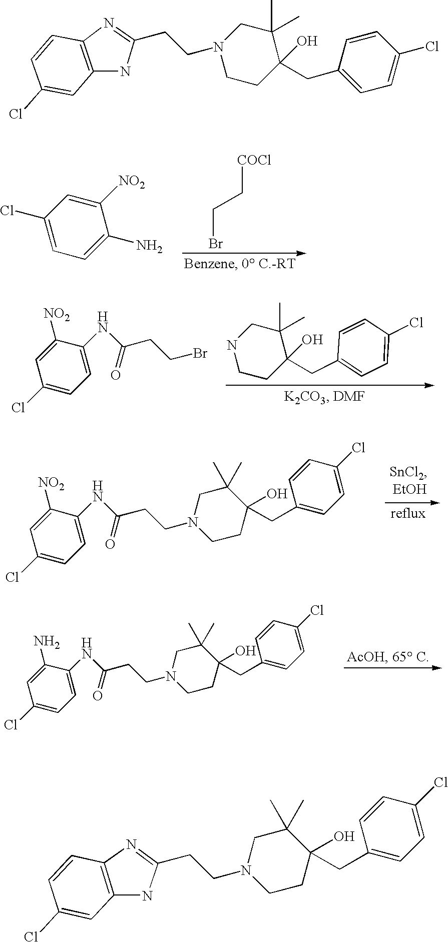

### Step-1: 3-Bromo-N-(4-chloro-2-nitro-phenyl)-propionamide

To a solution of 4-chloro-2-nitroaniline (500 mg, 3.3 mmol) in benzene was added 3-bromo-propionyl chloride (0.33 ml, 3.3 mmol) at 0° C. The resulting mixture was stirred overnight at room temperature then concentrated and diluted with water. The precipitate was isolated and dried. Recrystallization from benzene-hexane (4:1) afforded 300 mg (30%) of 3-Bromo-N-(4-chloro-2-nitro-phenyl)-propionamide. ¹H-NMR (400 MHz, DMSO-d₆) δ (ppm): 10.33 (s, 1H), 8.78 (d, 1H), 8.21 (d, 1H), 7.61 (m, 1H), 3.69 (t, 2H), 3.07 (t, 2H).

### Step-2: 3-[4-(4-Chloro-benzyl)-4-hydroxy-3,3-dimethyl-piperidin-1-yl]-N-(4-chloro-2-nitro-phenyl)-propionamide

To a solution of 3-Bromo-N-(4-chloro-2-nitro-phenyl)-propionamide (100 mg, 0.33 mmol) in DMF was added 4-(4-Chloro-benzyl)-3,3-dimethyl-piperidin-4-ol (82.5 mg, 0.33 mmol) and K₂CO₃ (28 mg, 0.17 mmol) at 10° C. The reaction mixture was stirred overnight at room temperature then diluted with water. The resulting precipitate was isolated, washed with water, washed with hexane and dried to afford 110 mg (69%) of 3-[4-(4-Chloro-benzyl)-4-hydroxy-3,3-dimethyl-piperidin-1-yl]-N-(4-chloro-2-nitro-phenyl)-propionamide. LC/MS [M+H]⁺: 480.2

### Step-3: N-(2-Amino-4-chloro-phenyl)-3-[4-(4-chloro-benzyl)-4-hydroxy-3,3-dimethyl-piperidin-1-yl]-propionamide

To a solution of 3-[4-(4-Chloro-benzyl)-4-hydroxy-3,3-dimethyl-piperidin-1-yl]-(4-chloro-2-nitro-phenyl)-propionamide (300 mg, 0.62 mmol) in ethanol was added SnCl₂.2H₂O (704 mg, 3.1 mmol) and the solution was heated to reflux for 4 hours. The reaction mixture was then concentrated, diluted with water and basified with aq. NaOH. The product was extracted with ethyl acetate, which was washed with water and brine solution. The organic layer was dried over sodium sulfate and concentrated to afford 80 mg (28%) of N-(2-Amino-4-chloro-phenyl)-3-[4-(4-chloro-benzyl)-4-hydroxy-3,3-dimethyl-piperidin-1-yl]-propionamide. LC/MS [M+H]⁺: 450.1

### Step-4: 1-[2-(6-Chloro-1H-benzoimidazol-2-yl)-ethyl]-4-(4-chloro-benzyl)-3,3-dimethyl-piperidin-4-ol

A solution of N-(2-Amino-4-chloro-phenyl)-3-[4-(4-chloro-benzyl)-4-hydroxy-3,3-dimethyl-piperidin-1-yl]-propionamide (40 mg, 0.08 mmol) in acetic acid (4 ml) and a drop of conc. HCl was heated at 70° C. overnight. The mixture was then concentrated, diluted with water and extracted with ethyl acetate. The organic layer was washed with water, dried over sodium sulfate, concentrated and purified through Si-gel column chromatography (6%-MeOH-DCM) to afford 20 mg (53%) of 1-[2-(6-Chloro-1H-benzoimidazol-2-yl)-ethyl]-4-(4-chloro-benzyl)-3,3-dimethyl-piperidin-4-ol. LC/MS [M+H]⁺: 432.2. ¹H-NMR (400 MHz, DMSO-d₆) δ (ppm): 7.52-7.12 (m, 7H), 4.50 (m, 1H), 3.48-2.66 (m, 12H), 1.02 (s, 3H), 0.92 (s, 3H). HPLC purity: 90.3%

### Example 66

### N-(5-Chloro-2-{3-[4-(4-chloro-phenyl)-4-hydroxy-3,3-dimethyl-piperidin-1-yl]-propoxy}-phenyl)-acetamide

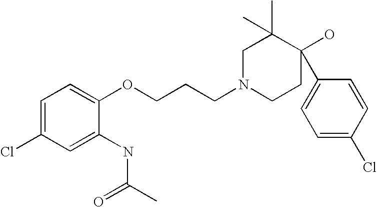

4-(4-Chloro-phenyl)-3,3-dimethyl-piperidin-4-ol was prepared as described in Example 10.

N-(5-Chloro-2-{3-[4-(4-chloro-phenyl)-4-hydroxy-3,3-dimethyl-piperidin-1-yl]-propoxy}-phenyl)-acetamide was prepared by a procedure similar to that described in Example 49. LC/MS [M+H]⁺: 465.2. ¹H-NMR (400 MHz, DMSO-d₆) δ (ppm): 9.09 (s, 1H), 8.04 (s, 1H), 7.46 (d, 2H), 7.33 (d, 2H), 7.06 (m, 2H), 4.64 (s, 1H), 4.09 (m, 2H), 2.67-1.89 (12H), 1.43 (m, 1H), 0.78 (s, s, 3H), 0.64 (s, 3H). HPLC purity: 93.5%

### Example 67

### N-(5-Chloro-2-{3-[4-(4-chloro-phenyl)-4-hydroxy-3,3-dimethyl-piperidin-1-yl]-propoxy}-4-methoxy-phenyl)-acetamide

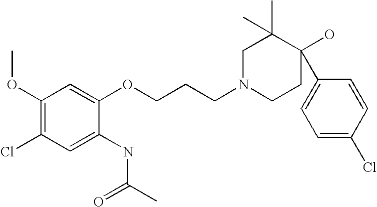

4-Chloro-5-methoxy-2-nitrophenol was prepared as described in Example 60.
4-(4-Chloro-phenyl)-3,3-dimethyl-piperidin-4-ol was prepared as described in Example 10.

N-(5-Chloro-2-{3-[4-(4-chloro-phenyl)-4-hydroxy-3,3-dimethyl-piperidin-1-yl]-propoxy}-4-methoxy-phenyl)-acetamide was prepared by a procedure similar to that described in Example 64. LC/MS [M+H]⁺: 495.4. ¹H-NMR (400 MHz, DMSO-d₆) δ (ppm): 9.00 (s, 1H), 7.83 (s, 1H), 7.46 (d, 2H), 7.34 (d, 2H), 6.81 (s, 1H), 4.65 (s, 1H), 4.13 (m, 2H), 3.84 (s, 3H), 3.20-1.90 (m, 12H), 1.43 (m, 1H), 0.79 (s, 3H), 0.64 (s, 3H). HPLC purity: 92.5%

### Example 68

### 4-(4-Chloro-benzyl)-1-[3-(4-chloro-2-pyrazol-1-yl-phenoxy)-propyl]-3,3-dimethyl-piperidin-4-ol

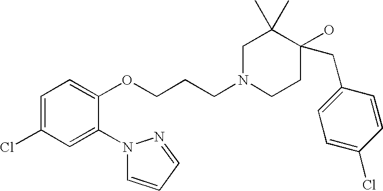

### Preparation of 1-(5-Chloro-2-ethoxy-phenyl)-1H-pyrazole

To a solution of 2-bromo-4-chlorophenol (500 mg, 2.41 mmol) in DMF was added Cs₂CO₃ (1.57 g, 4.82 mmol), pyrazole (230 mg, 3.37 mmol) and CuI (92 mg, 0.48 mmol) and the resulting mixture was heated at 80° C. overnight. The mixture was then diluted with water and extracted with ethyl acetate. The organic layer was washed with water, dried over sodium sulfate, concentrated and purified through Si-gel column chromatography (5% ethyl acetate-hexane) to afford 100 mg (21%) of 1-(5-Chloro-2-ethoxy-phenyl)-1H-pyrazole. LC/MS [M+H]⁺: 195.2

4-(4-Chloro-benzyl)-1-[3-(4-chloro-2-pyrazol-1-yl-phenoxy)-propyl]-3,3-dimethyl-piperidin-4-ol was prepared from 1-(5-Chloro-2-ethoxy-phenyl)-1H-pyrazole by a procedure similar to that described in Example 49. LC/MS [M+H]⁺: 488.4. ¹H-NMR (400 MHz, DMSO-d₆) δ (ppm): 8.25 d, 1H), 7.70 (t, 2H), 7.37 (m, 1H), 7.26 (5H), 6.5 (s, 1H), 4.13 (m, 2H), 3.82 (br s, 1H), 2.71-1.82 (m, 11H), 1.51 (m, 1H), 1.00 (s, 3H), 0.88 (s, 3H). HPLC purity: 92.0%.

### Example 69

### (5-Chloro-2-{3-[4-(4-chloro-benzyl)-4-hydroxy-3,3-dimethyl-piperidin-1-yl]-propoxy}-4-methoxy-phenyl)-urea

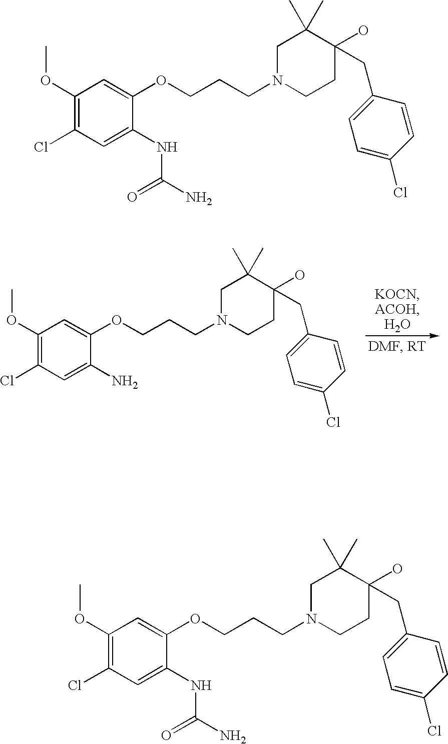

1-[3-(2-Amino-4-chloro-5-methoxy-phenoxy)-propyl]-4-(4-chloro-benzyl)-3,3-dimethyl-piperidin-4-ol was prepared as described in Example 60.

To a solution of 1-[3-(2-Amino-4-chloro-5-methoxy-phenoxy)-propyl]-4-(4-chloro-benzyl)-3,3-dimethyl-piperidin-4-ol (200 mg, 0.43 mmol) in DMF was added KOCN (70 mg, 0.86 mmol), AcOH (0.05 ml, 0.94 mmol) and water (0.05 ml). The reaction mixture was stirred at room temperature overnight then diluted with water and extracted with ethyl acetate. The organic layer was washed with water and dried over sodium sulfate. The organic layer was then concentrated and purified through Si-gel column chromatography (2.5% MeOH-DCM) to afford 30 mg (14%) of (5-Chloro-2-{3-[4-(4-chloro-benzyl)-4-hydroxy-3,3-dimethyl-piperidin-1-yl]-propoxy}-4-methoxy-phenyl)-urea. LC/MS [M+H]⁺: 509.9. ¹H-NMR (400 MHz, DMSO-d₆) δ (ppm): 8.97 (m, 1H), 8.08 (s, 1H), 7.70 (m, 1H), 7.29 (m, 3H), 6.77 (m, 1H), 6.25 (d, 2H), 4.11 (br s, 2H), 3.80 (s, 3H), 3.03-1.90 (11H), 1.54 (m, 1H), 1.03 (s, 3H), 0.90 (s, 3H). HPLC purity: 90.4%

### Example 70

### (5-Chloro-2-{3-[4-(4-fluoro-benzyl)-4-hydroxy-3,3-dimethyl-piperidin-1-yl]-propoxy}-4-methoxy-phenyl)-urea

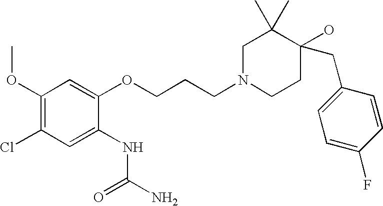

(5-Chloro-2-{3-[4-(4-fluoro-benzyl)-4-hydroxy-3,3-dimethyl-piperidin-1-yl]-propoxy}-4-methoxy-phenyl)-urea was prepared by a procedure similar to that described in Example 69. LC/MS [M+H]⁺: 493.9. ¹H-NMR (400 MHz, DMSO-d₆) δ (ppm): 9.08 (m, 1H), 8.08 (s, 1H), 7.70 (m, 1H), 7.24 (m, 2H), 7.06 (m, 2H), 6.76 (m, 1H), 6.25 (d, 214), 4.76 (m, 1H), 4.11 (br s, 2H), 3.80 (s, 3H), 3.31-1.89 (11H), 1.55 (m, 1H), 1.03 (s, 3H), 0.90 (s, 3H). HPLC purity: 92.2%.

### Example 71

### N-(5-Chloro-2-{3-[4-(4-chloro-phenylsulfanyl)-piperidin-1-yl]-propoxy}-4-methoxy-phenyl)-acetamide

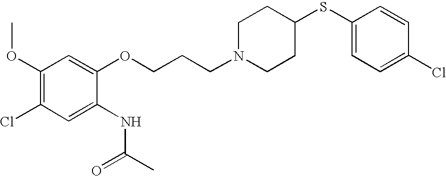

### Preparation of 4-(4-Chloro-phenylsulfanyl)-piperidine

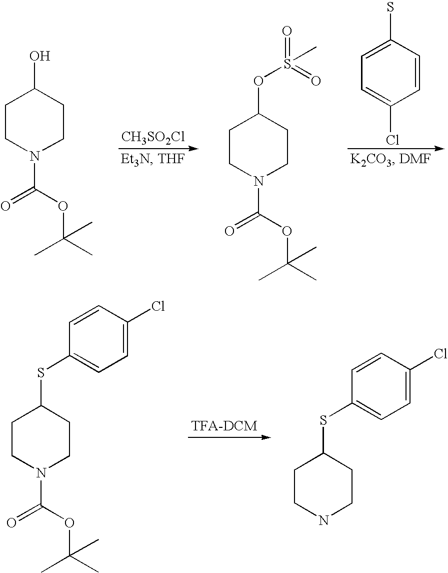

### Step-1: 4-Methanesulfonyloxy-piperidine-1-carboxylic acid tent-butyl ester

To a solution of 4-hydroxy-piperidine-1-carboxylic acid tert-butyl ester (2 g, 9.94 mmol) in THF was added triethylamine (6.94 ml, 49.7 mmol) at 0° C. Methanesulfonyl chloride (0.92 ml, 11.9 mmol) was added drop wise at 0° C. and the resulting mixture was stirred overnight at room temperature. The mixture was then concentrated, diluted with water and extracted with ethyl acetate. The organic layer was washed with water and dried over sodium sulfate and concentrated to afford 2.5 g (90.3%) of 4-methanesulfonyloxy-piperidine-1-carboxylic acid tert-butyl ester. ¹H-NMR (400 MHz, CDCl₃) δ (ppm): 4.86 (m, 1H), 3.68 (m, 2H), 3.29 (m, 2H), 3.02 (s, 3H), 1.96 (m, 2H), 1.81 (m, 2H), 1.44 (s, 9H).

### Step-2: 4-(4-Chloro-phenylsulfanyl)-piperidine-1-carboxylic acid tent-butyl ester

To a solution of 4-methanesulfonyloxy-piperidine-1-carboxylic acid tert-butyl ester (2.5 g, 8.59 mmol) in DMF was added 4-chlorothiophenol (1.68 g, 11.6 mmol) and K₂CO₃ (1.6 g, 11.6 mmol) at room temperature. The reaction mixture was stirred at 70° C. for 30 minutes then diluted with water and extracted with ethyl acetate. The organic layer was washed with water and dried over sodium sulfate. The organic layer was then concentrated and purified through Si-gel column (2.5%-ethyl acetate-hexane) to afford 2.5 g (86%) of 4-(4-Chloro-phenylsulfanyl)-piperidine-1-carboxylic acid tert-butyl ester. LC/MS [M+H]⁺: 328.3

### Step-3: 4-(4-Chloro-phenylsulfanyl)-piperidine

To a solution of 4-(4-Chloro-phenylsulfanyl)-piperidine-1-carboxylic acid tert-butyl ester (1 g, 3.05 mmol)) in DCM was added trifluoroacetic acid (2.35 ml, 30.5 mmol). The resulting solution was stirred for about 5 hours then concentrated and washed with dry ether. The TFA salt was diluted with a minimum volume of water and neutralized with aq. NaOH solution at 10° C. Extraction of the neutralized solution with ethyl acetate followed by concentration of the organics afforded 670 mg (96.4%) of 4-(4-Chloro-phenylsulfanyl)-piperidine. LC/MS [M+H]⁺: 228.0

N-(5-Chloro-2-{3-[4-(4-chloro-phenylsulfanyl)-piperidin-1-yl]-propoxy}-4-methoxy-phenyl)-acetamide was prepared by a procedure similar to that described in Example 64. LC/MS [M+H]⁺: 483.2. ¹H-NMR (400 MHz, DMSO-d₆) δ (ppm): 8.98 (s, 1H), 7.82 (s, 1H), 7.39 (br s, 4H), 6.80 (s, 1H), 4.08 (t, 2H), 3.84 (s, 3H), 3.26 (m, 1H), 2.79 (m, 2H), 2.43 (m, 2H), 2.04 (m, 5H), 1.87 (m, 4H), 1.48 (m, 2H). HPLC purity: 97.1%

### Example 72

### N-(5-Chloro-2-{3-[4-(4-chloro-benzenesulfonyl)-piperidin-1-yl]-propoxy}-4-methoxy-phenyl)-acetamide

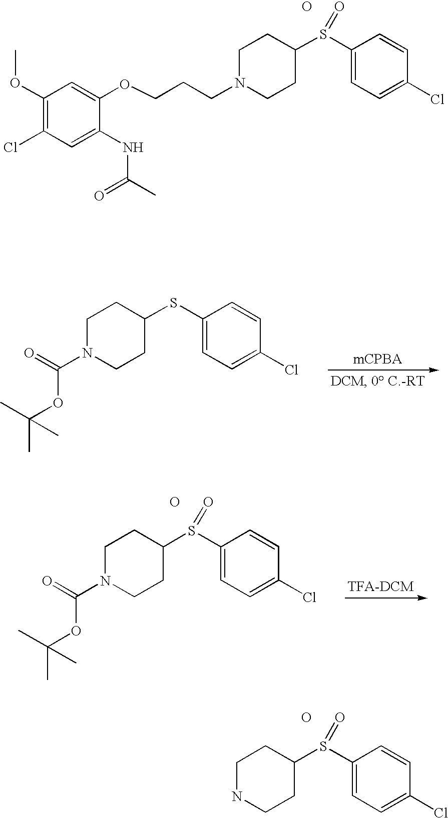

4-(4-Chloro-phenylsulfanyl)-piperidine-1-carboxylic acid tert-butyl ester was prepared as described in Example 71

### Step-1: 4-(4-Chloro-benzenesulfonyl)-piperidine-1-carboxylic acid tert-butyl ester

To a solution of 4-(4-Chloro-phenylsulfanyl)-piperidine-1-carboxylic acid tert-butyl ester (1.5 g, 4.57 mmol) in DCM was added m-chloro-perbenzoic acid (2.16 g, 9.14 mmol) portion wise at 0° C. The mixture was stirred at room temperature for one hour then concentrated, diluted with water and basified with 1(N) NaOH solution. The basified solution was extracted with ethyl acetate. The organic layer was washed with water, dried over sodium sulfate and concentrated to afford 1 g (63%) of 4-(4-Chloro-benzenesulfonyl)-piperidine-1-carboxylic acid tert-butyl ester. LC/MS [M+H]⁺: 360.4

### Step-2: 4-(4-Chloro-benzenesulfonyl)-piperidine

To a solution of 4-(4-Chloro-benzenesulfonyl)-piperidine-1-carboxylic acid tert-butyl ester (1 g, 2.78 mmol)) in DCM was added trifluoroacetic acid (2.14 ml) and stirred the solution for about 5 hours. The mixture was stirred at room temperature for one hour then concentrated and washed with dry ether. The TFA salt was diluted with a minimum volume of water and neutralized with aq. NaOH solution at 10° C. Extraction of the neutralized solution with ethyl acetate followed by concentration afforded 450 mg (62%) of 4-(4-Chloro-benzenesulfonyl)-piperidine. LC/MS [M+H]⁺: 260.2

N-(5-Chloro-2-{3-[4-(4-chloro-benzenesulfonyl)-piperidin-1-yl]-propoxy}-4-methoxy-phenyl)-acetamide was prepared by a procedure similar to that described in Example 64. LC/MS [M+H]⁺: 515.4. ¹H-NMR (400 MHz, DMSO-d₆) δ (ppm): 8.97 (s, 1H), 7.86-7.74 (m, 5H), 6.79 (s, 1H), 4.05 (t, 2H), 3.83 (s, 3H), 3.26 (m, 1H), 2.93 (m, 2H), 2.40 (m, 2H), 2.02 (s, 3H), 1.84 (m, 6H), 1.51 (m, 2H). HPLC purity: 90.1%.

### Example 73

### N-(5-Chloro-2-{3-[4-(4-chloro-phenoxy)-3,3-dimethyl-piperidin-1-yl]-propoxy}-4-methoxy-phenyl)-acetamide

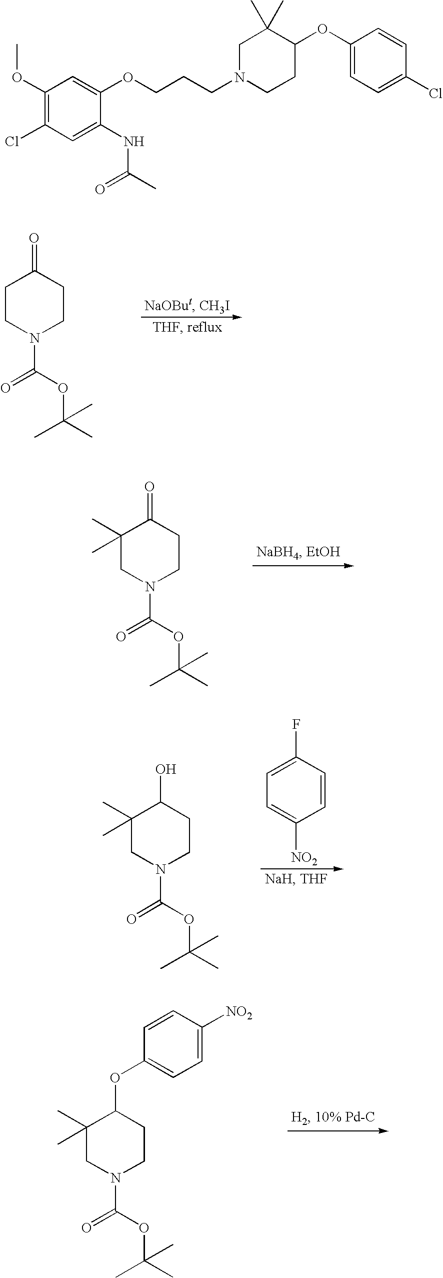

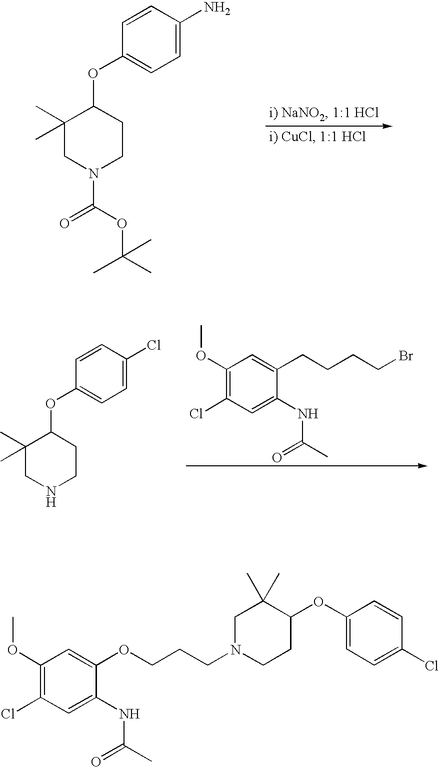

### Step-1: 3,3 dimethyl-4-oxo-piperidine-1-carboxylic acid tent-butyl ester

To a solution of 4-oxo-piperidine-1-carboxylic acid tert-butyl ester (30 g, 151 mmol) in THF was added sodium tert-butoxide (34.8 g, 362 mmol) portion wise at 0° C. The reaction mixture was stirred for 1 hour at room temperature. Methyl iodide (19.8 ml, 317 mmol) was added and the mixture was heated to reflux for 2 hours. The mixture was then concentrated, diluted with NH₄Cl/water and extracted with ethyl acetate. The organic layer was concentrated and purified over Si-gel (2% ethyl acetate-hexane) to afford 15.8 g (46%) of 3,3 dimethyl-4-oxo-piperidine-1-carboxylic acid tert-butyl ester. ¹H-NMR (400 MHz, CDCl₃) δ (ppm): 3.73 (t, 2H), 3.43 (br s, 2H), 2.49 (t, 2H), 1.49 (s, 9H), 1.13 (s, 6H).

### Step-2: 4-Hydroxy-3,3-dimethyl-piperidine-1-carboxylic acid tert-butyl ester

To a solution of 3,3 dimethyl-4-oxo-piperidine-1-carboxylic acid tert-butyl ester (2 g, 8.8 mmol) in ethanol (20 ml) was added sodium borohydride (670 mg, 17.6 mmol) at 0° C. and the mixture was stirred for 2 hour at room temperature. The excess NaBH₄ was quenched with cold water. The mixture was then concentrated, diluted with water and extracted with ethyl acetate. The organic layer was washed with water, dried over sodium sulfate and concentrated to afford 2 g (99.5%) of 4-hydroxy-3,3-dimethyl-piperidine-1-carboxylic acid tert-butyl ester. ¹H-NMR (400 MHz, CDCl₃): 3.86 (m, 1H), 3.49 (m, 1H), 3.40 (m, 1H), 3.05 (t, 1H), 2.73 (d, 1H), 1.73 (m, 1H), 1.49 (m, 1H), 1.44 (s, 9H), 1.39 (m, 1H), 0.95 (s, 3H), 0.87 (s, 3H).

### Step-3: 3,3-Dimethyl-4-(4-nitro-phenoxy)-piperidine-1-carboxylic acid tert-butyl ester

To a solution of 4-hydroxy-3,3-dimethyl-piperidine-1-carboxylic acid tert-butyl ester (500 mg, 2.18 mmol) in THF was added 60% sodium hydride (112 mg, 2.61 mmol) at 0° C. and the mixture was stirred the solution 1 h at room temperature. 1-fluorobenzene (0.25 ml, 2.18 mmol) was added and the mixture stirred overnight at room temperature. The mixture was then diluted with ice-water, extracted with ethyl acetate. The organic layer was washed with water, dried over sodium sulfate, concentrated and purified through column chromatography (Si-gel, 3% ethyl acetate-hexane) to afford 360 mg (47.4%) of 3,3-dimethyl-4-(4-nitro-phenoxy)-piperidine-1-carboxylic acid tert-butyl ester. ¹H-NMR (400 MHz, CDCl₃): 8.18 (d, 2H), 6.94 (d, 2H), 4.12 (m, 1H), 3.65 (m, 1H), 3.49 (d, 1H), 3.35 (m, 1H), 3.06 (d, 1H) 1.91 (m, 1H), 1.72 (m, 1H), 1.46 (s, 9H), 1.01 (s, 6H).

### Step-4: 4-(4-Amino-phenoxy)-3,3-dimethyl-piperidine-1-carboxylic acid tent-butyl ester

3,3-Dimethyl-4-(4-nitro-phenoxy)-piperidine-1-carboxylic acid tert-butyl ester (360 mg, 1.03 mmol) was hydrogenated with 10% Pd—C (50 mg) in ethanol for 2 h. The catalyst was removed by filtration through a celite bed. The reaction mixture was concentrated to afford 270 mg (82%) of 4-(4-amino-phenoxy)-3,3-dimethyl-piperidine-1-carboxylic acid tert-butyl ester. LC/MS [M+H]⁺: 320.8

### Step-5: 4-(4-Chloro-phenoxy)-3,3-dimethyl-piperidine

To a solution of 4-(4-amino-phenoxy)-3,3-dimethyl-piperidine-1-carboxylic acid tert-butyl ester (270 mg, 0.84 mmol) in HCl:H₂O (1:1) was added aq. NaNO₂ solution (76 mg, 1.09 mmol in 0.5 ml H₂O) drop wise at 0-5° C. The reaction mixture was stirred for 1 h at 0-5° C. The diazotized solution was added dropwise to a solution of CuCl (134 mg, 1.35 mmol) in 1 ml of HCl:H₂O (1:1) at ice-cold temperature. The reaction mixture was stirred overnight at room temperature. The reaction mixture was neutralized with 50% aq. NaOH solution then extracted with ethyl acetate. The organic layer was washed with water, dried over sodium sulfate, concentrated and purified through column chromatography (neutral alumina, 1% MeOH-DCM) to afford 44 mg (22%) of 4-(4-Chloro-phenoxy)-3,3-dimethyl-piperidine. LC/MS [M+H]⁺: 240.2

### Step-6: N-(5-Chloro-2-{3-[4-(4-chloro-phenoxy)-3,3-dimethyl-piperidin-1-yl]-propoxy}-4-methoxy-phenyl)-acetamide

To a solution of N-[2-(3-bromo-propoxy)-5-chloro-4-methoxy-phenyl]-acetamide (56 mg, 0.17 mmol) in DMF was added 4-(4-Chloro-phenoxy)-3,3-dimethyl-piperidine (40 mg, 0.17 mmol) and K₂CO₃ (46 mg, 0.33 mmol) and the resulting mixture was stirred overnight at room temperature. The mixture was then diluted with ice-water and extracted with ethyl acetate. The organic layer was washed with water, dried over sodium sulfate, concentrated and purified through column chromatography (Si-gel, 0.5% MeOH-DCM) to afford 18 mg (21%) of N-(5-Chloro-2-{3-[4-(4-chloro-phenoxy)-3,3-dimethyl-piperidin-1-yl]-propoxy}-4-methoxy-phenyl)-acetamide. LC/MS [M+H]⁺: 495.4. ¹H-NMR (400 MHz, DMSO-d₆) δ (ppm): 8.98 (s, 1H), 7.83 (s, 1H), 7.28 (d, 2H), 6.97 (d, 2H), 6.80 (s, 1H), 4.11 (t, 2H), 3.98 (m, 1H), 3.84 (s, 3H), 2.50 (m, 2H), 2.32 (m, 2H), 2.05 (s, 3H), 1.92-1.20 (m, 7H), 1.02 (s, 3H), 0.93 (s, 3H). HPLC: 94.4%

### Example 74

### N-(5-Chloro-2-{3-[4-(4-chloro-phenoxy)-piperidin-1-yl]-2-hydroxy-propoxy}-4-methoxy-phenyl)-acetamide

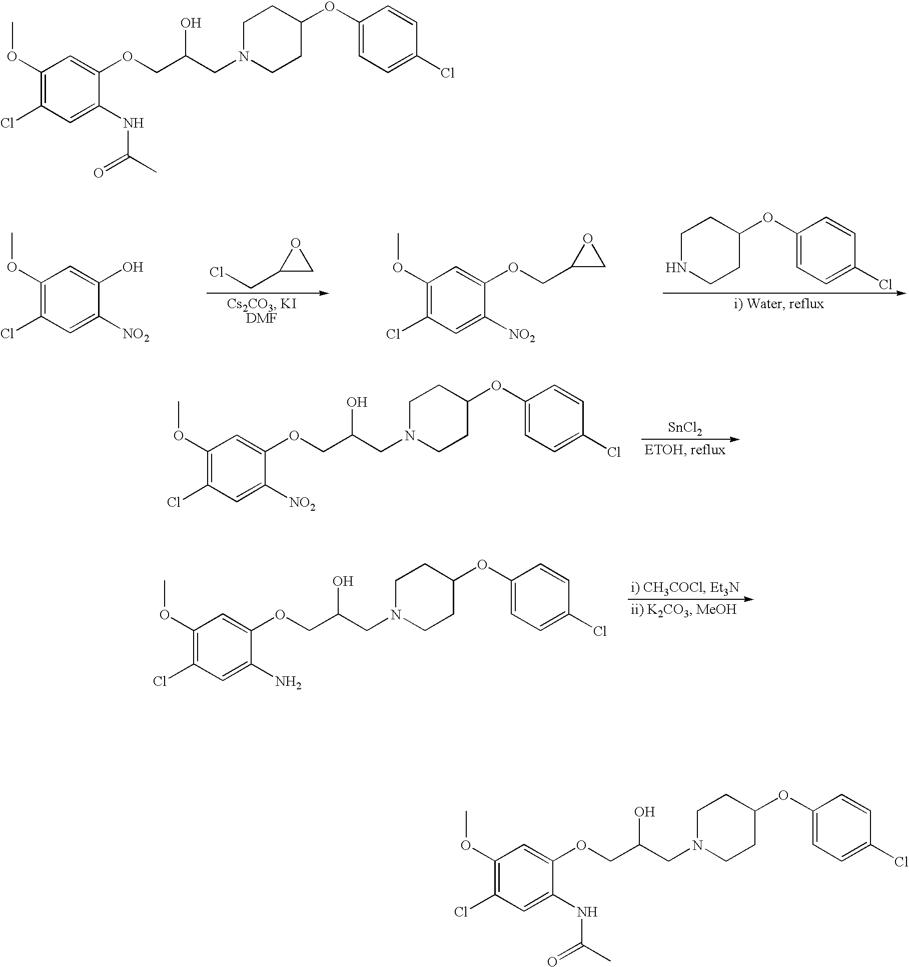

### Step-1: 2-(4-Chloro-5-methoxy-2-nitro-phenoxymethyl)-oxirane

To a solution of 4-chloro-5-methoxy-2-nitro-phenol (500 mg, 2.46 mmol) in DMF was added Cs₂CO₃ (1.2 g, 3.68 mmol), KI (catalytic amount, ˜20 mg) and epichlorohydrin (0.29 ml, 3.68 mmol). The reaction mixture was stirred for 4 h at 80° C. then diluted with ice-water. The resulting precipitate was isolated by filtration and dried to afford 400 mg (62%) of 2-(4-chloro-5-methoxy-2-nitro-phenoxymethyl)-oxirane. GCMS [m/z]: 259.0

### Step-2: 1-(4-Chloro-5-methoxy-2-nitro-phenoxy)-3-[4-(4-chloro-phenoxy)-piperidin-1-yl]-propan-2-01

To a solution of 2-(4-chloro-5-methoxy-2-nitro-phenoxymethyl)-oxirane (290 mg, 1.12 mmol) in water was added 4-(4-chloro-phenoxy)-piperidine (236 mg, 1.12 mmol) and the resulting mixture was heated to reflux overnight. The reaction mixture was then extracted with ethyl acetate. The organic layer was washed with water, dried over sodium sulfate, concentrated and purified through column chromatography (Si-gel, 1% MeOH-DCM) to afford 165 mg (31%) of 1-(4-Chloro-5-methoxy-2-nitro-phenoxy)-3-[4-(4-chloro-phenoxy)-piperidin-1-yl]-propan-2-ol. LC/MS [M+H]⁺: 471.4.

### Step-3: 1-(2-Amino-4-chloro-5-methoxy-phenoxy)-3-[4-(4-chloro-phenoxy)-piperidin-1-yl]-propan-2-ol

To a solution of 1-(4-chloro-5-methoxy-2-nitro-phenoxy)-3-[4-(4-chloro-phenoxy)-piperidin-1-yl]-propan-2-ol (190 mg, 0 4 mmol) in ethanol was added SnCl₂.2H₂O (456 mg, 2.02 mmol) and the resulting solution was heated to reflux for 4 hours. The mixture was then concentrated, diluted with water and basified with aq. NaOH. The product was extracted with ethyl acetate, which was washed with water and brine solution. The organic layer was dried over sodium sulfate and concentrated to afford 150 mg (85%) of 1-(2-amino-4-chloro-5-methoxy-phenoxy)-3-[4-(4-chloro-phenoxy)-piperidin-1-yl]-propan-2-ol. LC/MS [M+H]⁺: 441.6.

### Step-4: N-(5-Chloro-2-{3-[4-(4-chloro-phenoxy)-piperidin-1-yl]-2-hydroxy-propoxy}-4-methoxy-phenyl)-acetamide

To a solution of 1-(2-amino-4-chloro-5-methoxy-phenoxy)-3-[4-(4-chloro-phenoxy)-piperidin-1-yl]-propan-2-ol (150 mg. 0.34 mmol) in DCM was added triethyl amine (0.1 ml, 0.75 mmol) and acetyl chloride (0.05 ml, 0.75 mmol) at 0° C. The reaction mixture was stirred overnight at room temperature. The mixture was then diluted with water and the organic layer was separated, washed with water and dried over sodium sulfate. The organic layer was concentrated and purified through column chromatography (Si-gel, 0.8% MeoH-DCM). The pure diacylated product (36 mg) was dissolved in methanol and K₂CO₃ (50 mg was added. The reaction mixture was stirred for 20 min then concentrated and diluted with water. The product was extracted with ethyl acetate. The organic layer was washed with water, dried over sodium sulfate and concentrated to afford 27 mg (7%) of N-(5-chloro-2-{3-[4-(4-chloro-phenoxy)-piperidin-1-yl]-2-hydroxy-propoxy}-4-methoxy-phenyl)-acetamide. LC/MS [M+H]⁺: 483.3. ¹H-NMR (400 MHz, DMSO-d₆) δ (ppm): 9.02 (s, 1H), 7.98 (s, 1H), 7.30 (d, 2H), 6.97 (d, 2H), 6.84 (s, 1H), 5.09 (br s, 1H), 4.36 (br s, 1H), 4.09 (m, 1H), 3.93 (m, 1H), 3.89 (m, 1H), 3.84 (s, 3H), 2.78 (m, 2H), 2.32 (m, 2H), 2.06 (s, 3H), 2.03-1.59 (m, 5H). HPLC: 92.0%

### Example 75

### N-(5-Chloro-2-{3-[4-(4-cyano-phenoxy)-piperidin-1-yl]-propoxy}-4-methoxy-phenyl)-acetamide

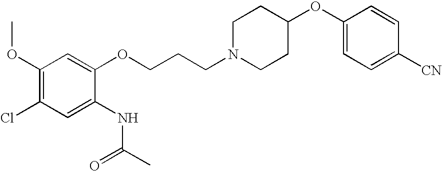

N-(5-Chloro-2-{3-[4-(4-cyano-phenoxy)-piperidin-1-yl]-propoxy}-4-methoxy-phenyl)-acetamide was prepared by a procedure similar to that described in Example 64. LC/MS [M+H]⁺: 457.9. ¹H-NMR (400 MHz, DMSO-d₆) δ (ppm): 8.97 (s, 1H), 7.82 (s, 1H), 7.73 (d, 2H), 7.12 (d, 2H), 6.82 (s, 1H), 4.54 (br s, 1H), 4.10 (t, 2H), 3.84 (s, 3H), 2.71 (br s, 2H), 2.50-1.47 (m, 13H). HPLC: 94.2%

### Example 76a

### N-(5-Chloro-2-{3-[4-(4-fluoro-phenoxy)-piperidin-1-yl]-propoxy}-4-methoxy-phenyl)-acetamide

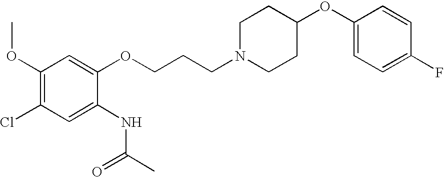

N-(5-Chloro-2-{3-[4-(4-fluoro-phenoxy)-piperidin-1-yl]-propoxy}-4-methoxy-phenyl)-acetamide was prepared by a procedure similar to that described in Example 64. LC/MS [M+H]⁺: 451.4. ¹H-NMR (400 MHz, DMSO-d₆) δ (ppm): 8.98 (s, 1H), 7.82 (s, 1H), 7.09 (t, 2H), 6.96 (m, 2H), 6.82 (s, 1H), 4.31 (br s, 1H), 4.10 (br s, 2H), 3.85 (s, 3H), 2.67 (m, 2H), 2.50 (m, 2H), 2.30 (m, 2H), 2.04 (s, 3H), 1.90 (m, 4H), 1.60 (m, 2H). HPLC: 95.5%.

### Example 76b

### N-(5-Chloro-2-{3-[4-(4-fluoro-benzyl)-4-hydroxy-3,3-dimethyl-piperidin-1-yl]-propoxy}-4-methoxy-phenyl)-acetamide

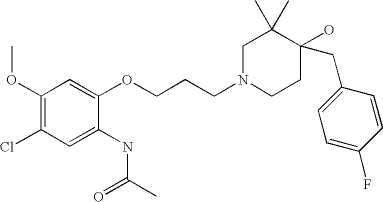

N-(5-Chloro-2-{3-[4-(4-fluoro-benzyl)-4-hydroxy-3,3-dimethyl-piperidin-1-yl]-propoxy}-4-methoxy-phenyl)-acetamide was prepared by a process similar to that described in example 60. LC/MS [M+H]⁺: 493.4. ¹H-NMR (400 MHz, DMSO-d₆) δ (ppm): 8.97 (s, 1H), 7.83 (s, 1H), 7.24 (m, 2H), 7.04 (m, 2H0, 6.79 (s, 1H), 4.09 (m, 2H), 3.83 (s, 3H), 3.48-2.08 (m, 9H), 2.04 (s, 3H), 1.87-1.10 (m, 4H), 1.03 (s, 3H), 0.90 (s, 3H). HPLC purity: 97.8%.

### Example 77

### N-(5-Chloro-2-{3-[4-(4-chloro-benzyl)-4-hydroxy-3,3-dimethyl-piperidin-1-yl]-propoxy}-4-isopropoxy-phenyl)-acetamide

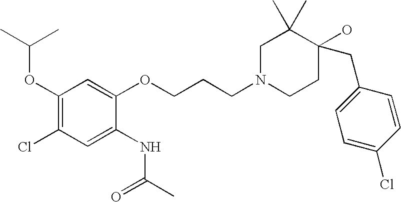

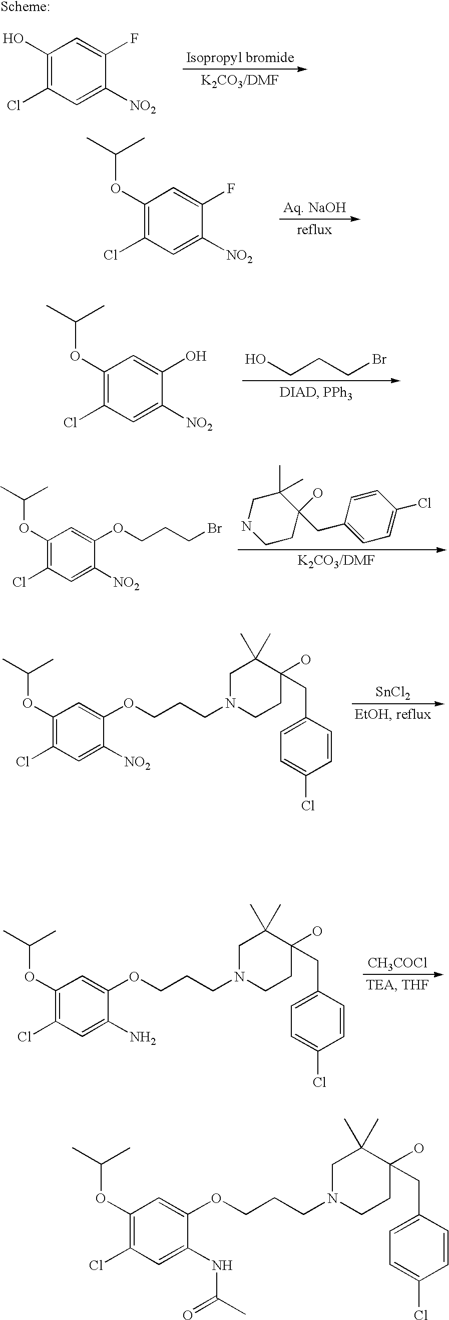

4-(4-Chloro-benzyl)-3,3-dimethyl-piperidin-4-ol was prepared by a process as described in example 43.

### Step-1:1-Chloro-4-fluoro-2-isopropoxy-5-nitro-benzene

To a solution of 2-Chloro-5-fluoro-4-nitro-phenol (1.3 g, 6.78 mmol) in DMF was added K₂CO₃ (1.87 g, 13.57 mmol) and isopropyl bromide (918 mg, 7.46 mmol). The reaction mixture was stirred overnight at 90° C. The reaction mixture was diluted with water and extracted with ethyl acetate. The organic layer was concentrated and purified over column chromatography (Si-gel (1% EtOAc-hexane) to afford 1.3 g (82%) 1-Chloro-4-fluoro-2-isopropoxy-5-nitro-benzene. GCMS [m/z]: 233.0. ¹H-NMR (400 MHz, CDCl₃) δ (ppm): 8.17 (d, 1H), 6.75 (d, 1H), 4.63 (m, 1H), 1.44 (d, 6H).

### Step-2: 4-Chloro-5-isopropoxy-2-nitro-phenol

A solution of 1-Chloro-4-fluoro-2-isopropoxy-5-nitro-benzene (1.5 g, 6.42 mmol) in dioxane was added 6 (N) NaOH solution was heated to reflux overnight. The pH of the solution was then adjusted to 1 with 6(N)HCl. The product was extracted with ethyl acetate, which was washed with water, saturated bicarbonate solution and brine. The organic layer was dried over Na₂SO₄ and concentrated under reduced pressure to afford 1.38 g (92.7%) of 4-Chloro-5-isopropoxy-2-nitro-phenol. GCMS [m/z]: 231.0. ¹H-NMR (400 MHz, CDCl₃) δ (ppm): 10.91 (s, 1H), 8.12 (s, 1H), 6.54 (s, 1H), 4.64 (m, 1H), 1.44 (d, 6H).

N-(5-Chloro-2-{3-[4-(4-chloro-benzyl)-4-hydroxy-3,3-dimethyl-piperidin-1-yl]-propoxy}-4-isopropoxy-phenyl)-acetamide was prepared by a process similar to that described in example No. 60. LC/MS [M+H]⁺: 537. ¹H-NMR (400 MHz, DMSO-d₆) δ (ppm): 9.00 (s, 1H), 7.83 (s, 1H), 7.29-7.22 (m, 4H), 6.80 (s, 1H), 4.62 (brs, 1H), 4.06 (s, 2H), 3.87 (s, 1H), 2.73-1.55 (12H), 1.26 (s, 6H), 1.02 (s, 3H), 0.89 (s, 3H). HPLC: 97.43%.

### Example 78

### N-(5-Chloro-2-{3-[4-(4-fluoro-benzyl)-4-hydroxy-3,3-dimethyl-piperidin-1-yl]-propoxy}-4-isopropoxy-phenyl)-acetamide

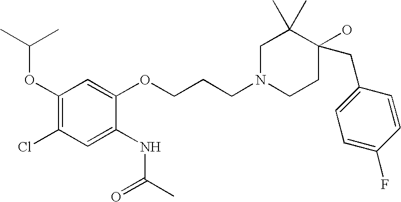

N-(5-Chloro-2-{3-[4-(4-fluoro-benzyl)-4-hydroxy-3,3-dimethyl-piperidin-1-yl]-propoxy}-4-isopropoxy-phenyl)-acetamide was prepared by a process similar to that described in example 77. LC/MS [M+H]⁺: 521.4. ¹H-NMR (400 MHz, DMSO-d₆) δ (ppm): 8.99 (bs, 1H), 7.83 (s, 1H), 7.24 (m, 2H), 7.04 (m, 2H), 6.80 (bs, 1H), 4.62 (m, 1H), 4.06 (m, 2H), 3.83 (bs, 1H), 2,70 (m, 1H), 2.61 (m, 1H), 2.49 (s, 3H), 2.21 (m, 2H), 2.05 (s, 3H), 1.86 (m, 2H), 1.33 (m, 1H), 1.24 (s, 6H), 1.02 (m, 2H), 0.89 (s, 3H), 0.85 (s, 3H). HPLC: 95.1%.

### Example 79

### N-(2-{3-[4-(4-Chloro-benzyl)-4-hydroxy-3,3-dimethyl-piperidin-1-yl]-propoxy}-4-methoxy-phenyl)-acetamide

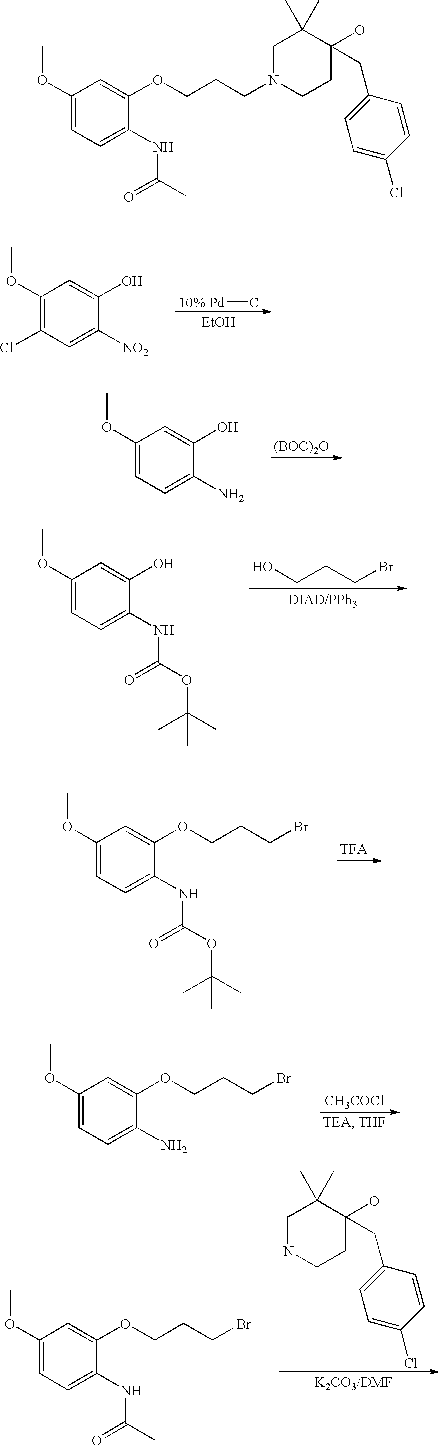

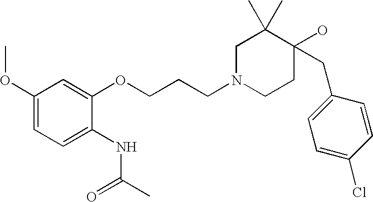

4-(4-Chloro-benzyl)-3,3-dimethyl-piperidin-4-ol was prepared by a process as described in example 43.

### Step-1: 2-Amino-5-methoxy-phenol

To a stirred solution of 4-Chloro-5-methoxy-2-nitro-phenol (1.0 gm, 5 mmol) in ethanol 10% Pd—C (100 mg) was added. The reaction mixture was then subjected to overnight hydrogenation using H₂ balloon. It was filtered and washed with ethanol. Then it was concentrated and neutralized with aq. NaOH. It was extracted with ethyl acetate and washed with water and brine. Finally it was dried and concentrated to afford 2-Amino-5-methoxy-phenol (700 mg). LC/MS [M+H]⁺: 139.8.

### Step-2: (2-Hydroxy-4-methoxy-phenyl)-carbamic acid tert-butyl ester

To a solution of 2-Amino-5-methoxy-phenol (700 mg, 5.03 mmol) in THF was added (BOC)₂O (1.3 ml, 5.53 mmol) at ice-cold condition and the reaction mixture was stirred overnight at room temperature. It was then concentrated and purified through column chromatography (Si-gel, 0.5% methanol-DCM) to afford 930 mg (77.3%) of (2-Hydroxy-4-methoxy-phenyl)-carbamic acid tert-butyl ester. ¹H-NMR (400 MHz, DMSO-d₆) δ (ppm): 9.62 (s, 1H), 7.75-6.31 (m, 4H), 3.65 (s, 3H), 1.43 (s, 9H).

### Step-3: [2-(3-Bromo-propoxy)-4-methoxy-phenyl]-carbamic acid tert-butyl ester

To a solution of tri-phenylphosphine (2.0 gm, 7.78 mmol) in THF was added DIAD (1.15 ml, 5.83 mmol) at cold condition and stirred for half an hour. Then (2-Hydroxy-4-methoxy-phenyl)-carbamic acid tert-butyl ester (930 mg, 3.88 mmol) and 3-Bromo-propan-1-ol (0.33 ml, 3.88 mmol) were added to the reaction mixture and stirred at room temperature overnight. It was concentrated and purified through column chromatography (Si-gel, 2% Meoh-DCM) to afford 730 mg (52.2%) of [2-(3-Bromo-propoxy)-4-methoxy-phenyl]carbamic acid tert-butyl ester. LC/MS [M+H]⁺: 360.4.

### Step-4: 2-(3-Bromo-propoxy)-4-methoxy-phenylamine

To a solution of [2-(3-Bromo-propoxy)-4-methoxy-phenyl]carbamic acid tert-butyl ester (200 mg, 0.55 mmol) in DCM was added TFA (1.0 ml, 12.0 mmol) at cold condition. The reaction mixture was then stirred at room temperature for 2 hours and concentrated. The reaction mixture was basified and extracted with ethyl acetate. Organic layer was washed with water, brine and concentrated to afford 135 mg (crude) of 2-(3-Bromo-propoxy)-4-methoxy-phenylamine. LC/MS [M+H]⁺: 260.2.

### Step-5: N-[2-(3-Bromo-propoxy)-4-methoxy-phenyl]-acetamide

To a solution of 2-(3-Bromo-propoxy)-4-methoxy-phenylamine (130 mg, 0.49 mmol) in THF was added triethylamine (0.08 ml, 0.55 mmol) at 0° C. Acetyl chloride (0.04 ml, 0.59 mmol) was then added to the reaction mixture and stirred at room temperature for 1 hour. It was concentrated and extracted with ethyl acetate. Organic layer was washed with water, brine and dried well. It was concentrated and purified through column chromatography (Si-gel, 1.5% MeOH-DCM) to afford (95 mg, 64.2%) of N-[2-(3-Bromo-propoxy)-4-methoxy-phenyl]-acetamide. LC/MS [M+H]⁺: 302.0.

### Step-6: N-(2-{3-[4-(4-Chloro-benzyl)-4-hydroxy-3,3-dimethyl-piperidin-1-yl]-propoxy}-4-methoxy-phenyl)-acetamide

To a solution of N-[2-(3-Bromo-propoxy)-4-methoxy-phenyl]-acetamide (95 mg, 0.31 mmol) in DMF was added K₂CO₃ (87 mg, 0.62 mmol) at 0° C. and stirred well. 4-(4-Chloro-benzyl)-3,3-dimethyl-piperidin-4-ol (80 mg, 0.31 mmol) was then added to the reaction mixture and stirred overnight. It was diluted with water and extracted with ethyl acetate. Organic layer was washed with water, brine and dried well. It was concentrated and purified through column chromatography (Si-gel, 1.5% MeOH-DCM) to afford 140 mg (95.1%) of N-(2-{3-[4-(4-Chloro-benzyl)-4-hydroxy-3,3-dimethyl-piperidin-1-yl]-propoxy}-4-methoxy-phenyl)-acetamide. LC/MS [M+H]⁺: 475.4. ¹H-NMR (400 MHz, DMSO-d₆) δ (ppm): 8.81 (s, 1H), 7.60-6.44 (m, 7H), 4.03-0.84 (m, 26H). HPLC: 91.5%.

### Example 80

### N-(5-Chloro-2-{3-[4-(4-chloro-phenyl)-4-hydroxy-3,3-dimethyl-piperidin-1-yl]-propoxy}-4-isopropoxy-phenyl)-acetamide

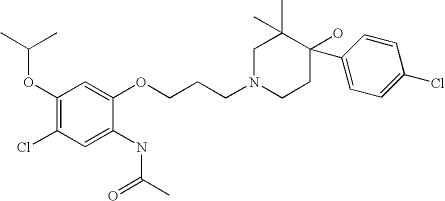

N-(5-Chloro-2-{3-[4-(4-chloro-phenyl)-4-hydroxy-3,3-dimethyl-piperidin-1-yl]-propoxy}-4-isopropoxy-phenyl)-acetamide was prepared by a procedure similar to that described in Example 67 and 77. LC/MS [M+H]⁺: 523.2. ¹H-NMR (400 MHz, DMSO-d₆) δ (ppm): 9.00 (s, 1H), 7.84 (s, 1H), 7.44 (d, 2H), 7.33 (d, 2H), 6.82 (s, 1H), 4.65 (s, 1H), 4.1 (t, 2H), 2.67-2.17 (m, 5H), 2.06 (s, 3H), 1.91 (s, 1H), 1.39 (m, 1H) 1.26 (d, 6H), 0.78 (s, 3H), 0.64 (s, 3H). HPLC: 93.4%

### Example 81

### N-(5-Chloro-2-{2-[4-(4-fluoro-phenoxy)-piperidin-1-yl]-2-oxo-ethoxy}-4-methoxy-phenyl)-acetamide

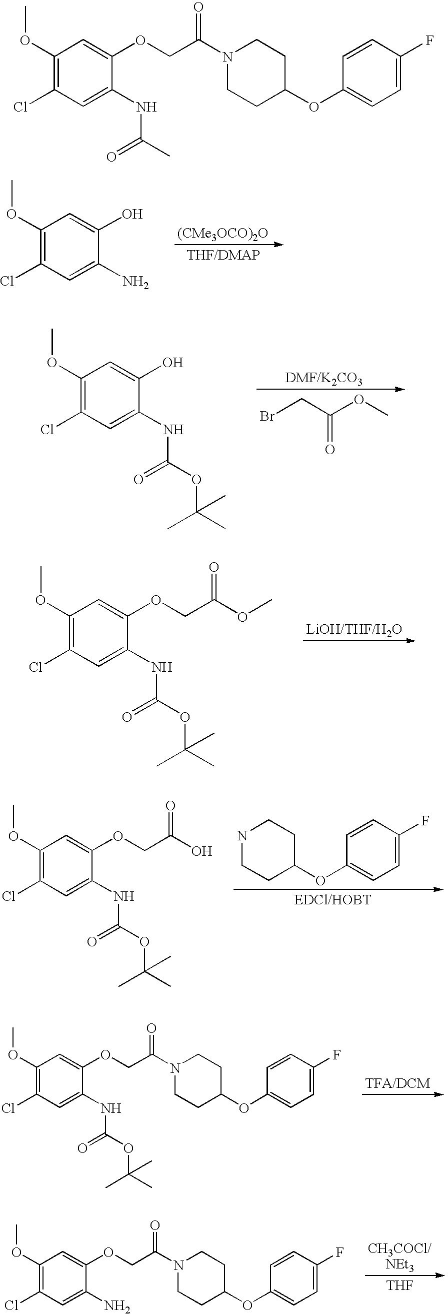

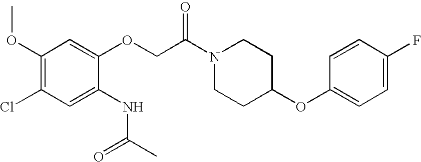

2-Amino-4-chloro-5-methoxy-phenol was prepared by a process similar to that described in example 60.

### Step-1: (5-Chloro-2-hydroxy-4-methoxy-phenyl)-carbamic acid tert-butyl ester

To a solution of 2-Amino-4-chloro-5-methoxy-phenol (200 mg, 1.15 mmol) in THF was added (BOC)₂O (0.29 ml, 1.27 mmol) at cold condition and the reaction mixture was refluxed for 2 hours. The reaction mixture was concentrated and purified through column chromatography (Si-gel, 4% ethyl acetate-hexane) afforded 195 mg (61.9%) of 5-Chloro-2-hydroxy-4-methoxy-phenyl)-carbamic acid tert-butyl ester.

¹H-NMR (400 MHz, CDCl₃) δ (ppm): 8.53 (brs, 1H), 6.92 (s, 1H), 6.59 (s, 1H), 6.39 (bs, 1H), 3.81 (s, 3H), 1.51 (d, 9H).

### Step-2: (2-tert-Butoxycarbonylamino-4-chloro-5-methoxy-phenoxy)-acetic acid methyl ester

To a solution of 5-Chloro-2-hydroxy-4-methoxy-phenyl)-carbamic acid tert-butyl ester (195 mg, 0.71 mmol) in DMF (1 ml) was added anhd. K₂CO₃ (197 mg, 1.42 mmol) and methyl bromoacetate (163 mg, 1.06 mmol) at 0° C. The reaction mixture was stirred at room temperature 2 h and diluted with ice-water, extracted with ethyl acetate, washed with water and brine solution. The organic layer was concentrated under reduced pressure to afford 235 mg (95.6%) of 2-tert-Butoxycarbonylamino-4-chloro-5-methoxy-phenoxy)-acetic acid methyl ester. LC/MS [M+H]⁺: 346.1. ¹H-NMR (400 MHz, CDCl₃) δ (ppm): 8.12 (s, 1H), 7.25 (s, H), 6.42 (s, H), 4.65 (m, 2H), 3.80 (m, 5H), 1.52 (s, 9H).

### Step-3: (2-tert-Butoxycarbonylamino-4-chloro-5-methoxy-phenoxy)-acetic acid

To a solution of 2-tert-Butoxycarbonylamino-4-chloro-5-methoxy-phenoxy)-acetic acid methyl ester (235 mg, 0.67 mmol) in THF-water (3:1) was added LiOH.H₂O (71 mg, 1.7 mmol) at ice-cold condition. The reaction mixture was allowed to stir at room temperature during one and half hour. The reaction mixture was evaporated and the crude mass was diluted with water, neutralized with AcOH and extracted with ethyl acetate. The organic layer was washed with water, brine and concentrated under reduced pressure. The crude mass was washed with hexane to afford 185 mg (83.2%) of (2-tert-butoxycarbonylamino-4-chloro-5-methoxy-phenoxy)-acetic acid. ¹H-NMR (400 MHz, CDCl₃) δ (ppm): 13.1 (bs, H), 8.08 (s, H), 7.73 (s, H), 6.87 (s, H), 4.80 (s, 2H), 3.80 (s, 3H), 1.36 (s, 9H).

### Step-4: (5-Chloro-2-{2-[4-(4-fluoro-phenoxy)-piperidin-1-yl]-2-oxo-ethoxy}-4-methoxy-phenyl)-carbamic acid tert-butyl ester

To a solution of (2-tert-butoxycarbonylamino-4-chloro-5-methoxy-phenoxy)-acetic acid (185 mg, 0.5 mmol) in DMF was added 4-(4-Fluoro-phenoxy)-piperidine (109 mg, 0.5 mmol), EDCI (139 mg, 0.73 mmol), HOBT (38 mg, 0.27 mmol) and DIPEA (216 g, 1.67 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 35% ethyl acetate-hexane) to afford 120 mg (47.2%) of 6,7-Dichloro-2-[4-cyano-4-(4-fluoro-benzyl)-piperidine-1-carbonyl]-2,3-dihydro-benzo[1,4]oxazine-4-carboxylic acid tert-butyl ester. LC/MS [M+H]⁺: 509.2. ¹H-NMR (400 MHz, CDCl₃) δ (ppm): 8.08 (bs, H), 7.50 (m, H), 6.96 (m, 2H), 6.84 (m, 2H), 6.61 (s, H), 4.76 (s, 2H), 4.46 (m, H), 3.83 (s, 3H), 3.74 (m, H), 3.65 (m, 2H), 3.44 (m, H), 2.08 (s, H), 1.52 (s, 9H), 0.90 (m, H), 0.86 (m, H).

### Step-5: 2-(2-Amino-4-chloro-5-methoxy-phenoxy)-1-[4-(4-fluoro-phenoxy)-piperidin-1-yl]-ethanone

Trifluoroacetic acid (269 mg, 2.4 mmol) was added to a solution of (5-Chloro-2-{2-[4-(4-fluoro-phenoxy)-piperidin-1-yl]-2oxo-ethoxy}-4-methoxy-phenyl)-carbamic acid tert-butyl ester (120 mg, 0.24 mmol) in DCM at 5-10° C. and the reaction mixture was stirred for 2-4 h at room temperature. The solution was then concentrated and neutralised with 1 (N) NaOH solution. The product was extracted with ethyl acetate and the organic layer was washed with water and brine solution. The organics were concentrated under reduced pressure to afford 85 mg (86.6%) of 2-(2-Amino-4-chloro-5-methoxyphenoxy)-1-[4-(4-fluoro-phenoxy)-piperidin-1-yl]-ethanone. LC/MS [M+H]⁺: 409.2. ¹H-NMR (400 MHz, DMSO-d₆) δ (ppm):

7.11 (t, 2H), 7.01 (m, 2H), 6.69 (d, 2H), 4.83 (s, 2H), 4.63 (s, H), 4.62 (m, H), 4.03 (q, 1H), 3.85 (m, 1H), 3.71 (s, 3H), 3.3 (m, 1H), 1.98 (m, 1H), 1.90 (m, 2H), 1.55 (dd, 2H), 1.17 (t, 1H).

### Step-6: N-(5-Chloro-2-{2-[4-(4-fluoro-phenoxy)-piperidin-1-yl]-2-oxo-ethoxy}-4-methoxy-phenyl)-acetamide

To a solution of 2-(2-Amino-4-chloro-5-methoxy-phenoxy)-1-[4-(4-fluoro-phenoxy)-piperidin-1-yl]-ethanone (85 mg, 0.21 mmol) in THF (5 ml) was dropwise added triethylamine (23 mg, 0.22 mmol) followed by acetyl chloride (18 mg, 0.22 mmol) at 0° C. The reaction mixture was stirred at room temperature for 15 mins. The reaction mixture was concentrated, diluted with water and extracted with ethyl acetate. The organic layer was concentrated under reduced pressure to afford 80 mg (84.5%) of N-(5-Chloro-2-{2-[4-(4-fluoro-phenoxy)-piperidin-1-yl]-2-oxo-ethoxy}-4-methoxy-phenyl)-acetamide. LC/MS [M+H]⁺: 451.2. ¹H-NMR (400 MHz, DMSO-d₆) δ (ppm): 9.55 (s, 1H), 7.97 (s, 1H), 7.11 (t, 2H), 7.00 (m, 2H), 6.86 (bs, 1H), 5.00 (s, 2H), 4.56 (bs, 1H), 3.86 (s, 3H), 3.63 (m, 1H), 3.31 (s, 2H), 2.18 (s, 3H), 1.90 (m, 2H), 1.53 (m, 1H), 1.35 (m, 1H), 1.23 (s, 1H). HPLC: 95.95%.

### Example 82

### (5-Chloro-2-{2-[4-(4-fluoro-phenoxy)-piperidin-1-yl]-2-oxo-ethoxy}-phenyl)-urea

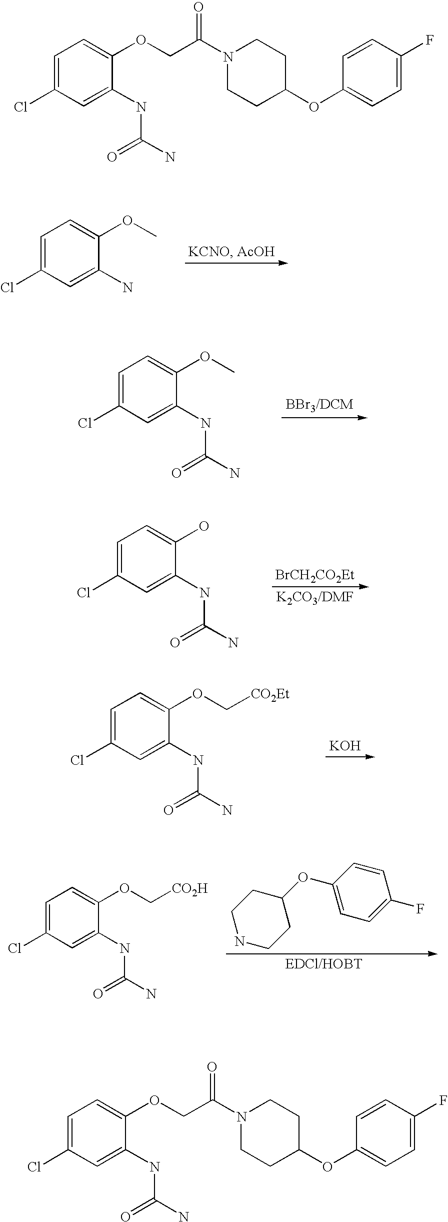

4-(4-Fluoro-phenoxy)-piperidine was prepared by a similar process as described in example 64.

### Step 1: (5-Chloro-2-methoxy-phenyl)-urea

To a solution of 5-chloro-2-methoxyaniline (20 g, 0.13 mol) in DMF was added acetic acid (17.4 ml, 0.3 mol) and potassium isocyanate (22.6 g, 0.28 mol). The solution was stirred overnight with water (3 eqv.) at room temperature. The solution was diluted with ice-water. The precipitated solid was filtered and dried. The crude solid was washed with hexane to afford 8.2 g (32%) of (5-Chloro-2-methoxy-phenyl)-urea. LC/MS [M+H]⁺: 201.2. ¹H-NMR (400 MHz, DMSO-d₆) δ (ppm): 8.20 (d, 1H), 8.09 (s, 1H), 6.96 (d, J=2.5 Hz, 1H), 6.89 (dd, J=8.7, 2.5 Hz, 1H), 6.32 (s, 2H), 3.83 (s, 3H).

### Step-2: (5-Chloro-2-hydroxy-phenyl)-urea

To a solution of (5-Chloro-2-methoxy-phenyl)-urea (9 g, 44.9 mmol) in DCM was added BBr₃ (8.5 ml, 89.7 mmol) at 0° C. The solution was stirred for 4 h at room temperature. The solution was diluted with water. The precipitated solid was filtered and dried. The crude solid was washed with hexane to afford 3.7 g (96%) of (5-Chloro-2-hydroxy-phenyl)-urea. LC/MS [M+H]⁺: 187.1. ¹H-NMR (400 MHz, DMSO-d₆) δ (ppm): 10.09 (s, 1H), 8.06 (d, J=2.0 Hz, 1H), 8.04 (s, 1H), 6.53 (m, 2H), 6.29 (s, 2H).

### Step 3: Methyl-(4-Chloro-2-ureido-phenoxy)-acetate

To a solution of 4-chloro-2-isoxazol-5-yl-phenol (3 g, 16 mmol) in DMF was added potassium carbonate (6.63 g, 48 mmol) and methyl bromoacetate (1.6 ml, 17.7 mmol) at 0° C. The solution was stirred overnight at room temperature. The DMF solution was diluted with ice-water and extracted with ethyl acetate. The organic layer was concentrated to afford 4 g (72%) of methyl-(4-Chloro-2-ureido-phenoxy)-acetate. LC/MS [M+H]⁺: 259.1. ¹H-NMR (400 MHz, DMSO-d₆) δ (ppm): 8.2 (s, 1H), 8.17 (s, 1H), 6.90-6.80 (m, 2H), 6.36 (s, 2H), 4.89 (s, 2H), 3.70 (s, 3H).

### Step-4: (4-Chloro-2-ureido-phenoxy)-acetic acid

To a solution of methyl-(4-Chloro-2-ureido-phenoxy)-acetate (4.15 g, 15 9 mmol) in THF-water (4:1) was added LiOH (660 mg, 15.9 mmol) at 0° C. and stirred for 4 h at room temperature. The reaction mixture was concentrated and dissolved in a minimum amount of water. The pH of the solution was adjusted to 2 with 1(N) HCl, extracted with ethyl acetate and the organic phase was concentrated to afford 3.4 g (78%) of (4-chloro-2-ureido-phenoxy)-acetic acid. LC/MS [M+H]⁺: 245. ¹H-NMR (400 MHz, DMSO-d₆) δ (ppm): 13.1 (brs, 1H), 8.2 (s, 1H), 8.12 (s, 1H), 6.86 (s, 2H), 6.35 (s, 2H), 4.6 (s, 2H).

### Step-5: (5-Chloro-2-{2-[4-(4-fluoro-phenoxy)-piperidin-1-yl]-2-oxo-ethoxy}-phenyl)-urea

To a solution of (4-Chloro-2-ureido-phenoxy)-acetic acid (100 mg, 0.41 mmol) in DMF was added 4-(4-Fluoro-phenoxy)-piperidine (80 mg, 0.41 mmol), EDCI (102 mg, 0.53 mmol), HOBT (28 mg, 0.21 mmol) and DIPEA (0.2 ml, 1.23 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 35% ethylacetate-hexane) to afford 83 mg (47.9%) of 5-Chloro-2-{2-[4-(4-fluoro-phenoxy)-piperidin-1-yl]-2-oxo-ethoxy}-phenyl)-urea. LC/MS [M+H]⁺: 422.1. ¹H-NMR (400 MHz, DMSO-d₆) δ (ppm): 8.19 (s, 1H), 8.14 (s, 1H), 7.11 (t, 2H, J=8.64), 7.01 (m, 2H), 6.86 (s, 2H), 6.35 (s, 2H), 4.96 (s, 2H), 4.56 (s, H), 4.07 (q, H, J=5.28), 3.81 (m, H), 3.67 (m, H), 3.31 (m, H), 3.17 (d, 2H, J=5.08), 1.90 (m, 2H). HPLC: 99.4%.

### Example 83

### 5-Chloro-2-{3-[4-(4-fluoro-benzyl)-4-hydroxy-3,3-dimethyl-piperidin-1-yl]-2-hydroxy-propoxy}-4-methoxy-benzoic acid

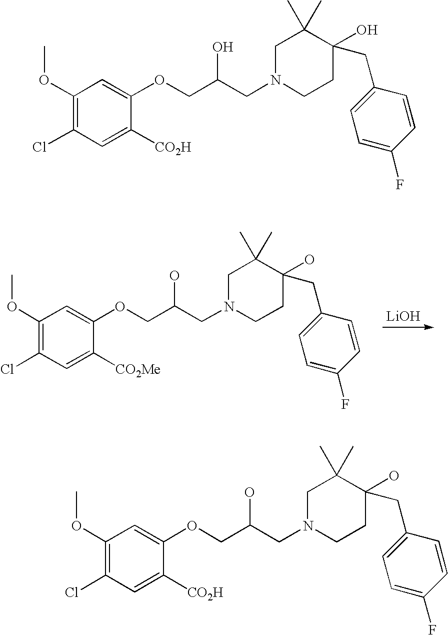

5-Chloro-2-{3-[4-(4-fluoro-benzyl)-4-hydroxy-3,3-dimethyl-piperidin-1-yl]-2-hydroxy-propoxy}-4-methoxy-benzoic acid methyl ester was prepared by a process similar to that described in example 34.

To a solution of 5-Chloro-2-{3-[4-(4-fluoro-benzyl)-4-hydroxy-3,3-dimethyl-piperidin-1-yl]-2-hydroxy-propoxy}-4-methoxy-benzoic acid methyl ester (150 mg, 0.29 mmol) in THF: H₂O (2:1) was added lithium hydroxide (82 mg, 1.95 mmol). The reaction mixture was stirred for 4 h and concentrated. The crude mass was neutralized with 50% aqueous HCl and extracted with ethyl acetate. The organic layer was concentrated and purified over column chromatography (Si-gel, 4-7% MeOH-DCM) to afford 25 mg (17%) of 5-Chloro-2-{3-[4-(4-fluoro-benzyl)-4-hydroxy-3,3-dimethyl-piperidin-1-yl]-2-hydroxy-propoxy}-4-methoxy-benzoic acid. LC/MS [M+H]⁺: 496.4 ¹H-NMR (400 MHz, CD₃OD) δ (ppm): 7.71 (s, 1H), 7.25 (m, 2H), 7.06 (m, 2H), 6.85 (s, 1H), 4.14 (m, 4H), 3.93 (s, 3H0, 2.75 (m, 4H), 2.64 (m, 4H), 1.23 (m, 1H), 1.16 (m, 1H), 1.08 (s, 3H), 0.92 (s, 3H). HPLC: 97.7%.

### Example 84

### 5-Chloro-2-{3-[4-(4-fluoro-benzyl)-4-hydroxy-3,3-dimethyl-piperidin-1-yl]-2-hydroxy-propoxy}-4-methoxy-N-methyl-benzamide

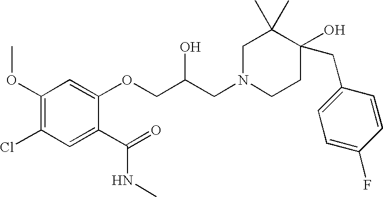

5-Chloro-2-{3-[4-(4-fluoro-benzyl)-4-hydroxy-3,3-dimethyl-piperidin-1-yl]-2-hydroxy-propoxy}-4-methoxy-N-methyl-benzamide was prepared by a process similar to that described in example 34. LC/MS [M+H]⁺: 509.4. ¹H-NMR (400 MHz, DMSO-d₆) δ (ppm): 8.27 (m, 1H), 7.82 (s, 1H0, 7.23 (m, 2H0, 7.04 (m, 2H), 6.86 (s, 1H0, 5.12 (m, 1H), 4.30 (m, 1H), 4.13 (m, 1H), 4.10 (bs, 1H), 3.92 (s, 3H), 3.81 (m, 1H), 2.80 (s, 3H), 2.79 (m, 1H), 2.66 (m, 1H), 2.39 (m, 4H), 2.15 (m, 1H), 2.07 (m, 1H), 1.53 (m, 1H), 1.33 (s, 1H), 1.23 (s, 3H), 1.14 (s, 3H). HPLC: 97.1%.

### Example 85

### 5-Chloro-2-{3-[4-(4-fluoro-benzyl)-4-hydroxy-3,3-dimethyl-piperidin-1-yl]-propoxy}-4-methoxy-N-methyl-benzamide

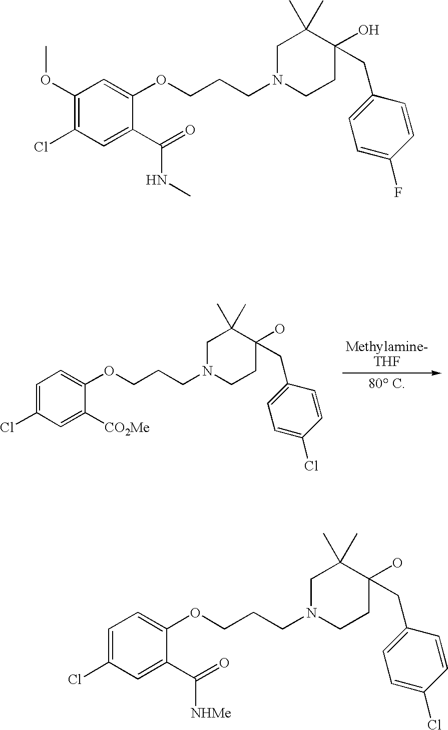

5-Chloro-2-{3-[4-(4-chloro-benzyl)-4-hydroxy-3,3-dimethyl-piperidin-1-yl]-propoxy}-benzoic acid methyl ester was prepared by a process similar to that described in example 55. 5-Chloro-2-{3-[4-(4-chloro-benzyl)-4-hydroxy-3,3-dimethyl-piperidin-1-yl]-propoxy}-benzoic acid methyl ester was heated with methylamine solution in THF in a sealed tube at 80° C. followed by purification to afford 5-Chloro-2-{3-[4-(4-fluoro-benzyl)-4-hydroxy-3,3-dimethyl-piperidin-1-Y^{1}}-propoxy]-4-methoxy-N-methyl-benzamide. LC/MS [M+H]⁺: 492.9. ¹H-NMR (400 MHz, DMSO-d₆) δ (ppm): 7.90 (m, 1H), 7.77 (s, 1H), 7.24 (m, 2H), 7.04 (m, 2H), 6.81 (s, 1H0, 4.22 (m, 2H), 3.92 (s, 3H), 3.81 (s, 1H), 2.80 (s, 3H), 2.74-2.59 (m, 3H), 2.35 (m, 3H), 2.23 (m, 1H), 2.08 (m, 1H), 1.95 (m, 1H), 1.93 (m, 2H), 1.33 (m, 1H), 1.03 (s, 3H), 0.90 (s, 3H). HPLC: 99.2%.

### Example 86

### 5-Chloro-2-{3-[4-(4-chloro-phenyl)-4-hydroxy-3,3-dimethyl-piperidin-1-yl]-propoxy}-4-methoxy-benzoic acid methyl ester

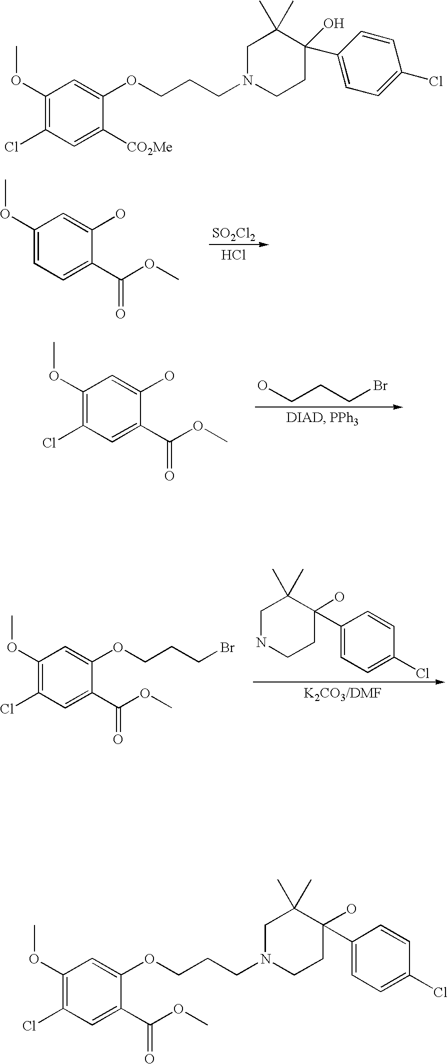

4-(4-Chloro-phenyl)-3,3-dimethyl-piperidin-4-ol was prepared by a process as described in example 10.

### Step-1: 5-Chloro-2-hydroxy-4-methoxy-benzoic acid methyl ester

To a solution of 2-Hydroxy-4-methoxy-benzoic acid methyl ester (5.0 g, 27.4 mmol) in DCM (25 ml), aq. HCl (1 ml of 2.5 M) and sulphonyl chloride (2.28 ml, 28.8 mmol) was added and the resulting mixture was refluxed for 2 hours. The reaction mixture was concentrated under reduced pressure and the solid thus obtained was stirred with 25 ml of methanol for 30 minutes. The precipitated solid was filtered, washed with methanol and dried under reduced pressure to afford 4.8 g (80.6%) of 5-Chloro-2-hydroxy-4-methoxy-benzoic acid methyl ester. ¹H-NMR (400 MHz, CDCl₃) δ (ppm): 10.74 (s, H), 7.74 (s, H), 6.74 (s, H), 3.89 (s, 3H), 3.86 (s, 3H).

### Step-2: 2-(3-Bromo-propoxy)-5-chloro-4-methoxy-benzoic acid methyl ester

To a solution of triphenyl phosphine (1.2 g, 4.61 mmol) in THF was added DIAD (0.7 ml, 3.46 mmol) at 0° C. and the mixture was stirred for 1 hour. A solution of 3-bromopropanol (0.2 ml, 2.3 mmol) and 5-Chloro-2-hydroxy-4-methoxy-benzoic acid methyl ester (500 mg, 2.3 mmol) in THF was then added at 0° C. and the mixture was stirred at room temperature overnight. The mixture was concentrated and purified through Si-gel column (5% ethyl acetate-hexane) to afford 1 g (60%) of 1-(3-Bromo-propoxy)-4-chloro-2-nitro-benzene. LC/MS [M+H]⁺: 323.1.

### Step-3: 5-Chloro-2-{3-[3-(4-chloro-phenyl)-3-hydroxy-4,4-dimethyl-piperidin-1-yl]-propoxy}-4-methoxy-benzoic acid methyl ester

To a solution of 2-(3-Bromo-propoxy)-5-chloro-4-methoxy-benzoic acid methyl ester (200 mg, 0.59 mmol) in DMF was added K₂CO₃ (164 mg, 1.18 mmol) and 4-(4-Chloro-phenyl)-3,3-dimethyl-piperidin-4-ol (143 mg, 0.59 mmol). The reaction mixture was stirred at room temperature overnight. The reaction mixture was diluted with water and extracted with ethyl acetate. The organic layer was washed with water and dried over sodium sulfate. The crude organics was concentrated under reduced pressure and purified over column chromatography (Si-gel, 1.5% MeOH-DCM) to afford 170 mg (57.8%) 5-Chloro-2-{3-[4-(4-fluoro-benzyl)-4-hydroxy-3,3-dimethyl-piperidin-1-yl]-propoxy}-4-methoxy-benzoic acid methyl ester.

LC/MS [M+H]⁺: 496.4. ¹H-NMR (400 MHz, DMSO-d₆) δ (ppm): 7.72 (s, 1H), 7.46 (d, 2H) 7.33 (d, 2H), 6.82 (s, 1H), 5.75 (s, 1H), 4.64 (s, 1H), 4.17 (s, 2H), 3.95 (s, 3H), 3.76 (s, 3H), 2.67 (bs, 1H), 2.56 (m, 2H) 2.40 (m, 2H), 2.25 (m, 2H), 1.89 (bs, 2H), 0.85 (s, 3H), 0.64 (s, 3H). HPLC: 95.1%.

### Example 87

### (5-Chloro-2-{3-[4-(4-fluoro-benzyl)-4-hydroxy-3,3-dimethyl-piperidin-1-yl]-propoxy}-4-methoxy-phenyl)-pyrrolidin-1-yl-methanone

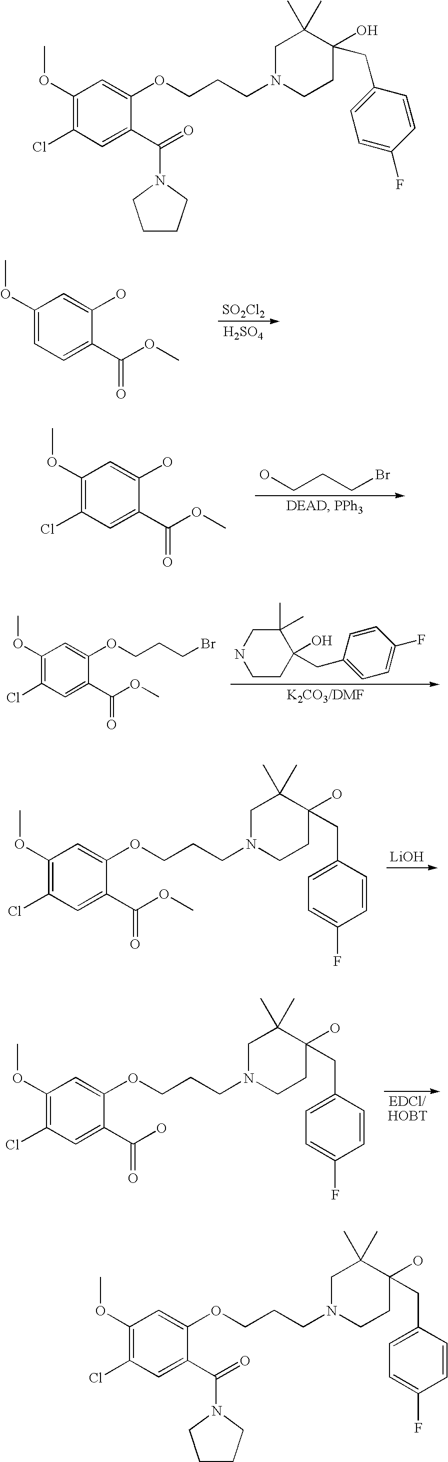

4-(4-Fluoro-benzyl)-3,3-dimethyl-piperidin-4-ol was prepared by a process similar to that described in example 30.

### Step-1: 5-Chloro-2-hydroxy-4-methoxy-benzoic acid methyl ester

To a solution of 2-Hydroxy-4-methoxy-benzoic acid methyl ester (5.0 g, 27.4 mmol) in DCM (25 ml), aq. HCl (1 ml of 2.5 M) and sulphonyl chloride (2.28 ml, 28.8 mmol) was added and the resulting mixture was refluxed for 2 hours. The reaction mixture was evaporated under reduced pressure and the solid thus obtained was stirred with 25 ml of methanol for 30 minutes. The precipitated solid was filtered, washed with methanol and dried under reduced pressure to afford 4.8 g (80.6%) of 5-Chloro-2-hydroxy-4-methoxy-benzoic acid methyl ester. ¹H-NMR (400 MHz, CDCl₃) δ (ppm): 10.74 (s, H), 7.74 (s, H), 6.74 (s, H), 3.89 (s, 3H), 3.86 (s, 3H).

### Step-2: 2-(3-Bromo-propoxy)-5-chloro-4-methoxy-benzoic acid methyl ester

To a solution of triphenyl phosphine (1.2 g, 4.61 mmol) in THF was added DIAD (0.7 ml, 3.46 mmol) at 0° C. and the mixture was stirred for 1 hour. A solution of 3-bromopropanol (0.2 ml, 2.3 mmol) and 5-Chloro-2-hydroxy-4-methoxy-benzoic acid methyl ester (500 mg, 2.3 mmol) in THF was then added at 0° C. and the mixture was stirred at room temperature overnight. The mixture was concentrated and purified through Si-gel column (5% ethyl acetate-hexane) to afford 1 g (60%) of 1-(3-Bromo-propoxy)-4-chloro-2-nitro-benzene. LC/MS [M+H]⁺: 338.4.

### Step-3: 5-Chloro-2-{3-[4-(4-fluoro-benzyl)-4-hydroxy-3,3-dimethyl-piperidin-1-yl]-propoxy}-4-methoxy-benzoic acid methyl ester

To a solution of 2-(3-Bromo-propoxy)-5-chloro-4-methoxy-benzoic acid methyl ester (400 mg, 1.2 mmol) in DMF was added K₂CO₃ (328 mg, 2.34 mmol) and 4-(4-Fluoro-benzyl)-3,3-dimethyl-piperidin-4-ol (281 mg, 1.2 mmol) were added. The reaction mixture was stirred at room temperature for overnight. The reaction mixture was diluted with water and extracted with ethyl acetate. The organic layer was washed with water, brine and concentrated to produce 550 mg (92.7%) of 5-Chloro-2-{3-[4-(4-fluoro-benzyl)-4-hydroxy-3,3-dimethyl-piperidin-1-yl]-propoxy}-4-methoxy-benzoic acid methyl ester. LC/MS [M+H]⁺: 494.4. ¹H-NMR (400 MHz, DMSO-d₆) δ (ppm): 7.71 (s, 1H), 7.23 (m, 2H), 7.04 (m, 2H), 6.80 (s, 1H), 4.13 (t, 2H), 3.93 (s, 3H), 3.74 (s, 3H), 2.73-1.84 (m, 12H), 1.01 (s, 3H), 0.89 (s, 3H).

### Step-4: 5-Chloro-2-{3-[4-(4-fluoro-benzyl)-4-hydroxy-3,3-dimethyl-piperidin-1-yl]-propoxy}-4-methoxy-benzoic acid

To a solution of 5-Chloro-2-{3-[4-(4-fluoro-benzyl)-4-hydroxy-3,3-dimethyl-piperidin-1-yl]-propoxy}-4-methoxy-benzoic acid methyl ester (400 mg, 0.81 mmol) in THF: H₂O (4:1) was added LiOH (380 mg, 9.0 mmol) and heated at 80° C. overnight. The reaction mixture was concentrated, diluted with water, neutralized with dilute HCl and extracted with ethyl acetate. The organic layer was washed with water and concentrated to afford 350 mg (90%) of 5-Chloro-2-{3-[4-(4-fluoro-benzyl)-4-hydroxy-3,3-dimethyl-piperidin-1-yl]-propoxy}-4-methoxy-benzoic acid. LC/MS [M+H]⁺: 480.2. ¹H-NMR (400 MHz, DMSO-d₆) δ (ppm): 7.65 (s, 1H), 7.24 (m, 2H), 7.05 (m, 2H), 6.74 (s, 1H), 4.09 (m, 2H), 3.88 (s, 3H), 2.75-1.15 (m, 12H), 1.01 (s, 3H), 0.90 (s, 3H).

### Step-5: 5-Chloro-2-{3-[4-(4-fluoro-benzyl)-4-hydroxy-3,3-dimethyl-piperidin-1-yl]-propoxy}-4-methoxy-phenyl)-pyrrolidin-1-yl-methanone

To a solution of 5-Chloro-2-{3-[4-(4-fluoro-benzyl)-4-hydroxy-3,3-dimethyl-piperidin-1-yl]-propoxy}-4-methoxy-benzoic acid (100 mg, 0.21 mmol) in DMF was added diisopropylethylamine (0.1 ml, 0.63 mmol) followed by addition of EDCI (100 mg, 0.52 mmol), HOBT (30 mg, 0.21 mmol) and pyrrolidine (0.02 ml, 0.21 mmol) at cold condition. The reaction mixture was stirred at room temperature for overnight, diluted with water and extracted with ethyl acetate. The organic layer was washed with water, brine and dried over Na₂SO₄. The organic layer was concentrated and purified by flash chromatography (Si-gel, 2% methanol-DCM) to afford 70 mg (62.5%) of 5-Chloro-2-{3-[4-(4-fluoro-benzyl)-4-hydroxy-3,3-dimethyl-piperidin-1-yl]-propoxy}-4-methoxy-phenyl)-pyrrolidin-1-yl-methanone. LC/MS [M+H]⁺: 533.6. ¹H-NMR (400 MHz, DMSO-d₆) δ (ppm): 7.24 (m, 2H), 7.19 (s, 1H), 7.04 (t, 2H), 6.78 (s, 1H), 4.09 (m, 2H), 3.89 (s, 3H), 3.41-1.05 (m, 20H), 1.03 (s, 3H), 0.90 (s, 3H). HPLC: 97.54%.

### Example 88

### 5-Chloro-2-{3-[4-(4-chloro-phenyl)-4-hydroxy-3,3-dimethyl-piperidin-1-yl]-propoxy}-4-methoxy-N-methyl-benzamide

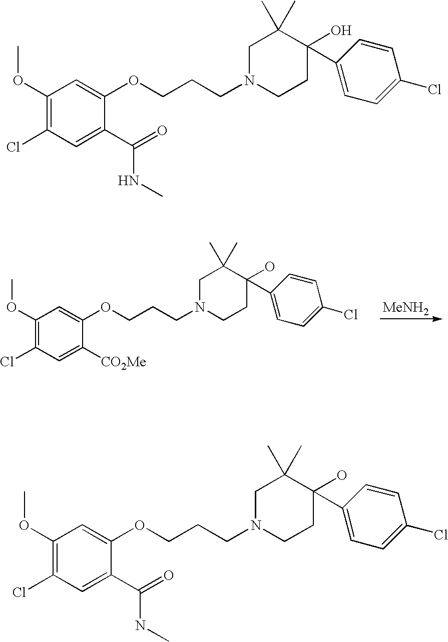

To a solution of 5-Chloro-2-{3-[4-(4-chloro-phenyl)-4-hydroxy-3,3-dimethyl-piperidin-1-yl]-propoxy}-4-methoxy-benzoic acid methyl ester (75 mg, 0.15 mmol) in ethanol was added 2(M) methylamine solution in ethanol and heated at 80° C. in a sealed tube overnight. The mixture was concentrated and purified over column chromatography (Si-gel, 3% MeOH-DCM) to afford 20 mg (27%) of 5-Chloro-2-{3-[4-(4-chloro-phenyl)-4-hydroxy-3,3-dimethyl-piperidin-1-yl]-propoxy}-4-methoxy-N-methyl-benzamide. LC/MS [M+H]⁺: 495.4. ¹H-NMR (400 MHz, DMSO-d₆) δ (ppm): 7.92 (s, 1H), 7.77 (s, 1H), 7.46 (d, 2H), 7.34 (d, 2H), 6.83 (s, 1H), 4.67 (s, 1H), 4.25 (s, 2H), 3.93 (s, 3H), 2.98 (s, 3H), 2.66 (m, 1H), 2.69 (m, 2H), 2.50 (s, 3H), 2.32 (bs, 1H), 1.98 (bs, 2H), 0.78 (s, 3H), 0.64 (s, 3H). HPLC: 92.8%.

### Example 89

### (5-Chloro-2-{3-[4-(4-chloro-phenyl)-4-hydroxy-3,3-dimethyl-piperidin-1-yl]-propoxy}-4-methoxy-phenyl)-pyrrolidin-1-yl-methanone

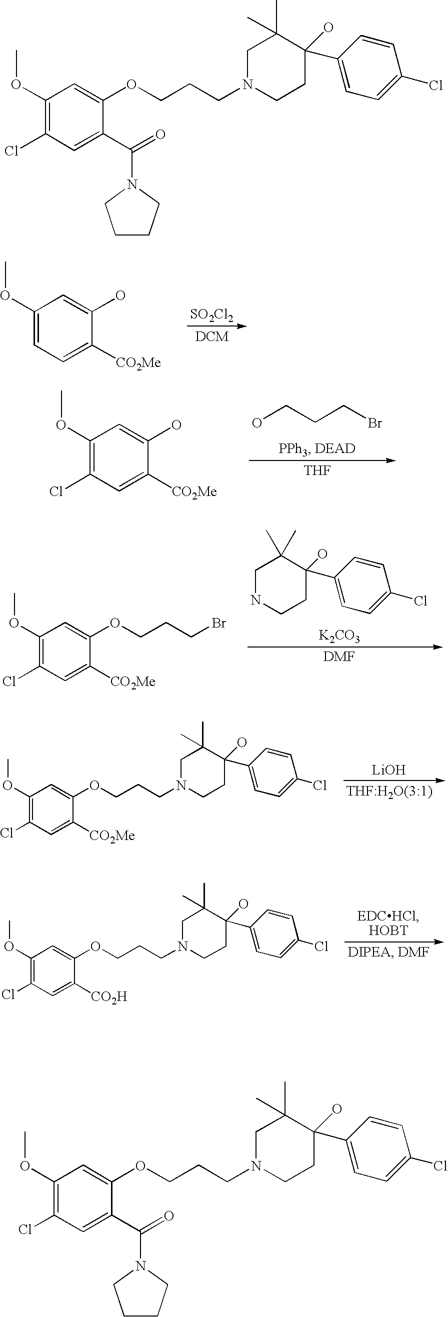

4-(4-Chloro-phenyl)-3,3-dimethyl-piperidin-4-ol was prepared by a process as described in example 10.

### Step-1: 5-Chloro-2-hydroxy-4-methoxy-benzoic acid methyl ester

To a solution of 2-Hydroxy-4-methoxy-benzoic acid methyl ester (5.0 g, 27.4 mmol) in DCM was added aq. HCl (1 ml of 2.5 M) and sulphuryl chloride (2.28 ml, 28.8 mmol). The resulting mixture was refluxed for 2 hours. The reaction mixture was evaporated under reduced pressure and the solid thus obtained was stirred with 25 ml of methanol for 30 minutes. Precipitated solid was filtered, washed with methanol and dried to afford 4.8 g (80.6%) of compound 5-Chloro-2-hydroxy-4-methoxy-benzoic acid methyl ester. GCMS (m/z)-216. ¹H-NMR (400 MHz, DMSO-d₆) δ (ppm): 10.74 (s, H), 7.74 (s, H), 6.74 (s, H), 3.89 (s, 3H), 3.86 (s, 3H).

### Step-2: 2-(3-Bromo-propoxy)-5-chloro-4-methoxy-benzoic acid methyl ester

To a solution of triphenyl phosphine (1.2 g, 4.61 mmol) in THF was added DIAD (0.7 ml, 3.46 mmol) at 0° C. and the mixture was stirred for 1 hour. A solution of 3-bromopropanol (0.2 ml, 2.3 mmol) and 5-Chloro-2-hydroxy-4-methoxy-benzoic acid methyl ester (500 mg, 2.3 mmol) in THF was then added at 0° C. and the mixture was stirred at room temperature overnight. The mixture was concentrated and purified through Si-gel column (5% ethyl acetate-hexane) to afford 1 g (60%) of 1-(3-Bromo-propoxy)-4-chloro-2-nitro-benzene. LC/MS [M+H]⁺: 338.4. ¹H-NMR (400 MHz, DMSO-d₆) δ (ppm): 7.74 (s, 1H), 6.86 (s, 1H), 4.22 (t, 2H), 3.95 (s, 3H), 3.77-3.74 (m, 5H), 2.26 (m, 2H).

### Step-3: 5-Chloro-2-{3-[4-(4-chloro-phenyl)-4-hydroxy-3,3-dimethyl-piperidin-1-yl]-propoxy}-4-methoxy-benzoic acid methyl ester

To a solution of 4-(4-chloro-phenyl)-3,3-dimethyl-piperidine-4-ol (430 mg, 1.78 mmol) in DMF was added anhydrous potassium carbonate (490 mg, 3.56 mmol) and 2-(3-Bromo-propoxy)-5-chloro-4-methoxy-benzoic acid methyl ester (600 mg, 1.78 m.mol). The reaction mixture was stirred for overnight and diluted with water. The organics was extracted with ethyl acetate and washed with water. The organic layer was concentrated under reduced pressure and the residue was purified by flash chromatography (silica-gel, 1.5% methanol-DCM) to afford 700 mg (79.6%) of 5-Chloro-2-{3-[4-(4-chloro-phenyl)-4-hydroxy-3,3-dimethyl-piperidin-1-yl]-propoxy}-4-methoxy-benzoic acid methyl ester. LC/MS [M+H]⁺:496.4. ¹H-NMR (400 MHz, CDCl₃) δ (ppm): 7.89 (s, 1H), 7.4-7.26 (m, 4H), 4.12 (t, 2H), 3.93 (s, 3H), 3.85 (s, 3H), 2.83-2.01 (m, 9H), 1.24 (s, 2H), 0.87 (s, 3H), 0.75 (s, 3H).

### Step-4: 5-Chloro-2-{3-[4-(4-chloro-phenyl)-4-hydroxy-3,3-dimethyl-piperidin-1-yl]-propoxy}-4-methoxy-benzoic acid

To a stirred solution of 5-Chloro-2-{3-[4-(4-chloro-phenyl)-4-hydroxy-3,3-dimethyl-piperidin-1-yl]-propoxy}-4-methoxy-benzoic acid methyl ester (300 mg, 0.61 m.mol) in THF: H2O (4:1) was added LiOH (254 mg, 6.06m.mol) at cold condition. The reaction mixture was refluxed for overnight and concentrated. The residual mass was adjusted to pH 5-6 with dil HCl and extracted with ethyl acetate. The organic layer was concentrated under reduced pressure to afford 280 mg (96.7%) of 5-Chloro-2-{3-[4-(4-chloro-phenyl)-4-hydroxy-3,3-dimethyl-piperidin-1-yl]-propoxy}-4-methoxy-benzoic acid. LC/MS [M+H]⁺: 482.0. ¹H-NMR (400 MHz, DMSO-d₆) δ (ppm): 7.74 (s, 1H), 7.46-7.36 (m, 4H), 6.82 (s, 1H), 5.1 (s, 1H), 4.17 (m, 3H), 3.92 (s, 3H), 3.06-1.15 (m, 9H), 0.80 (s, 3H), 0.69 (s, 3H). HPLC: 96.7%.

### Step-5: (5-Chloro-2-{3-[4-(4-chloro-phenyl)-4-hydroxy-3,3-dimethyl-piperidin-1-yl]-propoxy}-4-methoxy-phenyl)-pyrrolidin-1-yl-methanone

To a solution of 5-Chloro-2-{3-[4-(4-chloro-phenyl)-4-hydroxy-3,3-dimethyl-piperidin-1-yl]-propoxy}-4-methoxy-benzoic acid (100 mg, 0.21 m.mol) in DMF was added pyrrolidine (0.02 ml, 0.21m.mol), EDCI (120 mg, 0.62 mmol), HOBT (212 mg, 1.57 mmol) and DIPEA (0.11 ml, 0.31 mmol) at 5-10° C. and the mixture was stirred at room temperature for 14-16 hours. The reaction mixture was then diluted with water and extracted with ethyl acetate. The organic layer was concentrated under reduced pressure and the residue was purified by flash chromatography (silica-gel, 2% MeOH-DCM) to afford 50 mg (45%) of 5-Chloro-2-{3-[4-(4-chloro-phenyl)-4-hydroxy-3,3-dimethyl-piperidin-1-yl]-propoxy}-4-methoxy-phenyl)-pyrrolidin-1-yl-methanone. LC/MS [M+H]⁺: 535.3. ¹H-NMR (400 MHz, DMSO-d₆) δ (ppm): 7.47-7.32 (m, 4H), 7.2 (s, 1H), 6.8 (s, 1H), 4.65 (s, 1H) 4.12 (t, 3H), 3.9 (s, 3H), 3.43-2.35 (m, 12H), 1.83 (m, 6H), 0.78 (s, 3H), 0.64 (s, 3H). HPLC: 91.5%.

### Example 90

### 5-Chloro-2-{3-[4-(4-fluoro-benzyl)-4-hydroxy-3,3-dimethyl-piperidin-1-yl]-propoxy}-4-methoxy-benzoic acid

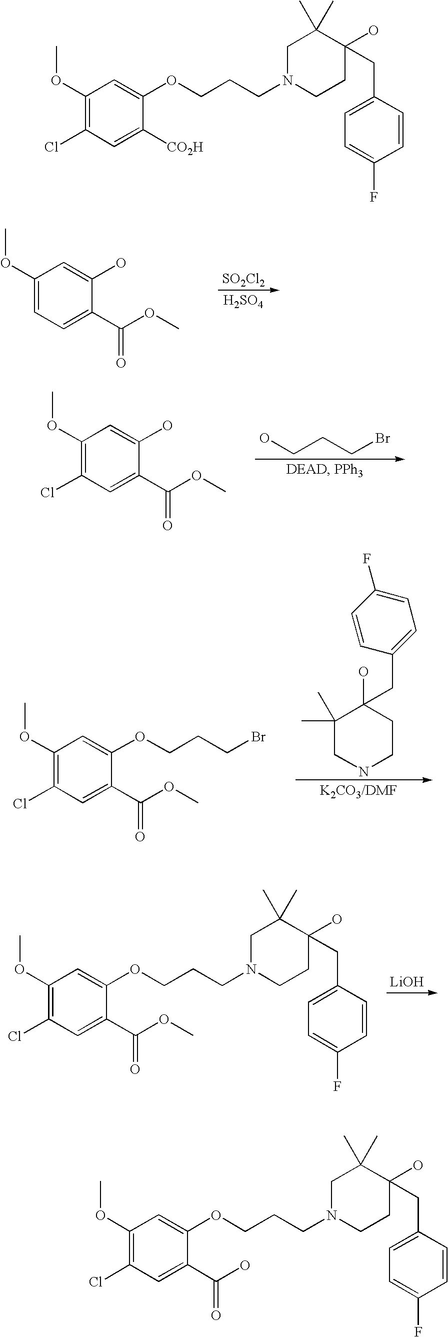

4-(4-Fluoro-benzyl)-3,3-dimethyl-piperidin-4-ol was prepared by a process similar to that described in example 30.

### Step-1: 5-Chloro-2-hydroxy-4-methoxy-benzoic acid methyl ester

To a solution of 2-Hydroxy-4-methoxy-benzoic acid methyl ester (5.0 g, 27.4 mmol) in DCM (25 ml), aq. HCl (1 ml of 2.5 M) and sulphonyl chloride (2.28 ml, 28.8 mmol) was added and the resulting mixture was refluxed for 2 hours. The reaction mixture was concentrated under reduced pressure and the solid thus obtained was stirred with 25 ml of methanol for 30 minutes. The solid was filtered, washed with methanol and dried under reduced pressure to afford 4.8 g (80.6%) of 5-Chloro-2-hydroxy-4-methoxy-benzoic acid methyl ester. ¹H-NMR (400 MHz, CDCl₃) δ (ppm): 10.74 (s, H), 7.74 (s, H), 6.74 (s, H), 3.89 (s, 3H), 3.86 (s, 3H).

### Step-2: 2-(3-Bromo-propoxy)-5-chloro-4-methoxy-benzoic acid methyl ester

To a solution of triphenyl phosphine (1.2 g, 4.61 mmol) in THF was added DIAD (0.7 ml, 3.46 mmol) at 0° C. and the mixture was stirred for 1 hour. A solution of 3-bromopropanol (0.2 ml, 2.3 mmol) and 5-Chloro-2-hydroxy-4-methoxy-benzoic acid methyl ester (500 mg, 2.3 mmol) in THF was then added at 0° C. and the mixture was stirred at room temperature overnight. The mixture was concentrated and purified through Si-gel column (5% ethyl acetate-hexane) to afford 1 g (60%) of 1-(3-Bromo-propoxy)-4-chloro-2-nitro-benzene. LC/MS [M+H]⁺: 338.4.

### Step-3: 5-Chloro-2-{3-[4-(4-fluoro-benzyl)-4-hydroxy-3,3-dimethyl-piperidin-1-yl]-propoxy}-4-methoxy-benzoic acid methyl ester

To a solution of 2-(3-Bromo-propoxy)-5-chloro-4-methoxy-benzoic acid methyl ester (400 mg, 1.2 mmol) in DMF was added K₂CO₃ (328 mg, 2.34 mmol) and 4-(4-Fluoro-benzyl)-3,3-dimethyl-piperidin-4-ol (281 mg, 1.2 mmol) were added. The reaction mixture was stirred at rt. for overnight. The reaction mixture was diluted with water and extracted with ethyl acetate. The organic layer was washed with water, brine and concentrated to produce 550 mg (92.7%) of 5-Chloro-2-{3-[4-(4-fluoro-benzyl)-4-hydroxy-3,3-dimethyl-piperidin-1-yl]-propoxy}-4-methoxy-benzoic acid methyl ester. LC/MS [M+H]⁺: 494.4. ¹H-NMR (400 MHz, DMSO-d₆) δ (ppm): 7.71 (s, 1H), 7.23 (m, 2H), 7.04 (m, 2H), 6.80 (s, 1H), 4.13 (t, 2H), 3.93 (s, 3H), 3.74 (s, 3H), 2.73-1.84 (m, 12H), 1.01 (s, 3H), 0.89 (s, 3H).

### Step-4: 5-Chloro-2-{3-[4-(4-fluoro-benzyl)-4-hydroxy-3,3-dimethyl-piperidin-1-yl]-propoxy}-4-methoxy-benzoic acid

To a solution of 5-Chloro-2-{3-[4-(4-fluoro-benzyl)-4-hydroxy-3,3-dimethyl-piperidin-1-yl]-propoxy}-4-methoxy-benzoic acid methyl ester (400 mg, 0.81 mmol) in THF: H₂O (4:1) was added LiOH (380 mg, 9.0 mmol) and heated at 80° C. for overnight. The reaction mixture was concentrated, diluted with water, neutralized with dilute HCl and extracted with ethyl acetate. The organic layer was washed with water and concentrated to afford 350 mg (90%) of 5-Chloro-2-{3-[4-(4-fluoro-benzyl)-4-hydroxy-3,3-dimethyl-piperidin-1-yl]-propoxy}-4-methoxy-benzoic acid. LC/MS [M+H]⁺: 480.2.

¹H-NMR (400 MHz, DMSO-d₆) δ (ppm): 7.65 (s, 1H), 7.24 (m, 2H), 7.05 (m, 2H), 6.74 (s, 1H), 4.09 (m, 2H), 3.88 (s, 3H), 2.75-1.15 (m, 12H), 1.01 (s, 3H), 0.90 (s, 3H). HPLC: 94.5%.

### Example 91

### 5-Chloro-2-{3-[4-(4-fluoro-benzyl)-4-hydroxy-3,3-dimethyl-piperidin-1-yl]-propoxy}-4-hydroxy-N-methyl-benzamide

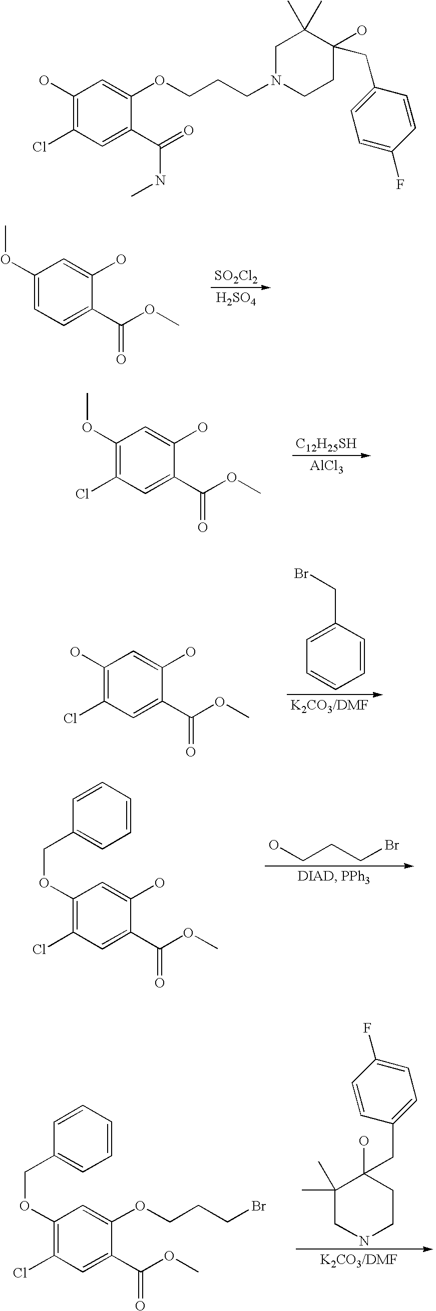

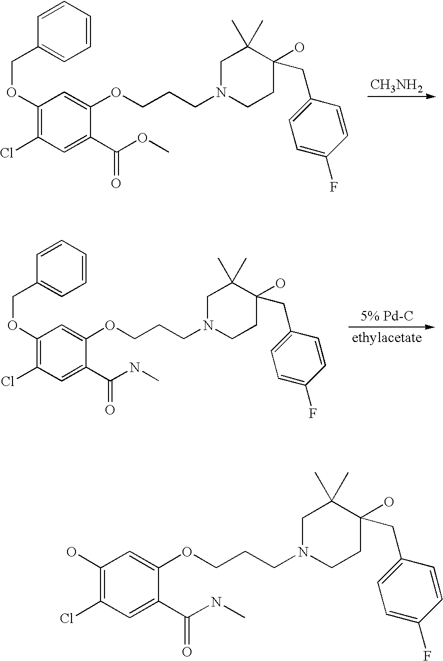

4-(4-Fluoro-benzyl)-3,3-dimethyl-piperidin-4-ol was prepared by a process similar to that described in example 30.

### Step-1: 5-Chloro-2-hydroxy-4-methoxy-benzoic acid methyl ester

To a solution of 2-Hydroxy-4-methoxy-benzoic acid methyl ester (5.0 g, 27.4 mmol) in DCM (25 ml), aq. HCl (1 ml of 2.5 M) and sulphonyl chloride (2.28 ml, 28.8 mmol) was added and the resulting mixture was refluxed for 2 hours. The reaction mixture was concentrated under reduced pressure and the solid thus obtained was stirred with 25 ml of methanol for 30 minutes. The solid was filtered, washed with methanol and dried under reduced pressure to afford 4.8 g (80.6%) of 5-Chloro-2-hydroxy-4-methoxy-benzoic acid methyl ester. LC/MS [M+H]⁺: 217.2. ¹H-NMR (400 MHz, CDCl₃) δ (ppm): 10.74 (s, H), 7.74 (s, H), 6.74 (s, H), 3.89 (s, 3H), 3.86 (s, 3H).

### Step-2: 5-Chloro-2-hydroxy-4-methoxy-benzoic acid methyl ester

2-Hydroxy-4-methoxy-benzoic acid methyl ester (500 mg, 2.3 mmol) was dissolved in 1-Dodecanethiol (2.5 ml) and heated to 40° C. for 1 hour. To this slurry was added AlCl₃ (750 mg, 2.4 eq.) portion wise. The reaction mixture was then heated at 40° C. for 1 hour. It was then quenched with ice and stirred at room temperature for overnight. It was then extracted with ethyl acetate, washed with water and brine. It was then dried over Na₂SO₄ and concentrated. The organic layer was concentrated and washed with hexane and dried to afford 300 mg (64.4%) of 5-Chloro-2-hydroxy-4-methoxy-benzoic acid methyl ester. ¹H-NMR (400 MHz, DMSO-d₆) δ (ppm): 11.28 (brs, 1H), 10.55 (s, 1H), 7.69 (s, 1H), 6.51 (s, 1H), 3.84 (s, 3H).

### Step-3: 4-Benzyloxy-5-chloro-2-hydroxy-benzoic acid methyl ester

5-Chloro-2-hydroxy-4-methoxy-benzoic acid methyl ester (1.0 gm, 4.9 mmol) in DMF was added K₂CO₃ (740.0 mg) and bromomethyl-benzene (0.7 ml) were added. The reaction mixture was heated at 70° C. for 2 hours. Then it was diluted with water and extracted with ethyl acetate. It was then washed with water and brine. It was dried over Na₂SO₄. It was concentrated and purified through column chromatography using (Si-gel, 1.5% ethyl acetate-hexane) to afford 1.05 g (73.2%) of 4-Benzyloxy-5-chloro-2-hydroxy-benzoic acid methyl ester. LC/MS [M+H]⁺: 293.3. ¹H-NMR (400 MHz, DMSO-d₆) δ (ppm): 10.88 (s, 1H), 7.83 (s, 1H), 7.45-7.25 (m, 4H), 7.25 (s, 1H), 6.54 (s, 1H), 5.15 (s, 2H), 3.91 (s, 3H).

### Step-4: 4-Benzyloxy-2-(3-bromo-propoxy)-5-chloro-benzoic acid methyl ester

Triphenylphosphine (550 mg, 2.05 mmol) in THF was added DIAD (0.3 ml, 1.54 mmol) drop wise at cold condition. The reaction mixture was stirred for half an hour. To the white suspension was added 4-benzyloxy-5-chloro-2-hydroxy-benzoic acid methyl ester (300 mg, 1.02 mmol) and 3-bromo-propan-1-ol (0.09 ml, 1.02 mmol) at a time. The reaction mixture was stirred at room temperature for overnight. It was then concentrated and purified through column chromatography (Si-gel, 4% etyl acetate-hexane) to afford 250 mg (59.2%) of 4-Benzyloxy-2-(3-bromo-propoxy)-5-chloro-benzoic acid methyl ester. LC/MS [M+H]⁺: 415.1. ¹H-NMR (400 MHz, DMSO-d₆) δ (ppm): 7.76 (s, 1H), 7.50-7.36 (m, 5H), 7.00 (s, 1H), 5.33 (s, 2H), 4.20 (m, 2H), 3.74 (m, 5H), 2.24 (m, 2H).

### Step-5: 4-Benzyloxy-5-chloro-2-{3-[4-(4-fluoro-benzyl)-4-hydroxy-3,3-dimethyl piperidin-1-yl]-propoxy}-benzoic acid methyl ester

To a solution of 4-benzyloxy-2-(3-bromo-propoxy)-5-chloro-benzoic acid methyl ester (200 mg, 0.48 mmol) in DMF was added K₂CO₃ (134 mg, 0.97 mmol) and 4-(4-Fluoro-benzyl)-3,3-dimethyl-piperidin-4-ol (115 mg, 0.48 mmol). The reaction mixture was stirred at room temperature overnight, diluted with water and extracted with ethyl acetate. The organic layer was concentrated and purified through column chromatography (Si-gel, 2.5% methanol-DCM) to afford 210 mg (76.2%) of 4-Benzyloxy-5-chloro-2-{3-[4-(4-fluoro-benzyl)-4-hydroxy-3,3-dimethyl piperidin-1-yl]-propoxy}-benzoic acid methyl ester. LC/MS [M+H]⁺: 570.5.

### Step-6: 4-Benzyloxy-5-chloro-2-{3-[4-(4-fluoro-benzyl)-4-hydroxy-3,3-dimethyl-piperidin-1-yl]-propoxy}-N-methyl-Benz amide

To a solution of 4-Benzyloxy-5-chloro-2-{3-[4-(4-fluoro-benzyl)-4-hydroxy-3,3-dimethyl piperidin-1-yl]-propoxy}-benzoic acid methyl ester (200 mg, 0.35 mmol) in ethanol was added a solution of methylamine in ethanol solution and heated at 70° C. for overnight in sealed tube. It was cooled and concentrated. The organics was diluted with water and extracted with ethyl acetate, washed with water and brine. It was dried over Na₂SO₄ and concentrated to afford 185 mg (92.7%) of 4-Benzyloxy-5-chloro-2-{3-[4-(4-fluoro-benzyl)-4-hydroxy-3,3-dimethyl-piperidin-1-yl]-propoxy}-N-methyl-Benz amide. LC/MS [M+H]⁺: 569.6.

### Step-7: 5-Chloro-2-{3-[4-(4-fluoro-benzyl)-4-hydroxy-3,3-dimethyl-piperidin-1-yl]-propoxy}-4-hydroxy-N-methyl-benzamide

4-Benzyloxy-5-chloro-2-{3-[4-(4-fluoro-benzyl)-4-hydroxy-3,3-dimethyl-piperidin-1-yl]-propoxy}-N-methyl-Benzamide (185 mg, 0.32 mmol) was dissolved in ethyl acetate was added 5% Pd—C (10 mg) and hydrogenated for 5 hours. The reaction mixture was filtered, concentrated and purified through column chromatography (Si-gel, 5% methanol-DCM) to afford 100 mg (64.2%) of 5-Chloro-2-{3-[4-(4-fluoro-benzyl)-4-hydroxy-3,3-dimethyl-piperidin-1-yl]-propoxy}-4-hydroxy-N-methyl-benzamide. LC/MS [M+H]⁺: 479.4. ¹H-NMR (400 MHz, DMSO-d₆) δ (ppm): 10.76 (brs, 1H), 7.84-6.66 (m, 7H), 4.07 (m, 2H), 3.83 (brs, 1H), 2.79-1.14 (m, 15H), 1.04 (s, 3H), 0.91 (s, 3H). HPLC: 94.2%.

### Example 92

### 5-Chloro-2-{3-[4-(4-chloro-phenyl)-4-hydroxy-3,3-dimethyl-piperidin-1-yl]-propoxy}-4-methoxy-N,N-dimethyl-benzamide

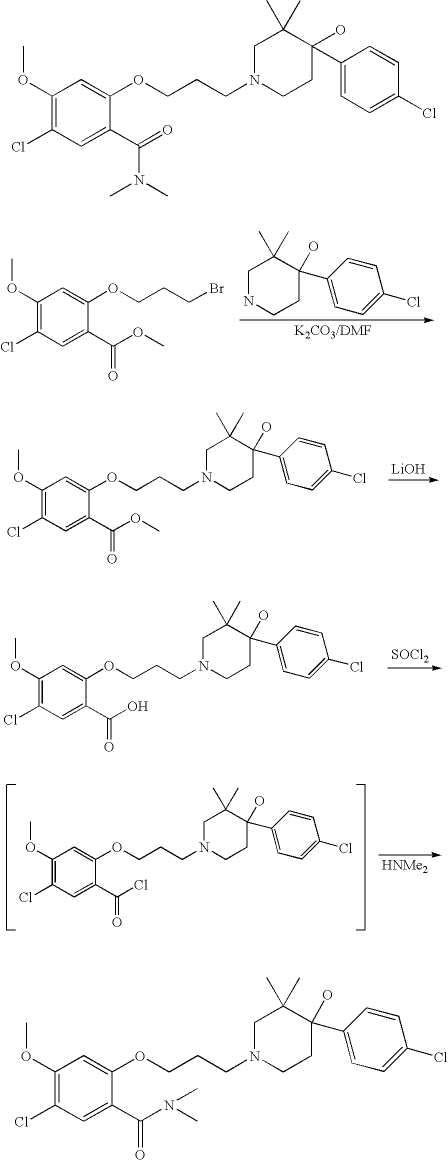

4-(4-Chloro-phenyl)-3,3-dimethyl-piperidin-4-ol was prepared by a process as described in example 10.

### Step-1: 5-Chloro-2-{3-[4-(4-chloro-phenyl)-4-hydroxy-3,3-dimethyl-piperidin-1-yl]-propoxy}-4-methoxy-benzoic acidmethyl ester

To a solution of 2-(3-Bromo-propoxy)-5-chloro-4-methoxy-benzoic acid methyl ester (600 mg, 1.78 mmol) in DMF was added K₂CO₃ (490 mg, 3.56 mmol) and 4-(4-Chloro-phenyl)-3,3-dimethyl-piperidin-4-ol (427 mg, 1.78 mmol). The reaction mixture was stirred at room temperature overnight, diluted with water and extracted with ethyl acetate. The organic layer was washed with water and brine. It was then concentrated and purified through column chromatography (Si-gel, 1.5% methanol-DCM) to afford 700 mg (79.2%) of 5-Chloro-2-{3-[4-(4-chloro-phenyl)-4-hydroxy-3,3-dimethyl-piperidin-1-yl]-propoxy}-4-methoxy-benzoic acidmethyl ester. LC/MS [M+H]⁺: 496.4.

### Step-2: 5-Chloro-2-{3-[4-(4-chloro-phenyl)-4-hydroxy-3,3-dimethyl-piperidin-1-yl]-propoxy}-4-methoxy-benzoic acid

To a solution of 5-Chloro-2-{3-[4-(4-chloro-phenyl)-4-hydroxy-3,3-dimethyl-piperidin-1-yl]-propoxy}-4-methoxy-benzoic acid methyl ester (300 mg, 0.60 mmol) in THF: H₂O (4:1) was added LiOH (255 mg, 6.0 mmol) The reaction mixture was heated at 80° C. for overnight, concentrated and diluted with water. The aq. solution was neutralized and extracted with ethyl acetate. It was washed with water and brine. The organic layer was dried and concentrated to afford 280 mg (96.7%) of 5-Chloro-2-{3-[4-(4-chloro-phenyl)-4-hydroxy-3,3-dimethyl-piperidin-1-yl]-propoxy}-4-methoxy-benzoic acid. LC/MS [M+H]⁺: 482.1.

### Step-3: 5-Chloro-2-{3-[4-(4-chloro-phenyl)-4-hydroxy-3,3-dimethyl-piperidin-1-yl]-propoxy}-4-methoxy-N,N-dimethyl-benzamide

5-Chloro-2-{3-[4-(4-chloro-phenyl)-4-hydroxy-3,3-dimethyl-piperidin-1-yl]-propoxy}-4-methoxy-benzoic acid (90 mg, 0.19 mmol) in DCM was added SOCl₂ (0.02 ml) at cold condition and reaction mixture was stirred at room temperature for 2 hours. The reaction mixture was concentrated under reduced pressure and trapped with toluene twice. The organics in DCM was added dimethylamine (1 ml) and stirred at RT for overnight. Concentrated the reaction mixture and extracted with ethyl acetate. the organic layer was washed with water and brine. Finally it was dried over Na₂SO₄ and concentrated. It was purified through colun chromatography (Si-gel, 2.75% methanol-DCM) to afford 25 mg (26.3%) of 5-Chloro-2-{3-[4-(4-chloro-phenyl)-4-hydroxy-3,3-dimethyl-piperidin-1-yl]-propoxy}-4-methoxy-N,N-dimethyl-benzamide. LC/MS [M+H]⁺: 509.2. ¹H-NMR (400 MHz, DMSO-d₆) δ (ppm): 7.47-7.18 (m, 5H), 6.80 (s, 1H), 4.64 (s, 1H), 4.12 (m, 2H), 3.90 (s, 3H), 2.95 (s, 3H), 2.78 (s, 3H), 2.67-1.40 (m, 10H), 0.79 (s, 3H), 0.64 (s, 3H). HPLC: 93.8%.

### Example 93

### 5-Chloro-2-{3-[3-(4-chloro-phenyl)-3-hydroxy-pyrrolidin-1-yl]-propoxy}-4-methoxy-N-methyl-benzamide

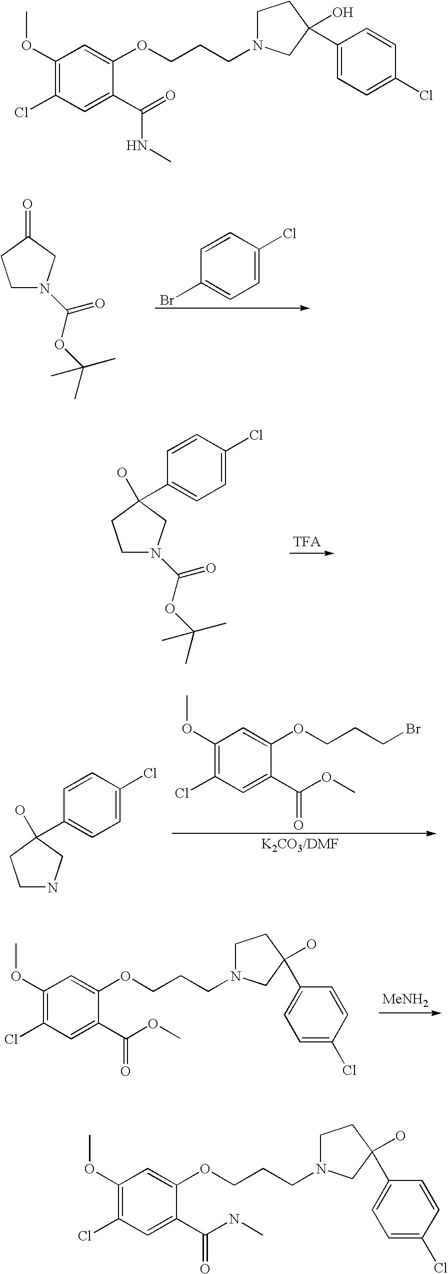

2-(3-Bromo-propoxy)-5-chloro-4-methoxy-benzoic acid methyl ester was prepared by a process as described in example 85.

### Step-1: 3-(4-Chloro-phenyl)-3-hydroxy-pyrrolidine-1-carboxylic acid tert-butyl ester

1-Bromo-4-chloro-benzene (517 mg, 2.699 mmol) was dissolved in dry THF. To it n-BuLi (2.0 ml, 2.5 eq.) was added drop wise at −78° C. It was stirred for 1 hour to afford a white suspension. Then 3-Oxo-pyrrolidine-1-carboxylic acid tert-butyl ester (200 mg, 1.079 mmol) was added to the reaction mixture at −78° C. It was then stirred at −78° C. for 2 hours. Then it was quenched with aq. ammonium chloride solution. It was then extracted with ethylacetate. The organic layer was then washed with water and brine. It was then dried over Na₂SO₄. It was concentrated and purified through column chromatography (Si-gel, 7% ethyl acetate-hexane) to afford 140 mg (42.9%) of 3-(4-Chloro-phenyl)-3-hydroxy-pyrrolidine-1-carboxylic acid tert-butyl ester. LC/MS [M+H]⁺: 298.4.

### Step-2: 3-(4-Chloro-phenyl)-pyrrolidin-3-ol

3-(4-Chloro-phenyl)-3-hydroxy-pyrrolidine-1-carboxylic acid tert-butyl ester (140 mg, 0.47 mmol) was dissolved in dry DCM and to it TFA (0.4 ml, 4.7 mmol) was added drop wise at cold condition. It was then stirred at RT for 2 hours. Then it was concentrated and diluted with water. It was basified using aq. NaOH and extracted with ethyl acetate. Organic layer was then washed with water and brine. Finally it was dried over Na₂SO₄ and concentrated to afford 70 mg (75.3%) of 3-(4-Chloro-phenyl)-pyrrolidin-3-ol. LC/MS [M+H]⁺: 198.4.

### Step-3: 5-Chloro-2-{3-[3-(4-chloro-phenyl)-3-hydroxy-pyrrolidin-1-yl]-propoxy}-4-methoxy-benzoic acid methyl ester

To a solution of 2-(3-Bromo-propoxy)-5-chloro-4-methoxy-benzoic acid methyl ester (120 mg, 0.35 mmol) in DMF was added K₂CO₃ (100 mg, 0.71 mmol), and 3-(4-Chloro-phenyl)-pyrrolidin-3-ol (70 mg, 0.35 mmol) were added. The reaction mixture was then stirred at room temperature overnight. The reaction mixture was diluted with water and extracted with ethyl acetate. The organic layer was washed with water, brine and concentrated. The crude was purified through column chromatography (Si-gel, 0.5% methanol-DCM) to afford 36 mg (22.3%) of 5-Chloro-2-{3-[3-(4-chloro-phenyl)-3-hydroxy-pyrrolidin-1-yl]-propoxy}-4-methoxy-benzoic acid methyl ester. LC/MS [M+H]⁺: 454.4.

### Step-4: 5-Chloro-2-{3-[3-(4-chloro-phenyl)-3-hydroxy-pyrrolidin-1-yl]-propoxy}-4-methoxy-N-methyl-benzamide

To a solution of 5-Chloro-2-{3-[3-(4-chloro-phenyl)-3-hydroxy-pyrrolidin-1-yl]-propoxy}-4-methoxy-benzoic acid methyl ester (35 mg, 0.08 mmol) in ethanol was added methylamine in ethanol solution and heated at 70° C. for overnight. The reaction mixture as concentrated and extracted with ethyl acetate, washed with water and brine. The organic layer was concentrated and purified through column chromatography over neutral Al₂O₃ using 0.5% methanol-DCM to afford 25 mg (71.6%) of 5-Chloro-2-{3-[3-(4-chloro-phenyl)-3-hydroxy-pyrrolidin-1-yl]-propoxy}-4-methoxy-N-methyl-benzamide.

LC/MS [M+H]⁺: 452.9. ¹H NMR (400 MHz, DMSO-d₆): 7.93 (m, 1H), 7.78 (s, 1H), 7.49 (d, 2H), 7.33 (d, 2H), 6.84 (s, 1H), 5.32 (s, 1H), 4.27 (m, 2H), 3.93 (s, 3H), 2.80-1.33 (m, 19H). HPLC: 93.29%.

### Example 94

### 5-Chloro-2-{3-[3-(4-chloro-benzyl)-3-hydroxy-pyrrolidin-1-yl]-propoxy}-4-methoxy-N-methyl-benzamide

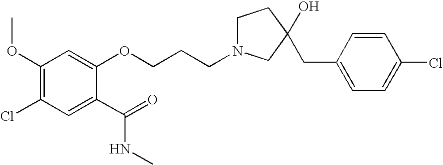

### Preparation of 3-(4-Chloro-benzyl)-pyrrolidin-3-ol

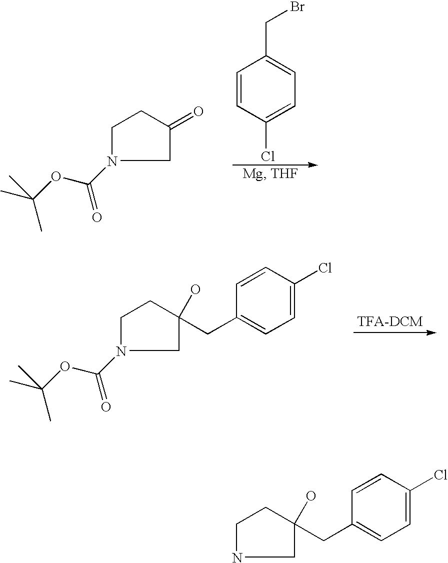

### Step-1: 4-(4-Fluoro-benzyl)-4-hydroxy-3,3-dimethyl-piperidine-1-carboxylic acid tent-butyl ester

To a solution of 4-chlorobenzyl bromide (832 mg, 4.05 mmol) in ether was added Mg turning (97 mg, 4.05 mmol) and the resulting mixture was stirred at room temperature for 1 hour. To the resulting Grignard solution was added a solution of 3-Oxo-pyrrolidine-1-carboxylic acid tert-butyl ester (500 mg, 2.7 mmol) in THF drop wise at room temperature. The reaction mixture was heated to reflux overnight, cooled then diluted with aqueous saturated NH₄Cl solution and extracted with ethyl acetate. The organic layer was washed with water, dried over sodium sulfate, concentrated and purified over Si-gel (30% ether-hexane) to afford 250 mg (30%) of 3-(4-Chloro-benzyl)-3-hydroxy-pyrrolidine-1-carboxylic acid tert-butyl ester. LC/MS [M+H]⁺: 312.4.

### Step-2: 3-(4-Chloro-benzyl)-pyrrolidin-3-ol

To a solution of 3-(4-Chloro-benzyl)-3-hydroxy-pyrrolidine-1-carboxylic acid tert-butyl ester (250 mg, 0.8 mmol)) in DCM was added trifluoroacetic acid (3.8 ml, 50 mmol). The resulting mixture was stirred for about 5 hours then concentrated and washed with dry ether. The TFA salt was diluted with a minimum volume of water, neutralized with aq. NaOH solution at 5-10° C. and extracted with ethyl acetate. The organic layer was dried over sodium sulfate and concentrated to afford 90 mg (53%) of 3-(4-Chloro-benzyl)-pyrrolidin-3-ol. LC/MS [M+H]⁺: 212.2.

5-Chloro-2-{3-[3-(4-chloro-benzyl)-3-hydroxy-pyrrolidin-1-yl]-propoxy}-4-methoxy-N-methyl-benzamide was prepared by the process similar to that described in example 85.

LC/MS [M+H]⁺: 467.4. ¹H-NMR (400 MHz, DMSO-d₆) δ (ppm): 7.92 (s, 1H), 7.77 (s, 1H), 7.27 (s, 4H), 6.82 (s, 1H), 4.96 (m, 2H), 4.63 (s, 1H), 4.23 (m, 2H), 3.92 (s, 3H), 2.79 (s, 3H), 2.76-1.33 (m, 9H). HPLC: 96.4%.

### Example 95

### N-[5-Chloro-2-(2-{[1-(4-fluoro-benzyl)-4-hydroxy-piperidin-4-ylmethyl]-amino}-ethoxy)-4-methoxy-phenyl]-acetamide

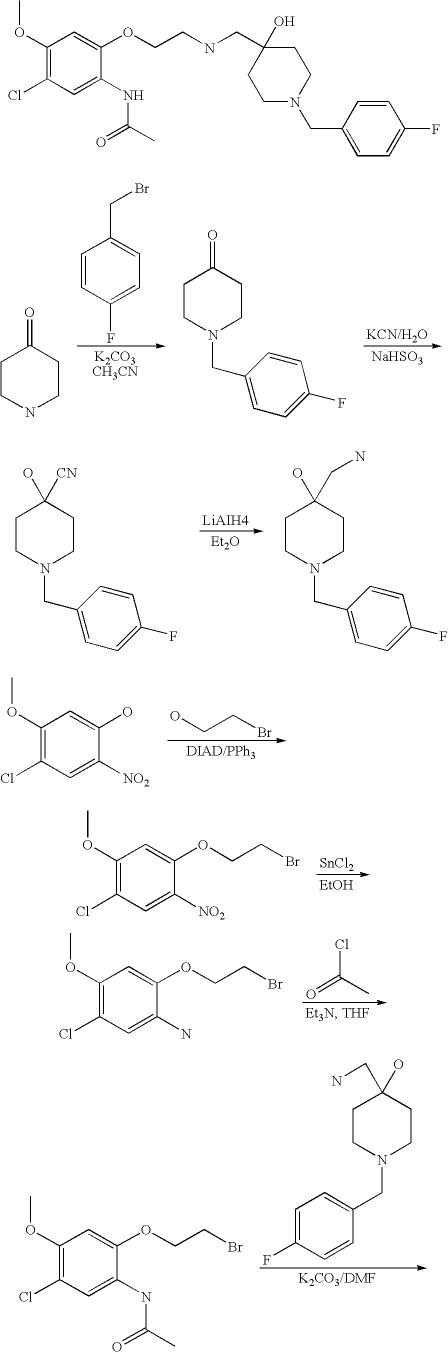

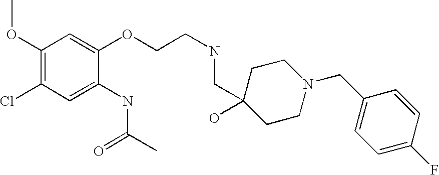

### Step-1:1-(4-Fluoro-benzyl)-piperidin-4-one

To a stirred solution of Piperidin-4-one (2 g, 13.0 mmol) in acetonitrile was added K₂CO₃ (5.40 g, 39.0 mmol) and 1-Bromomethyl-4-fluoro-benzene (1.5 ml, 11.7 mmol) at 0° C. The reaction mixture was stirred at room temperature for overnight. The reaction mixture was diluted with water and extracted with chloroform. The organic layer was washed with water, dilutes NaOH and dried over Na₂SO₄. The organic layer was concentrated in reduced pressure to afford 2.93 g of 1-(4-Fluoro-benzyl)-piperidin-4-one. LC/MS [M+H]⁺: 208.0.

### Step-2: 1-(4-Fluoro-benzyl)-4-hydroxy-piperidine-4-carbonitrile

To a solution of 1-(4-Fluoro-benzyl)-piperidin-4-one (2.93 g, 10.9 mmol) in water was added potassium cyanide (2.6 g, 25.2 mmol) and aqueous solution of NaHSO3 (2.6 g, 25.2 mmol). The reaction mixture was stirred for 1 hour. The reaction mixture was extracted with chloroform and washed with water. The organic layer was concentrated to afford 3.3 g of 1-(4-Fluoro-benzyl)-4-hydroxy-piperidine-4-carbonitrile.

¹H-NMR (400 MHz, CDCl₃) δ (ppm): 7.30 (m, H), 7.25 (m, H), 6.99 (m, 2H), 3.48 (s, 2H), 2.71 (m, 2H), 2.55 (bs, H), 2.39 (m, 2H), 2.09 (m, 2H), 1.87 (m, 2H).

### Step-3: 4-Aminomethyl-1-(4-fluoro-benzyl)-piperidin-4-ol

To a solution of 1-(4-Fluoro-benzyl)-4-hydroxy-piperidine-4-carbonitrile (1 g, 4.2 mmol) in diethyl ether was added lithium aluminium hydride (324 mg, 8.5 mmol) portionwise. The reaction mixture was stirred at room temperature for 2 hours and quenched (0.3 ml water, 0.3 ml 15% NaOH, 0.6 ml water). The precipitated solid was filtered off through celite bed. The organic layer was concentrated under reduced pressure to afford 730 mg of 4-Aminomethyl-1-(4-fluoro-benzyl)-piperidin-4-ol. LC/MS [M+H]⁺: 239.4.

### Step-4: 1-(2-Bromo-ethoxy)-4-chloro-5-methoxy-2-nitro-benzene

DEAD (0.8 ml, 3.7 mmol) was added to the THF solution of PPh₃ (1.3 g, 4.91 mmol) at 0° C. The reaction mixture was stirred for half an hour. Then to it 2-bromoethanol (0.2 ml, 2.46 mmol) and 4-chloro-5-methoxy-2-nitrophenol (500 mg 2.46 mmol) were added one by one. It was then stirred at rt. for overnight. Then it was concentrated and purified by column chromatography (Si-gel, 10% ethyl acetate-hexane) afforded 480 mg (62.9%) of 1-(2-Bromo-ethoxy)-4-chloro-5-methoxy-2-nitro-benzene.

¹H-NMR (400 MHz, DMSO-d₆) δ (ppm): 8.1 (s, 1H), 7.0 (s, 1H), 4.60 (m, 2H), 4.0 (s, 3H), 3.84 (m, 2H).

### Step-5: 2-(2-Bromo-ethoxy)-5-chloro-4-methoxy-phenylamine

To a solution of 1-(2-Bromo-ethoxy)-4-chloro-5-methoxy-2-nitro-benzene (470 mg, 1.51 mmol) in ethanol was added (1.7 gm, 7.57 mmol) SnCl₂. Then it was refluxed for 3 hours at 100° C. It was then concentrated and diluted with water. It was basified and extracted with ethyl acetate. Then it was washed with water, brine and dried over Na₂SO₄. It was concentrated to afford 400 mg (94.2%) of 2-(2-Bromo-ethoxy)-5-chloro-4-methoxy-phenylamine. LC/MS [M+H]⁺: 281.0.

### Step-6: N-[2-(2-Bromo-ethoxy)-5-chloro-4-methoxy-phenyl]-acetamide

To a solution of 2-(2-Bromo-ethoxy)-5-chloro-4-methoxy-phenylamine (500 mg, 1.78 mmol) in THF was added (0.3 ml, 1.960 mmol) triethyl amine at 0° C. Acetyl chloride (0.15 ml, 1.960 mmol) was then added to the reaction mixture drop wise. The reaction mixture was then stirred at rt. for 1 hour. Then it was concentrated and extracted with ethyl acetate. It was washed with water, brine and dried over Na₂SO₄ to produce 400 mg (69.6%) N-[2-(2-Bromo-ethoxy)-5-chloro-4-methoxy-phenyl]-acetamide. LC/MS [M+H]⁺: 323.3.

### Step-7: N-[5-Chloro-2-(2-{[7-(4-fluoro-benzyl)-4-hydroxy-piperidin-4-ylmethyl]-amino}-ethoxy)-4-methoxy-phenyl]-acetamide

To a solution of N-[2-(2-Bromo-ethoxy)-5-chloro-4-methoxy-phenyl]-acetamide (200 mg, 0.62 mmol) was dissolved in dry DMF. Then to it K₂CO₃ (172 mg, 1.24 mmol), and amine (150 mg, 0.62 mmol) were added. The reaction mixture was then stirred at rt. for overnight. It was diluted with water and extracted with ethyl acetate. Then it was washed with water and brine. It was then concentrated and purified by column chromatography (Si-gel, 10% methanol-DCM) to produce 80 mg (26.9%) of N-[5-Chloro-2-(2-{[1-(4-fluoro-benzyl)-4-hydroxy-piperidin-4-ylmethyl]-amino}-ethoxy)-4-methoxy-phenyl]-acetamide. LC/MS [M+H]⁺: 480.4. ¹H-NMR (400 MHz, DMSO-d₆) δ (ppm): 9.08 (s, 1H), 7.89 (s, 1H), 7.32-7.09 (m, 4H), 6.85 (s, 1H), 4.14 (brs, 2H), 3.84 (s, 3H), 3.42 (brs, 2H), 2.96 (m, 2H), 2.5-2.3 (m, 6-H), 2.04 (s, 3H), 1.5 (m, 4H). HPLC: 96.2%.

### Example 96

### 5-Chloro-2-(2-{[1-(4-fluoro-benzyl)-4-hydroxy-piperidin-4-ylmethyl]-amino}-ethoxy)-4-methoxy-N-methyl-benzamide

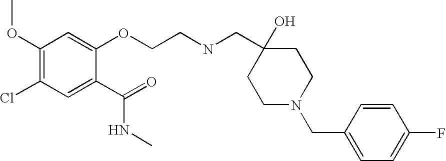

4-Aminomethyl-1-(4-fluoro-benzyl)-piperidin-4-ol was prepared by a process described in example 95.

5-Chloro-2-(2-{[1-(4-fluoro-benzyl)-4-hydroxy-piperidin-4-ylmethyl]-amino}-ethoxy)-4-methoxy-N-methyl-benzamide was prepared by a process similar to that described in example 85. LC/MS [M+H]⁺: 480.4. ¹H-NMR (400 MHz, DMSO-d₆) δ (ppm): 8.30 (s, 1H), 7.79 (s, 1H), 7.29 (m, 2H), 7.11 (m, 2H0, 6.87 (s, 1H), 4.26 (t, 2H), 4.08 (s, 1H), 3.92 (s, 3H), 3.37 (m, 2H), 2.96 (t, 2H), 2.79 (d, 3H), 2.40 (m, 2H), 2.27 (m, 2H), 1.51 (m, 5H). HPLC: 97.3%.

### Example 97

### N-(5-Chloro-2-{3-[4-(4-chloro-phenyl)-3-hydroxy-pyrrolidin-1-yl]-propoxy}-4-methoxy-phenyl)-acetamide

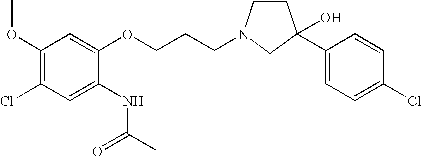

3-(4-Chloro-phenyl)-pyrrolidin-3-ol was prepared by a process as described in example 93. N-(5-Chloro-2-{3-[3-(4-chloro-phenyl)-3-hydroxy-pyrrolidin-1-yl]-propoxy}-4-methoxy-phenyl)-acetamide was prepared by a process similar to that described in example 67. LC/MS [M+H]⁺: 453.3. ¹H-NMR (400 MHz, CDCl₃) δ (ppm): 8.32 (bs, 1H), 7.51 (bs, 1H), 7.42 (d, 2H), 7.31 (m, 2H), 7.25 (s, 2H), 6.51 (s, 1H), 4.12 (t, 2H), 3.86 (s, 3H), 3.21 (m, 1H), 3.03 (d, 1H), 2.76 (t, 2H), 2.58 (m, 2H), 2.32 (m, 1H), 2.21 (m, 1H), 2.10 (s, 3H), 2.05 (m, 2H), 1.24 (m, 1H). HPLC: 93.2%.

### Example 98

### N-(5-Chloro-2-{3-[3-(4-chloro-benzyl)-3-hydroxy-pyrrolidin-1-yl]-propoxy}-4-methoxy-phenyl)-acetamide

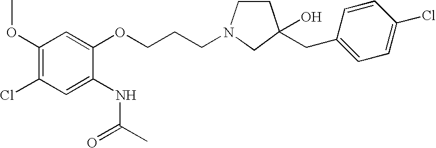

3-(4-Chloro-benzyl)-pyrrolidin-3-ol was prepared by a process as described in example 94. N-(5-Chloro-2-{3-[3-(4-chloro-benzyl)-3-hydroxy-pyrrolidin-1-yl]-propoxy}-4-methoxy-phenyl)-acetamide was prepared by a process similar to that described in example 78. LC/MS [M+H]⁺: 467.4. ¹H-NMR (400 MHz, DMSO-d₆) δ (ppm): 8.96 (s, 1H), 7.82 (m, 1H), 7.28 (s, 4H0, 6.81 (s, 1H), 4.66 (bs, 2H), 4.11 (m, 2H), 3.83 (s, 3H), 2.76 (m, 2H), 2.5 (s, 3H), 2.04 (s, 3H), 1.87-1.59 (m, 6H). HPLC: 94.3%.

### Example 99 and 100

### 5-Chloro-4-dimethylamino-2-{3-[4-(4-fluoro-benzyl)-4-hydroxy-3,3-dimethyl-piperidin-1-yl]-propoxy}-N-methyl-benzamide and 5-Chloro-2-{3-[4-(4-fluoro-benzyl)-4-hydroxy-3,3-dimethyl-piperidin-1-yl]-propoxy}-N-methyl-4-methylamino-benzamide

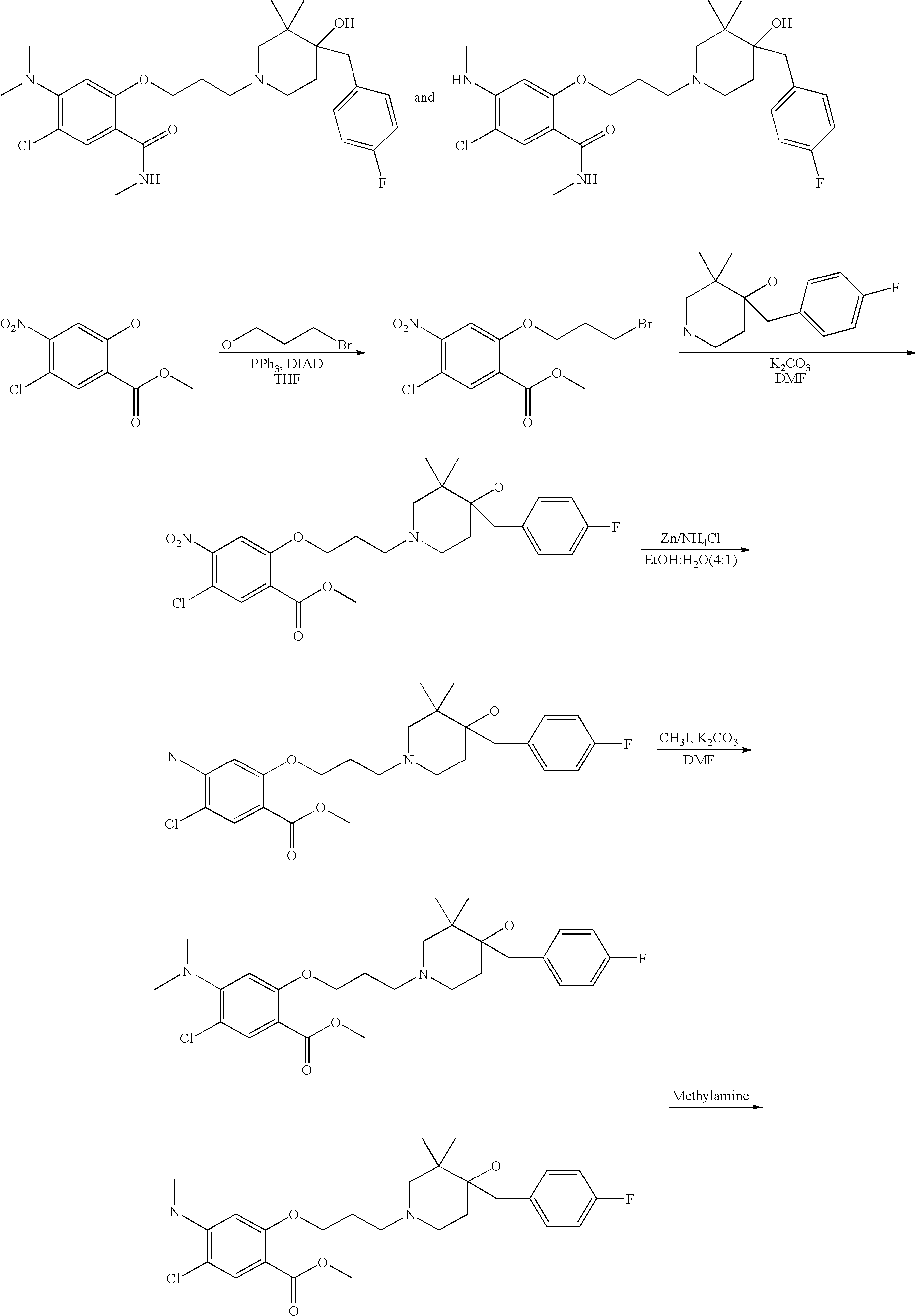

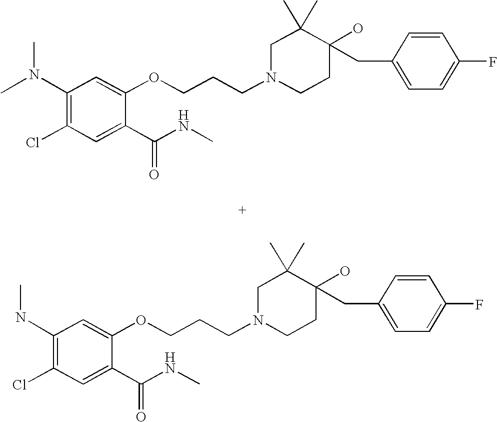

### Step-1: 2-(3-Bromo-propoxy)-5-chloro-4-nitro-benzoic acid methyl ester

To a stirred solution of PPh₃ (340 mg, 1.3 m.mol) in THF, DIAD (0.2 ml, 0.97 m.mol) was added to it at 0° C. The reaction mixture was stirred for half an hour. Then to it 3-bromopropanol (0.06 ml, 0.65 mmol) and 5-Chloro-2-hydroxy-4-nitro-benzoic acid methyl ester (150 mg, 0.65 mmol) were added. It was then stirred for overnight at rt. concentrated the solvent under reduced pressure. Thus obtained crude was purified with column chromatography (Si-gel, 4% ethyl acetate-hexane) to afford 210 mg (45.8%) of 2-(3-Bromo-propoxy)-5-chloro-4-nitro-benzoic acid methyl ester. ¹H-NMR (400 MHz, CDCl₃) δ (ppm): 7.95 (s, 1H), 7.45 (s, 1H), 4.23 (t, 2H), 3.90 (s, 3H), 3.66 (t, 2H), 2.37 (m, 2H).

### Step-2: 5-Chloro-2-{3-[4-(4-fluoro-benzyl)-4-hydroxy-3,3-dimethyl-piperidin-1-yl]-propoxy}-4-nitro-benzoic acid methyl ester

To a stirred solution of 2-(3-Bromo-propoxy)-5-chloro-4-nitro-benzoic acid methyl ester (150 mg, 0.43 mmol) in DMF was added 4-(4-Fluoro-benzyl)-3,3-dimethyl-piperidin-4-ol (100 mg, 3 mmol) and K₂CO₃ (120 mg, 0.85 mmol) The reaction was stirred overnight at room temperature and diluted with ice water. The organics was extracted with ethyl acetate, washed with water, brine, dried over Na₂SO₄ and concentrated under reduced pressure. Thus obtained crude was purified with column chromatography (Si-gel, 1.5% methanol-DCM) to afford 120 mg (54.8%) of 5-Chloro-2-{3-[4-(4-fluoro-benzyl)-4-hydroxy-3,3-dimethyl-piperidin-1-yl]-propoxy}-4-nitro-benzoic acid methyl ester. LC/MS [M+H]⁺: 508.9.

### Step-3: 4-Amino-5-chloro-2-{3-[4-(4-fluoro-benzyl)-4-hydroxy-3,3-dimethyl-piperidin-1-yl]-propoxy}-benzoic acid methyl ester

Zn-powder (225 mg, 3.44 mmol) and NH₄Cl (89 mg, 1.46 mmol) were added to a solution of 5-Chloro-2-{3-[4-(4-fluoro-benzyl)-4-hydroxy-3,3-dimethyl-piperidin-1-yl]-propoxy}-4-nitro-benzoic acid methyl ester (250 mg, 0.49 mmol) in ethanol-water (4:1) and the mixture was heated to reflux for 4 hours. The reaction mixture was then filtered through celite bed. The organic layer was concentrated, diluted with water and the product was extracted with ethyl acetate. The organic layer was washed with water, dried over sodium sulfate and concentrated to afford 240 mg (100%) of 4-Amino-5-chloro-2-{3-[4-(4-fluoro-benzyl)-4-hydroxy-3,3-dimethyl-piperidin-1-yl]-propoxy}-benzoic acid methyl ester. LC/MS [M+H]⁺: 479.2.

### Step-4: Mixture of 5-Chloro-4-dimethylamino-2-{3-[4-(4-fluoro-benzyl)-4-hydroxy-3,3-dimethyl-piperidin-1-yl]-propoxy}-benzoic acid methyl ester 5-Chloro-2-{3-[4-(4-fluoro-benzyl)-4-hydroxy-3,3-dimethyl-piperidin-1-yl]-propoxy}-4-methylamino-benzoic acid methyl ester

K₂CO₃ (87 mg, 0.63 mmol) and methyl iodide (0.03 ml, 0.45 mmol) were added to a solution of 4-Amino-5-chloro-2-{3-[4-(4-fluoro-benzyl)-4-hydroxy-3,3-dimethyl-piperidin-1-yl]-propoxy}-benzoic acid methyl ester (100 mg, 0.21 mmol) in DMF. The reaction mixture was heated for 3 hour at 40° C. then diluted with water and extracted with ethyl acetate. The organic layer was washed with water and dried over sodium sulfate. The reaction mixture was concentrated to afford 106 mg of a mixture of 6-chloro-7-dimethylamino-2,3-dihydro-benzo[1,4]oxazine-2,4-dicarboxylic acid 4-tert-butyl ester 2-ethyl ester 5-Chloro-4-dimethylamino-2-{3-[4-(4-fluoro-benzyl)-4-hydroxy-3,3-dimethyl-piperidin-1-yl]-propoxy}-benzoic acid methyl ester and 5-Chloro-2-{3-[4-(4-fluoro-benzyl)-4-hydroxy-3,3-dimethyl-piperidin-1-yl]-propoxy}-4-methylamino-benzoic acid methyl ester.

### Step-5

To a solution of a mixture of 6-chloro-7-dimethylamino-2,3-dihydro-benzo[1,4]oxazine-2,4-dicarboxylic acid 4-tert-butyl ester 2-ethyl ester 5-Chloro-4-dimethylamino-2-{3-[4-(4-fluoro-benzyl)-4-hydroxy-3,3-dimethyl-piperidin-1-yl]-propoxy}-benzoic acid methyl ester and 5-Chloro-2-{3-[4-(4-fluoro-benzyl)-4-hydroxy-3,3-dimethyl-piperidin-1-yl]-propoxy}-4-methylamino-benzoic acid methyl ester (200 mg) in ethanol was added a solution of methylamine in ethanol. The reaction mixture was heated at 70° C. in a sealed tube overnight. The reaction mixture was cooled and concentrated under reduced pressure. The crude was purified through HPLC to afford 6 mg of 5-Chloro-4-dimethylamino-2-{3-[4-(4-fluoro-benzyl)-4-hydroxy-3,3-dimethyl-piperidin-1-yl]-propoxy}-N-methyl-benzamide and 11 mg of 5-Chloro-2-{3-[4-(4-fluoro-benzyl)-4-hydroxy-3,3-dimethyl-piperidin-1-yl]-propoxy}-N-methyl-4-methylamino-benzamide.

5-Chloro-4-dimethylamino-2-{3-[4-(4-fluoro-benzyl)-4-hydroxy-3,3-dimethyl-piperidin-1-yl]-propoxy}-N-methyl-benzamide was prepared by the process similar to that described in example 84. LC/MS [M+H]⁺: 506.4. LCMS purity: 93.6%.

### 5-Chloro-2-{3-[4-(4-fluoro-benzyl)-4-hydroxy-3,3-dimethyl-piperidin-1-yl]-propoxy}-N-methyl-4-methylamino-benzamide. LC/MS [M+H]⁺: 492.3. HPLC: 91.1%.

### Example 101

### N-[5-Chloro-2-(2-{[1-(4-chloro-benzyl)-4-hydroxy-piperidin-4-ylmethyl]-amino}-ethoxy)-4-methoxy phenyl]-acetamide

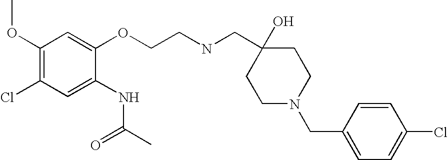

N-[5-Chloro-2-(2-{[1-(4-chloro-benzyl)-4-hydroxy-piperidin-4-ylmethyl]-amino}-ethoxy)-4-methoxy-phenyl]-acetamide was prepared by a process similar to that described in example 95. LC/MS [M+H]⁺: 496.2. ¹H-NMR (400 MHz, DMSO-d₆) δ (ppm): 9.09 (m, 1H), 7.90 (m, 11-1), 7.36 (d, 2H), 7.30 (d, 2H), 6.85 (s, H), 4.17 (bs, 2H), 3.84 (s, 3H), 3.44 (bs, 2H), 3.09 (m, H), 2.99 (bs, 2H), 2.42 (m, 2H), 2.32 (m, 2H), 2.05 (s, 3H), 1.51-0.83 (m, 7H). HPLC: 96.7%.

### Example 102

### 5-Chloro-2-{3-[3-(4-fluoro-benzyl)-3-hydroxy-pyrrolidin-1-yl]-propoxy}-4-methoxy-N-methyl-benzamide

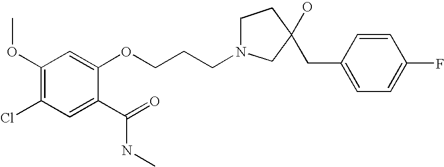

5-Chloro-2-{3-[3-(4-fluoro-benzyl)-3-hydroxy-pyrrolidin-1-yl]-propoxy}-4-methoxy-N-methyl-benzamide was prepared by a process similar to that described in example 94. LC/MS [M+H]⁺: 451.2. ¹H-NMR (400 MHz, DMSO-d₆) δ (ppm): 7.93 (bs, 1H), 7.76 (s, 1H), 7.27 (t, 2H), 7.05 (t, 2H), 6.82 (s, 1H), 4.23 (t, 2H), 3.92 (s, 3H), 2.79 (s, 3H), 1.95 (m, 4H), 1.90 (m, 1H), 1.84 (m, 8H). HPLC: 93.5%.

### Example 103

### N-(5-Chloro-2-{3-[3-(4-fluoro-benzyl)-3-hydroxy-pyrrolidin-1-yl]-propoxy}-4-methoxy-phenyl)-acetamide

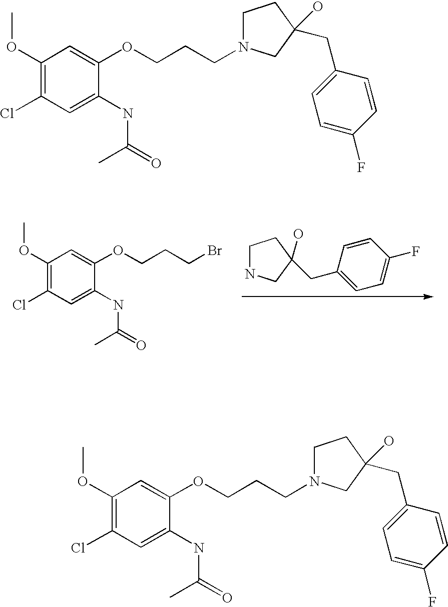

3-(4-Fluoro-benzyl)-pyrrolidin-3-ol was prepared by a process similar to that described in example 94.
N-[2-(3-Bromo-propoxy)-5-chloro-4-methoxy-phenyl]-acetamide was prepared by a process as described in example 95.

To a solution of N-[2-(3-Bromo-propoxy)-5-chloro-4-methoxy-phenyl]-acetamide (120 mg, 0.36 mmol) was dissolved in DMF was added K₂CO₃ (100 mg, 0.71 mmol) and 3-(4-Fluoro-benzyl)-pyrrolidin-3-ol (70 mg, 0.36 mmol). The reaction mixture was stirred at room temperature overnight. It was diluted with water and extracted with ethyl acetate. The organic layer was washed with water, brine and concentrated. The crude organics was purified over column chromatography (Si-gel, 6% methanol-DCM) to afford 60 mg (37.1%) of N-(5-Chloro-2-{3-[3-(4-fluoro-benzyl)-3-hydroxy-pyrrolidin-1-yl]-propoxy}-4-methoxy-phenyl)-acetamide. LCMS: 451.1. ¹H-NMR (400 MHz, DMSO-d₆) δ (ppm): 8.98 (m, 1H), 7.82 (s, 1H), 7.29-7.06 (m, 4H), 6.81 (s, 1H), 4.60 (m, 1H), 4.10 (m, 2H), 3.84 (s, 3H), 2.96-1.33 (s, 15H). HPLC: 92.4%.

### Example 104

### 5-Chloro-2-(2-{[7-(4-chloro-benzyl)-4-hydroxy-piperidin-4-ylmethyl]-amino}-ethoxy)-4-methoxy-N-methyl-benzamide

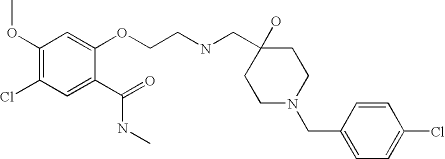

4-Aminomethyl-1-(4-fluoro-benzyl)-piperidin-4-ol was prepared by a process described in example 95.

5-Chloro-2-(2-{[1-(4-chloro-benzyl)-4-hydroxy-piperidin-4-ylmethyl]-amino}-ethoxy)-4-methoxy-N-methyl-benzamide was prepared by a process similar to that described in example 94. LC/MS [M+H]⁺: 496.4. ¹H-NMR (400 MHz, DMSO-d₆) δ (ppm): 8.29 (d, 1H), 7.79 (s, 1H), 7.32 (dd, 4H), 6.87 (s, 1H), 4.57 (bs, 2H), 4.26 (m, 1H), 3.93 (s, 3H), 3.41 (s, 2H), 2.97 (s, 2H), 2.89 (s, 3H), 2.38 (m, 2H), 2.30 (m, 2H), 1.50 (m, 4H), 1.23 (s, 3H). HPLC: 89.3%.

### Example 105

### 5-Chloro-2-{2-[8-(4-fluoro-benzyl)-2-oxo-1-oxa-3,8-diaza-spiro[4.5]dec-3-yl]-ethoxy}-4-methoxy-N-methyl-benzamide

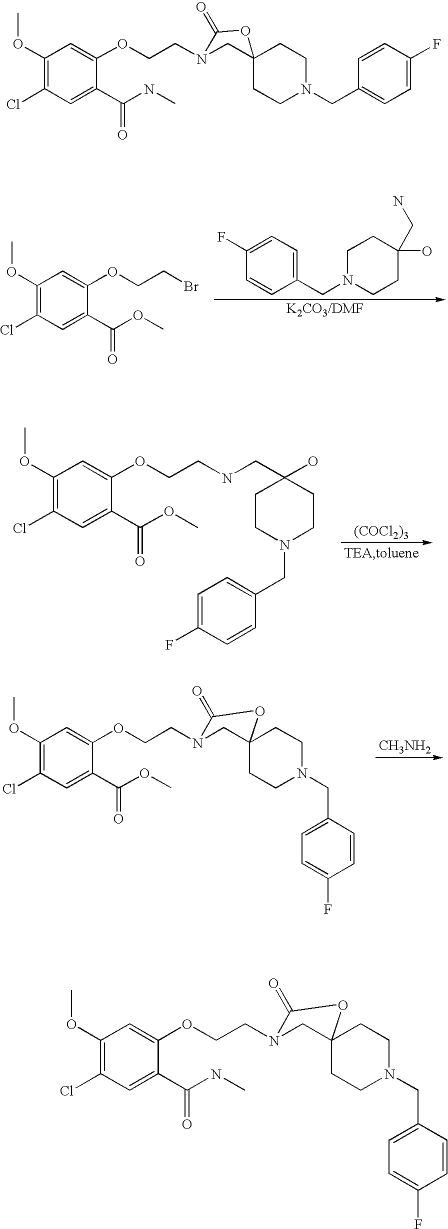

4-Aminomethyl-1-(4-fluoro-benzyl)-piperidin-4-ol was prepared by a process described in example 95.
2-(2-Bromo-ethoxy)-5-chloro-4-methoxy-benzoic acid methyl ester was prepared by a process as described in example 96.

### Step-1: 5-Chloro-2-(2-{[1-(4-fluoro-benzyl)-4-hydroxy-piperidin-4-ylmethyl]-amino}-ethoxy)-4-methoxy-benzoic acid methyl ester

To a solution of 2-(2-Bromo-ethoxy)-5-chloro-4-methoxy-benzoic acid methyl ester (200 mg, 0.62 mmol) was dissolved in dry DMF. Then to it K₂CO₃ (2.0 eq, 172 mg, 1.24 mmol), and amine (1.0 eq, 150 mg, 0.62 mmol) were added. The reaction mixture was then stirred at rt. for overnight. It was diluted with water and extracted with ethyl acetate. Then it was washed with water and brine. It was then concentrated and purified by column chromatography using (0-10%) methanol/DCM in SiO₂ to afford 150 mg (50.3%) of 5-Chloro-2-(2-{[1-(4-fluoro-benzyl)-4-hydroxy-piperidin-4-ylmethyl]-amino}-ethoxy)-4-methoxy-benzoic acid methyl ester. LC/MS [M+H]⁺: 481.4.

### Step-2: 2-(2-{[4-Acetoxy-1-(4-fluoro-benzyl)-piperidin-4-ylmethyl]-amino}ethoxy)-5-chloro-4-methoxy-benzoic acid methyl ester

5-Chloro-2-(2-{[1-(4-fluoro-benzyl)-4-hydroxy-piperidin-4-ylmethyl]-amino}-ethoxy)-4-methoxy-benzoic acid methyl ester (75 mg, 0.16 mmol) was dissolved in dry toluene and to it triethylamine (0.06 ml, 0.47 mmol) was added at 0° C. To it COCl₂ (25 mg, 0.08 mmol) was added and it was heated at 100° C. for 4 hour. It was then distilled and crude product was washed with hexane to afford 60 mg (71.7%) of 2-(2-{[4-Acetoxy-1-(4-fluoro-benzyl)-piperidin-4-ylmethyl]-amino}ethoxy)-5-chloro-4-methoxy-benzoic acid methyl ester. LC/MS [M+H]⁺: 507.4.

### Step-3: 5-Chloro-2-{2-[8-(4-fluoro-benzyl)-2-oxo-1-oxa-3,8-diazaspiro[4.5]dec-3-yl]-ethoxy}-4-methoxy-N-methyl-benzamide

2-(2-{[4-Acetoxy-1-(4-fluoro-benzyl)-piperidin-4-ylmethyl]-amino}ethoxy)-5-chloro-4-methoxy-benzoic acid methyl ester (60 mg, 0.12 mmol) was dissolved in minimum volume ethanol and taken in a sealed tube. To it methylamine in ethanol solution was added and it was heated at 70° C. for overnight. It was cooled and distilled. Then it was extracted with ethyl acetate, washed with water and brine. It was dried over Na₂SO₄. Then it was concentrated and purified through column chromatography (Si-gel, 2.5% methanol-DCM) to produce 30 mg (49.4%) 5-Chloro-2-{2-[8-(4-fluoro-benzyl)-2-oxo-1-oxa-3,8-diazaspiro[4.5]dec-3-yl]-ethoxy}-4-methoxy-N-methyl-benzamide. LC/MS [M+H]⁺: 506.4. ¹H-NMR (400 MHz, DMSO-d₆) δ (ppm): 7.87 (m, 2H), 7.31 (m, 2H), 7.13 (m, 2H), 6.84 (s, 1H), 4.32 (brs, 2H), 3.93 (s, 3H), 3.66-1.33 (m, 17H). HPLC: 91.1%.

### Example 106

### 5-Chloro-2-[4-(4-fluoro-benzyl)-[1,4]oxazepan-2-ylmethoxy]-4-methoxy-N-methyl-benzamide

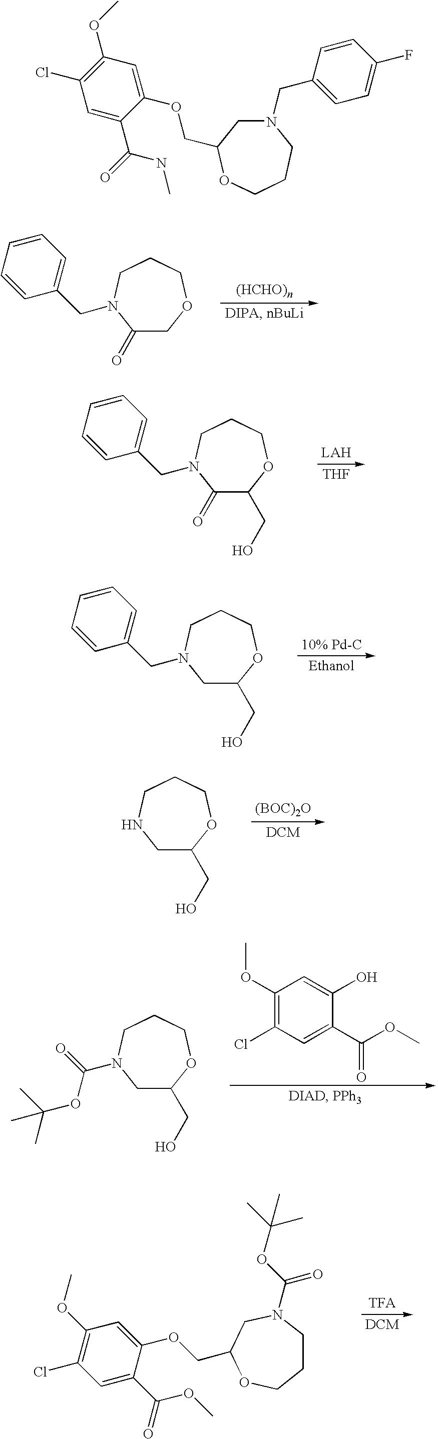

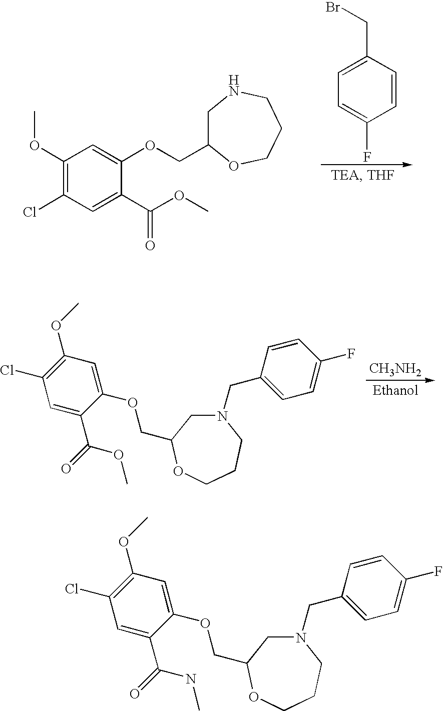

### Step-1: 4-Benzyl-2-hydroxymethyl-[1,4]oxazepan-3-one

DIPA (1.7 ml, 0.012 mol) was added to a dry THF solution. To it 1.57 (M) nBuLi (8.7 ml, 0.013 mol) was added at 0° C. A straw yellow colour observed after 1 hour stirring at rt. Then reaction temperature was made −78° C. and THF solution of 4-Benzyl[1,4]oxazepan-3-one (2 gm, 0.009 mol) was added to it. A blackish red colour appeared. It was stirred at −30° C. for 1 hour. Then paraformaldehyde (1 gm) was added to the reaction mixture under argon atmosphere and stirred at room temperature for 3 hours. The reaction mixture was quenched with saturated NH₄Cl solution and extracted with ethylacetate. The organic layer was washed with water, brine and concentrated. The cruse mass was purified through flash chromatography (Si-gel, 42%) ethyl acetate-hexane) to afford 960 mg (41.9%) of 4-Benzyl-2-hydroxymethyl-[1,4]oxazepan-3-one. LC/MS [M+H]⁺: 236.2.

### Step 2: 4-Benzyl-[1,4]oxazepan-2-yl)-methanol

To a solution of 4-Benzyl-2-hydroxymethyl-[1,4]oxazepan-3-one (1.2 gm, 0.005 mol) in THF was added lithium aluminum hydride (390 mg, 0.01 mmol) portion wise at cold condition. It was then refluxed for 3 hours and quenched by 0.4 ml water, 0.4 ml 15% NaOH solution followed by 0.8 ml water. The reaction mixture was stirred at cold condition for 30 minutes and filtered and washed with ethyl acetate. The organic layer was concentrated to afford 1.0 g (88.6%) of 4-Benzyl-[1,4]oxazepan-2-yl)-methanol. LC/MS [M+H]⁺: 222.0.

### Step-3: [1,4]Oxazepan-2-yl-methanol

To a solution of 4-Benzyl-[1,4]oxazepan-2-yl)-methanol (1 gm, 0.004 mol) in ethanol 10% Pd—C was added. The reaction mixture was then subjected to hydrogenation for 4 hours. Then it was filtered and concentrated to produce (550 mg, 93.2%) [1,4]Oxazepan-2-yl-methanol. LC/MS [M+H]⁺: 132.0.

### Step-4: 2-Hydroxymethyl-[1,4]oxazepane-4-carboxylic acid tert-butyl ester

To a solution of [1,4]Oxazepan-2-yl-methanol (550 mg, 4.2 mmol) in DCM was added triethylamine (1.2 ml, 8.4 mmol) and (BOC)₂O (1.1 eq, 1 ml, 4.61 mmol) at 0° C. was added and stirred the solution at room temperature for overnight. It was quenched by aq. NaHCO₃ solution and extracted with ethyl acetate. The organic layer was then washed with water and brine. Then it was dried over Na₂SO₄ and concentrated to produce (800 mg, 82.6%) of 2-Hydroxymethyl-[1,4]oxazepane-4-carboxylic acid tert-butyl ester. ¹H NMR (400 MHz, DMSO-d₆): 4.68 (m, 1H), 3.95 (m, 1H), 3.70 (m, 1H), 3.54-2.98 (m, 6H), 1.74 (m, 2H), 1.46 (m, 1H), 1.39 (s, 4H).

### Step-5: 2-(4-Chloro-5-methoxy-2-methoxycarbonyl-phenoxymethyl)-[1,4]oxazepane-4-carboxylic acid tert-butyl ester

Triphenylphosphine (1.8 gm, 6.92 mol) was dissolved in dry THF. To this DIAD (1.02 ml, 5.19 mol) was added drop wise at cold condition. The solution was stirred for half an hour and was added 2-hydroxymethyl-[1,4]oxazepane-4-carboxylic acid tert-butyl ester (750 mg, 3.46 mol) and 5-Chloro-2-hydroxy-4-methoxy-benzoic acid methyl ester (800 mg, 3.46 mol) at a time. The reaction mixture was stirred at room temperature for overnight, concentrated and purified through flash chromatography (Si-gel, 14% ethyl acetate-hexane) to afford 1.1 g (74%) of 2-(4-Chloro-5-methoxy-2-methoxycarbonyl-phenoxymethyl)-[1,4]oxazepane-4-carboxylic acid tert-butyl ester. LC/MS [M+H]⁺: 430.2.

### Step-6: 5-Chloro-4-methoxy-2-([1,4]oxazepan-2-ylmethoxy)-benzoic acid methyl ester

To a solution of 2-(4-Chloro-5-methoxy-2-methoxycarbonyl-phenoxymethyl)-[1,4]oxazepane-4-carboxylic acid tert-butyl ester (1.1 gm, 3.3 mmol) in DCM was added TFA (2.0 ml) and stirred at room temperature for 2 hours. The solution was concentrated, diluted with water, basified with aq. NaOH solution and extracted with ethyl acetate. The organic layer was washed with water, brine and concentrated. The crude mass was purified by flash chromatography (Si-gel, 10% methanol-DCM) to afford 360 mg (33%) 5-Chloro-4-methoxy-2-([1,4]oxazepan-2-ylmethoxy)-benzoic acid methyl ester.

LC/MS [M+H]⁺: 330.4.

### Step-7: 5-Chloro-2-[4-(4-fluoro-benzyl)-[1,4]oxazepan-2-ylmethoxy]-4-methoxy-benzoic acid methyl ester

To a solution 5-Chloro-4-methoxy-2-([1,4]oxazepan-2-ylmethoxy)-benzoic acid methyl ester (90 mg, 0.27 mmol) in THF was added triethylamine (0.1 ml, 0.82 mmol) drop wise at 0° C. followed by addition of 1-Bromomethyl-4-fluoro-benzene (0.05 ml, 0.27 mmol) and stirred at room temperature overnight. The reaction mixture was concentrated and extracted with ethyl acetate. The organic layer was washed with water, brine and concentrated. The crude organic mass was purified through flash chromatography (Si-gel, 1% methanol DCM) to afford 100 mg (84.6%) of 5-Chloro-2-[4-(4-fluoro-benzyl)-[1,4]oxazepan-2-ylmethoxy]-4-methoxy-benzoic acid methyl ester.

LC/MS [M+H]⁺: 438.4.

### Step-8: 5-Chloro-2-[4-(4-fluoro-benzyl)-[1,4]oxazepan-2-ylmethoxy]-4-methoxy-N-methyl-benzamide

To a solution of 5-Chloro-2-[4-(4-fluoro-benzyl)-[1,4]oxazepan-2-ylmethoxy]-4-methoxy-benzoic acid methyl ester (100 mg, 0.23 mmol) in ethanol was added a solution of methylamine in ethanol and heated at 70° C. for overnight in a sealed tube. The reaction mixture was concentrated and extracted with ethyl acetate. The organic layer was concentrated and washed with hexane to afford 60 mg (59.7%) of 5-Chloro-2-[4-(4-fluoro-benzyl)-[1,4]oxazepan-2-ylmethoxy]-4-methoxy-N-methyl-benzamide. LC/MS [M+H]⁺: 437.4. ¹H-NMR (400 MHz, CDCl₃) δ(ppm): 8.10 (s, 1H), 8.09 (bs, 1H), 7.29 (m, 2H), 6.98 (m, 2H), 6.35 (s, 1H), 4.05-1.90 (m, 19H). HPLC purity: 98.4%.

### Human CCR1Binding Assay (Ki Determination in 96 Well Format)

### Competition-Equilibrium Binding Assay

After trypsinization, CHO-K1-Gα16 (CHO-K1 from ATCC, Gα16 cloned and expressed in CHO-K1 cells in CBT) cells expressing human wild-type CCR1 were harvested using calcium and magnesium free HBSS (INVITROGEN) buffer and then resuspended at 400,000 cells/mL in binding buffer (RPMI1640, pH 7.4, 0.2% BSA, Complete protease inhibitor cocktail without EDTA from Roche) Competition-binding assays were performed in 96 well plates (Corning non binding surface flat clear bottom white 96 well), using 25 μL of cell suspension (10,000/well), 25 μL/well of 100 pM solution of [125I]-RANTES (2200 Ci/mmol; Perkin Elmer NEX292) as tracer for human CCR1 receptor, 25 μL/well of RPMI buffer or various concentration of competitor/compound diluted in RPMI and 25 μL/well of PVT-WGA SPA beads (GE) (0.1 mg/well). Each well contained a final volume of 100 μL with a final DMSO concentration of 0.5%. The 96 well plates were incubated for 2 hours at room temperature in the dark without shaking. Bound radioligand was counted with a beta scintillation counter (Wallac, Micro-Beta Trilux counter) for 1 min per well. Total binding of [125I]-RANTES was measured in the absence and presence of competitor (compound), the nonspecific binding was measured with a 33-fold excess of BX471 (10 μM final). In order to determine binding parameters (Ki and IC₅₀), the counts results were analyzed using the one-site competition curve fitting functions in GraphPad PRISM, v. 4.0 (San Diego, Calif.).

The compounds of the present invention typically exhibit potency values of greater than 50% inhibition at 20 μM in this competitive binding assay.

The entire disclosures of all applications, patents and publications, cited above and below, are hereby incorporated by reference.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed is:

1. A compound having the chemical formula: 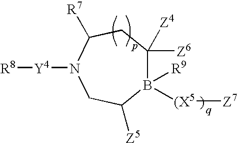
wherein
R⁸ is aryl-X⁴—, heteroaryl-X⁴—, aryl, heterocycle or heteroaryl;
X⁴ is —O—, —S—, —S(O)—, —S(O)₂—, —S(O)₂NR^{o}— or —NR^{p}, where R^{o} and R^{p} are each, independently, hydrogen or alkyl;
Y⁴ is —C(O)—, —(CH₂)₂—, —(CH₂)₃—, —CH₂C(O)—, —(CH₂)₂C(O)—, —C(O)CH₂—, —C(O)—(CH₂)₂— or —CH₂CH(OH)CH₂—;
R⁷ is hydrogen or alkyl;
B is —C— or —O—;
R⁹ is hydrogen, hydroxyl or cyano;
X⁵ is —O—, —NR^{e}—, —S—, —S(O)— or —S(O)₂ where R^{e} is hydrogen or alkyl;
Z⁷ is aryl, heteroaryl, arylalkyl, heteroarylalkyl;
p is 0 or 1;
q is 0 or 1;
Z⁴, Z⁵ and Z⁶ are each, independently, hydrogen, alkyl, arylalkyl, heteroarylalkyl, -(alkylene)-J-aryl or -(alkylene)-J-heteroaryl, where J is —O— or —NR^{q}— and R^{q} is hydrogen or alkyl;
provided, however, that when R⁹ is hydroxyl or cyano, then q is 0, B is —C—, and Z⁴, Z⁶ are both alkyl or both hydrogen;
provided, however, that when R⁹ is H, then q is 1, p is 0, and B is —C—;
provided, however, that when B is —O—, and both R⁹ and —X⁵—Z⁷ are absent; then Z⁵ is not hydrogen when p is 0;
provided, however, that when B is —O—, R⁹ and —X⁵—Z⁷ are absent and p is 1; then either Z⁴, Z⁶ or Z⁵ are both not hydrogen;
provided, however, that when B is —C—, Z⁴, Z⁶ are both hydrogen, and p is 0, then R⁹ is cyano, q is 0, Z⁷ is arylalkyl, and Y⁴ is —(CH₂)₃—;
wherein, when present, any aryl, heterocycle or heteroaryl group may optionally be substituted by halogen, hydroxy, cyano, nitro, amino, alkylamino, dialkylamino, arylamino, diarylamino, amido, alkylamido, dialkylamido, carboxyl, alkyl, halogenated alkyl, alkenyl, alkynyl, cycloalkyl, cycloalkylalkyl, aryl, arylalkyl, heteroaryl, heteroarylalkyl, heterocycle, heterocyclealkyl, aroyl, acyl, alkoxy, aryloxy, heteroaryloxy, cycloalkyloxy, cycloalkylalkyloxy, arylalkyloxy, heteroarylalkyloxy, alkythio, arylthio, alkylsulfinyl, alkylsulfonyl, arylsulfinyl, arylsulfonyl, heteroarylsulfinyl, heteroarylsulfonyl, alkoxycarbonyl, aryloxycarbonyl, heteroaryloxycarbonyl, —NR^{r}C(O)R^{s}, —NR^{t}SO₂R^{u}, where R^{pr} and R^{t} are each, independently, hydrogen or alkyl, R^{s} is amino, aminoalkyl, alkyl or cycloalkyl, and R^{u} is hydrogen or alkyl; and combinations thereof;
and pharmaceutically acceptable salts or solvates or N-oxides thereof, or solvates of pharmaceutically acceptable salts thereof, or solvates of N-oxides thereof, or isomers, or prodrugs thereof;
with the proviso that said compound is not
1-[2-(4-fluorophenoxy)ethyl]-N-(4-fluorophenyl)-4-piperidinamine,
N-(4-chlorophenyl)-1-[2-(4-fluorophenoxy)ethyl]-4-piperidinamine,
N-(4-chlorophenyl)-1-[3-(4-fluorophenoxy)propyl]-4-piperidinamine,
N-(4-chlorophenyl)-1-(3-phenoxypropyl-4-piperidinamine,
N-(4-fluoro-2-methylphenyl)-1-[3-(4-fluorophenoxy)propyl]-4-piperidinamine,
1-[3-(4-fluorophenoxy)propyl]-N-(4-fluorophenyl)-N-methyl-4-piperidinamine,
1-[3-(4-fluorophenoxy)propyl]-N-(4-fluorophenyl)-4-piperidinamine,
N-(5-Chloro-2-{3-[4-(4-chloro-phenoxy)-piperidin-1-yl]-propoxy}-4-methoxy-phenyl)-acetamide,
N-(5-Chloro-2-{3-[4-(4-chloro-phenylsulfanyl)-piperidin-1-yl]-propoxy}-4-methoxy-phenyl)-acetamide,
N-(5-Chloro-2-{3-[4-(4-chloro-benzenesulfonyl)-piperidin-1-yl]-propoxy}-4-methoxy-phenyl)-acetamide,
N-(5-Chloro-2-{3-[4-(4-chloro-phenoxy)-3,3-dimethyl-piperidin-1-yl]-propoxy}-4-methoxy-phenyl)-acetamide,
N-(5-Chloro-2-{3-[4-(4-chloro-phenoxy)-piperidin-1-yl]-2-hydroxy-propoxy}-4-methoxy-phenyl)-acetamide,
N-(5-Chloro-2-{3-[4-(4-cyano-phenoxy)-piperidin-1-yl]-propoxy}-4-methoxy-phenyl)-acetamide,
N-(5-Chloro-2-{3-[4-(4-fluoro-phenoxy)-piperidin-1-yl]-propoxy}-4-methoxy-phenyl)-acetamide,
or a pharmaceutically acceptable salt thereof.

2. A compound according to claim 1, selected from:
(5-Chloro-2-{3-[4-(4-fluoro-benzyl)-4-hydroxy-3,3-dimethyl-piperidin-1-yl]-propoxy}-phenyl)-urea,
(5-Chloro-2-{3-[4-(4-chloro-phenyl)-4-hydroxy-3,3-dimethyl-piperidin-1-yl]-propoxy}-phenyl)-urea,
(5-Chloro-2-{3-[4-(4-fluoro-phenyl)-4-hydroxy-3,3-dimethyl-piperidin-1-yl]-propoxy}-phenyl)-urea,
(5-Chloro-2-{3-[4-(4-chloro-benzyl)-4-hydroxy-3,3-dimethyl-piperidin-1-yl]-propoxy}-phenyl)-urea,
(5-Chloro-2-{3-[4-(4-fluoro-benzyl)-4-cyano-3,3-dimethyl-piperidin-1-yl]-propoxy}-phenyl)-urea,
1-[3-(4-Chloro-2-isoxazol-5-yl-phenoxy)-propyl]-4-(4-fluoro-benzyl)-3,3-dimethyl-piperidin-4-ol,
4-(4-Chloro-benzyl)-1-[3-(4-chloro-2-isoxazol-5-yl-phenoxy)-propyl]-3,3-dimethyl-piperidin-4-ol,
1-[3-(5-Chloro-benzoimidazol-1-yl)-propyl]-4-(4-chloro-benzyl)-3,3-dimethyl-piperidin-4-ol,
1-[2-(5-Chloro-benzoimidazol-1-yl)-ethyl]-4-(4-chloro-benzyl)-3,3-dimethyl-piperidin-4-ol,
N-(5-Chloro-2-{3-[4-(4-chloro-benzyl)-4-hydroxy-3,3-dimethyl-piperidin-1-yl]-propoxy}-phenyl)-acetamide,
N-(5-Chloro-2-{3-[4-(4-chloro-benzyl)-4-hydroxy-3,3-dimethyl-piperidin-1-yl]-propoxy}-phenyl)-2-dimethylamino-acetamide,
N-(5-Chloro-2-{3-[4-(4-chloro-benzyl)-4-hydroxy-3,3-dimethyl-piperidin-1-yl]-propoxy}-phenyl)-isobutyramide,
N-(5-Chloro-2-{3-[4-(4-chloro-benzyl)-4-hydroxy-3,3-dimethyl-piperidin-1-yl]-propoxy}-phenyl)-2,2-dimethyl-propionamide,
Cyclobutanecarboxylic acid (5-chloro-2-{3-[4-(4-chloro-benzyl)-4-hydroxy-3,3-dimethyl-piperidin-1-yl]-propoxy}-phenyl)-amide,
N-(5-Chloro-2-{3-[4-(4-chloro-benzyl)-4-hydroxy-3,3-dimethyl-piperidin-1-yl]-propoxy}-phenyl)-methanesulfonamide,
5-Chloro-2-{3-[4-(4-chloro-benzyl)-4-hydroxy-3,3-dimethyl-piperidin-1-yl]-propoxy}-benzoic acid,
5-Chloro-2-{3-[4-(4-chloro-benzyl)-4-hydroxy-3,3-dimethyl-piperidin-1-yl]-propoxy}-benzamide,
1-[3-(5-Chloro-benzoimidazol-1-yl)-propyl]-4-(4-chloro-phenyl)-3,3-dimethyl-piperidin-4-ol,
1-[3-(5-Chloro-benzoimidazol-1-yl)-propyl]-4-(4-fluoro-benzyl)-piperidine-4-carbonitrile,
N-(5-Chloro-2-{3-[4-(3,4-difluoro-benzyl)-4-hydroxy-3,3-dimethyl-piperidin-1-yl]-propoxy}-phenyl)-acetamide,
N-(5-Chloro-2-{3-[4-(4-chloro-benzyl)-4-hydroxy-3,3-dimethyl-piperidin-1-yl]-propoxy}-4-methoxy-phenyl)-acetamide,
N-(5-Chloro-2-{3-[4-(4-chloro-3-methoxy-benzyl)-4-hydroxy-3,3-dimethyl-piperidin-1-yl]-propoxy}-phenyl)-acetamide,
5-Chloro-2-{3-[4-(4-chloro-benzyl)-4-hydroxy-3,3-dimethyl-piperidin-1-yl]-propoxy}-N,N-dimethyl-benzamide,
5-Chloro-2-{3-[4-(4-chloro-benzyl)-4-hydroxy-3,3-dimethyl-piperidin-1-yl]-propoxy}-N-methyl-benzamide,
1-[2-(6-Chloro-1H-benzoimidazol-2-yl)-ethyl]-4-(4-chloro-benzyl)-3,3-dimethyl-piperidin-4-ol,
N-(5-Chloro-2-{3-[4-(4-chloro-phenyl)-4-hydroxy-3,3-dimethyl-piperidin-1-yl]-propoxy}-phenyl)-acetamide,
N-(5-Chloro-2-{3-[4-(4-chloro-phenyl)-4-hydroxy-3,3-dimethyl-piperidin-1-yl]-propoxy}-4-methoxy-phenyl)-acetamide,
4-(4-Chloro-benzyl)-1-[3-(4-chloro-2-pyrazol-1-yl-phenoxy)-propyl]-3,3-dimethyl-piperidin-4-ol,
(5-Chloro-2-{3-[4-(4-chloro-benzyl)-4-hydroxy-3,3-dimethyl-piperidin-1-yl]-propoxy}-4-methoxy-phenyl)-urea,
(5-Chloro-2-{3-[4-(4-fluoro-benzyl)-4-hydroxy-3,3-dimethyl-piperidin-1-yl]-propoxy}-4-methoxy-phenyl)-urea,
(5-Chloro-2-{2-[(2R,5S)-2-(4-fluoro-benzyl)-5-methyl-morpholin-4-yl]-2-oxo-ethoxy}-phenyl)-urea,
(5-Chloro-2-{2-[(2R,5R)-2-(4-fluoro-phenoxymethyl)-5-methyl-morpholin-4-yl]-2-oxo-ethoxy}-phenyl)-urea,
(5-Chloro-2-{2-[(2S,5R)-2-(4-fluoro-phenoxymethyl)-5-methyl-morpholin-4-yl]-2-oxo-ethoxy}-phenyl)-urea,
[5-Chloro-2-(2-{(2S,5R)-2-[2-(4-fluoro-phenyl)-ethyl]-5-methyl-morpholin-4-yl}-2-oxo-ethoxy)-phenyl]-urea,
5-Chloro-2-(2-{(2R,5R)-2-[2-(4-fluoro-phenyl)-ethyl]-5-methyl-morpholin-4-yl)-2-oxo-ethoxy}-phenyl)-urea,
(5-Chloro-2-{3-[(2R,5S)-2-(4-fluoro-benzyl)-5-methyl-morpholin-4-yl]-3-oxo-propoxy}-phenyl)-urea,
(5-Chloro-2-{3-[(2S,5S)-2-(4-fluoro-benzyl)-5-methyl-morpholin-4-yl]-3-oxo-propoxy}-phenyl)-urea,
(5-Chloro-2-{2-[(2R,5S)-2-(4-fluoro-benzyl)-5-methyl-morpholin-4-yl]-ethoxy}-phenyl)-urea,
1-[(2R,5S)-2-(4-Fluoro-benzyl)-5-methyl-morpholin-4-yl]-2-(4-fluoro-phenoxy)-ethanone,
2-[4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
2-[(R)-4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
2-[(S)-4-(4-Chloro-phenyl)-4-hydroxy-3,3-dimethyl-piperidin-1-yl]-1-[(2R,5S)-2-(4-fluoro-benzyl)-5-methyl-morpholin-4-yl]-ethanone,
4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
(S)-4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
(R)-4-(4-Chloro-phenyl)-1-{2-[(2R,5S)-2-(4-fluoro-benzyl)-5-methyl-morpholin-4-yl]-ethyl}-3,3-dimethyl-piperidin-4-ol,
5-Chloro-2-{3-[(2R,5S)-2-(4-fluoro-benzyl)-5-methyl-morpholin-4-yl]-propoxy}-phenyl)-urea,
(5-Chloro-2-{2-[2-(4-fluoro-benzyl)-5-methyl-morpholin-4-yl]-2-oxo-ethoxy}-phenyl)-urea,
(5-Chloro-2-{2-[2-(4-fluoro-phenoxymethyl)-5-methyl-morpholin-4-yl]-2-oxo-ethoxy}-phenyl)-urea,
[5-Chloro-2-(2-{2-[2-(4-fluoro-phenyl)-ethyl]-5-methyl-morpholin-4-yl}-2-oxo-ethoxy)-phenyl]-urea,
(5-Chloro-2-{3-[2-(4-fluoro-benzyl)-5-methyl-morpholin-4-yl]-3-oxo-propoxy}-phenyl)-urea,
(5-Chloro-2-{2-[2-(4-fluoro-benzyl)-5-methyl-morpholin-4-yl]-ethoxy}-phenyl)-urea,
1-[2-(4-Fluoro-benzyl)-5-methyl-morpholin-4-yl]-2-(4-fluoro-phenoxy)-ethanone,
2-[4-(4-Chloro-phenyl)-4-hydroxy-3,3-dimethyl-piperidin-1-yl]-1-[2-(4-fluoro-benzyl)-5-methyl-morpholin-4-yl]-ethanone,
[2-(4-fluoro-benzyl)-5-methyl-morpholin-4-yl]-propan-1-one-3-[4-(4-Chloro-phenyl)-4-hydroxy-3,3-dimethyl-piperidine],
5-Chloro-2-{3-[2-(4-fluoro-benzyl)-5-methyl-morpholin-4-yl]-propoxy}-phenyl)-urea,
(5-Chloro-2-{3-[2-(4-fluoro-benzyl)-[1,4]oxazepan-4-yl]-3-oxo-propoxy}-phenyl)-urea,
(5-Chloro-2-{2-[2-(4-fluoro-benzyl)-[1,4]oxazepan-4-yl]-2-oxo-ethoxy}-phenyl)-urea,
(5-Chloro-2-{3-[2-(4-fluoro-benzyl)-[1,4]oxazepan-4-yl]-propoxy}-phenyl)-urea,
3-[4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
3-[(S)-4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
3-[(R)-4-(4-Chloro-phenyl)-4-hydroxy-3,3-dimethyl-piperidin-1-yl]-1-[(R)-2-(4-fluoro-benzyl)-[1,4]oxazepan-4-yl]-propan-1-one,
4-(4-Chloro-phenyl)-1-{2-[(R)-2-(4-fluoro-benzyl)-[1,4]oxazepan-4-yl]-ethyl}-3,3-dimethyl-piperidin-4-ol,
(S)-4-(4-Chloro-phenyl)-1-{2-[(R)-2-(4-fluoro-benzyl)-[1,4]oxazepan-4-yl]-ethyl}-3,3-dimethyl-piperidin-4-ol,
(R)-4-(4-Chloro-phenyl)-1-{2-[(R)-2-(4-fluoro-benzyl)-[1,4]oxazepan-4-yl]-ethyl}-3,3-dimethyl-piperidin-4-ol,
(5-Chloro-2-{2-[7-(4-fluoro-benzyl)-[1,4]oxazepan-4-yl]-2-oxo-ethoxy}-phenyl)-urea,
(5-Chloro-2-{3-[7-(4-fluoro-benzyl)-[1,4]oxazepan-4-yl]-3-oxo-propoxy}-phenyl)-urea,
(5-Chloro-2-{3-[7-(4-fluoro-benzyl)-[1,4]oxazepan-4-yl]-propoxy}-phenyl)-urea,
(5-Chloro-2-{2-[7-(4-fluoro-benzyl)-[1,4]oxazepan-4-yl]-ethoxy}-phenyl)-urea,
4-[3-(4-Chloro-phenoxy)-propyl]-2-(4-fluoro-benzyl)-[1,4]oxazepane,
(5-Chloro-2-{2-[2-(4-fluoro-benzyl)-[1,4]oxazepan-4-yl]-ethoxy}-phenyl)-urea,
3-[2-(4-Fluoro-benzyl)-[1,4]oxazepan-4-yl]-N-(3-fluoro-phenyl)-propionamide,
N-(3-Chloro-phenyl)-3-[2-(4-fluoro-benzyl)-[1,4]oxazepan-4-yl]-propionamide,
N-(5-Chloro-2-methoxy-phenyl)-3-[2-(4-fluoro-benzyl)-[1,4]oxazepan-4-yl]-propionamide,
(5-Chloro-2-{3-[2-(3,4-difluoro-benzyl)-[1,4]oxazepan-4-yl]-3-oxo-propoxy}-phenyl)-urea,
(5-Chloro-2-{3-[2-(3,4-difluoro-benzyl)-[1,4]oxazepan-4-yl]-propoxy}-phenyl)-urea,
(5-Chloro-2-{3-[2-(3-fluoro-benzyl)-[1,4]oxazepan-4-yl]-3-oxo-propoxy}-phenyl)-urea,
(5-Chloro-2-{3-[2(3-fluoro-benzyl)-[1,4]oxazepan-4-yl]-propoxy}-phenyl)-urea,
2,6-Dichloro-N-{3-[2-(4-fluoro-benzyl)-[1,4]oxazepan-4-yl]-propyl}-benzenesulfonamide,
4-[3-(2-Bromo-4-chloro-phenoxy)-propyl]-2-(4-fluoro-benzyl)-[1,4]oxazepane,
5-chloro-2-{(R)-3-[(R)-2-(4-fluoro-benzyl)-[1,4]oxazepan-4-yl]-2-hydroxy-propoxy}-benzamide,
5-chloro-2-{(S)-3-[(R)-2-(4-fluoro-benzyl)-[1,4]oxazepan-4-yl]-2-hydroxy-propoxy}-benzamide,
5-chloro-2-{(R)-3-[(S)-2-(4-fluoro-benzyl)-[1,4]oxazepan-4-yl]-2-hydroxy-propoxy}-benzamide,
5-chloro-2-{(S)-3-[(S)-2-(4-fluoro-benzyl)-[1,4]oxazepan-4-yl]-2-hydroxy-propoxy}-benzamide,
N-(3-Chloro-2-methyl-phenyl)-2-[2-(4-fluoro-benzyl)-[1,4]oxazepan-4-yl]-acetamide,
4-[3-(2,6-Dichloro-phenoxy)-propyl]-2-(4-fluoro-benzyl)-[1,4]oxazepane,
2,6-Dichloro-N-{2-[2-(4-fluoro-benzyl)-[1,4]oxazepan-4-yl]-ethyl}-benzenesulfonamide,
N-{3-[2-(4-Fluoro-benzyl)-[1,4]oxazepan-4-yl]-propyl}-4-methoxy-benzenesulfonamide,
4-Chloro-N-{3-[2-(4-fluoro-benzyl)-[1,4]oxazepan-4-yl]-propyl}-benzenesulfonamide,
5-Chloro-2-{3-[2-(4-fluoro-benzyl)-[1,4]oxazepan-4-yl]-propoxy}-benzoic acid,
(5-Chloro-2-{3-[2-(4-chloro-benzyl)-[1,4]oxazepan-4-yl]-propoxy}-phenyl)-urea,
2-(4-Chloro-2-isoxazol-5-yl-phenoxy)-1-[7-(4-fluoro-benzyl)-[1,4]oxazepan-4-yl]-ethanone,
3-[4-(4-Chloro-phenyl)-4-hydroxy-3,3-dimethyl-piperidin-1-yl]-1-[2-(4-fluoro-benzyl)-[1,4]oxazepan-4-yl]-propan-1-one,
4-(4-Chloro-phenyl)-1-{2-[2-(4-fluoro-benzyl)-[1,4]oxazepan-4-yl]-ethyl}-3,3-dimethyl-piperidin-4-ol,
4-chloro-2-{3-[2-(4-fluoro-benzyl)-[1,4]oxazepan-4-yl]-2-hydroxy-propoxy}-benzamide,
N-(5-Chloro-2-{3-[4-(4-fluoro-benzyl)-4-hydroxy-3,3-dimethyl-piperidin-1-yl]-propoxy}-4-methoxy-phenyl)-acetamide,
N-(5-Chloro-2-{3-[4-(4-chloro-benzyl)-4-hydroxy-3,3-dimethyl-piperidin-1-yl]-propoxy}-4-isopropoxy-phenyl)-acetamide,
N-(5-Chloro-2-{3-[4-(4-fluoro-benzyl)-4-hydroxy-3,3-dimethyl-piperidin-1-yl]-propoxy}-4-isopropoxy-phenyl)-acetamide,
N-(2-{3-[4-(4-Chloro-benzyl)-4-hydroxy-3,3-dimethyl-piperidin-1-yl]-propoxy}-4-methoxy-phenyl)-acetamide,
N-(5-Chloro-2-{3-[4-(4-chloro-phenyl)-4-hydroxy-3,3-dimethyl-piperidin-1-yl]-propoxy}-4-isopropoxy-phenyl)-acetamide,
N-(5-Chloro-2-{2-[4-(4-fluoro-phenoxy)-piperidin-1-yl]-2-oxo-ethoxy}-4-methoxy-phenyl)-acetamide,
(5-Chloro-2-{2-[4-(4-fluoro-phenoxy)-piperidin-1-yl]-2-oxo-ethoxy}-phenyl)-urea,
5-Chloro-2-{3-[4-(4-fluoro-benzyl)-4-hydroxy-3,3-dimethyl-piperidin-1-yl]-2-hydroxy-propoxy}-4-methoxy-benzoic acid,
5-Chloro-2-{3-[4-(4-fluoro-benzyl)-4-hydroxy-3,3-dimethyl-piperidin-1-yl]-2-hydroxy-propoxy}-4-methoxy-N-methyl-benzamide,
5-Chloro-2-{3-[4-(4-fluoro-benzyl)-4-hydroxy-3,3-dimethyl-piperidin-1-yl]-propoxy}-4-methoxy-N-methyl-benzamide,
5-Chloro-2-{3-[4-(4-chloro-phenyl)-4-hydroxy-3,3-dimethyl-piperidin-1-yl]-propoxy}-4-methoxy-benzoic acid methyl ester,
(5-Chloro-2-{3-[4-(4-fluoro-benzyl)-4-hydroxy-3,3-dimethyl-piperidin-1-yl]-propoxy}-4-methoxy-phenyl)-pyrrolidin-1-yl-methanone,
5-Chloro-2-{3-[4-(4-chloro-phenyl)-4-hydroxy-3,3-dimethyl-piperidin-1-yl]-propoxy}-4-methoxy-N-methyl-benzamide,
(5-Chloro-2-{3-[4-(4-chloro-phenyl)-4-hydroxy-3,3-dimethyl-piperidin-1-yl]-propoxy}-4-methoxy-phenyl)-pyrrolidin-1-yl-methanone,
5-Chloro-2-{3-[4-(4-fluoro-benzyl)-4-hydroxy-3,3-dimethyl-piperidin-1-yl]-propoxy}-4-methoxy-benzoic acid,
5-Chloro-2-{3-[4-(4-fluoro-benzyl)-4-hydroxy-3,3-dimethyl-piperidin-1-yl]-propoxy}-4-hydroxy-N-methyl-benzamide,
5-Chloro-2-{3-[4-(4-chloro-phenyl)-4-hydroxy-3,3-dimethyl-piperidin-1-yl]-propoxy}-4-methoxy-N,N-dimethyl-benzamide,
5-Chloro-4-dimethylamino-2-{3-[4-(4-fluoro-benzyl)-4-hydroxy-3,3-dimethyl-piperidin-1-yl]-propoxy}-N-methyl-benzamide,
5-Chloro-2-{3-[4-(4-fluoro-benzyl)-4-hydroxy-3,3-dimethyl-piperidin-1-yl]-propoxy}-N-methyl-4-methylamino-benzamide,
and pharmaceutically acceptable salts thereof, solvates thereof, and solvates of pharmaceutically acceptable salts thereof.

3. A compound chosen from:
5-Chloro-2-{3-[3-(4-chloro-phenyl)-3-hydroxy-pyrrolidin-1-yl]-propoxy}-4-methoxy-N-methyl-benzamide,
5-Chloro-2-{3-[3-(4-chloro-benzyl)-3-hydroxy-pyrrolidin-1-yl]-propoxy}-4-methoxy-N-methyl-benzamide,
N-[5-Chloro-2-(2-{[1-(4-fluoro-benzyl)-4-hydroxy-piperidin-4-ylmethyl]-amino}-ethoxy)-4-methoxy-phenyl]-acetamide,
5-Chloro-2-(2-{[1-(4-fluoro-benzyl)-4-hydroxy-piperidin-4-ylmethyl]-amino}-ethoxy)-4-methoxy-N-methyl-benzamide,
N-(5-Chloro-2-{3-[3-(4-chloro-phenyl)-3-hydroxy-pyrrolidin-1-yl]-propoxy}-4-methoxy-phenyl)-acetamide,
N-(5-Chloro-2-{3-[3-(4-chloro-benzyl)-3-hydroxy-pyrrolidin-1-yl]-propoxy}-4-methoxy-phenyl)-acetamide,
N-[5-Chloro-2-(2-{[1-(4-chloro-benzyl)-4-hydroxy-piperidin-4-ylmethyl]-amino}-ethoxy)-4-methoxy-phenyl]-acetamide,
5-Chloro-2-{3-[3-(4-fluoro-benzyl)-3-hydroxy-pyrrolidin-1-yl]-propoxy}-4-methoxy-N-methyl-benzamide,
N-(5-Chloro-2-{3-[3-(4-fluoro-benzyl)-3-hydroxy-pyrrolidin-1-yl]-propoxy}-4-methoxy-phenyl)-acetamide,
5-Chloro-2-{2-[1-(4-chloro-benzyl)-4-hydroxy-piperidin-4-ylmethyl]-amino}-ethoxy)-4-methoxy-N-methyl-benzamide,
5-Chloro-2-{2-[8-(4-fluoro-benzyl)-2-oxo-1-oxa-3,8-diaza-spiro[4.5]dec-3-yl]ethoxy}-4-methoxy-N-methyl-benzamide,
5-Chloro-2-[4-(4-fluoro-benzyl)-[1,4]oxazepan-2-ylmethoxy]-4-methoxy-N-methyl-benzamide,
and pharmaceutically acceptable salts thereof, solvates thereof, and solvates of pharmaceutically acceptable salts thereof.

4. A pharmaceutical composition comprising a compound according to claim 1 and a pharmaceutically acceptable carrier.

5. A method for treating a condition that responds to a CC chemokine receptor ligand comprising administering to a patient in need thereof an effective amount of a composition according to claim 4.

6. The method of claim 5, wherein the CC chemokine receptor ligand is a CCR1 antagonist.

7. The method according to claim 5, wherein the condition is an inflammatory disorder or obesity.

8. The method according to claim 7, wherein the condition is obesity.
